(19) United States
(12) Patent Application Publication
(10) Pub. No.: US 2023/0157952 A1
(43) Pub. Date: May 25, 2023

(54) METHOD FOR IMPROVING THE STABILITY OF A PHARMACEUTICAL COMPOSITION COMPRISING A HIGH PENETRATION DRUG, AND THE PHARMACEUTICAL COMPOSITION OBTAINED THEREFROM

(71) Applicant: Techfields Inc., Princeton, NJ (US)

(72) Inventors: Chongxi Yu, Princeton, NJ (US); Lina Xu, Suzhou (CN)

(73) Assignee: Techfields Inc., Princeton, NJ (US)

(21) Appl. No.: 17/906,637

(22) PCT Filed: Mar. 22, 2021
(86) PCT No.: PCT/CN2021/082173

§ 371 Date: Sep. 19, 2022

(30) Foreign Application Priority Data
Mar. 20, 2020 (CN) PCT/CN2020/080477

(51) Int. Cl. A61K 9/08; A61K 47/12; A61K 47/20; A61K 45/06

(52) U.S. Cl. CPC A61K 9/08; A61K 47/12; A61K 47/20; A61K 45/06

(57) ABSTRACT

Provided are pharmaceutical compositions comprising at least one high penetration drug (HPD) that has at least one protonated amino group in its molecular and is capable of penetrating across one or more biological barriers in high rates, methods for improving the stability of the pharmaceutical compositions, and methods of using the pharmaceutical compositions for preventing, diagnosing and/or treating condition or disease in human, animals and plants.

### FIELD OF THE INVENTION

This invention relates to pharmaceutical compositions comprising at least one high penetration drug (HPD) that has at least one protonated amino group in its molecule and is capable of penetrating across one or more biological barriers in high rates, methods for improving the stability of the pharmaceutical composition, and methods of using the pharmaceutical composition for preventing, diagnosing and/or treating condition or disease in human, animals and plants.

### BACKGROUND

Active agents or drugs that are effective in vitro may not be as effective in vivo due to the delivery difficulties in vivo, in particular, their limited penetration ability across one or more biological barriers before reaching the site of action where diseases occur in vivo, then the agents or drugs will stay in general circulation for a long time, and liver, kidneys, and other organs will metabolize the agents or drugs before they reach the site of action where diseases occur.

Currently many drugs are administered through systematic route, such as oral or parenteral administration, to reach an action site of a condition or disease. Since higher dosage of drugs is required to reach a distal location in the systematic administration, drugs delivered by such route may cause adverse reactions.

For example, non-steroidal anti-inflammatory drugs (NSAIDs) are widely used for treatment of acute or chronic conditions where pain and inflammation are present. Although NSAIDs are absorbed in the stomach and intestinal mucosa, oral administration usually accompanies adverse drug reactions such as gastrointestinal (GI) effects and renal effects. For instance, aspirin is known to cause gastric mucosal cell damage. The side effects of NSAIDs appear to be dose-dependent, and in many cases severe enough to pose the risk of dyspepsia, gastroduodenal bleeding, gastric ulcerations, gastritis, ulcer perforation, and even death.

The gastrointestinal, skin, and other biologic membranes have lipophilic barriers. Most of drugs that can penetrate biologic membranes in significant rates are lipophilic; however, the gastrointestinal juice, the blood system, and the moisture on the skin are mostly water and the lipophilic agents or drugs are very difficult to be dissolved in these systems.

In the previous patent applications (PCT/IB2006/052732, PCT/IB2006/052318, PCT/IB2006/052732, PCT/IB2006/052318, PCT/IB2006/052461, PCT/IB2006/052815, PCT/IB2006/052563, PCT/IB2006/052575, PCT/IB2006/053091, PCT/IB2006/053090, PCT/IB2006/053594, PCT/IB2006/052549, PCT/IB2006/053619 PCT/IB2006/054170, PCT/IB2006/054724, PCT/IB2006/053741, PCT/IB2007/050122, PCT/IB2007/050322, PCT/IB2007/052090, PCT/US2009/066884, PCT/CN2010/072561, PCT/CN2010/073743, PCT/CN2013/072693, PCT/CN2013/072728), the applicant disclosed many compositions of novel HPDs that are lipophilic and hydrophilic and can dissolve in both lipid and water and penetrate the lipid or aqueous barriers.

However, many of these novel HPDs are not very stable in aqueous conditions and cannot be stored for a long time that is required for a reasonable shelf life of the pharmaceutical products. Therefore, there is a need to improve the stability of HPDs or compositions so that they are capable of being delivered efficiently and effectively to an action site of a condition (e.g., a disease) to prevent, reduce or treat the condition in a biological subject.

### CONTENTS OF THE INVENTION

In one aspect, the present invention provides a method for improving the stability of a pharmaceutical composition which comprises an HPD and a pharmaceutically acceptable carrier, the method comprising: packaging the HPD and the pharmaceutically acceptable carrier separately; and reconstituting a solution of the pharmaceutical composition by mixing the HPD with the pharmaceutically acceptable carrier when a patient intends to use it; characterized in that the pH of the reconstitution solution of the pharmaceutical composition is maintained in the range of about 2 to about 6.

In the context of the invention, HPD refers to a prodrug that has at least one protonated amine group in its molecule and is capable of penetrating across one or more biological barriers in high rates, e.g. 10 times, 50 times, 100 times, 200 times, 300 times, 500 times, or even 1,000 times higher than the penetration rate of the corresponding parent drug.

Advantageously, the HPD comprises one or two protonated amine groups in its molecule when being administered to the patient.

In an advantageous embodiment, the pharmaceutically acceptable carrier is an aqueous carrier. The pharmaceutically acceptable carrier may be water, alcohol, acetone, or dimethyl sulfoixide (DMSO), or a mixture thereof. Preferably, the pharmaceutically acceptable carrier is an aqueous solution containing 0-70% ethanol by volume. More preferably, the pharmaceutically acceptable carrier is an aqueous solution containing 10-35% ethanol by volume.

Advantageously, the pharmaceutical composition is applied transdermally as a spray solution.

In an advantageous embodiment, the method according to the present invention further comprises a step of storing the reconstitution solution in a refrigerator at a temperature of 2-8° C.

In the method according to the present invention, the pharmaceutical composition may also comprise a pH adjusting and buffering agent. In an advantageous embodiment, the HPD is high penetration peptide and the pH adjusting and buffering agent is sodium, potassium, calcium, lithium, or magnesium salt of an organic acid. Preferably, the pH adjusting and buffering agent is sodium, potassium or lithium salt of acetic acid, propionic acid, butyric acid, valeric acid, benzoic acid, lactic acid, salicylic acid, citric acid, ascorbic acid, succinic acid, or maleic acid.

Advantageously, the pH of the reconstitution solution of the pharmaceutical composition is 3-6, preferably 3-5, more preferably 3.5-4.5.

Advantageously, the concentration of the HPD in the reconstitution solution is 1%-30% by weight, preferably 1%-20% by weight, more preferably 3%-10% by weight.

In an advantageous embodiment, the HPD is selected from the group consisting of 2-(diethylamino)ethyl 2-(6-methoxy-2-naphthyl)propionate.HCl, 2-(diethylamino)ethyl (R,S)-2-(2-fluoro-4-biphenyl)propionate.HCl, 2-(diethylamino)ethyl 2-(p-isobutylphenyl)propionate.HCl, 2-(diethylamino)ethyl 1-(4-chlorobenzoyl)-5-methoxy-2-methyl-1H-indole-3-acetate.HCl, 2-(diethylamino)ethyl 5-fluoro-2-methyl-1-[[4-(methylsulfinyl)phenyl]methylene]-1H-indene-3-acetate.HCl, 2-(diethylamino)ethyl 1-methyl-5-(4-methylbenzoyl)-1H-pyrrole-2-acetate.HCl, 2-(diethylamino)ethyl 5-(4-chlorobenzoyl)-1,4-dimethyl-1H-pyrrole-2-acetate.HCl, 2-(diethylamino)ethyl 3-(6-methoxy-2-naphthyl)propionate.HCl, 2-(diethylamino)ethyl 4-(4-chlorophenyl)-2-phenyl-5-thiazoleacetate.HCl, 2-(diethylamino)ethyl 1-(4-chlorobenzoyl-5-methoxy-2-methyl-1H-indole-3-acetoxyacetate.HCl, 2-(diethylamino)ethyl [(1-benzyl-1H-indazol-3-yl)oxy]acetate.HCl, 2-(diethylamino)ethyl 2-[(4-chlorophenyl)-5-benzoxazole]propionate.HCl, 2-(diethylamino)ethyl 4,5-diphenyl-2-oxazolepropionate.HCl, 2-(diethylamino)ethyl 4-[bis(2-chloroethyl)amino]benzenebutyrate.HCl, 2-(diethylamino)ethyl 4-[bis(2-methylsulfonylethyl)amino]benzenebutyrate.HCl, 2-(diethylamino)ethyl acetylsalicylate.HCl, and 2-(diethylamino)ethyl 5-(2,4-difluorophenyl)-2-acetoxybenzoate.HCl. Advantageously, the concentration of the HPD in the reconstitution solution is 3-8% by weight, the pH of reconstitution solution is 3-5 and the pharmaceutically acceptable carrier is an aqueous solution containing 15-35% ethanol by volume.

In another advantageous embodiment, the HPD is selected from the group consisting of H-Val-Pro-Gly-Pro-Arg(NO₂)—OCH(CH₃)₂.HCl, H-Ala-Pro-Gly-Pro-Arg(NO₂)—OCH₂CH₃.HCl, H-Val-Pro-Asp[OCH(CH₃)₂]-Pro-Arg(NO₂)—OCH(CH₃)₂.HCl, H-Tyr-Gly-Gly-Phe-Leu-OCH(CH₃)₂.HCl, and H-Tyr-Gly-Gly-Phe-Met-OCH(CH₃)₂.HCl. Advantageously, the concentration of the HPD in the reconstitution solution is 3-8% by weight, the pH of reconstitution solution is 3-5, the pH adjusting and buffering agent is sodium acetate and the pharmaceutically acceptable carrier is an aqueous solution containing 15-35% ethanol by volume.

The HPD according to the present invention is stable at room temperature and can be stored for more than two years when kept in dry condition. By means of the method according to the present invention, the pharmaceutical composition comprising an HPD and a pharmaceutically acceptable carrier when reconstituted as a solution can be stored for a reasonable shelf life, e.g. more than one month, or even more than two months.

In another aspect, the present invention provides the pharmaceutical compositions obtained from any embodiments of the above method.

In another aspect, the present invention provides methods of using the pharmaceutical compositions disclosed for preventing, diagnosing and/or treating condition or disease in human, animals and plants.

In another aspect, the present invention provides treatment kits based on the improved methods and HPD compositions to ensure convenience of administration and stability of the pharmaceutical compositions obtained.

Other aspects and advantages of the invention will be better understood in view of the following detailed description, examples, and claims.

### DETAILED DESCRIPTION OF THE INVENTION

When a drug is administered in solid, semisolid, or suspension form, the rate of absorption is often controlled by how the drug particles dissolve in the fluid or moisture at the site of administration (PDR Generics, 1996, second edition, Medical Economics, Montvale, N.J., pg 21). HPDs that were disclosed in previous patent applications have two structural features in common: a lipophilic portion and a hydrophilic portion comprising a primary, secondary, or tertiary amine group in protonated form. They have a very high solubility in gastric juice, blood system, or moistures on the skin and have a high solubility in oil, which enables them to penetrate biological membranes easily. These features make the formulation of the HPDs much simpler.

Transdermal delivery systems help to avoid directly hurting the gastro-intestinal tract and inactivation of the drugs caused by the “first pass metabolism” in the gastro-intestinal tract and liver. It can provide local delivery of appropriate concentrations of a drug to the intended site of action without systemic exposure. Fishman et al. (U.S. Pat. No. 7,052,715) indicated that an additional problem associated with oral medications is that the concentration levels achieved in the bloodstream must be significant in order to effectively treat distal areas of pain or inflammation. These levels are often much higher than would be necessary if it were possible to accurately target the particular site of pain or injury. By controlling the rate of release, transdermal delivery systems enable drugs to reach constantly optimal therapeutic blood levels to increase effectiveness and reduce the side effects of drugs.

The HPDs may adopt the form of pro-drugs. A good pro-drug should be able to release the parent drug easily in plasma and/or in other organs/tissues. A very good linker between the functional unit (parent drug) and the transportational (or transporting) unit (with at least one amino group) is an ester bond which can be cleaved in most tissues in a short time. Before the drug can penetrate skin, GI system, or other biological barriers, it should be dissolved in some solvent, which should not hurt skin, GI system, or other biological barriers. For oral administration, a solid formulation is suitable because the GI system can keep the drug inside and the plenty of GI juices can dissolve the drug, but oral administration has the disadvantage of the “first pass metabolism”, and 100% of the drugs/pro-drugs will pass the GI system and may hurt the GI system severely. For transdermal administration, the drug should be dissolved or suspended on some medium. Most organic solvent will hurt skin, and water is the best solvent for topical and transdermal administration. The hydrolysis of ester in water can be accelerated by both acids and bases, and strong acidic and basic condition will hurt skin or other biological barriers. Because the amino group in the transporting unit is a base and would help hydrolyze the ester bond, most of the amino groups should be kept in the protonated form.

In one aspect, the present disclosure provides a method for improving the stability of a pharmaceutical composition which comprises a high penetration drug substance and a pharmaceutically acceptable carrier, the method comprising:
packaging the high penetration drug substance and the pharmaceutically acceptable carrier in separate containers; and
reconstituting a solution of the pharmaceutical composition by mixing the high penetration drug substance with the pharmaceutically acceptable carrier prior to administration to a patient in need thereof;
characterized in that the pH of the reconstitution solution of the pharmaceutical composition is kept within the range of 2 to 6.

In some embodiments, sometimes preferred, the high penetration drug substance comprises one or two protonated amine groups in its molecule when being administered to the patient.

In some embodiments, sometimes preferred, the pharmaceutically acceptable carrier is an aqueous carrier.

In some embodiments, sometimes preferred, the pharmaceutically acceptable carrier is water, alcohol, acetone, DMSO, or a mixture thereof.

In some embodiments, sometimes preferred, the pharmaceutically acceptable carrier is an aqueous solution containing 0-70% ethanol by volume.

In some embodiments, sometimes preferred, the pharmaceutically acceptable carrier is an aqueous solution containing 10-35% ethanol by volume.

In some embodiments, sometimes preferred, the reconstitution solution is applied transdermally as a spray solution.

In some embodiments, sometimes preferred, the methods further includes storing the reconstitution solution in a refrigerator at a temperature of 2-8° C.

In some embodiments, sometimes preferred, the pharmaceutical composition further comprises a pH adjusting and buffering agent in the pharmaceutically acceptable carrier.

In some embodiments, sometimes preferred, the high penetration drug is high penetration peptide; and the pH adjusting and buffering agent is a sodium, potassium, calcium, lithium, or magnesium salt of an organic acid.

In some embodiments, sometimes preferred, the pH adjusting and buffering agent is sodium, potassium, or lithium salt of an organic acid selected from the group consisting of acetic acid, propionic acid, butyric acid, valeric acid, benzoic acid, lactic acid, salicylic acid, citric acid, ascorbic acid, succinic acid, and maleic acid.

In some embodiments, the pH of the reconstitution solution of the pharmaceutical composition is in the range of 3 to 6.

In some embodiments, sometimes preferred, the pH of the reconstitution solution of the pharmaceutical composition is in the range of 3 to 5.

In some embodiments, sometimes more preferred, the pH of the reconstitution solution of the pharmaceutical composition is 3.5-4.5.

In some embodiments, the concentration of the high penetration drug in the reconstitution solution is in the range of 1%-30% by weight.

In some embodiments, sometimes preferred, the concentration of the high penetration drug in the reconstitution solution is in the range of 1%-20% by weight.

In some embodiments, sometimes more preferred, the concentration of the high penetration drug in the reconstitution solution is in the range of 3%-10% by weight.

In some embodiments, the high penetration drug substance is selected from the group consisting of 2-(diethylamino)ethyl 2-(6-methoxy-2-naphthyl) propionate.HCl, 2-(diethylamino)ethyl (R,S)-2-(2-fluoro-4-biphenyl)propionate.HCl, 2-(diethylamino)ethyl 2-(p-isobutylphenyl)propionate.HCl, 2-(diethylamino)ethyl 1-(4-chlorobenzoyl)-5-methoxy-2-methyl-1H-indole-3-acetate.HCl, 2-(diethylamino)ethyl 5-fluoro-2-methyl-1-[[4-(methylsulfinyl)phenyl]methylene]-1H-indene-3-acetate.HCl, 2-(diethylamino)ethyl 1-methyl-5-(4-methylbenzoyl)-1H-pyrrole-2-acetate.HCl, 2-(diethylamino)ethyl 5-(4-chlorobenzoyl)-1,4-dimethyl-1H-pyrrole-2-acetate.HCl, 2-(diethylamino)ethyl 3-(6-methoxy-2-naphthyl)propionate.HCl, 2-(diethylamino)ethyl 4-(4-chlorophenyl)-2-phenyl-5-thiazoleacetate.HCl, 2-(diethylamino)ethyl 1-(4-chlorobenzoyl-5-methoxy-2-methyl-1H-indole-3-acetoxyacetate.HCl, 2-(diethylamino)ethyl [(1-benzyl-1H-indazol-3-yl)oxy]acetate.HCl, 2-(diethylamino)ethyl 2-[(4-chlorophenyl)-5-benzoxazole]propionate.HCl, 2-(diethylamino)ethyl 4,5-diphenyl-2-oxazolepropionate.HCl, 2-(diethylamino)ethyl 4-[bis(2-chloroethyl)amino]benzenebutyrate.HCl, 2-(diethylamino)ethyl 4-[bis(2-methylsulfonylethyl)amino]benzenebutyrate.HCl, 2-(diethylamino)ethyl acetylsalicylate.HCl, and 2-(diethylamino)ethyl 5-(2,4-difluorophenyl)-2-acetoxybenzoate.HCl.

In some embodiments, sometimes preferred, the concentration of the high penetration drug in the reconstitution solution is 3-8% by weight, the pH of reconstitution solution is 3-5, and the pharmaceutically acceptable carrier is 15-35% ethanol in water by volume.

In some embodiments, the high penetration drug is selected from the group consisting of H-Val-Pro-Gly-Pro-Arg(NO₂)—OCH(CH₃)₂.HCl, H-Ala-Pro-Gly-Pro-Arg(NO₂)—OCH₂CH₃.HCl, H-Val-Pro-Asp[OCH(CH₃)₂]-Pro-Arg(NO₂)—OCH(CH₃)₂.HCl, H-Tyr-Gly-Gly-Phe-Leu-OCH(CH₃)₂.HCl, and H-Tyr-Gly-Gly-Phe-Met-OCH(CH₃)₂.HCl.

In some embodiments, sometimes preferred, the concentration of the high penetration drug in the reconstitution solution is 3-8%, the pH of reconstitution solution is 3-5, the pH adjusting and buffering agent is sodium acetate, and the pharmaceutically acceptable carrier is 15-35% ethanol in water by volume.

In another aspect, the present disclosure provides a pharmaceutical composition obtained from any embodiment of the methods disclosed.

In another aspect, the present disclosure provides a method of treating a disease or disorder in a subject in need thereof, comprising administering to the subject a therapeutically effective amount of a pharmaceutical composition prepared according to any embodiment of the methods disclosed.

In some embodiments, sometimes preferred, the pharmaceutical composition is a freshly prepared reconstitution solution by mixing the high penetration drug substance with the pharmaceutically acceptable carrier from separate containers, according to any embodiment of the methods disclosed.

In another aspect, the present disclosure provides a treatment kit comprising: a high penetration drug substance in a first container, a pharmaceutically acceptable carrier in a second container, and a pH adjusting and buffering agent in the first container, the second container, or a separate third container, wherein the high penetration drug substance comprises one or two protonated amine groups, and wherein the high penetration drug substance, the pharmaceutically acceptable carrier, and the pH adjusting and buffering agent can be mixed together to form a reconstitution solution ready for administration to a subject in need thereof.

In some embodiments, sometimes preferred, the reconstitution solution has a pH in the range of 2 to 6 and is stable for storage at a temperature in the range of 2-20° C. for a period of time prior to administration to the subject in need thereof.

In some embodiments, sometimes preferred, the high penetration drug substance is selected from the group consisting of 2-(diethylamino)ethyl 2-(6-methoxy-2-naphthyl) propionate.HCl, 2-(diethylamino)ethyl (R,S)-2-(2-fluoro-4-biphenyl)propionate.HCl, 2-(diethylamino)ethyl 2-(p-isobutylphenyl)propionate.HCl, 2-(diethylamino)ethyl 1-(4-chlorobenzoyl)-5-methoxy-2-methyl-1H-indole-3-acetate.HCl, 2-(diethylamino)ethyl 5-fluoro-2-methyl-1-[[4-(methylsulfinyl)phenyl]methylene]-1H-indene-3-acetate.HCl, 2-(diethylamino)ethyl 1-methyl-5-(4-methylbenzoyl)-1H-pyrrole-2-acetate.HCl, 2-(diethylamino)ethyl 5-(4-chlorobenzoyl)-1,4-dimethyl-1H-pyrrole-2-acetate.HCl, 2-(diethylamino)ethyl 3-(6-methoxy-2-naphthyl)propionate.HCl, 2-(diethylamino)ethyl 4-(4-chlorophenyl)-2-phenyl-5-thiazoleacetate.HCl, 2-(diethylamino)ethyl 1-(4-chlorobenzoyl-5-methoxy-2-methyl-1H-indole-3-acetoxyacetate.HCl, 2-(diethylamino)ethyl [(1-benzyl-1H-indazol-3-yl)oxy]acetate.HCl, 2-(diethylamino)ethyl 2-[(4-chlorophenyl)-5-benzoxazole]propionate.HCl, 2-(diethylamino)ethyl 4,5-diphenyl-2-oxazolepropionate.HCl, 2-(diethylamino)ethyl 4-[bis(2-chloroethyl)amino]benzenebutyrate.HCl, 2-(diethylamino)ethyl 4-[bis(2-methylsulfonylethyl)amino]benzenebutyrate.HCl, 2-(diethylamino)ethyl acetylsalicylate.HCl, 2-(diethylamino)ethyl 5-(2,4-difluorophenyl)-2-acetoxybenzoate.HCl, H-Val-Pro-Gly-Pro-Arg(NO₂)—OCH(CH₃)₂.HCl, H-Ala-Pro-Gly-Pro-Arg(NO₂)—OCH₂CH₃.HCl, H-Val-Pro-Asp[OCH(CH₃)₂]-Pro-Arg(NO₂)—OCH(CH₃)₂.HCl, H-Tyr-Gly-Gly-Phe-Leu-OCH(CH₃)₂.HCl, and H-Tyr-Gly-Gly-Phe-Met-OCH(CH₃)₂.HCl; and the pharmaceutically acceptable carrier is a mixture of an aliphatic C₁-C₆ alcohol and water.

In some embodiments, sometimes preferred, the concentration of the high penetration drug in the reconstitution solution is 3-8%, the pH of reconstitution solution is 3-5, the pH adjusting and buffering agent is sodium acetate, and the pharmaceutically acceptable carrier is 15-35% ethanol in water by volume.

In another aspect, the present disclosure provides treatment of a disease or disorder in a subject using the treatment kits prepared according to the any embodiment of the methods disclosed. Such treatment kits can be used for administration of the pharmaceutical composition to the subject by a healthcare professional or for convenient self-administration by the subject, as the case may be.

The disease or disorder that can be treated by the pharmaceutical compositions provided by the present disclosure can be any disease or disorder to which the high penetration drug substance can provide desired therapeutic effects with advantages of high penetration rate through certain biological barriers. Some nonlimiting examples of the diseases or disorders have been mentioned in the present disclosure, which are all encompassed by the present invention.

Another aspect of the invention relates to a method of using a composition of the invention, or a pharmaceutical composition thereof in treating a condition in a biological subject. The method comprises administrating the pharmaceutical composition to the biological subject.

Some examples of the conditions the method can treat include conditions that can be treated by the parent drug of the HPD. For example, without limitation, stroke, arthritis, depression, Alzheimer's disease, Parkinson's disease, migraine, sexual dysfunction, sepsis, drug-resistant bacterial infections, epilepsy, diabetes, psoriasis, lupus erythematosus, ulcerative enteritis, asthma, lower and upper respiratory tract infections, allergic rhinitis, allergic conjunctivitis, itchiness, and runny nose.

The one or more HPDs or a pharmaceutical composition thereof can be administered to a biological subject by any administration route known in the art, including without limitation, oral, enteral, buccal, nasal, topical, rectal, vaginal, aerosol, transmucosal, epidermal, transdermal, dermal, ophthalmic, pulmonary, subcutaneous, and/or parenteral administration. The pharmaceutical compositions can be administered in a variety of unit dosage forms depending upon the method of administration.

A parenteral administration refers to an administration route that typically relates to injection which includes but is not limited to intravenous, intramuscular, intraarterial, intrathecal, intracapsular, intraorbital, intra cardiac, intradermal, intraperitoneal, transtracheal, subcutaneous, subcuticular, intraarticular, subcapsular, subarachnoid, intraspinal, and/or intrasternal injection and/or infusion.

The one or more HPDs or a pharmaceutical composition thereof can be given to a subject in the form of formulations or preparations suitable for each administration route. The formulations useful in the methods of the invention include one or more HPDs, one or more pharmaceutically acceptable carriers therefor, and optionally other therapeutic ingredients. The formulations may conveniently be presented in unit dosage form and may be prepared by any methods well known in the art of pharmacy. The amount of active ingredient which can be combined with a carrier material to produce a single dosage form will vary depending upon the subject being treated and the particular mode of administration. The amount of an HPD which can be combined with a carrier material to produce a pharmaceutically effective dose will generally be that amount of an HPD which produces a therapeutic effect.

Methods of preparing these formulations or compositions include the step of bringing into association an HPD with one or more pharmaceutically acceptable carriers and, optionally, one or more accessory ingredients. In general, the formulations are prepared by uniformly and intimately bringing into association an HPD with liquid carriers.

Liquid dosage forms for oral, transdermal or topical administration include pharmaceutically acceptable emulsions, microemulsions, solutions, suspensions, syrups and elixirs. In addition to the HPD, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming and preservative agents.

Suspensions, in addition to the HPD, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for the topical or transdermal or epidermal or dermal administration of an HPD composition include powders, sprays, ointments, pastes, creams, lotions, gels, solutions, patches and inhalants. The active component may be mixed under sterile conditions with a pharmaceutically acceptable carrier, and with any preservatives, buffers, or propellants which may be required. The ointments, pastes, creams and gels may contain, in addition to the HPD composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to the HPD composition, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane. The best formulations for the topical or transdermal administration are pure water, solution, aqueous solution, ethanol and water solution, and isopropanol and water solution.

Transdermal patches can also be used to deliver HPD compositions to a target site. Such formulations can be made by dissolving or dispersing the agent in the proper medium.

Absorption enhancers can also be used to increase the flux of the HPD compositions across the skin. The rate of such flux can be controlled by either providing a rate controlling membrane or dispersing the HPD compositions in a polymer matrix or gel.

Formulations suitable for parenteral administration comprise an HPD in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacterostats, solutes which render the formulation isotonic with the blood of the intended recipient or suspending or thickening agents.

Examples of suitable aqueous and nonaqueous carriers which may be employed in the formulations suitable for parenteral administration include water, ethanol, polyols (e.g., such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Formulations suitable for parenteral administration may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Injectable depot forms are made by forming microencapsule matrices of an HPD or in biodegradable polymers such as polylactide-polyglycolide. Depending on the ratio of the HPD to polymer, and the nature of the particular polymer employed, the rate of drug release can be controlled. Examples of other biodegradable polymers include poly (orthoesters) and poly (anhydrides). Depot injectable formulations are also prepared by entrapping the HPD in liposomes or microemulsions which are compatible with body tissue.

In certain embodiments, one or more HPDs or a pharmaceutical composition thereof is delivered to an action site in a therapeutically effective dose. As is known in the art of pharmacology, the precise amount of the pharmaceutically effective dose of an HPD that will yield the most effective results in terms of efficacy of treatment in a given patient will depend upon, for example, the activity, the particular nature, pharmacokinetics, pharmacodynamics, and bioavailability of a particular HPD, physiological condition of the subject (including race, age, sex, weight, diet, disease type and stage, general physical condition, responsiveness to a given dosage and type of medication), the nature of pharmaceutically acceptable carriers in a formulation, the route and frequency of administration being used, and the severity or propensity of the condition that is to be treated. However, the above guidelines can be used as the basis for fine-tuning the treatment, e.g., determining the optimum dose of administration, which will require no more than routine experimentation consisting of monitoring the subject and adjusting the dosage. Remington: The Science and Practice of Pharmacy (Gennaro ed. 20.sup.th edition, Williams & Wilkins PA, USA) (2000).

In certain embodiments, a combination of one or more HPDs and/or other drug(s) is applied to the subject for the desired use (e.g. treatment, screening, etc.).

When applying a combination of a plurality of drugs (e.g. one or more HPDs and/or other drug(s)) to a subject, each drug may be applied separately, or one or more of the drugs may be applied at the same time as separate drugs (e.g. spraying two or more drugs at substantially the same time without mixing the drugs before spraying), or one or more drugs can be mixed together before applying to the subject, or any combination of the above application methods. The drugs may be applied in any order possible.

In certain embodiments, since an HPD of the invention is capable of crossing one or more biological barriers, the HPD can be administered locally (e.g., topically or transdermally) to reach a location where a condition occurs without the necessity of a systematic administration (e.g., oral or parenteral administration). A local administration and penetration of an HPD allows the HPD to reach the same level of local concentration of an agent or drug with much less amount or dosage of HPD in comparison to a systematic administration of a parent agent or drug; alternatively, a higher level of local concentration which may not be afforded in the systematic administration, or if possible, requires significantly higher dosage of an agent in the systematic administration. The high local concentration of the HPD or its parent agent if being cleaved enables the treatment of a condition more effectively or much faster than a systematically delivered parent agent and the treatment of new conditions that may not be previously possible or observed. The local administration of the HPD may allow a biological subject to reduce potential suffering from a systemic administration, e.g., adverse reactions associated with the systematic exposure to the agent, gastrointestinal/renal effects. Additionally, the local administration may allow the HPD to cross a plurality of biological barriers and reach systematically through, for example, general circulation and thus avoid the needs for systematic administration (e.g., injection) and obviate the pain associated with the parenteral injection.

In certain embodiments, an HPD or a pharmaceutical composition according to the invention can be administered systematically (e.g., orally, transdermally, or parenterally). The HPD or the active agent (e.g., drug or metabolite) of the HPD may enter the general circulation with a faster rate than the parent agent and gain faster access to the action site of a condition. Additionally, the HPD can cross a biological barrier (e.g., blood brain barrier and blood milk barrier) which has not been penetrated if a parent agent is administered alone and thus offer novel treatment of conditions that were previously not possible or observed.

In another embodiment of this aspect, the liquid formulation obtained is a formulation according to any one of the embodiments described herein, or any combination thereof.

When the term “about” is applied to a parameter, such as pH, concentration, or the like, it indicates that the parameter can vary by +10%, sometimes preferably within ±5%, and sometimes more preferably within ±2%. As would be understood by a person skilled in the art, when a parameter is not critical, a number is often given only for illustration purpose, instead of being limiting.

The term “a”, “an”, or “the” as used herein, represents both singular and plural forms. In general, when either a singular or a plural form of a noun is used, it denotes both singular and plural forms of the noun.

The term “treating” as used herein means curing, alleviating, inhibiting, or preventing. The term “treat” as used herein means cure, alleviate, inhibit, or prevent. The term “treatment” as used herein means cure, alleviation, inhibition or prevention.

The term “biological subject,” or “subject” as used herein means an organ, a group of organs that work together to perform a certain task, an organism, or a group of organisms. The term “organism” as used herein means an assembly of molecules that function as a more or less stable whole and has the properties of life, such as animal, plant, fungus, or micro-organism.

The term “animal” as used herein means a eukaryotic organism characterized by voluntary movement. Examples of animals include, without limitation, vertebrata (e.g. human, mammals, birds, reptiles, amphibians, fishes, marsipobranchiata and leptocardia), tunicata (e.g. thaliacea, appendicularia, sorberacea and ascidioidea), articulata (e.g. insecta, myriapoda, malacapoda, arachnida, pycnogonida, merostomata, crustacea and annelida), gehyrea (anarthropoda), and helminthes (e.g. rotifera). Preferably, the subject is human or a mammalian animal, such as cats, dogs, horses, monkey, or the like.

The term “plant” as used herein means organisms belonging to the kindom Plantae. Examples of plant include, without limitation, seed plants, bryophytes, ferns and fern allies. Examples of seed plants include, without limitation, cycads, ginkgo, conifers, gnetophytes, angiosperms. Examples of bryophytes include, without limitation, liverworts, hornworts and mosses. Examples of ferns include, without limitation, ophioglossales (e.g. adders-tongues, moonworts, and grape-ferns), marattiaceae and leptosporangiate ferns. Examples of fern allies include, without limitation, lycopsida (e.g. clubmosses, spikemosses and quillworts), psilotaceae (e.g. lycopodiophyta and whisk ferns) and equisetaceae (e.g. horsetails).

The term “fungus” as used herein means a eukaryotic organism that is a member of the kingdom Fungi. Examples of fungus include, without limitation, chytrids, blastocladiomycota, neocallimastigomycota, zygomycota, glomeromycota, ascomycota and basidiomycota.

The term “microorganism” as used herein means an organism that is microscopic (e.g. with length scale of micrometer). Examples of microorganism include, without limitation, bacteria, fungi, archaea, protists and microscopic plants (e.g. green algae) and microscopic animals (e.g. plankton, planarian and amoeba).

### I. Examples of HPDs

Some structure examples of the high biological barrier (skin, blood-brain barrier, blood-milk barrier, and other biological barriers) penetration drugs are listed as follows:

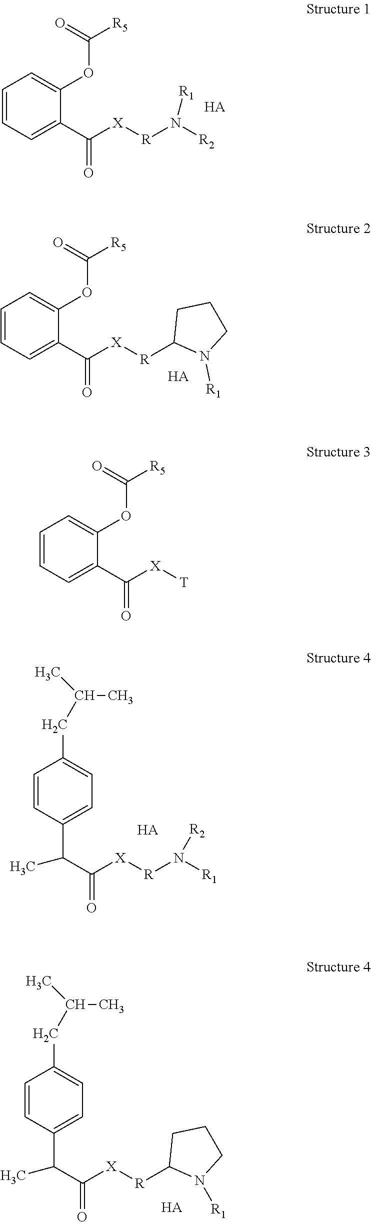

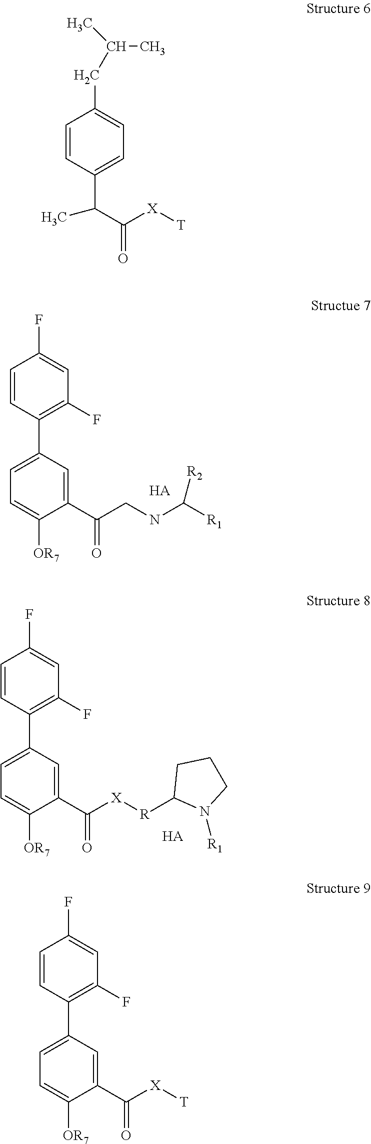

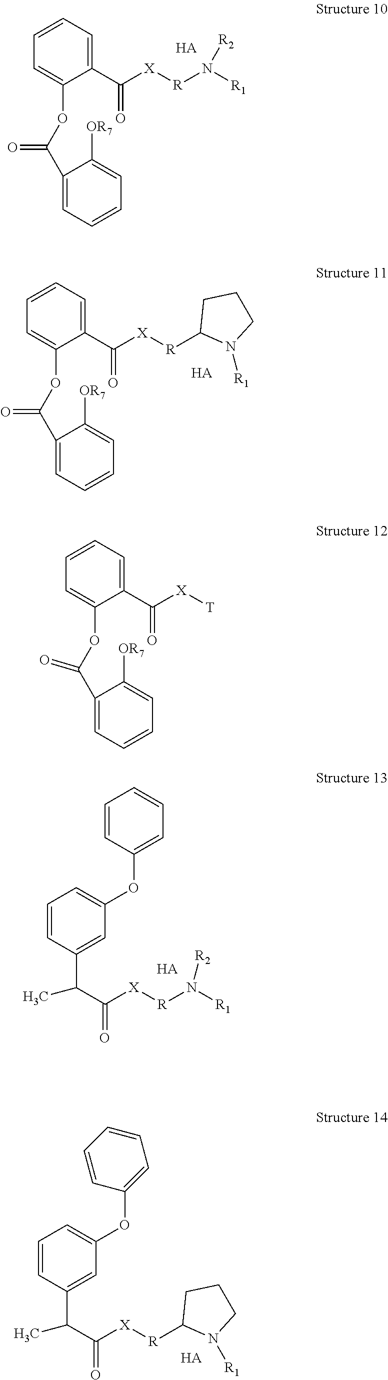

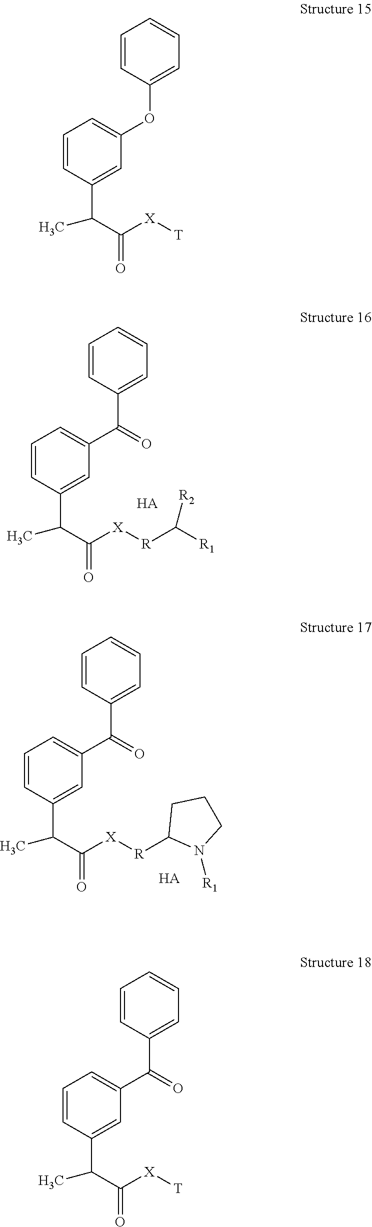

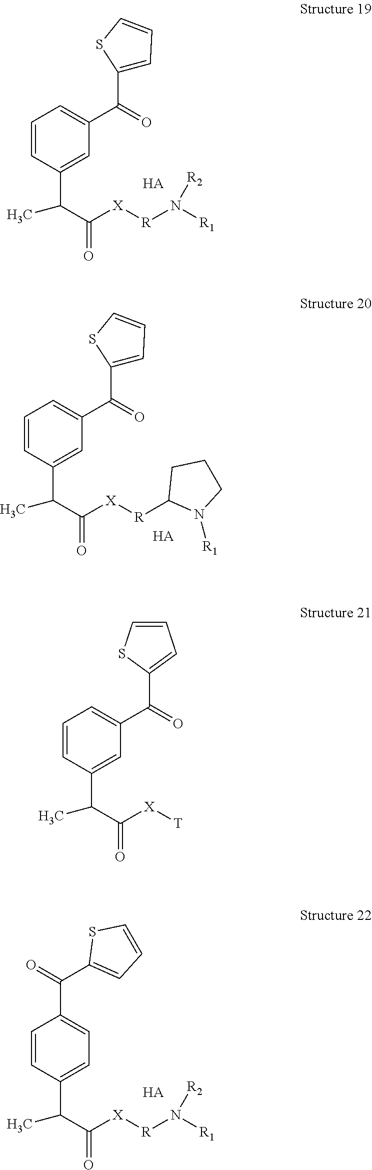

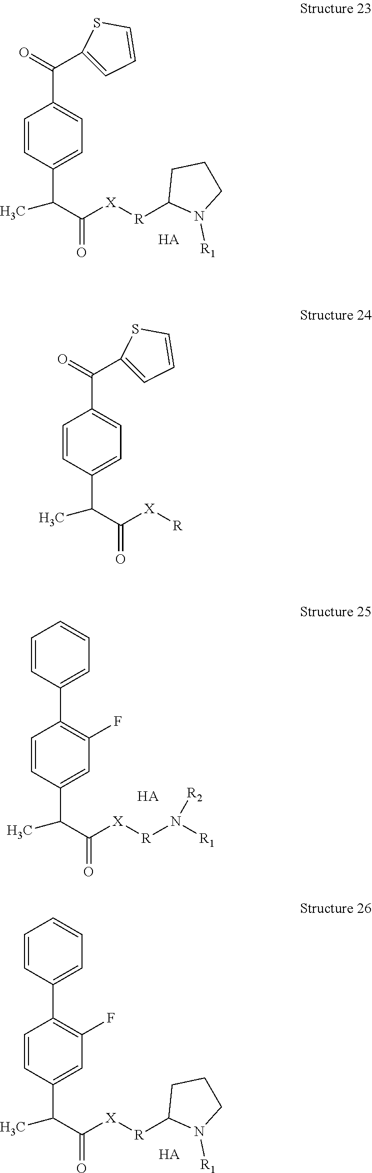

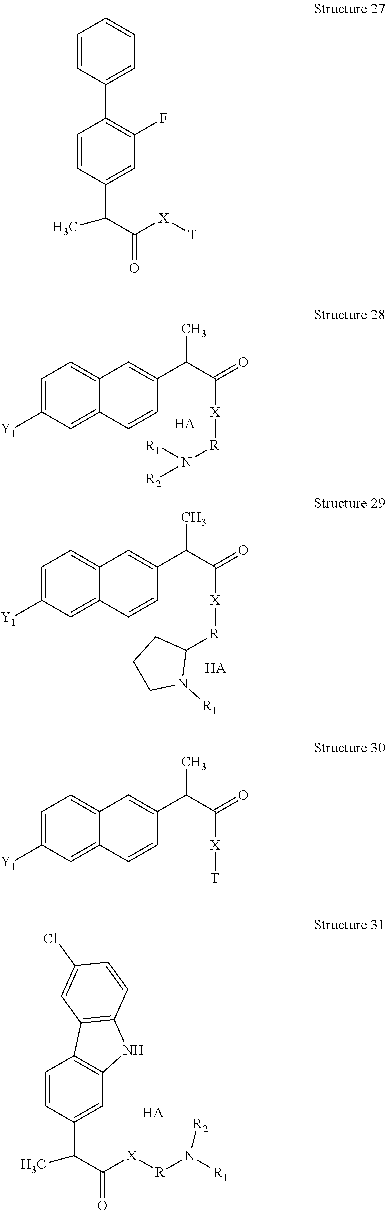

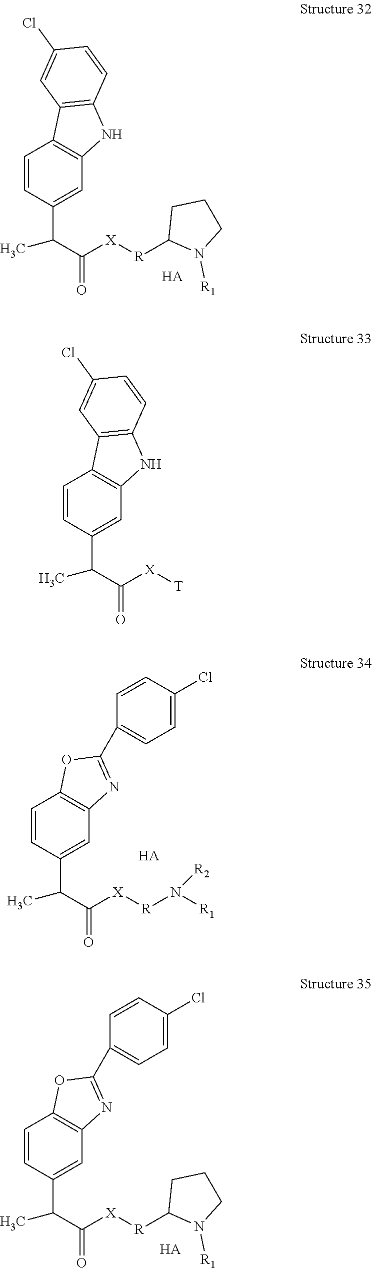

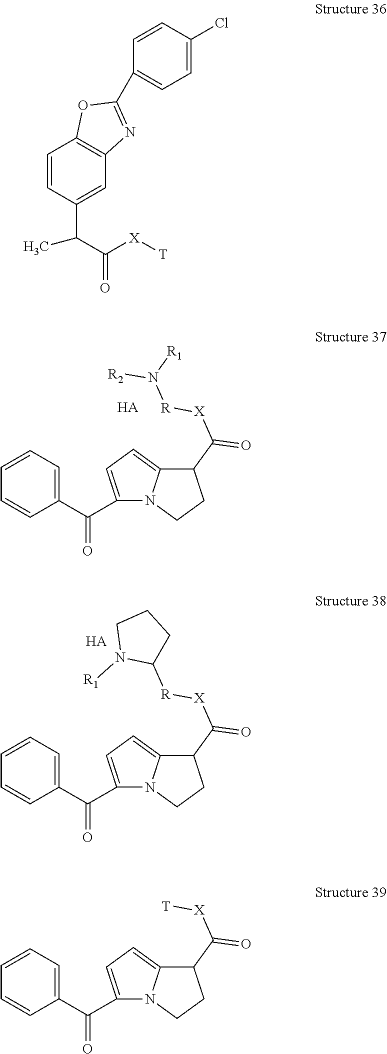

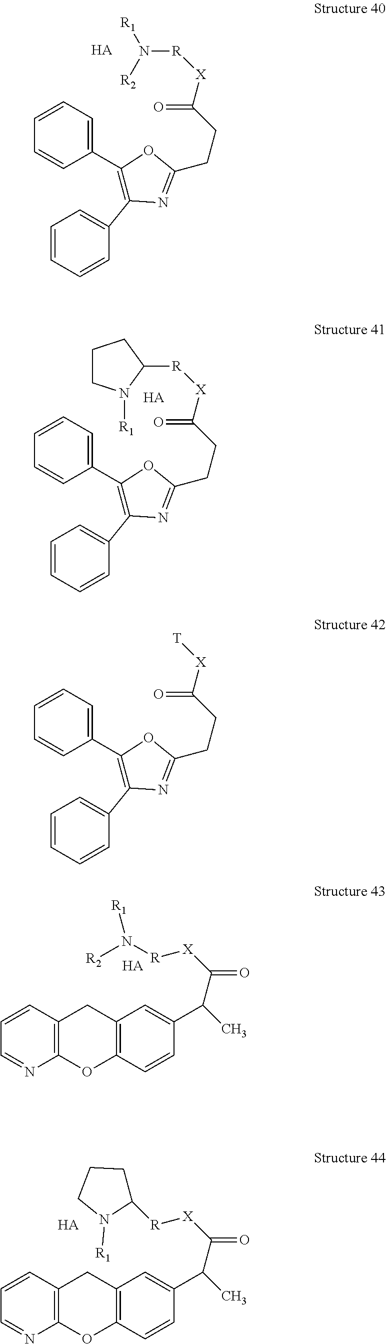

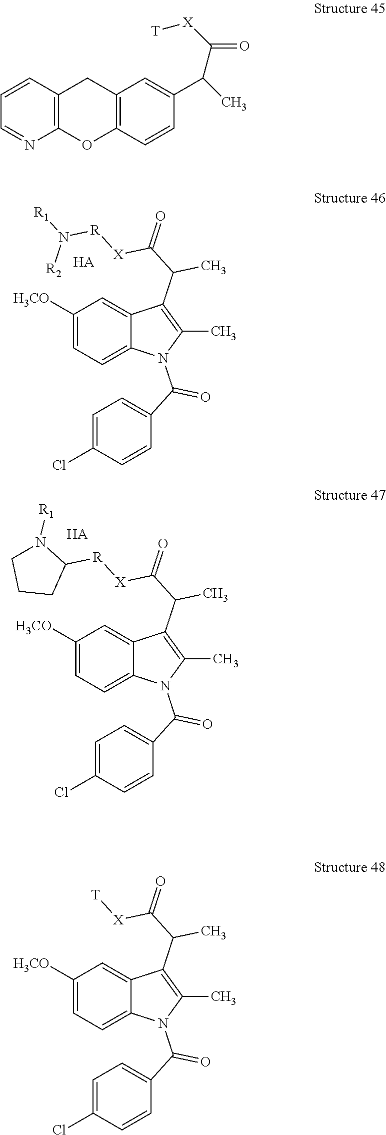

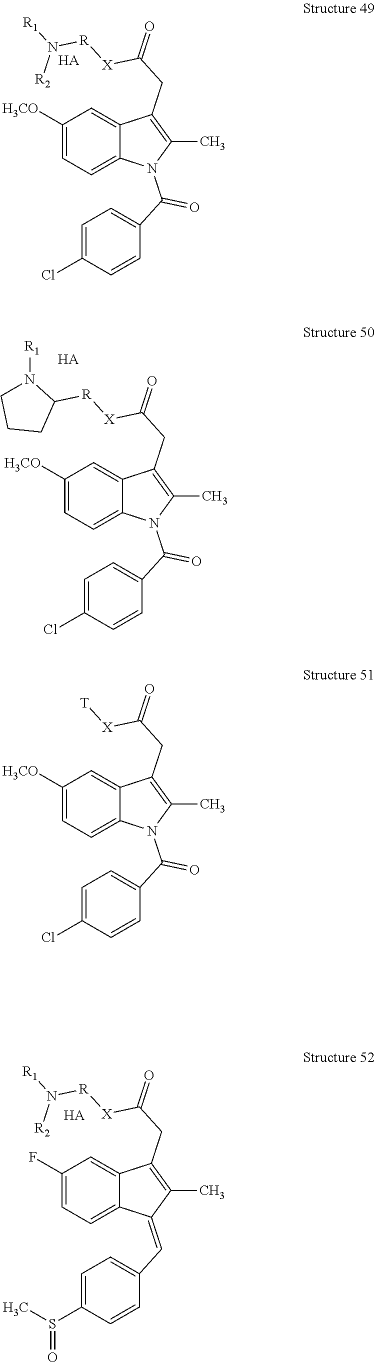

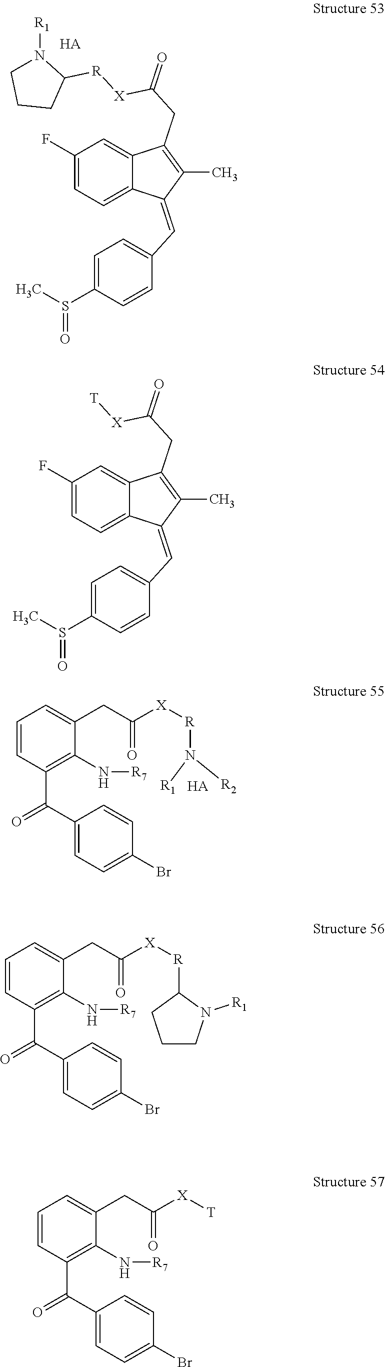

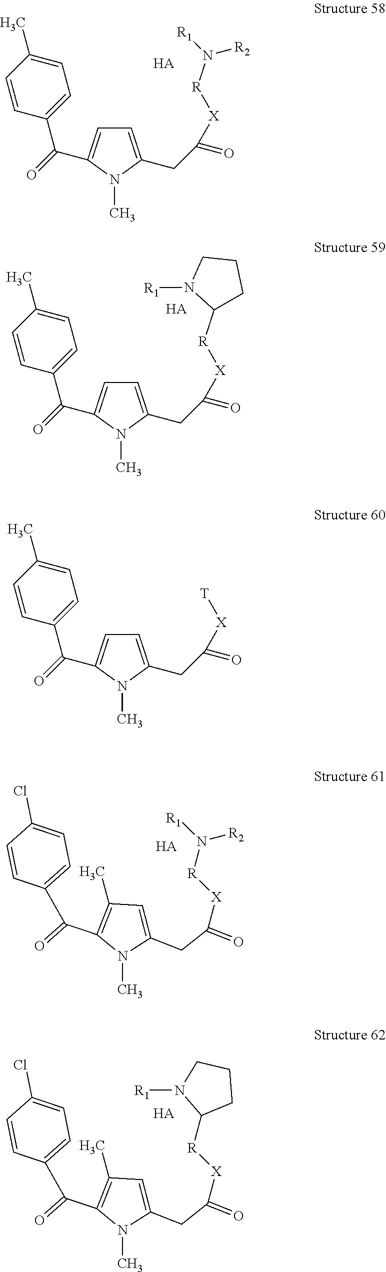

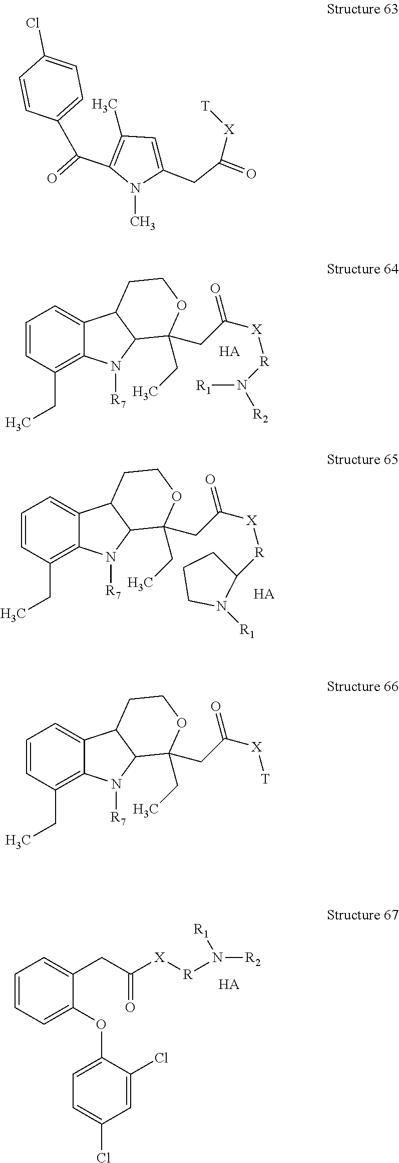

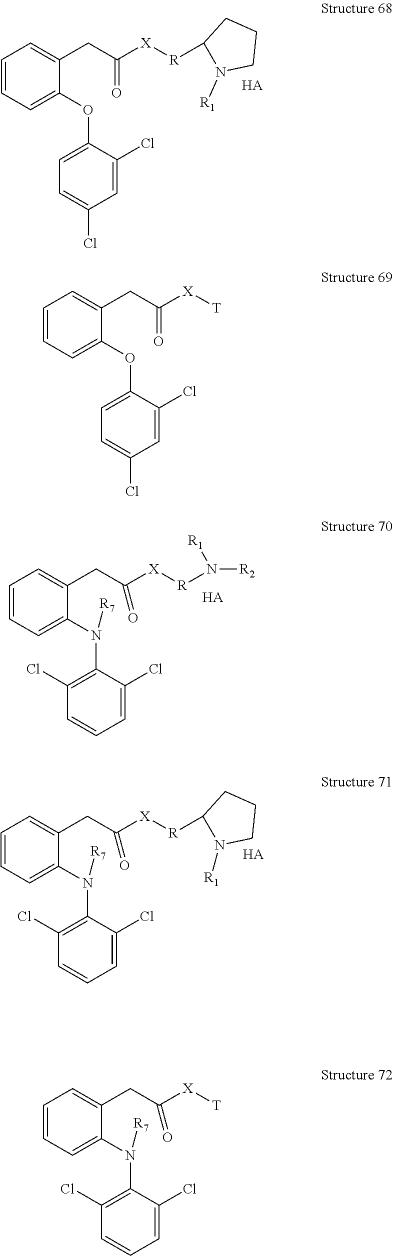

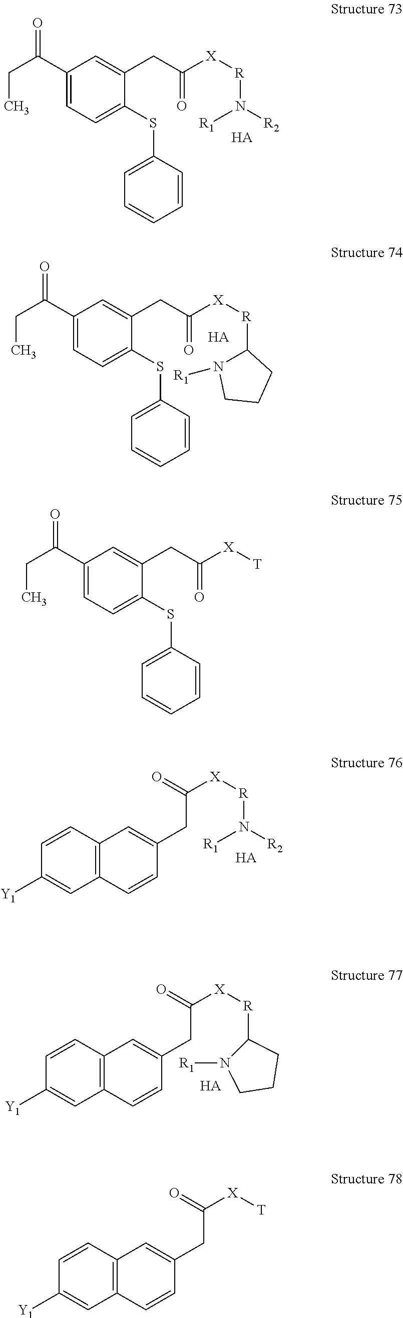

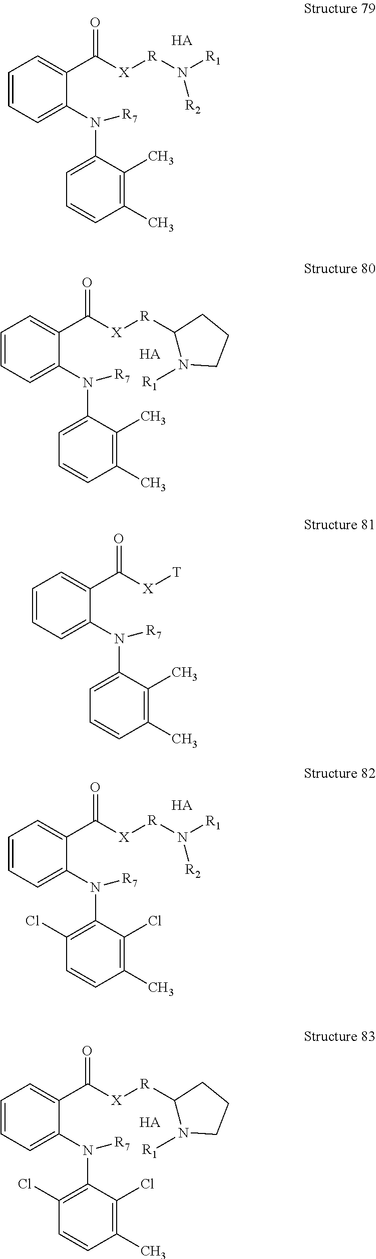

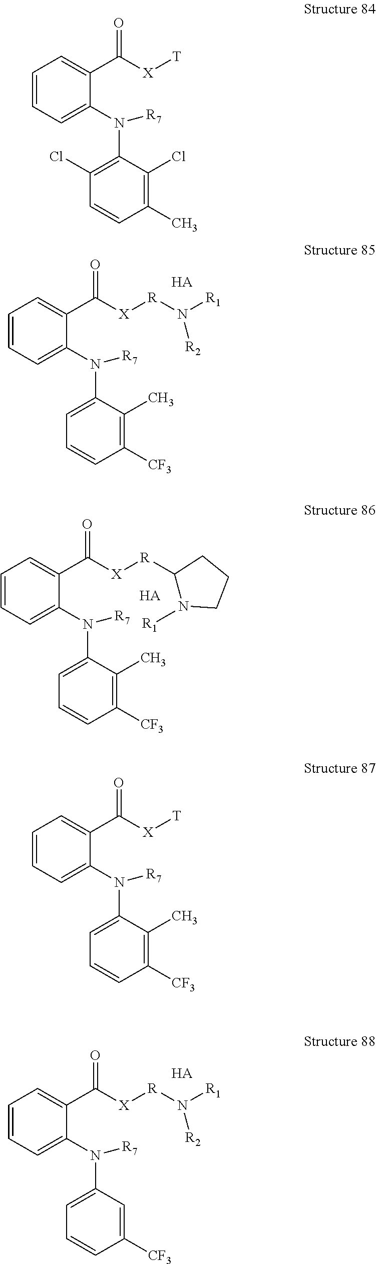

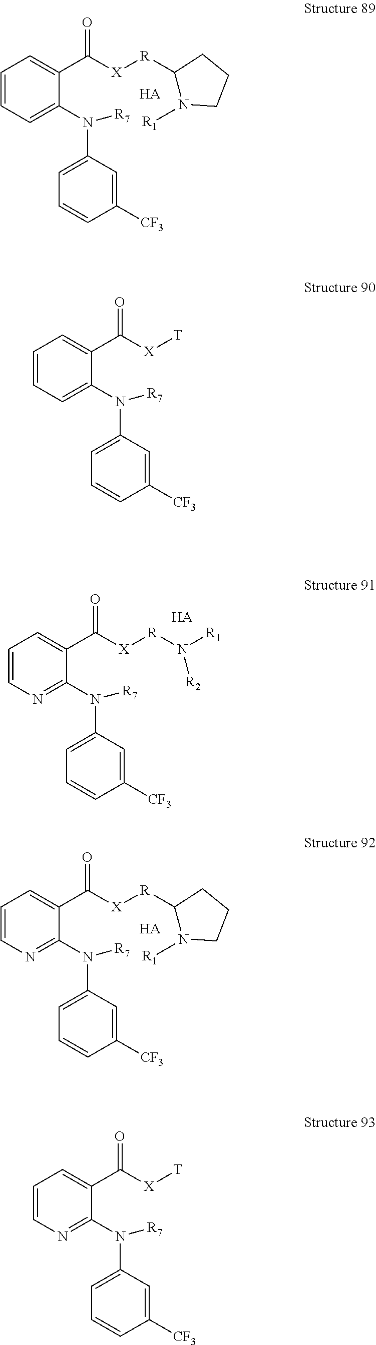

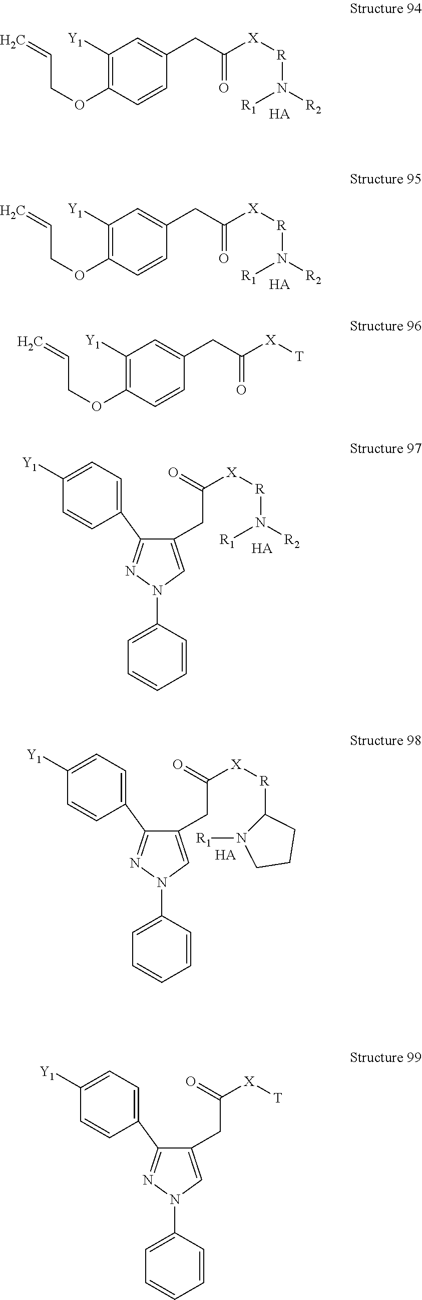

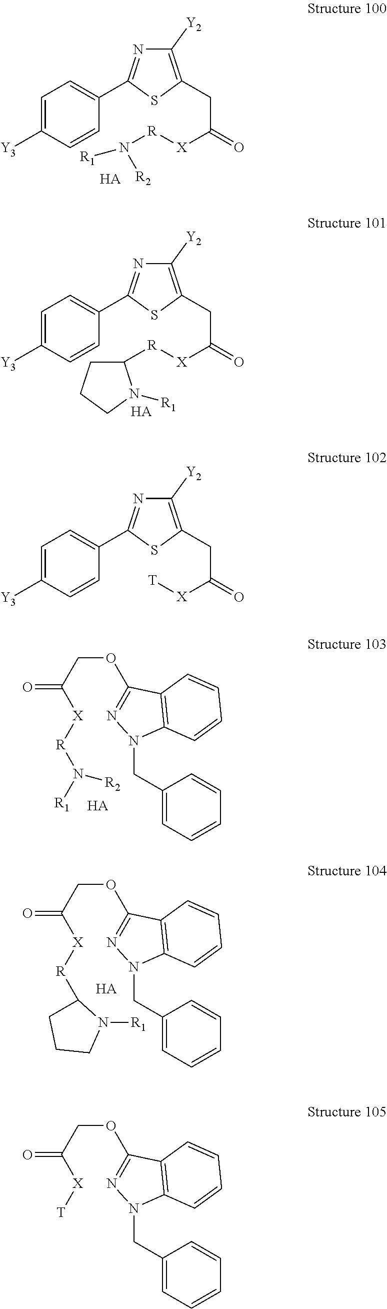

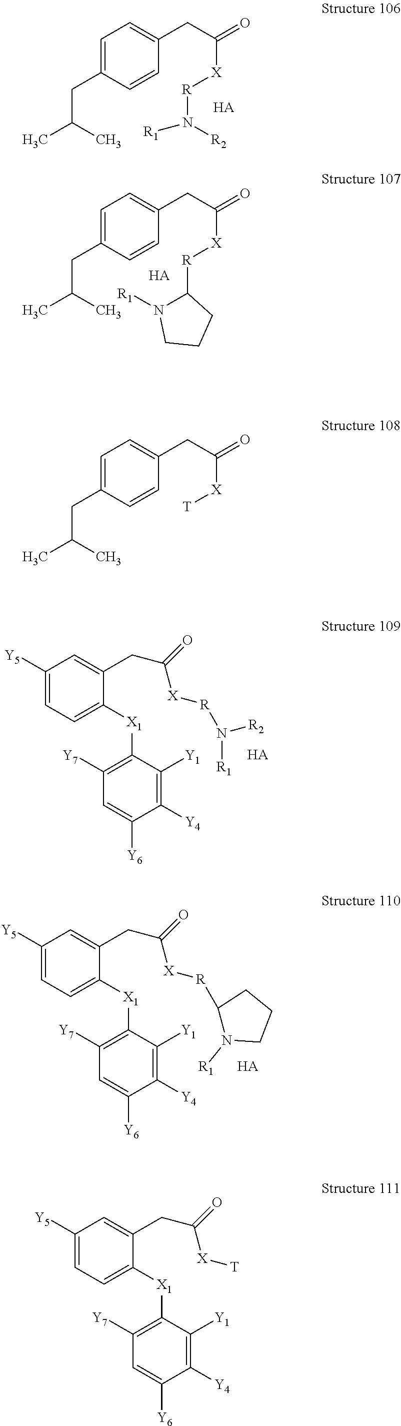

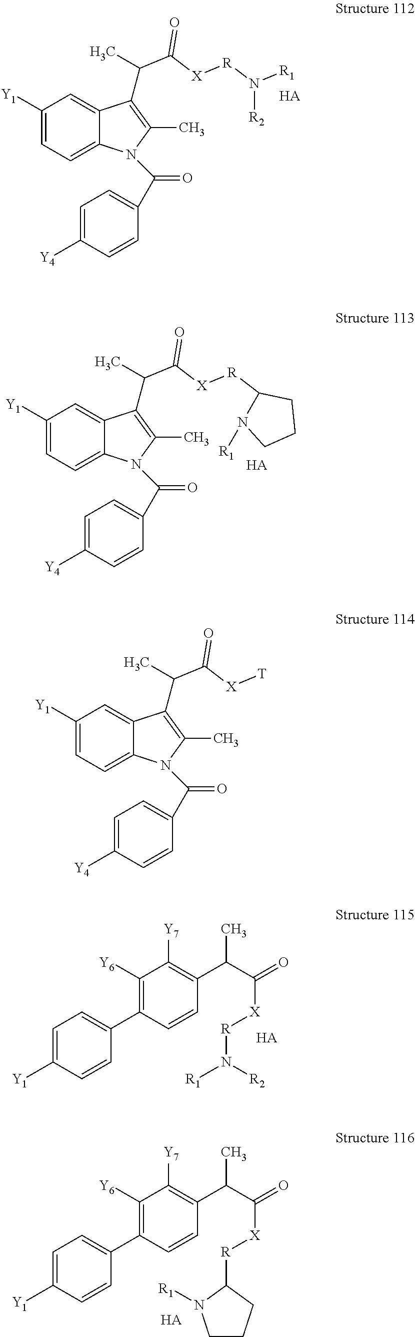

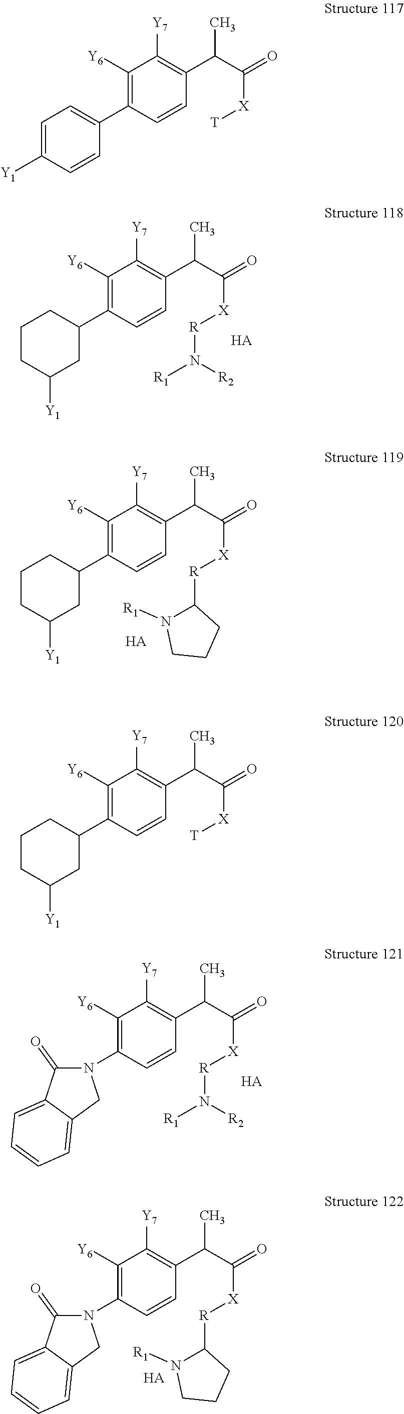

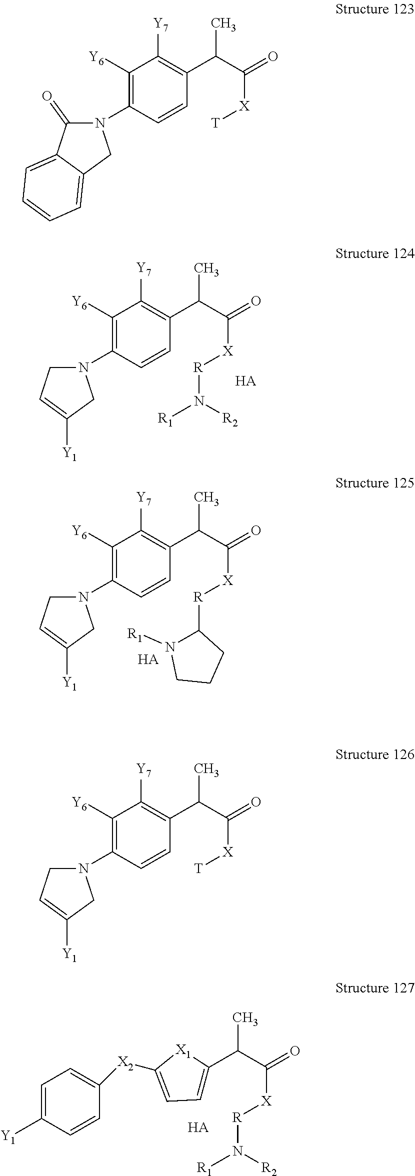

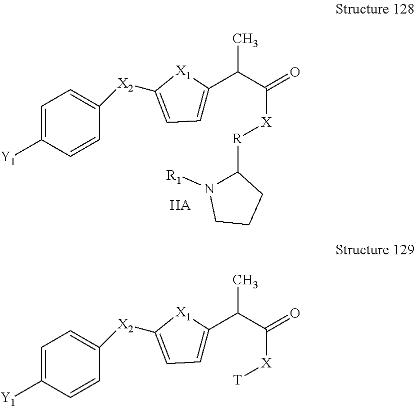

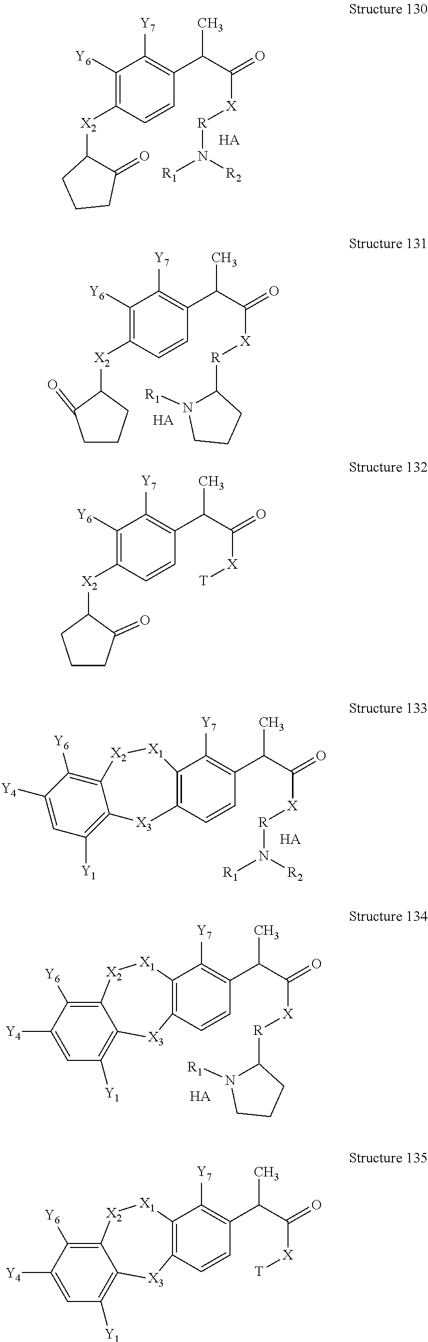

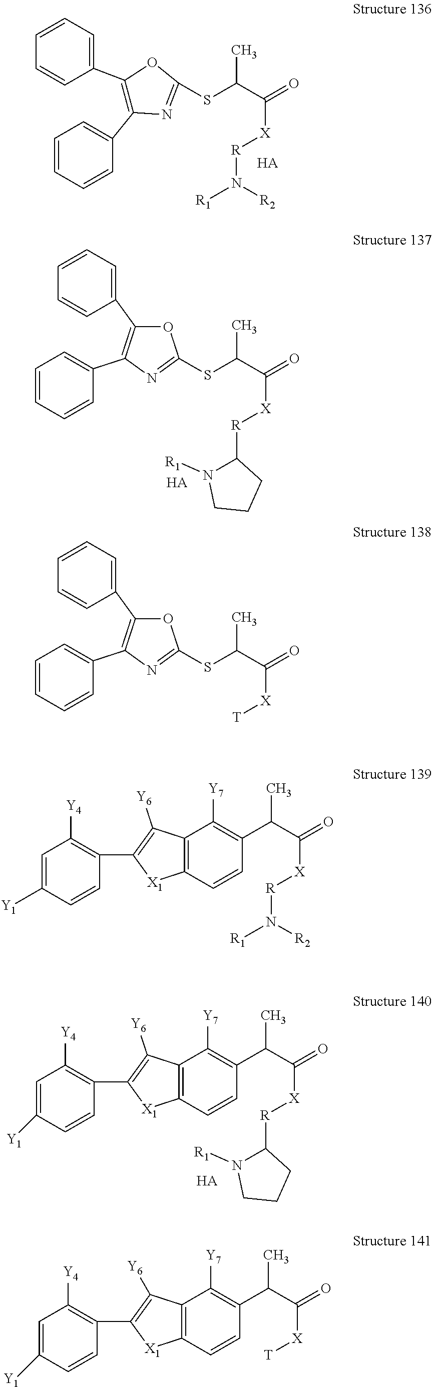

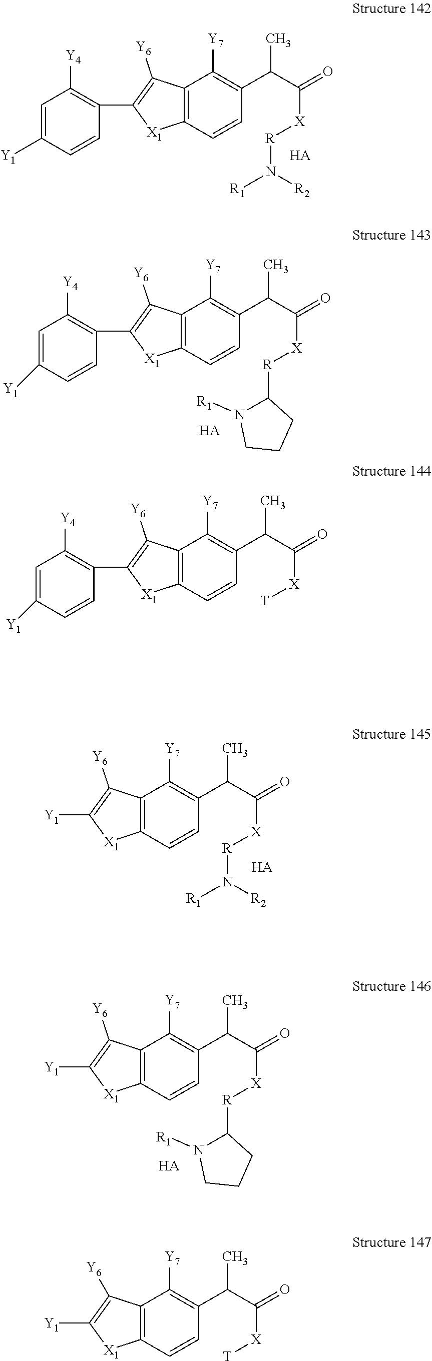

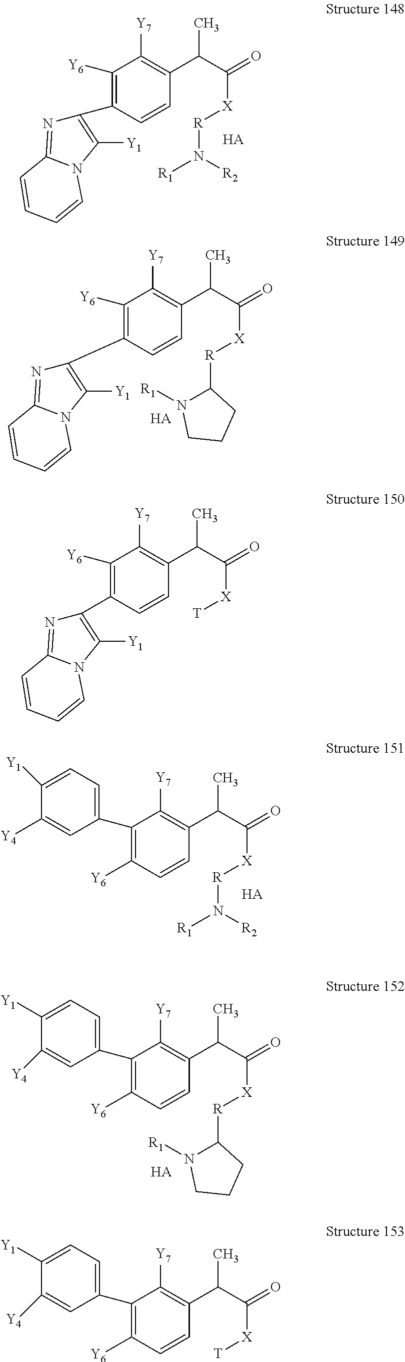

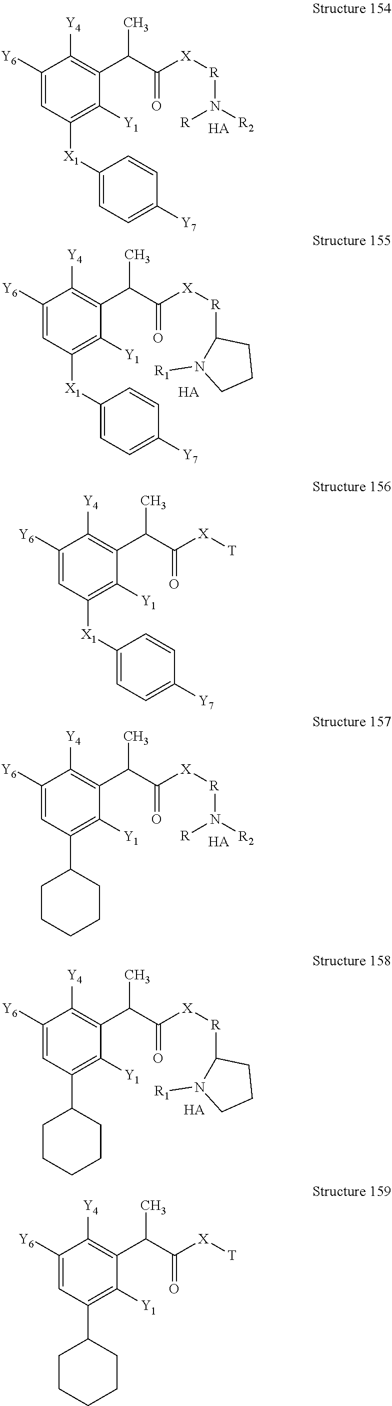

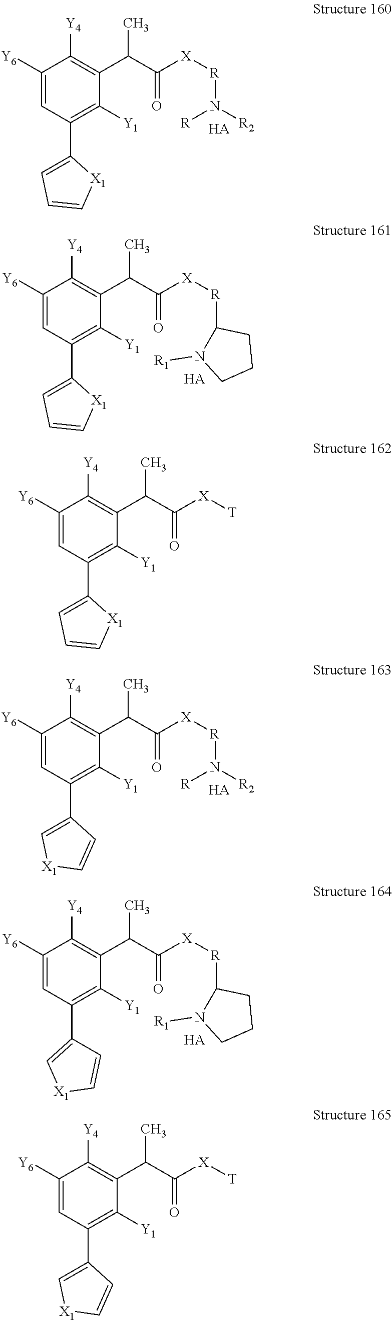

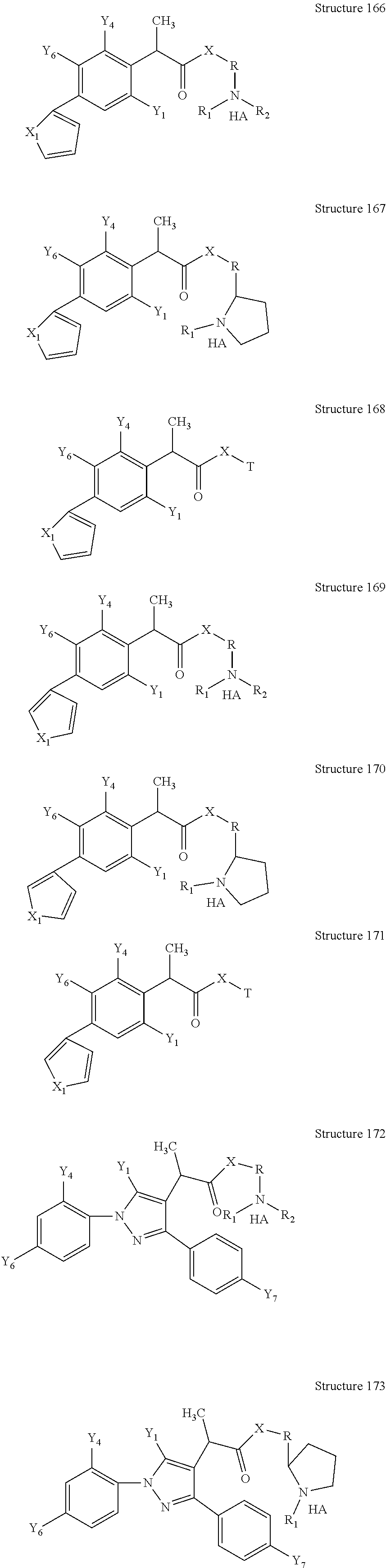

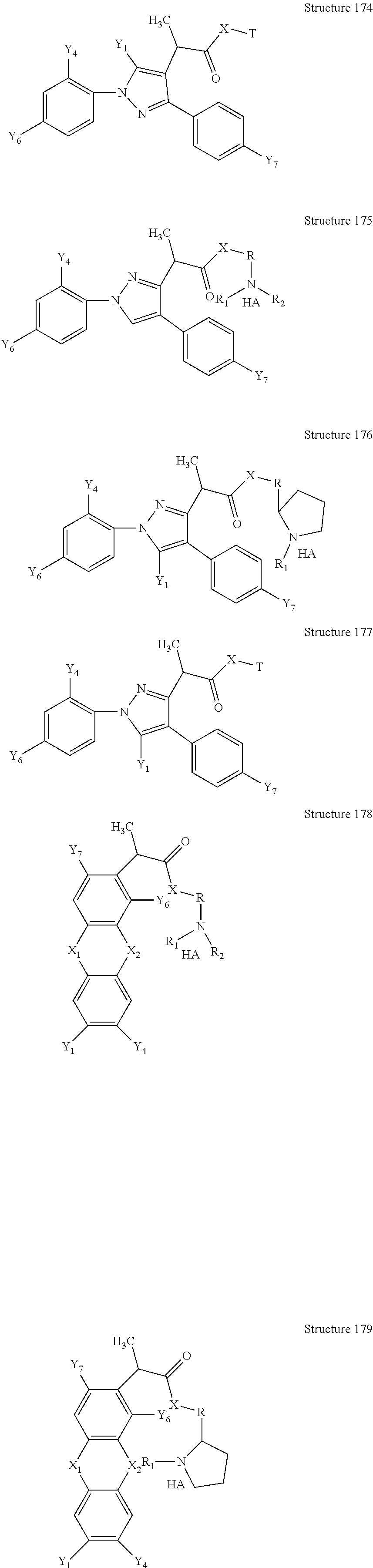

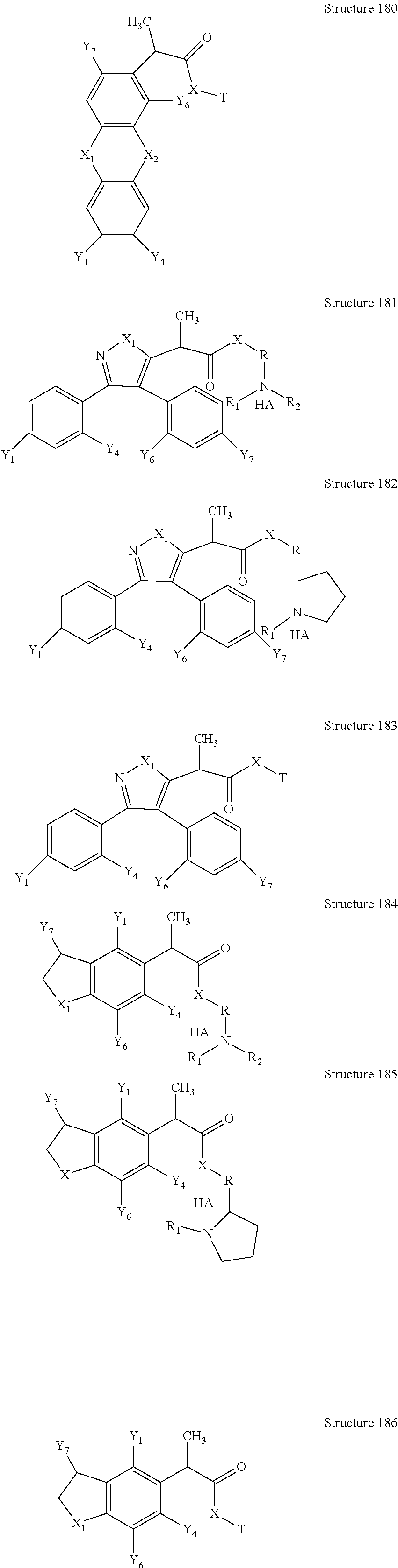

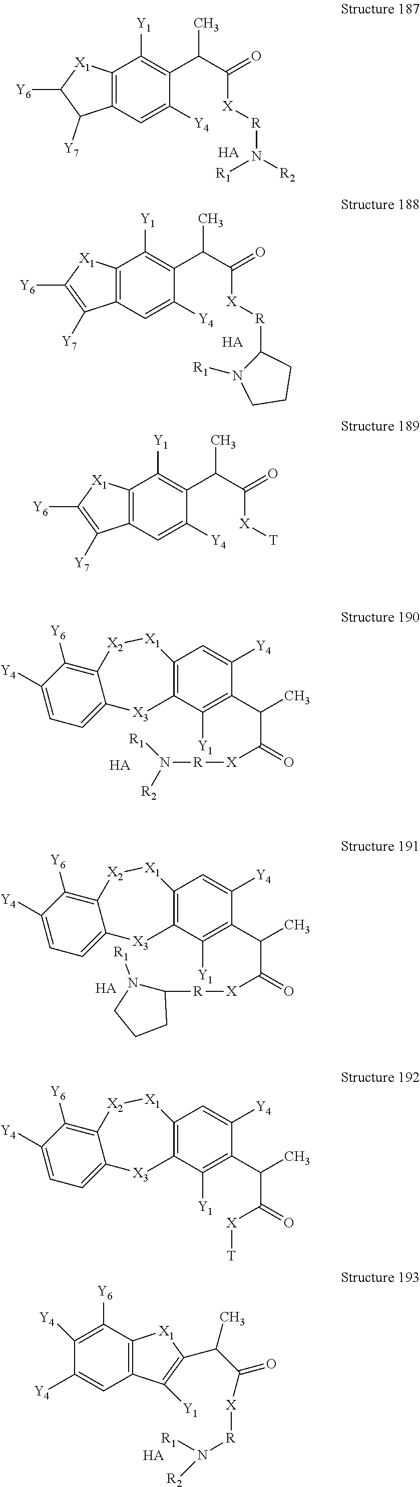

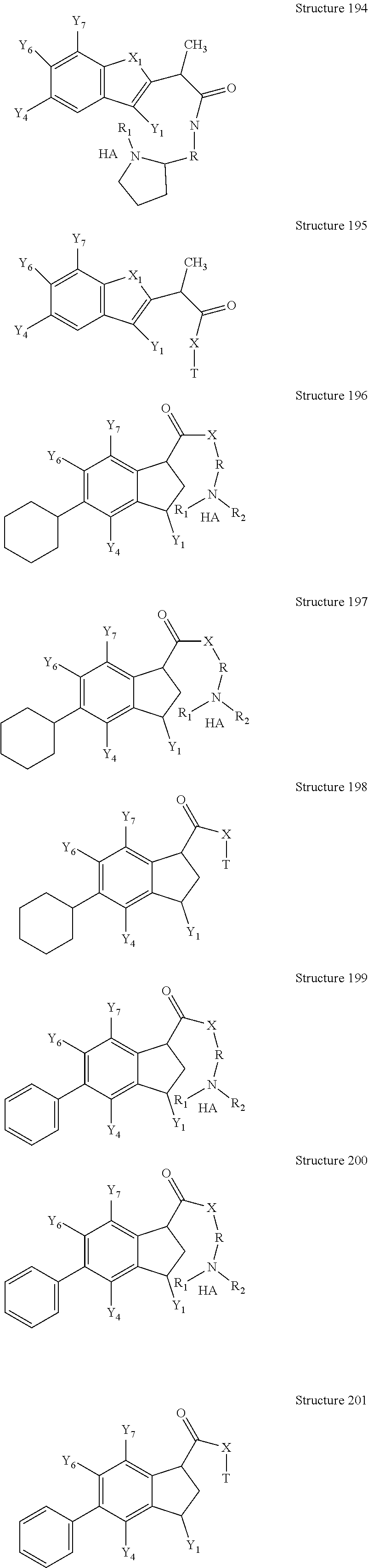

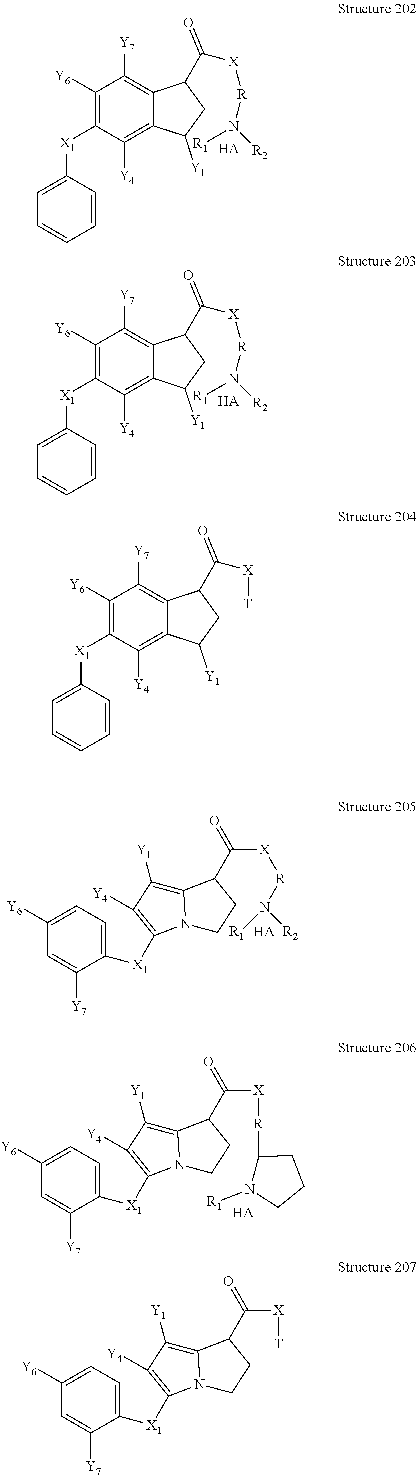

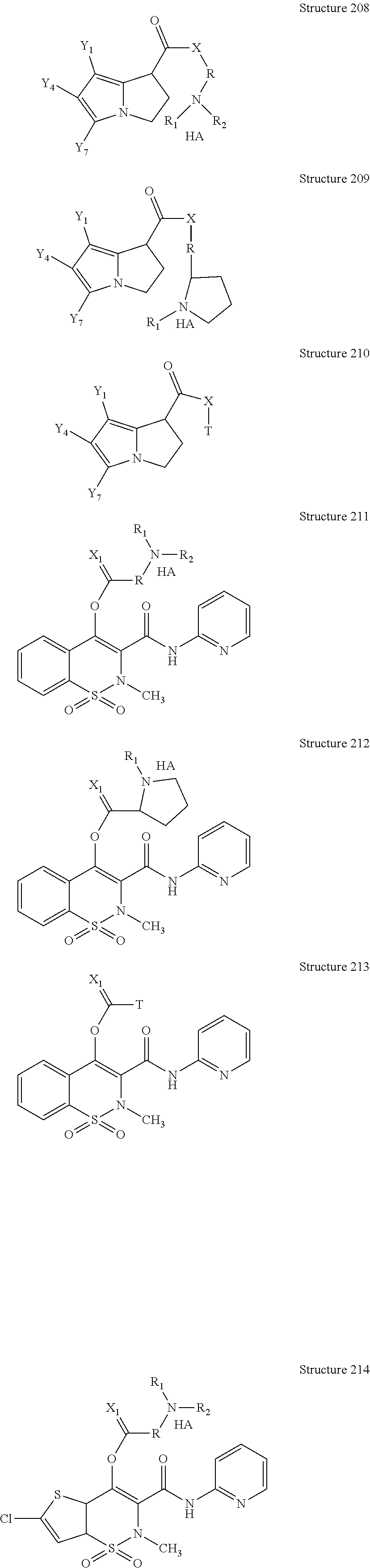

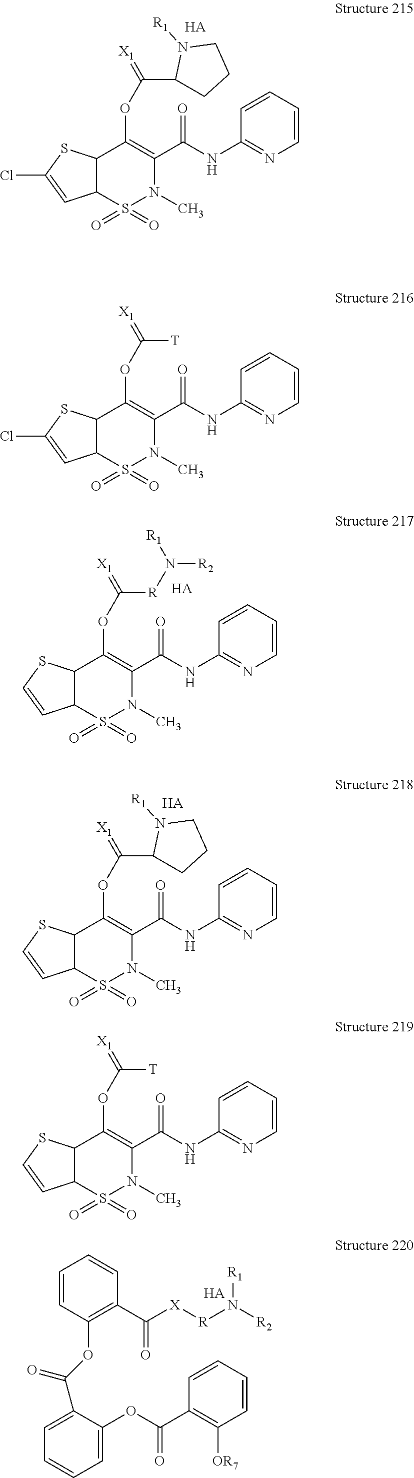

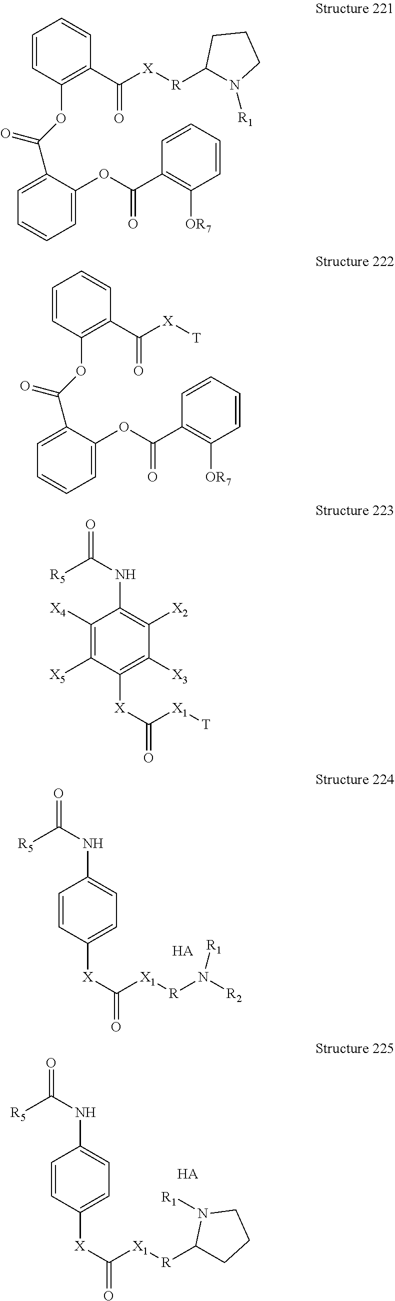

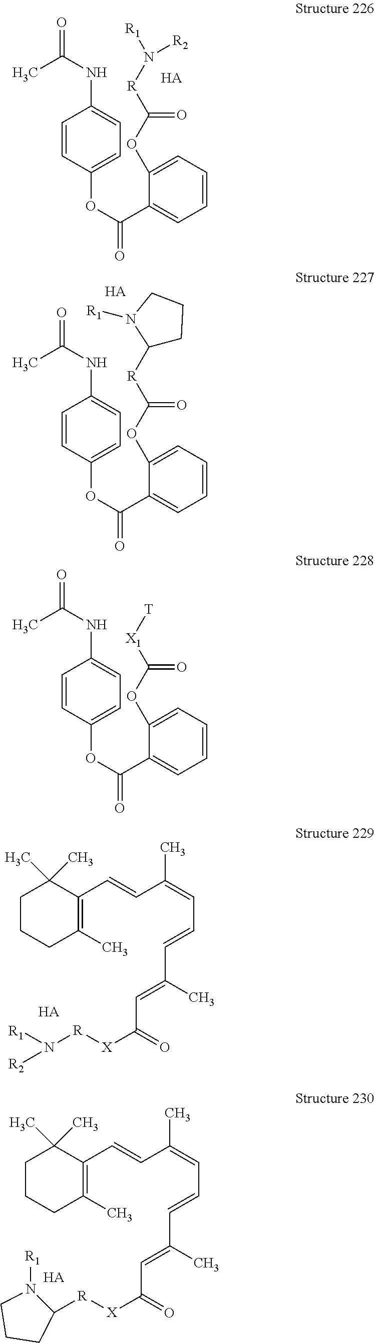

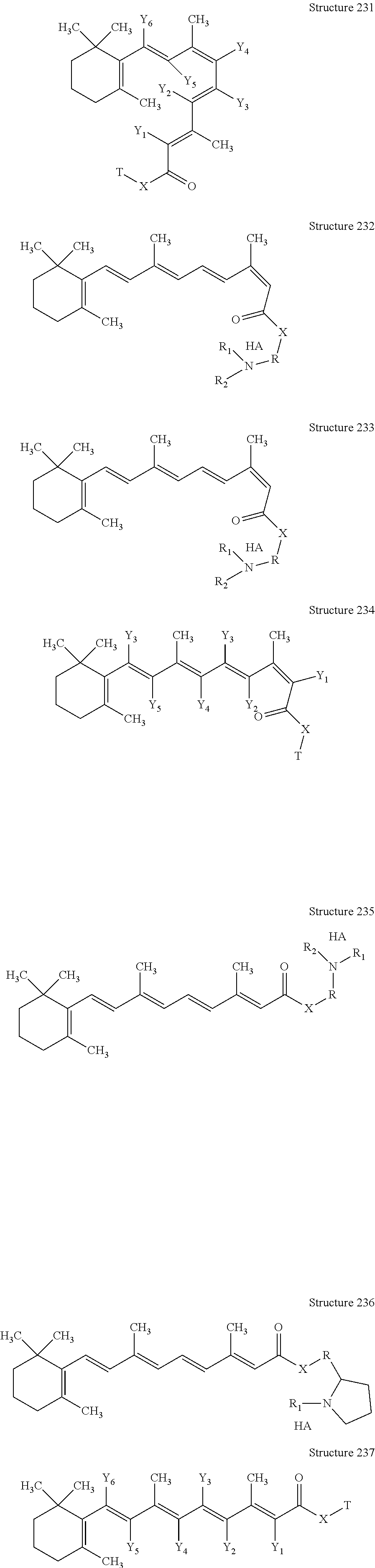

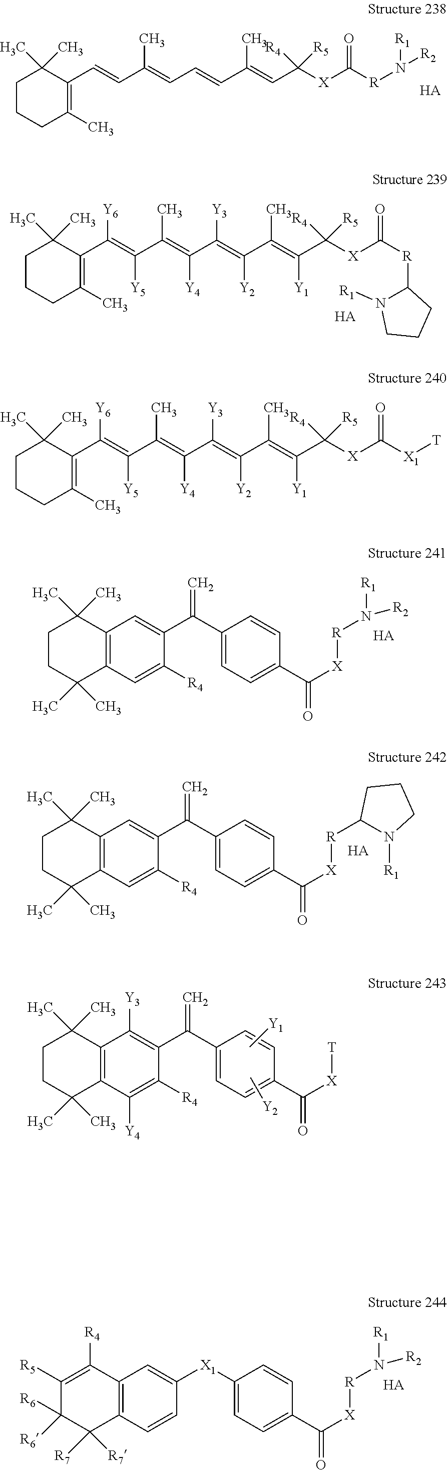

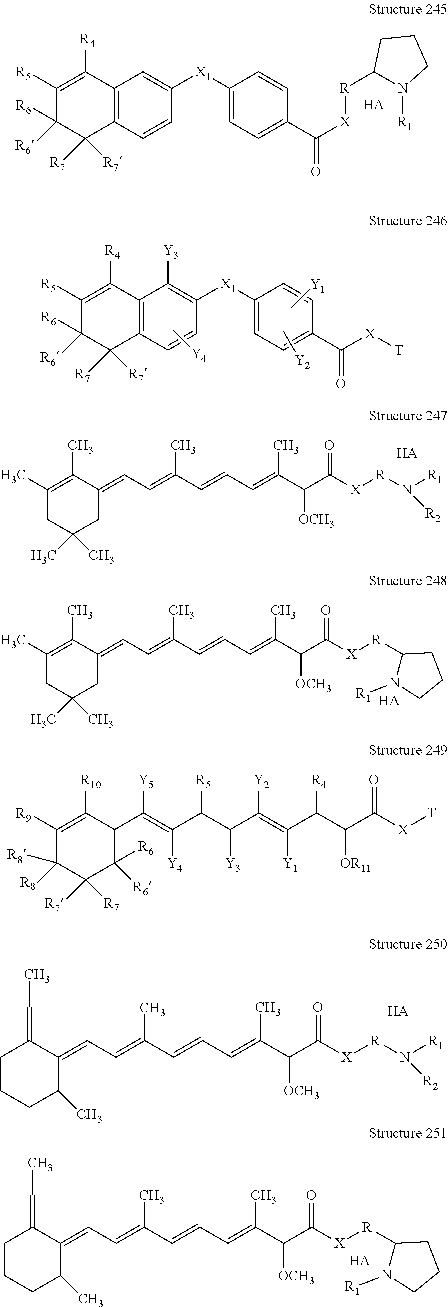

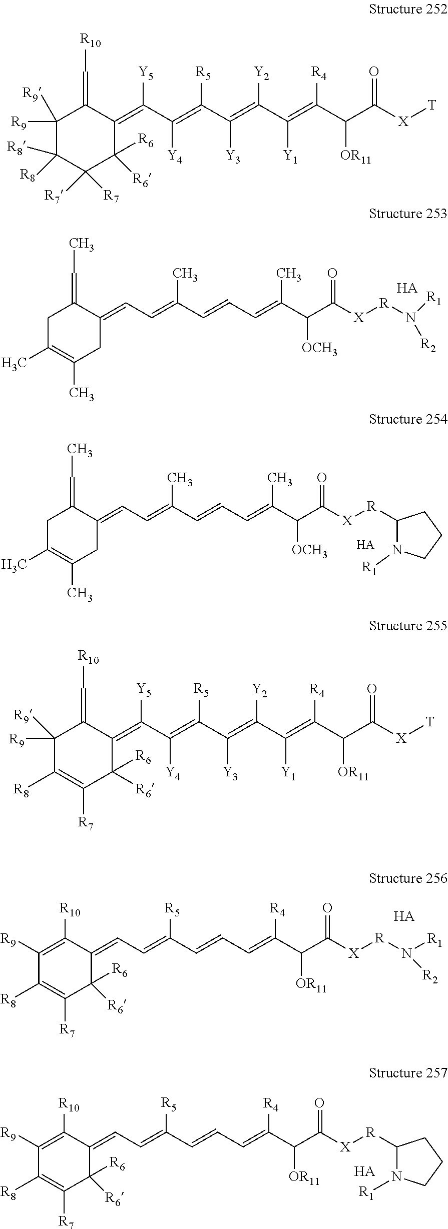

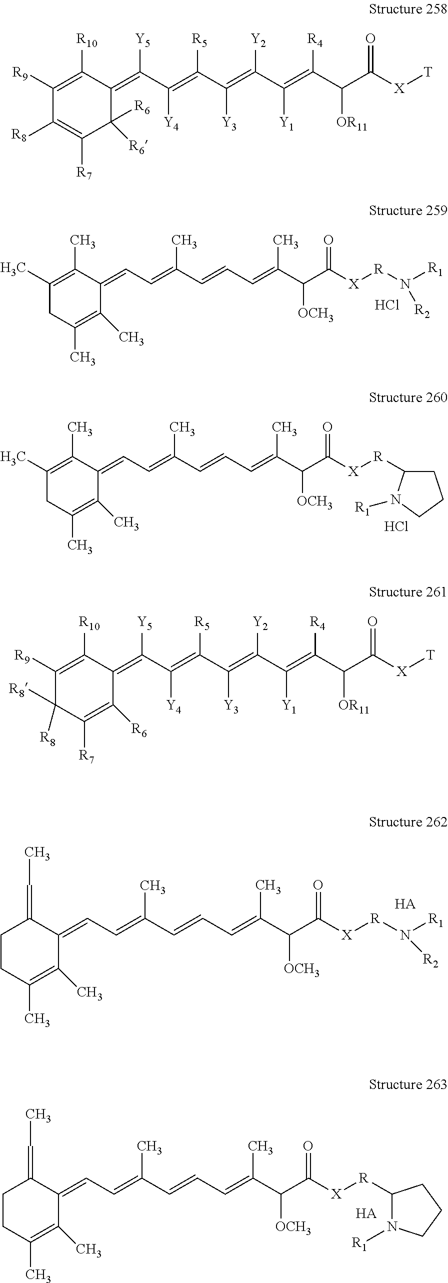

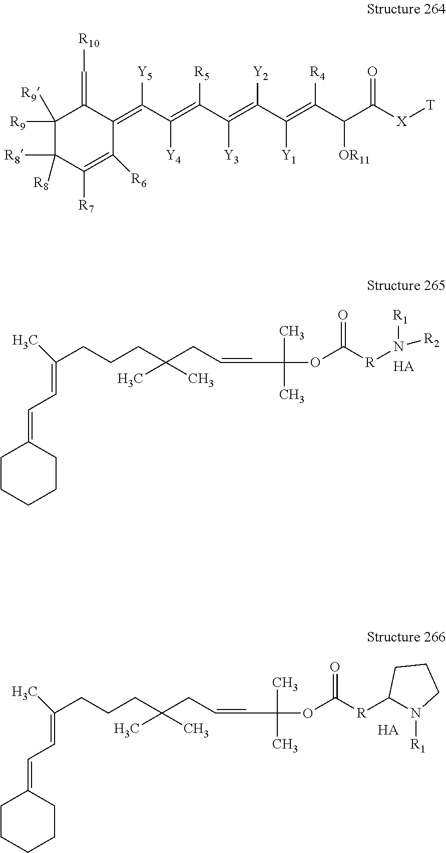

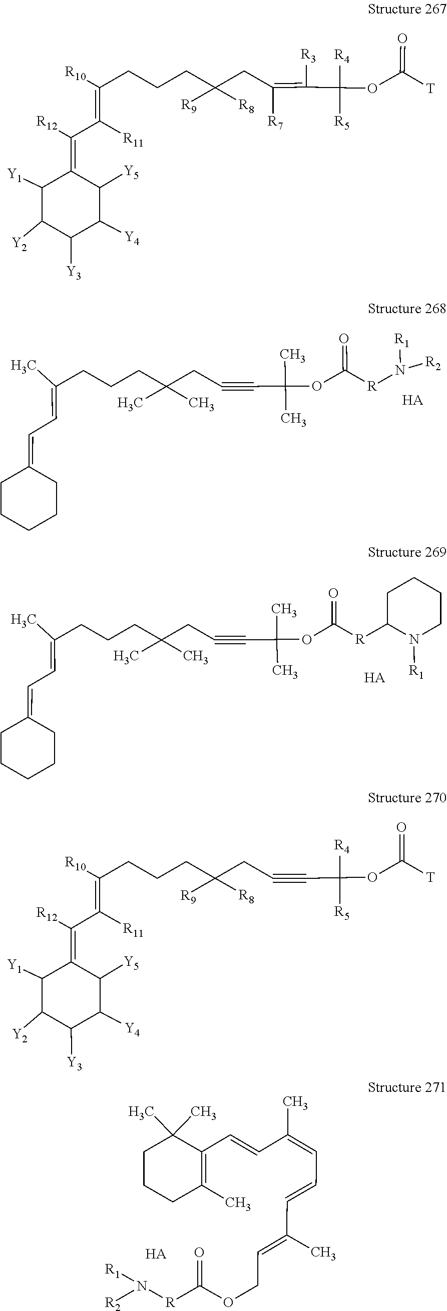

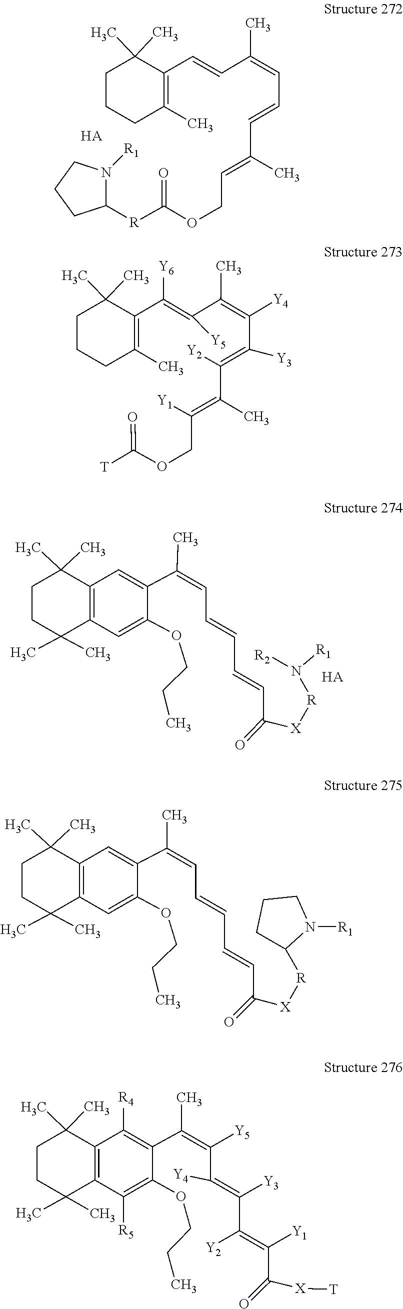

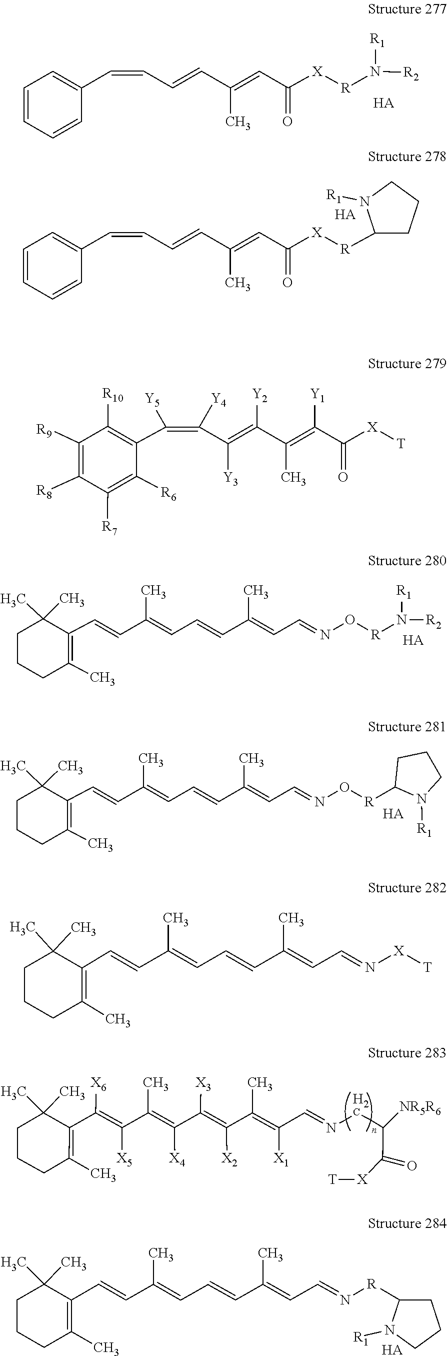

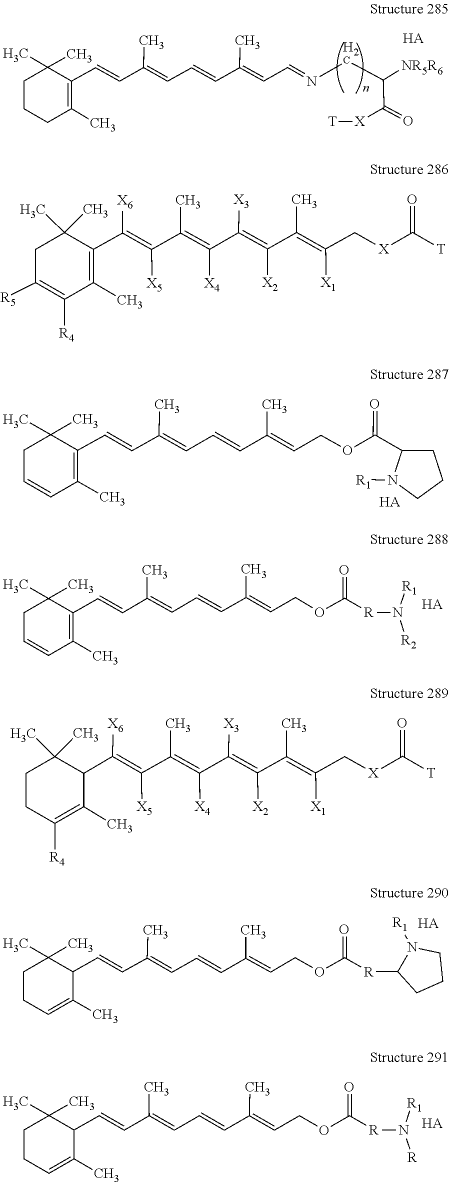

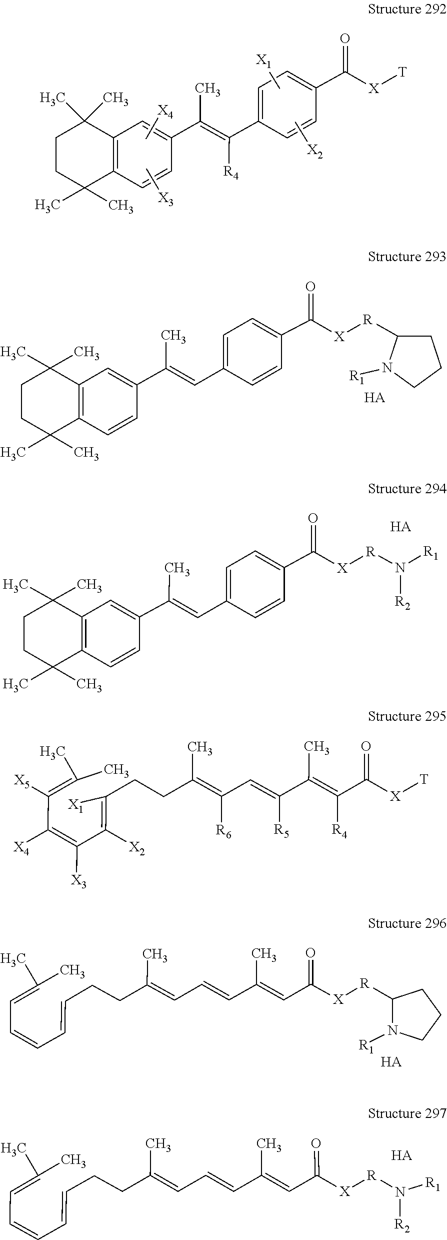

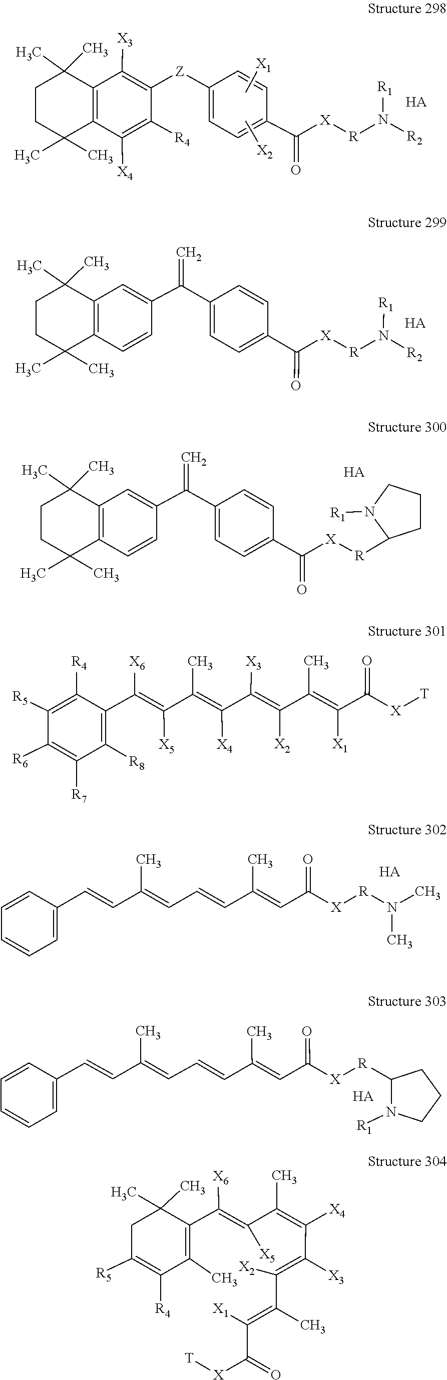

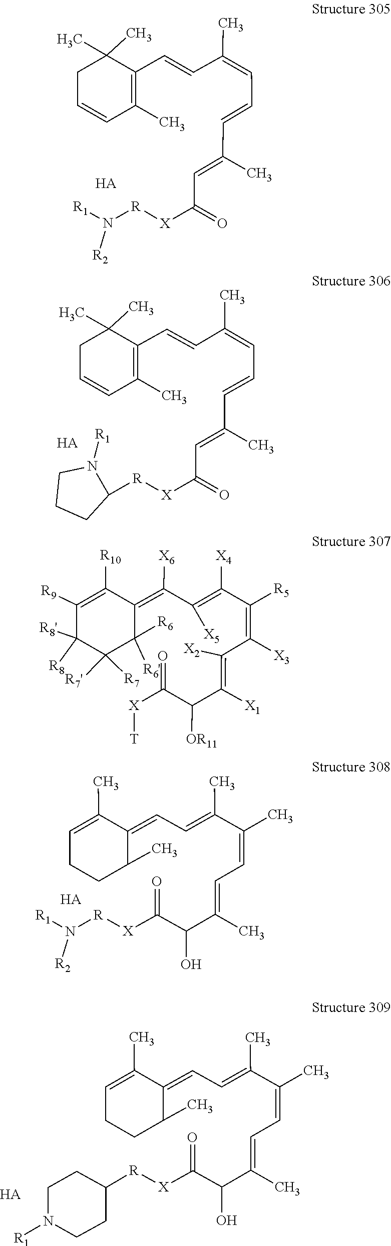

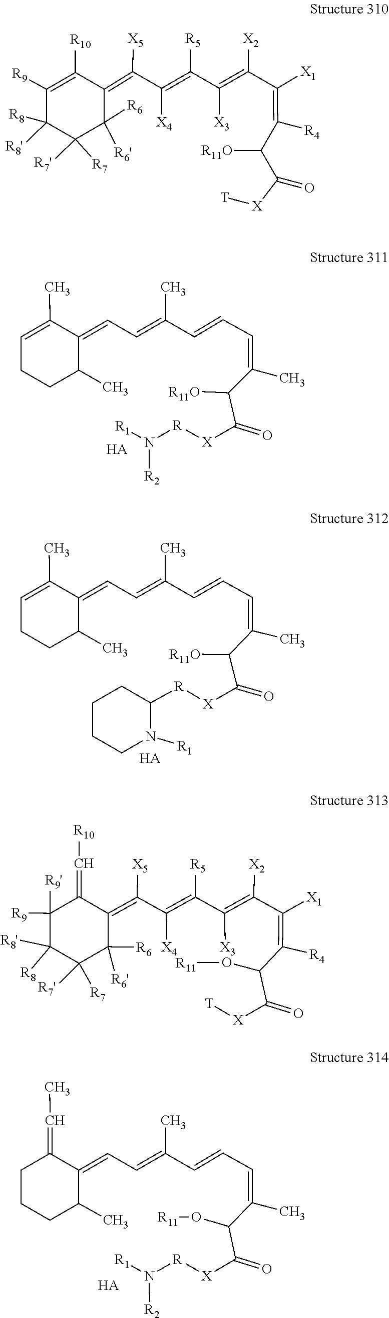

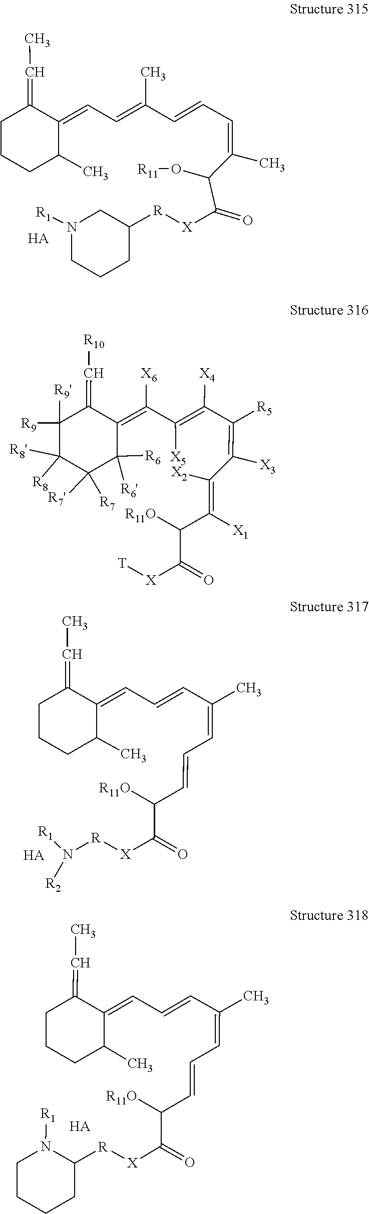

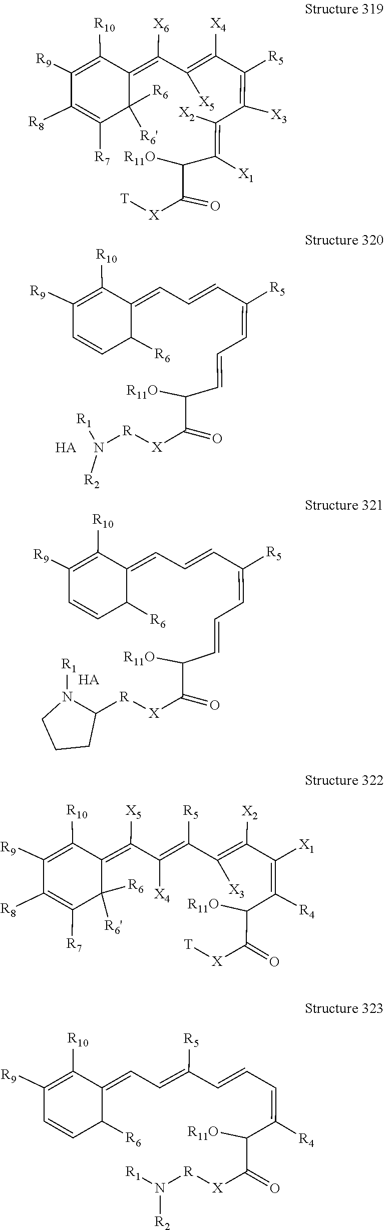

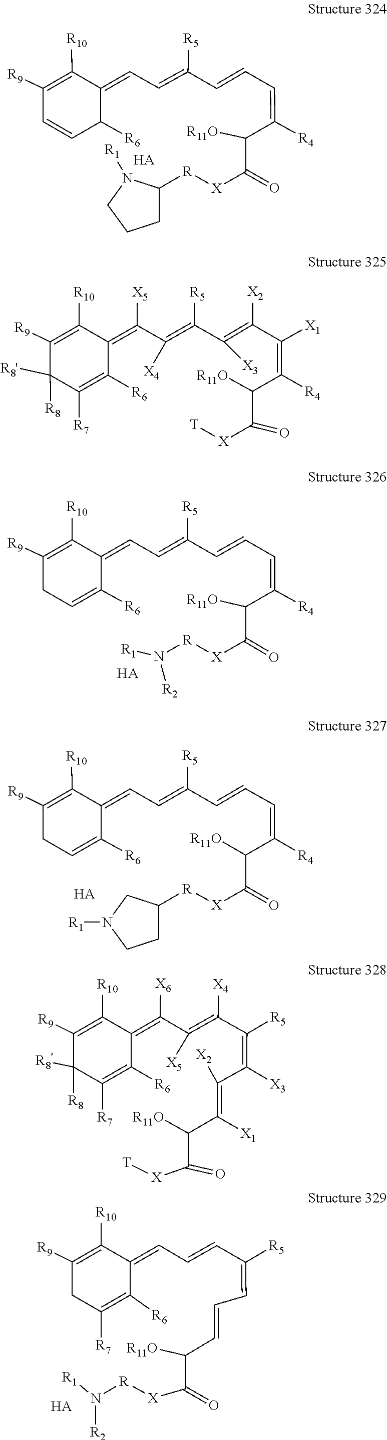

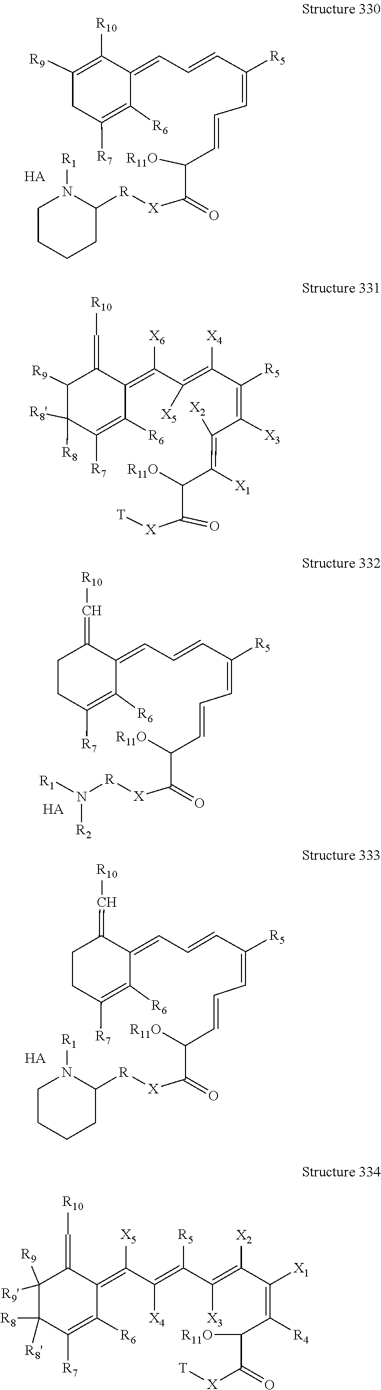

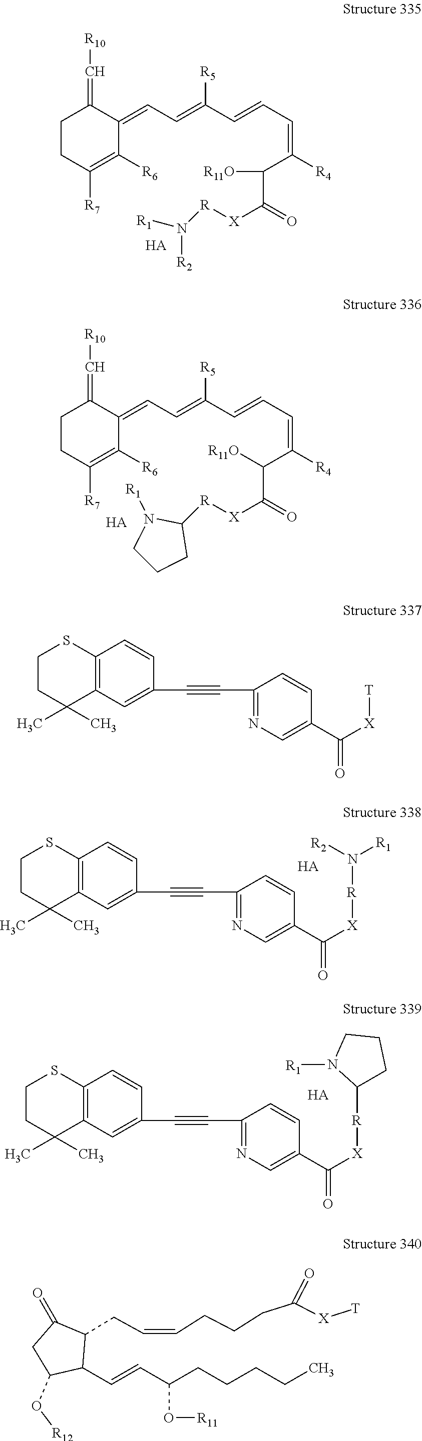

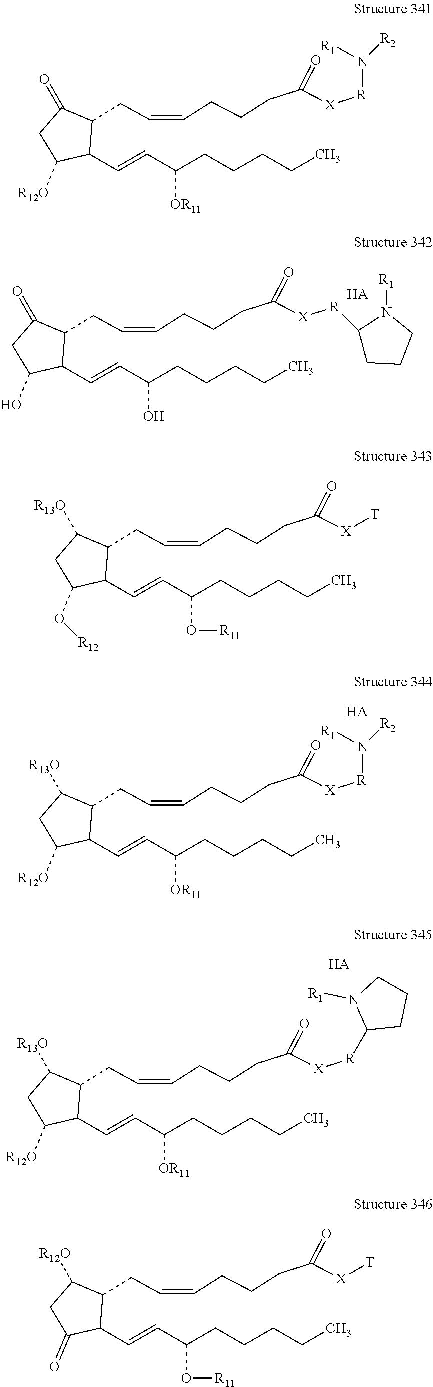

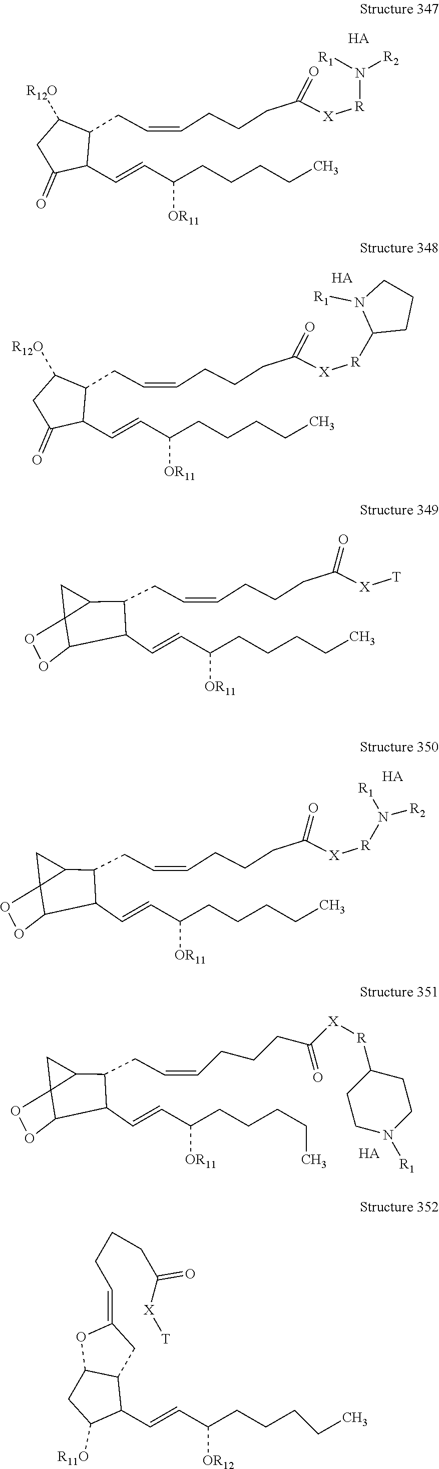

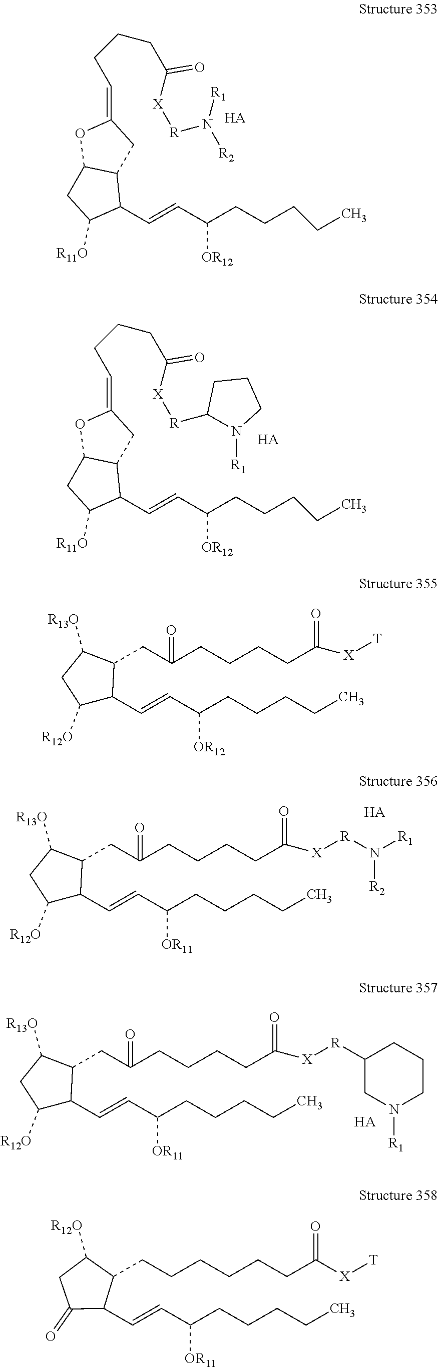

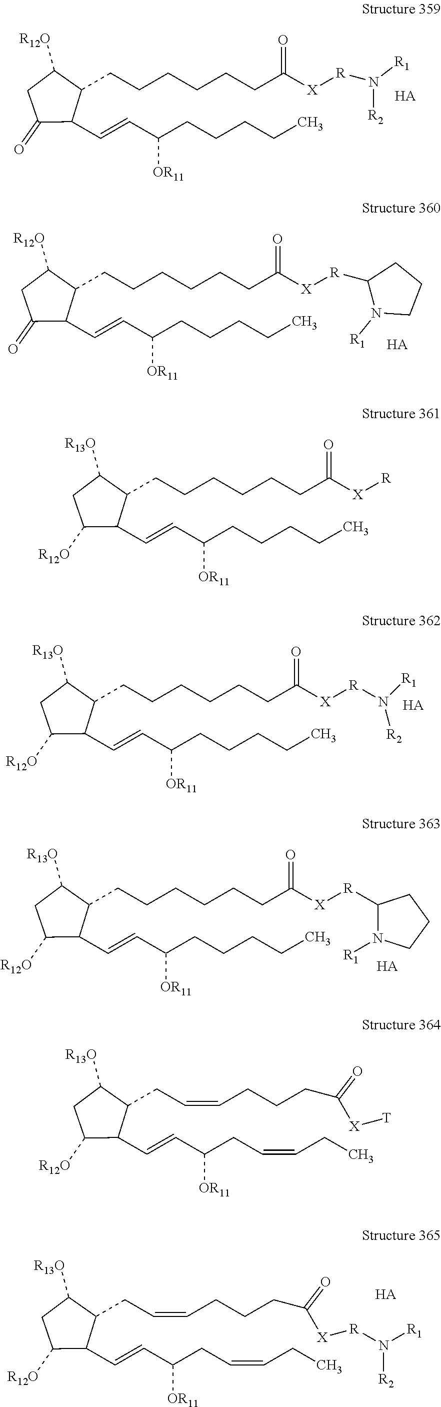

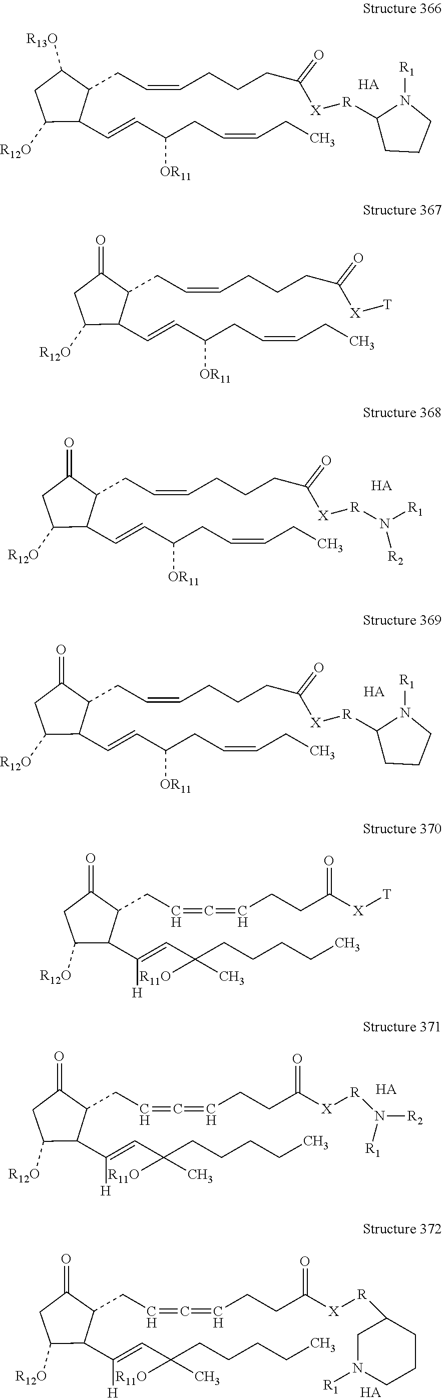

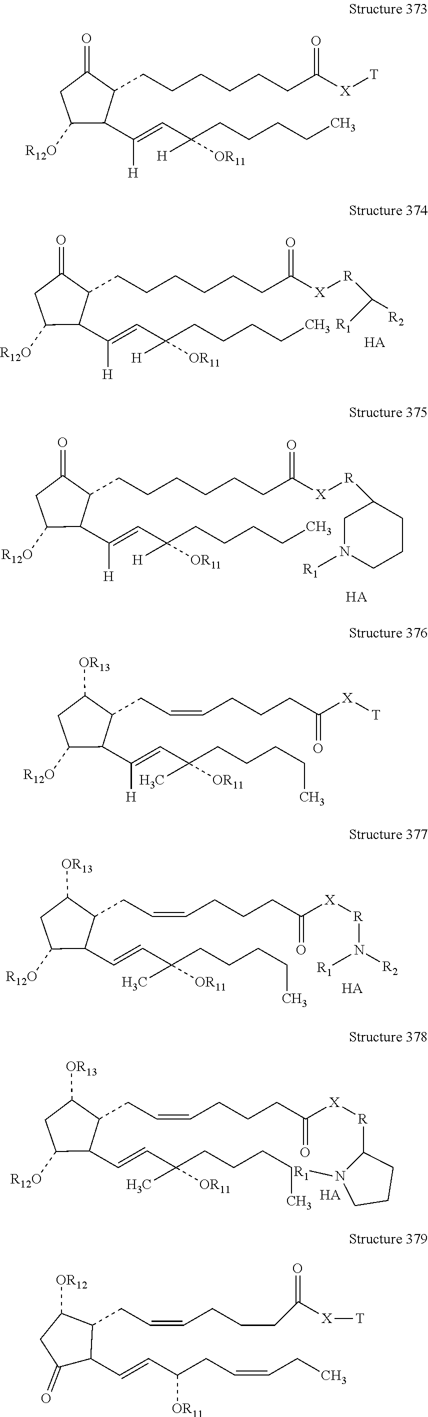

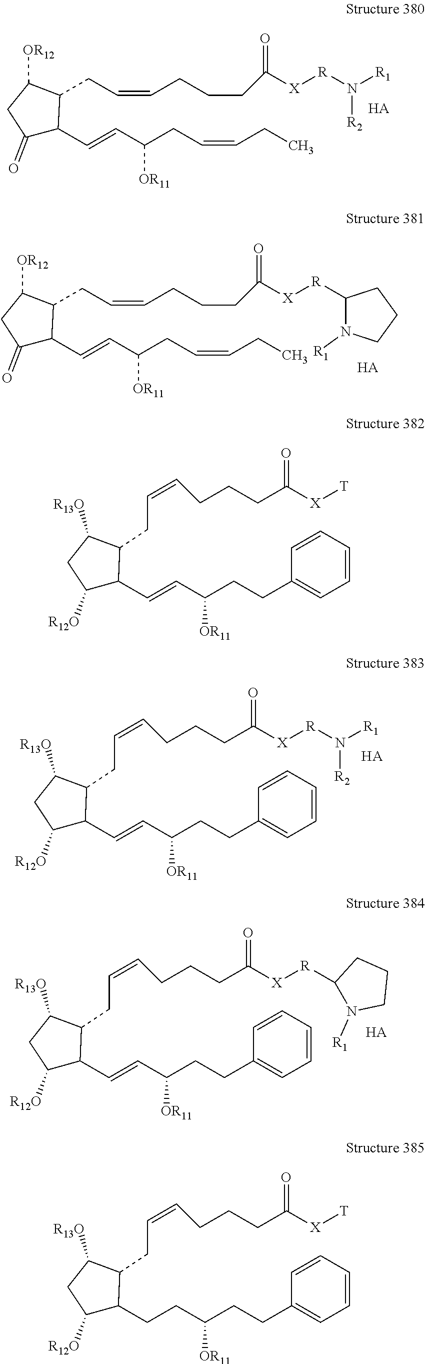

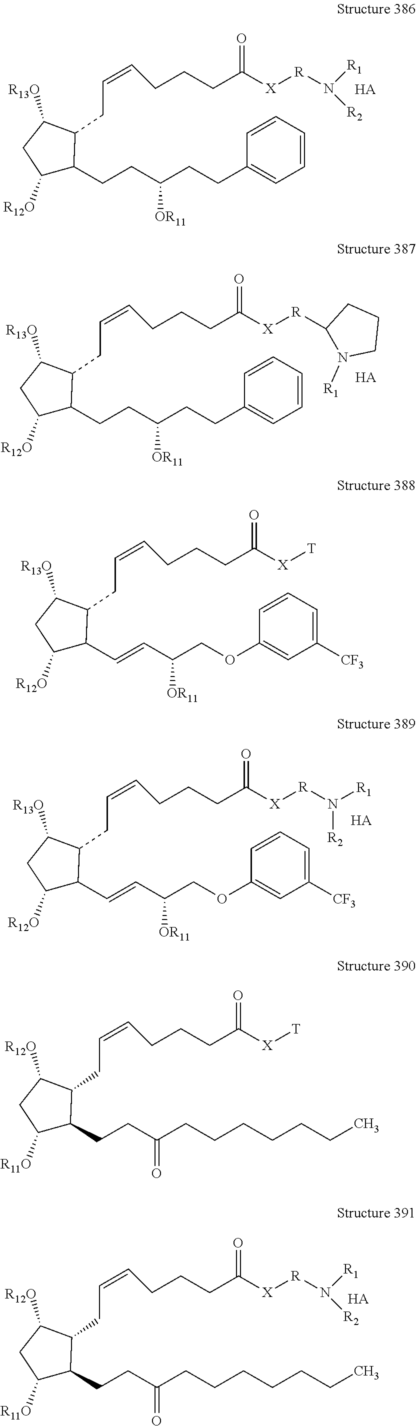

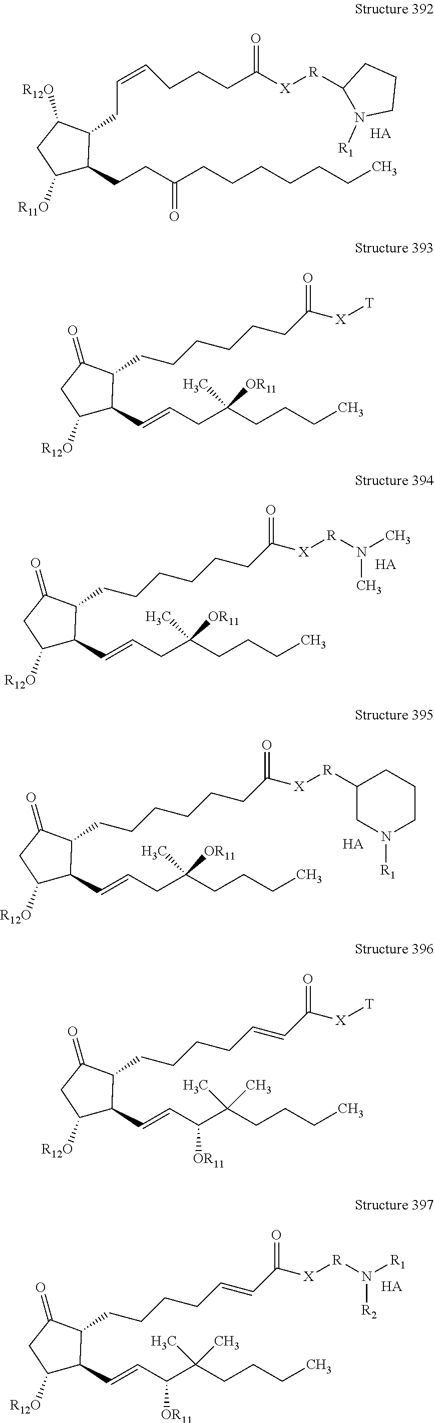

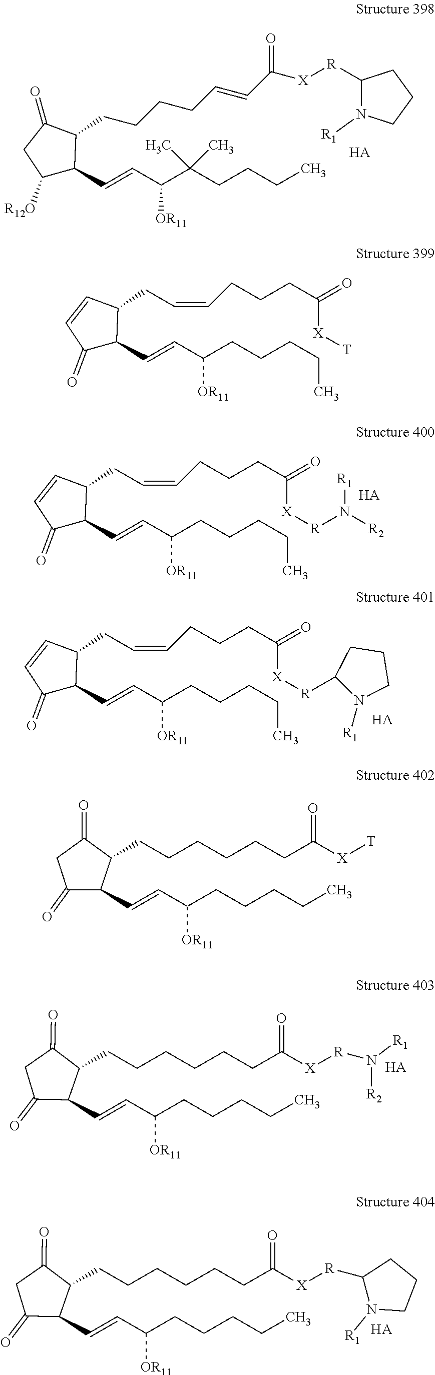

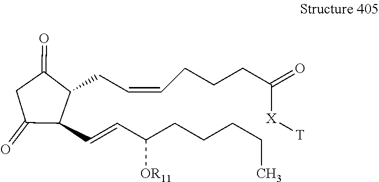

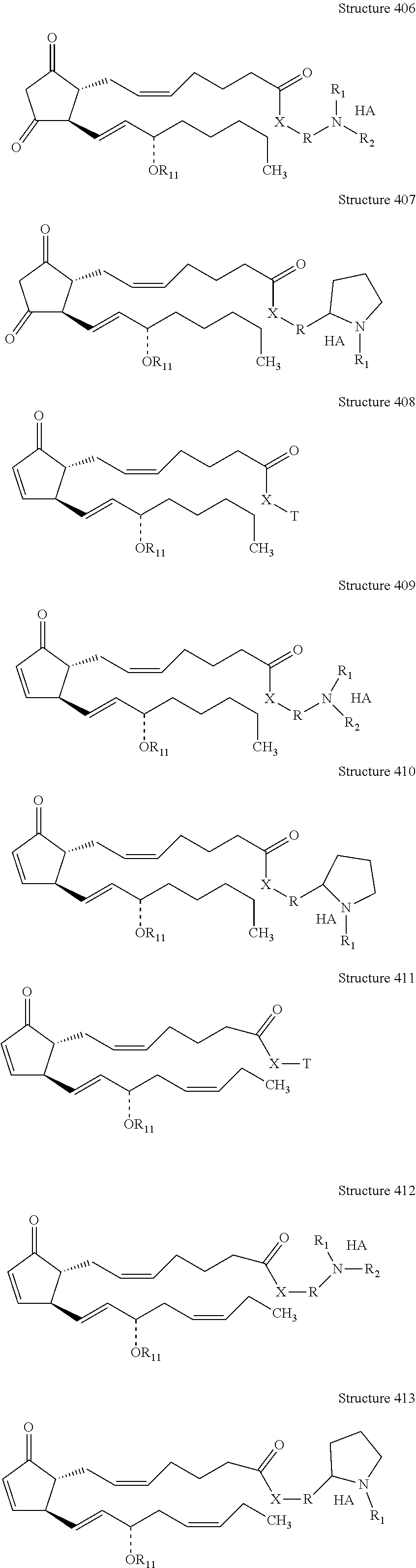

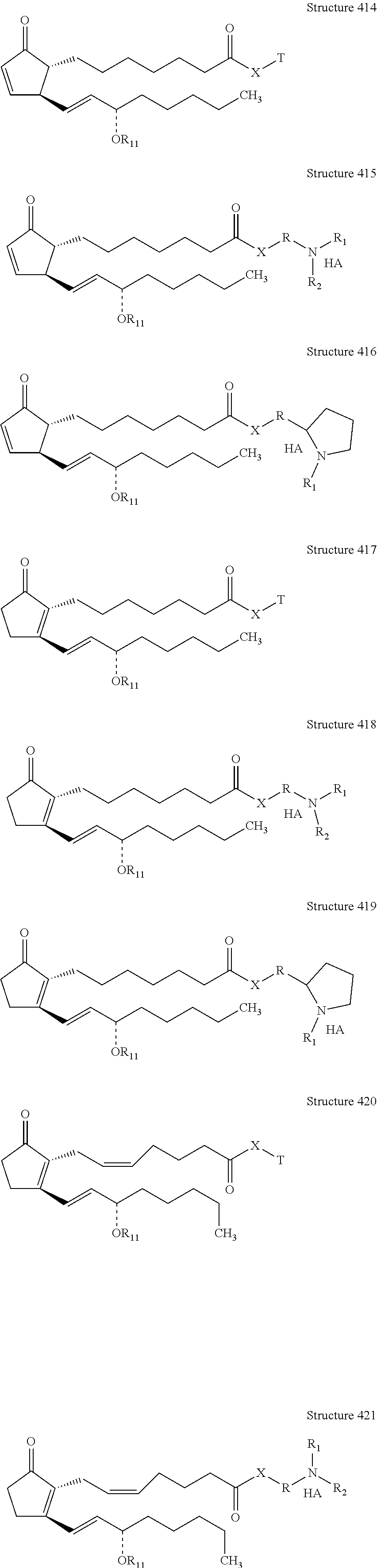

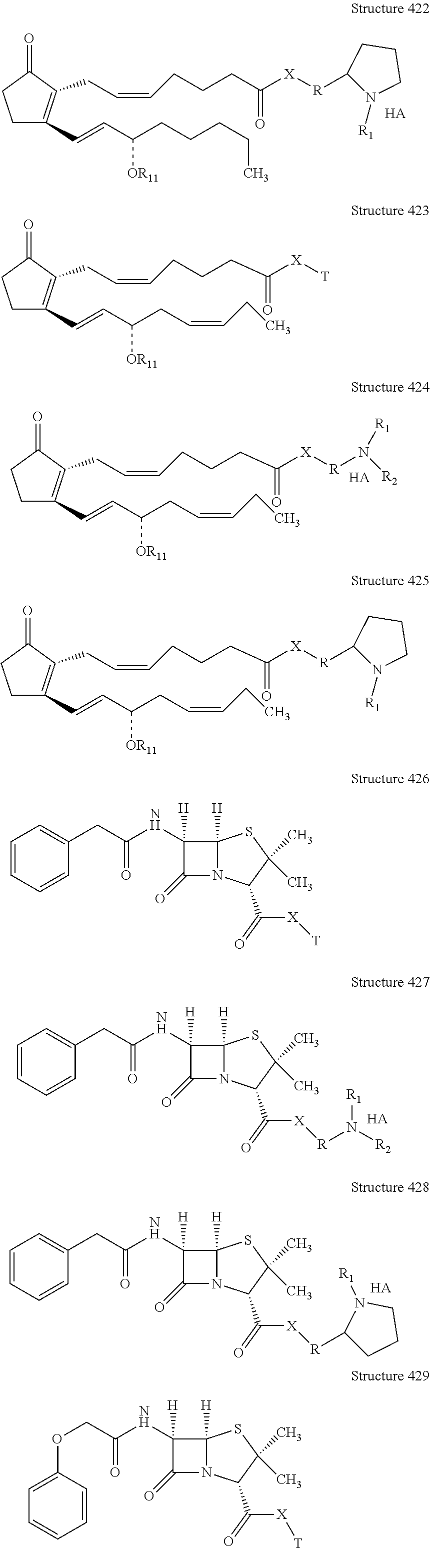

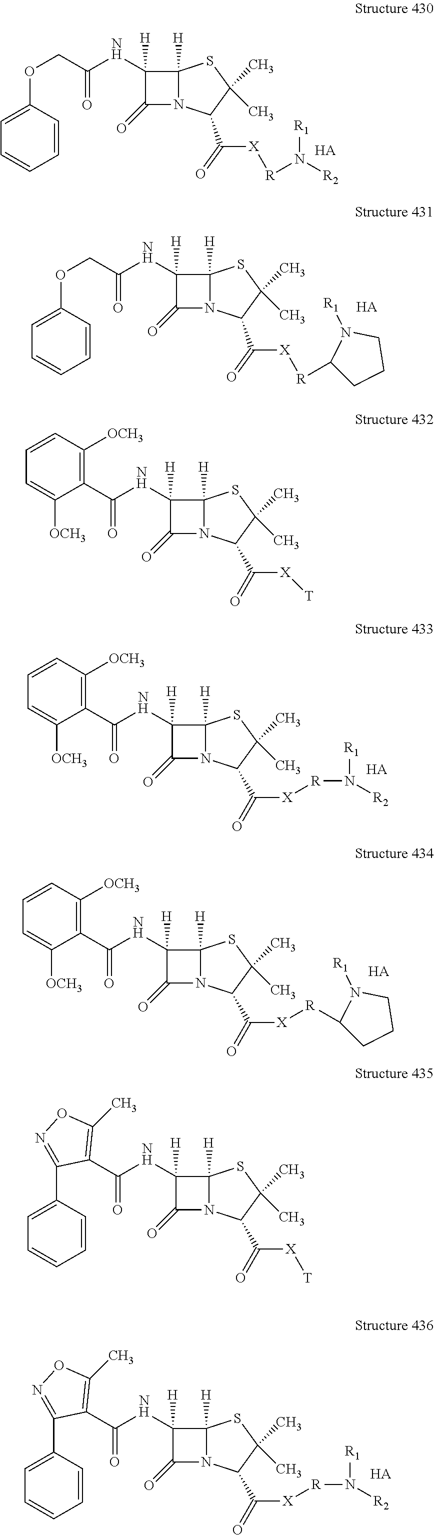

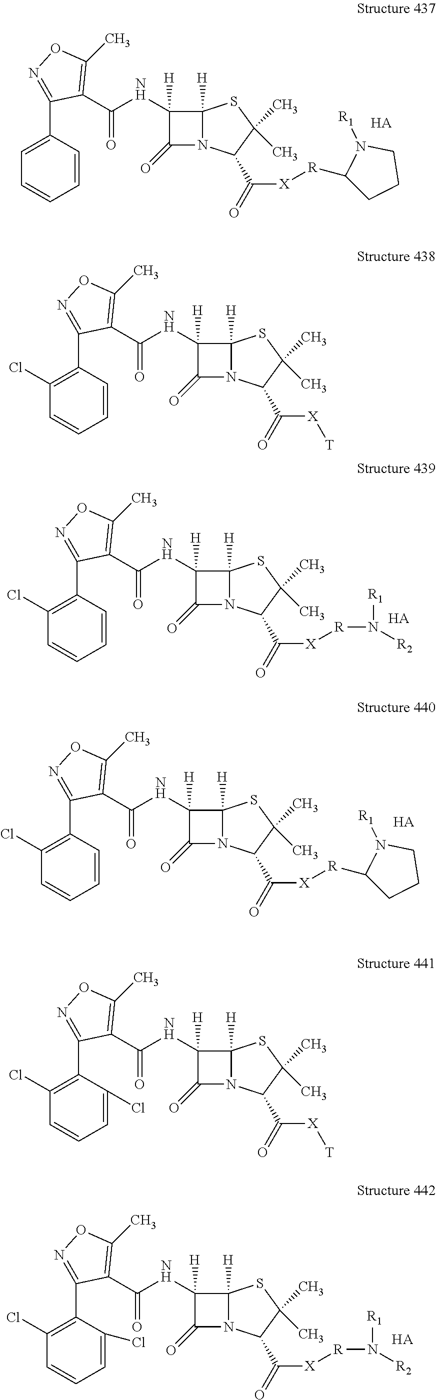

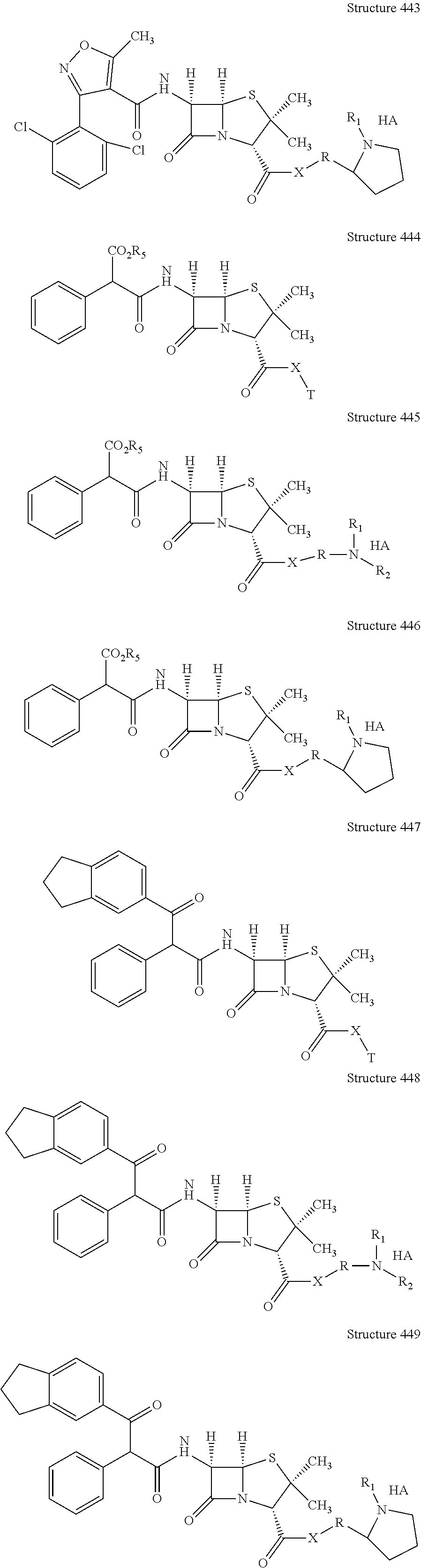

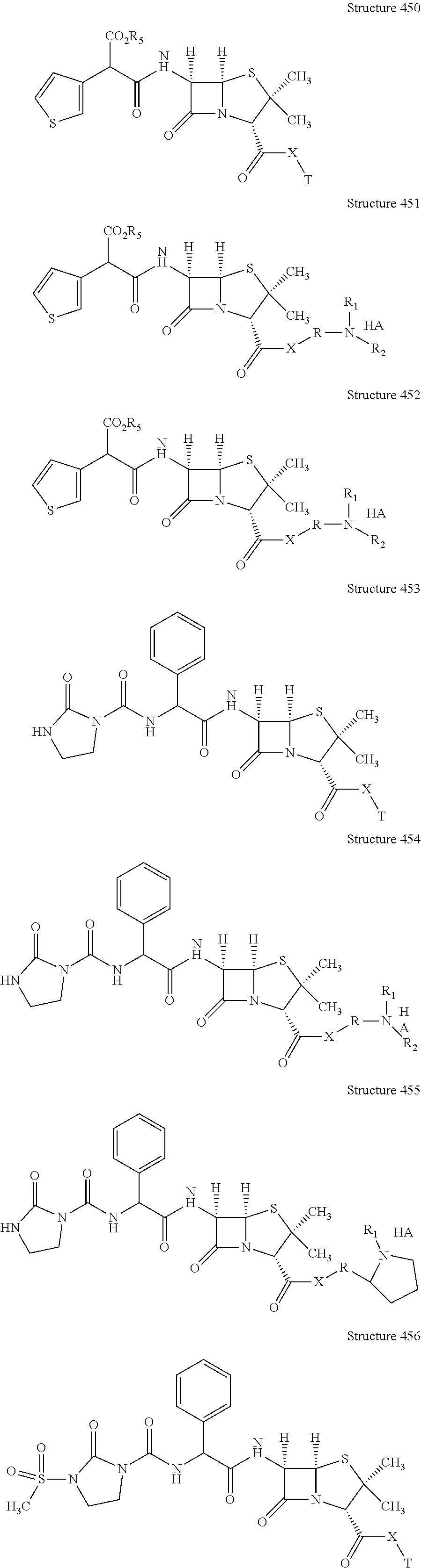

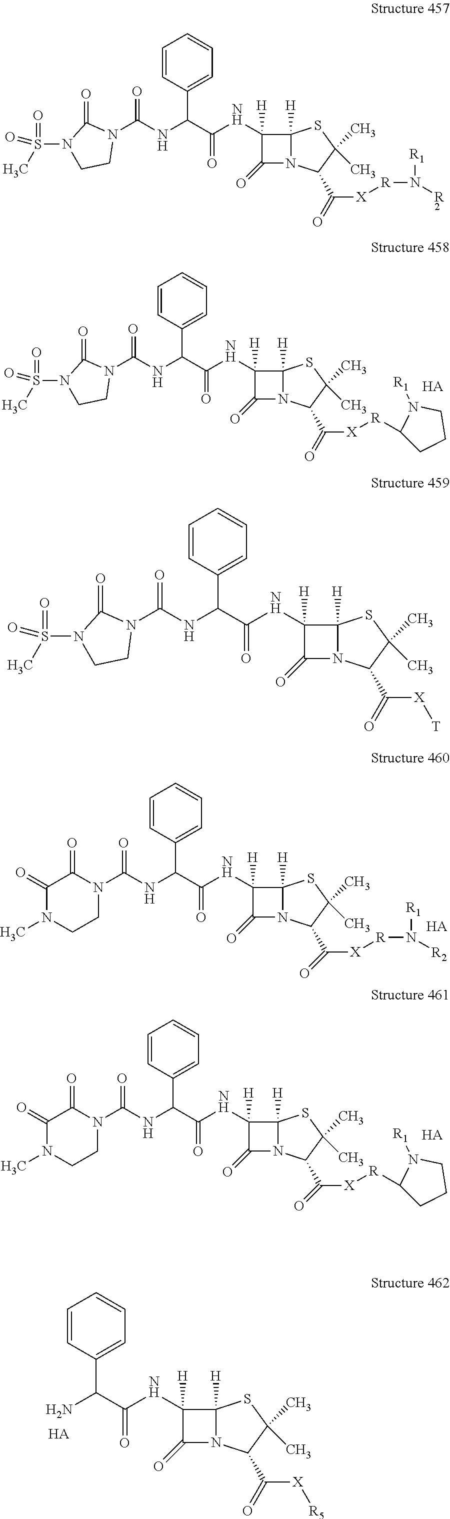

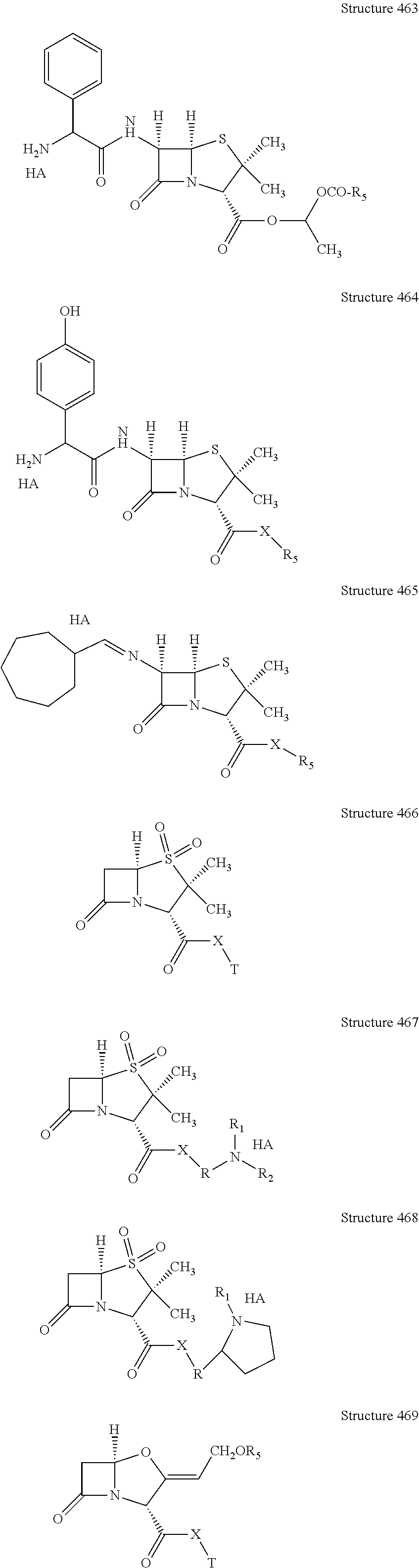

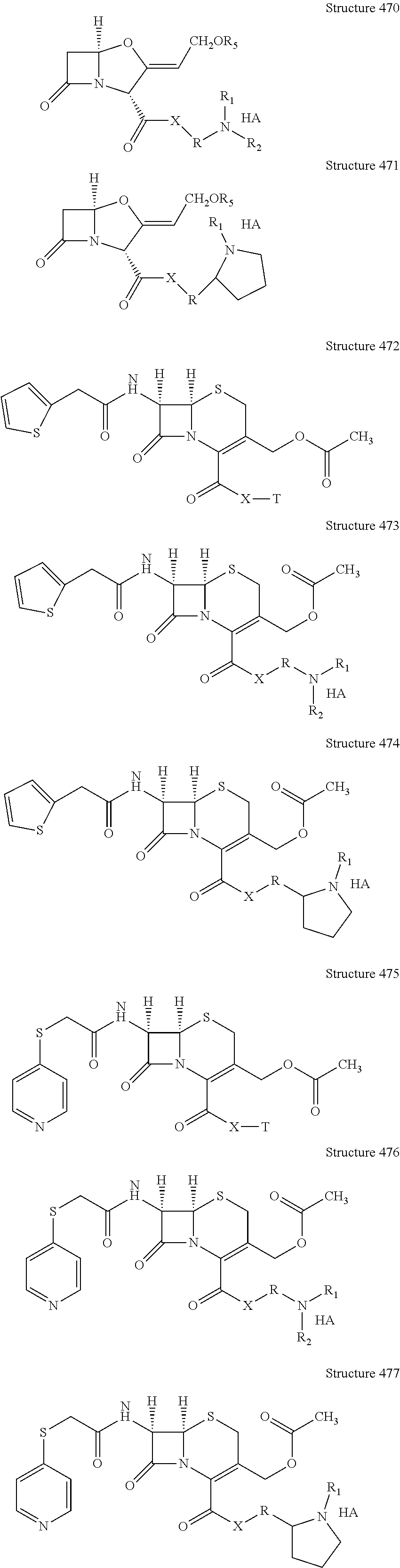

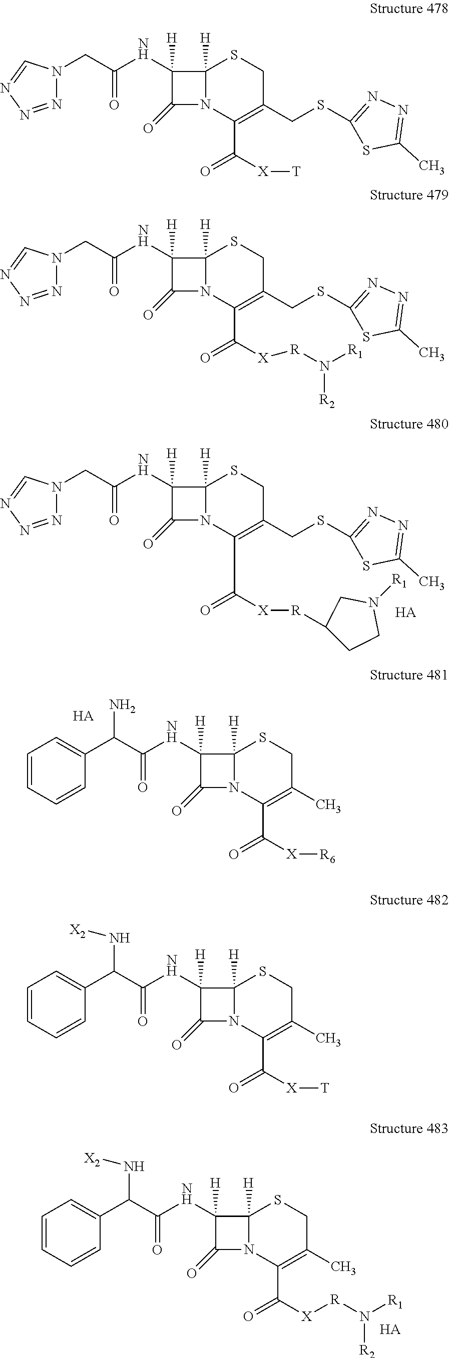

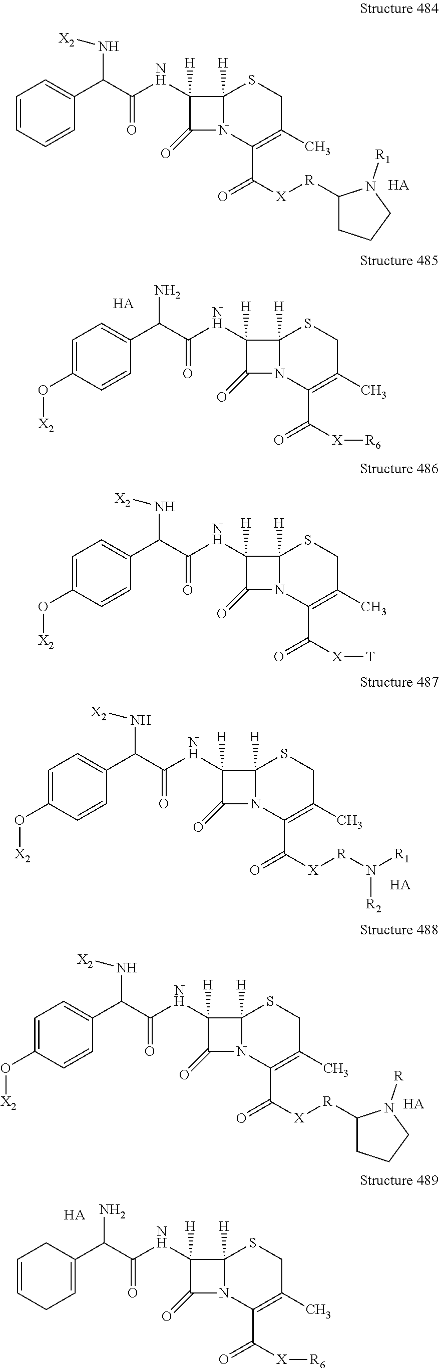

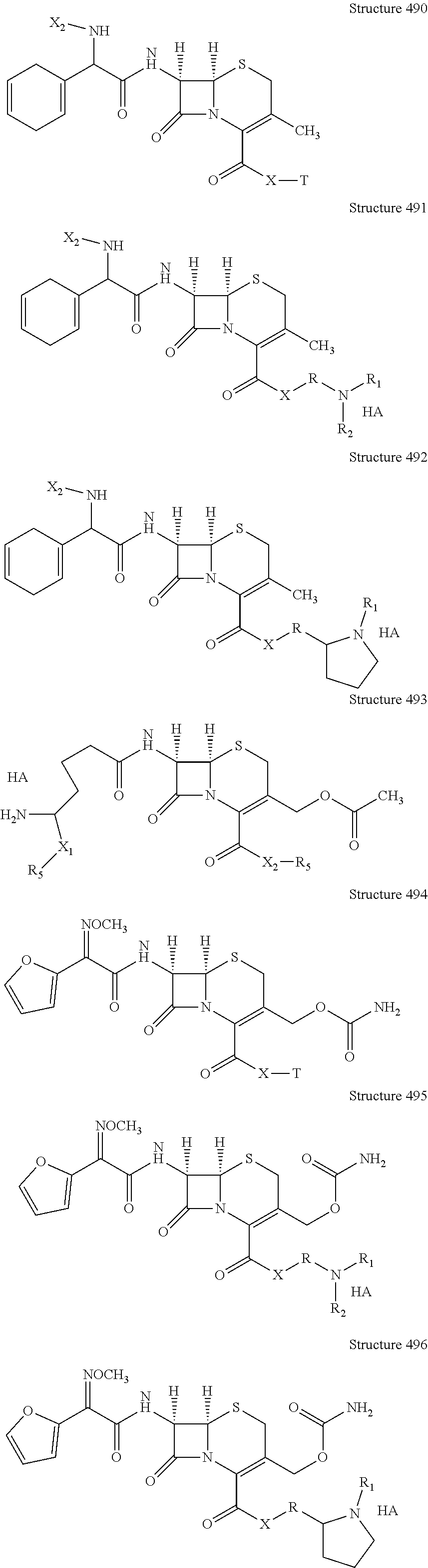

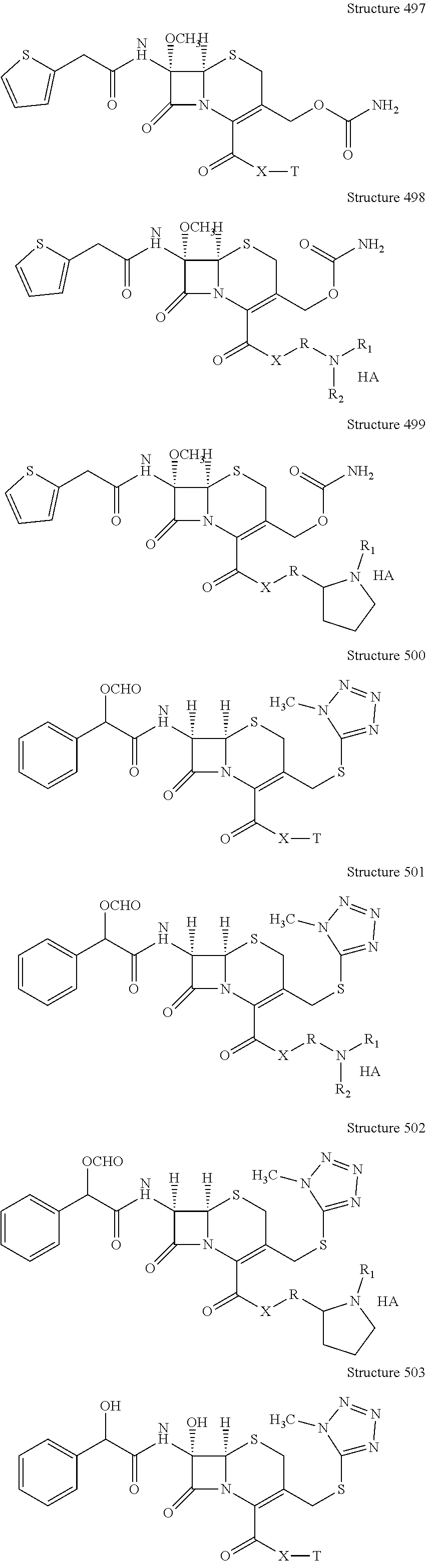

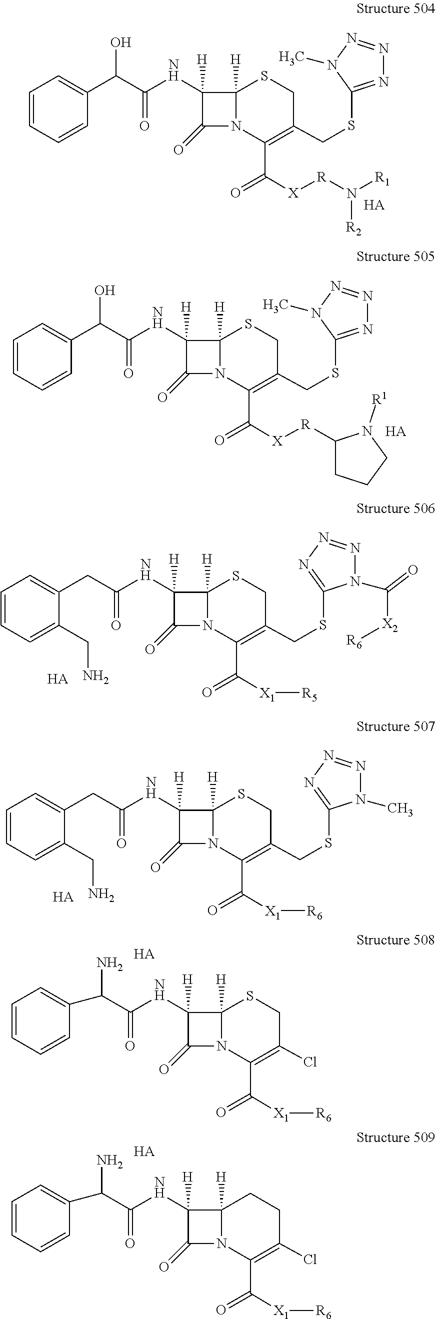

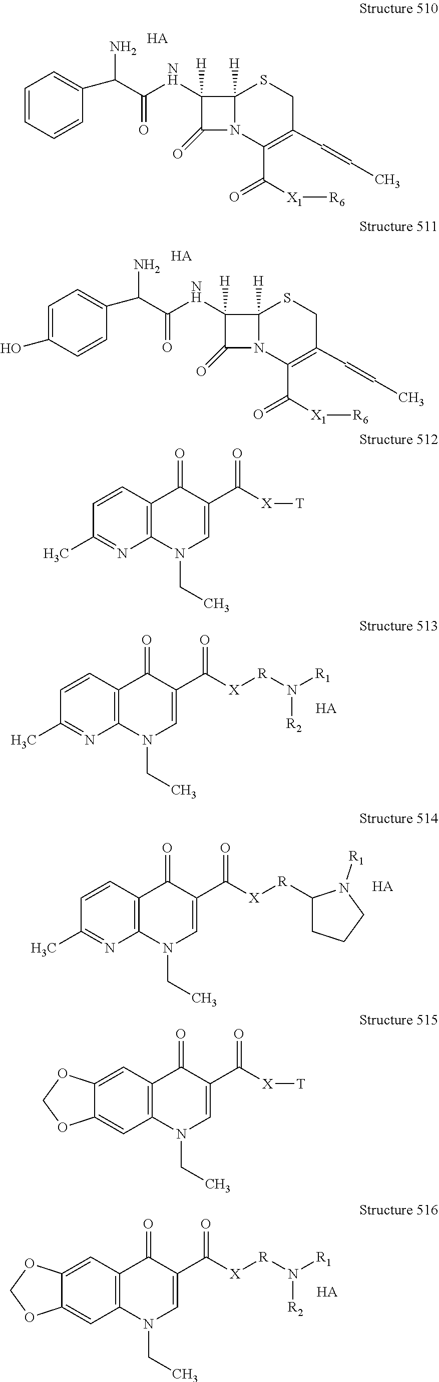

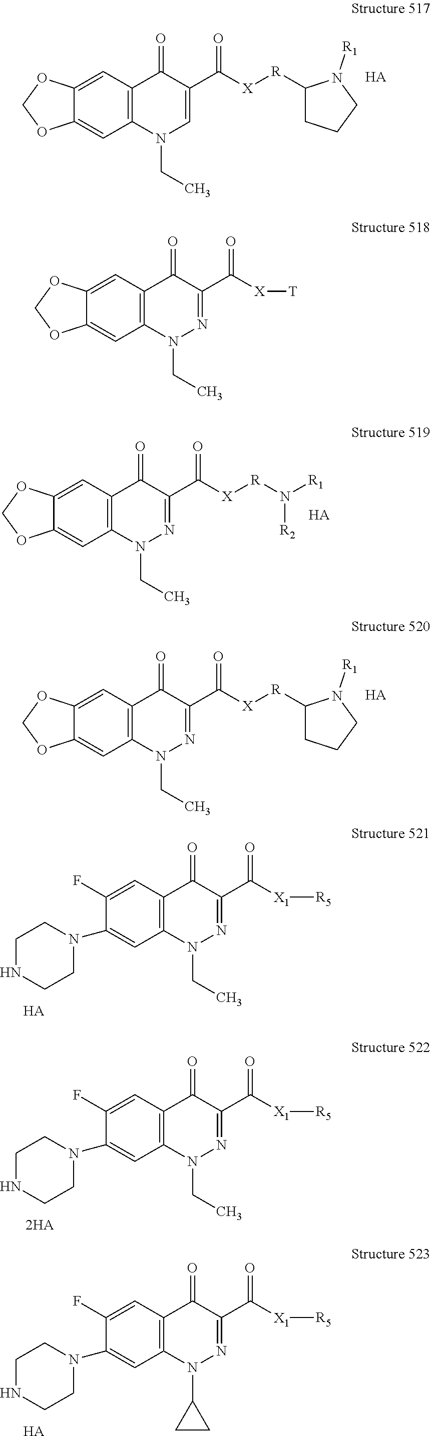

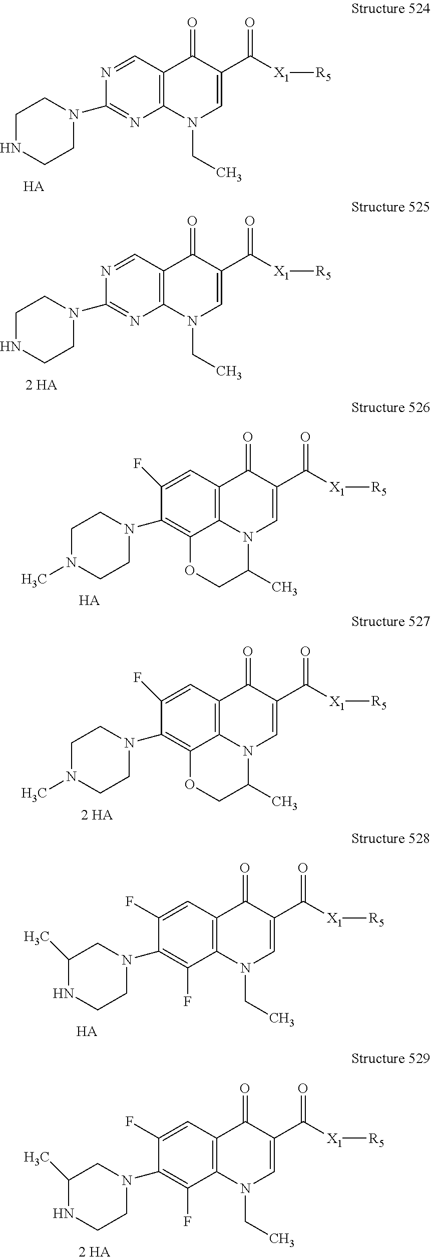

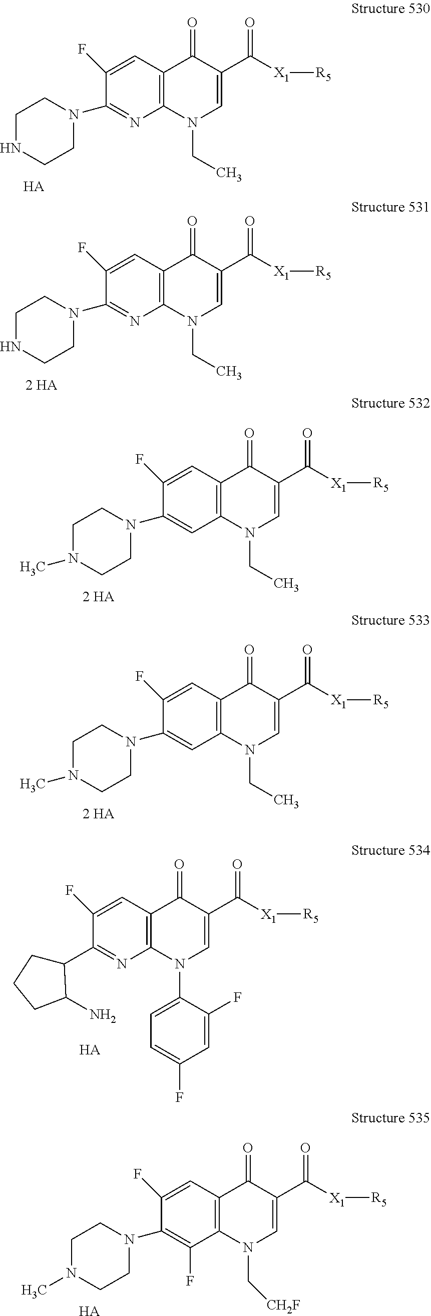

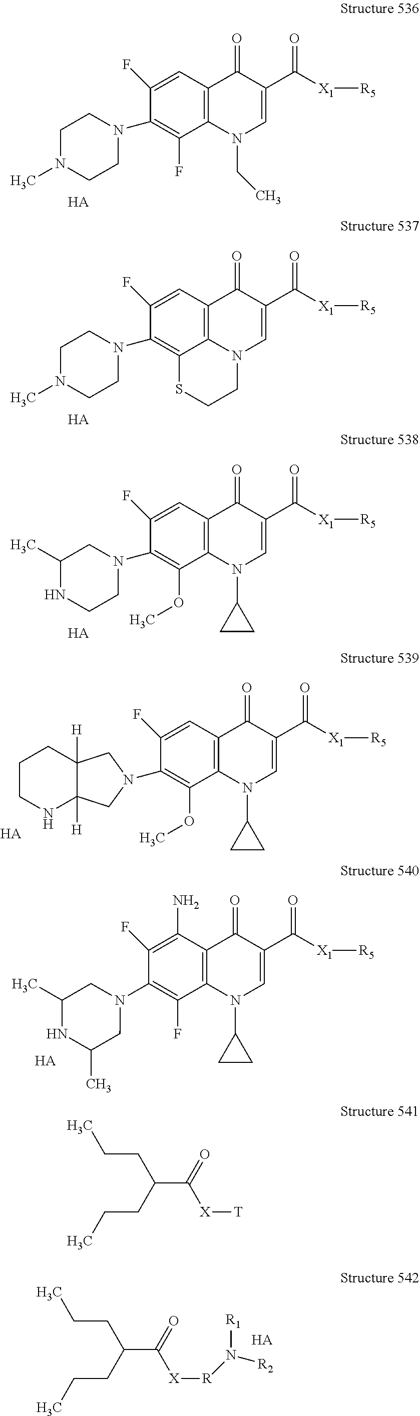

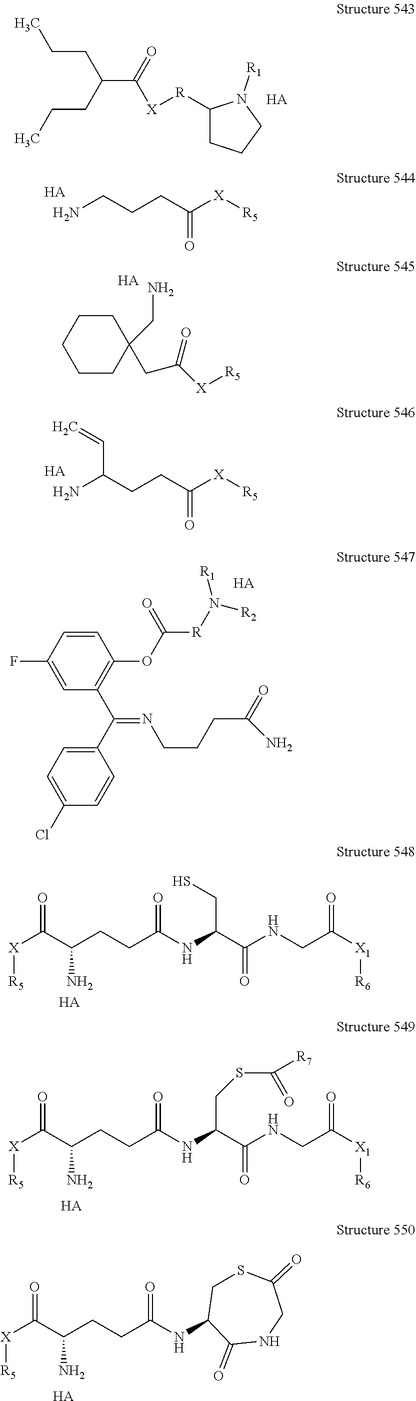

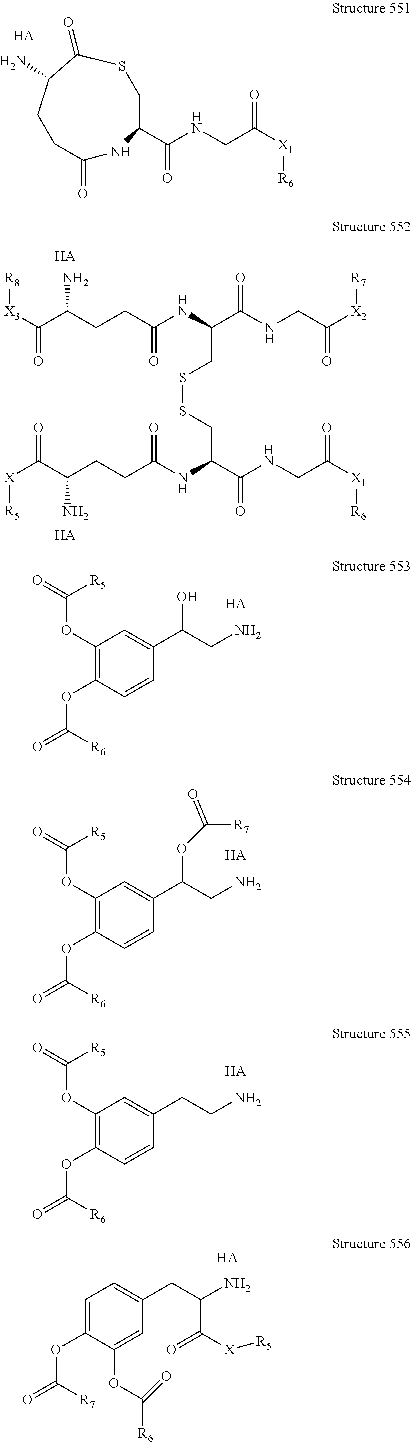

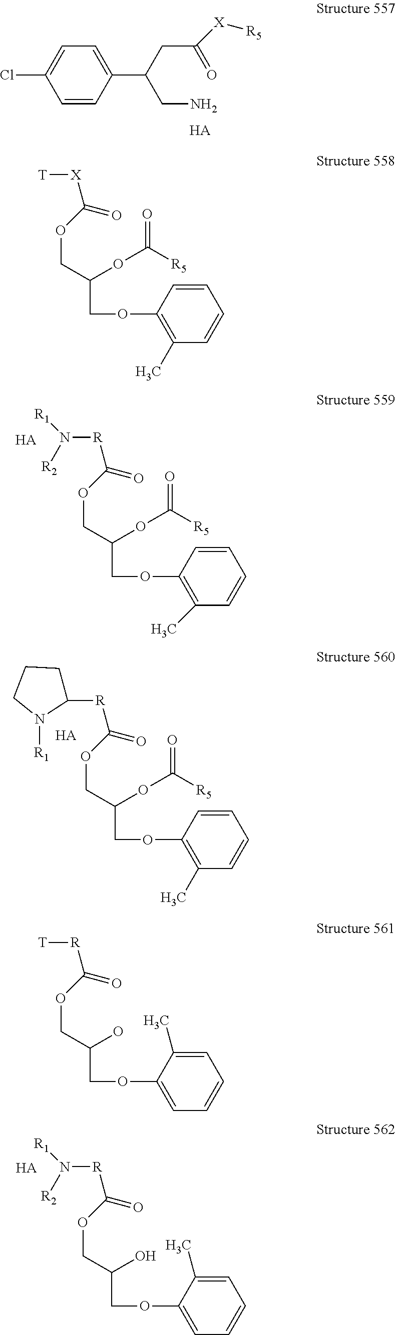

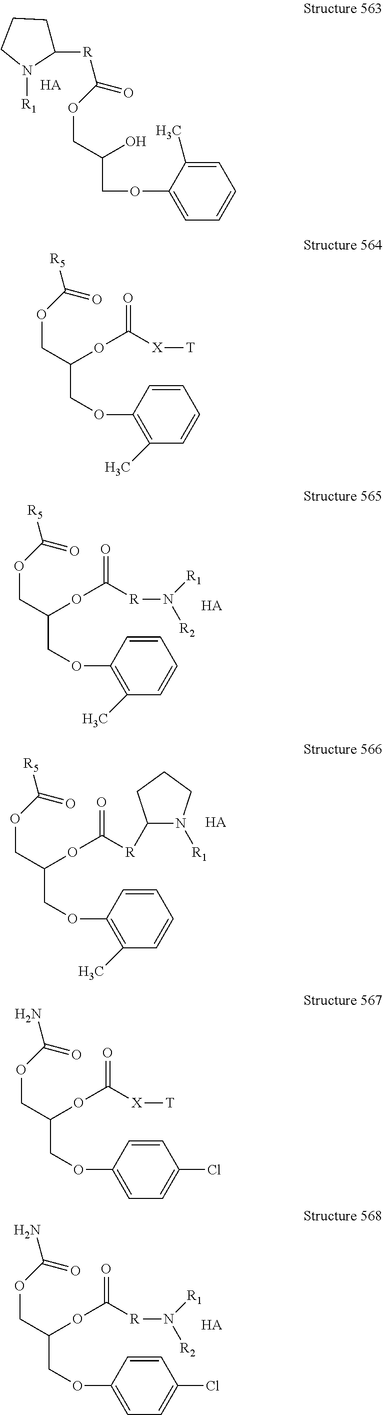

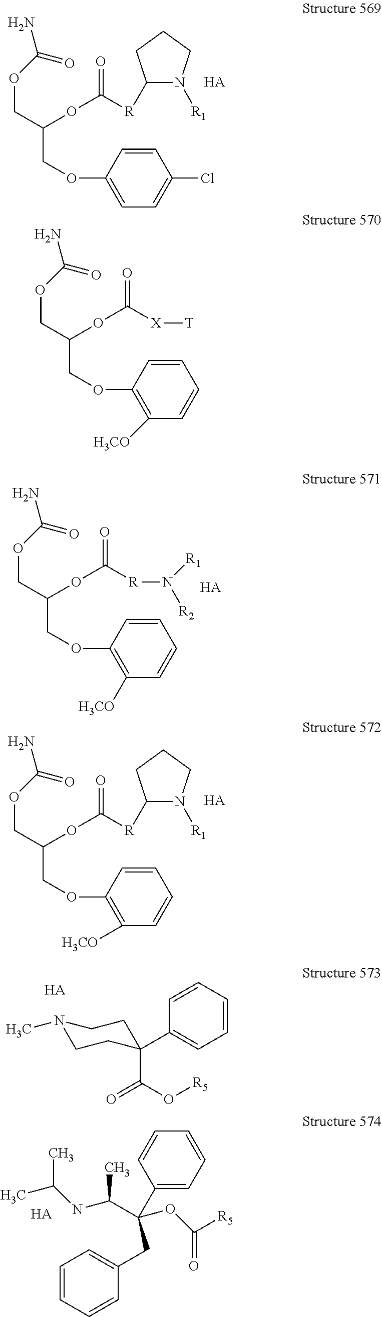

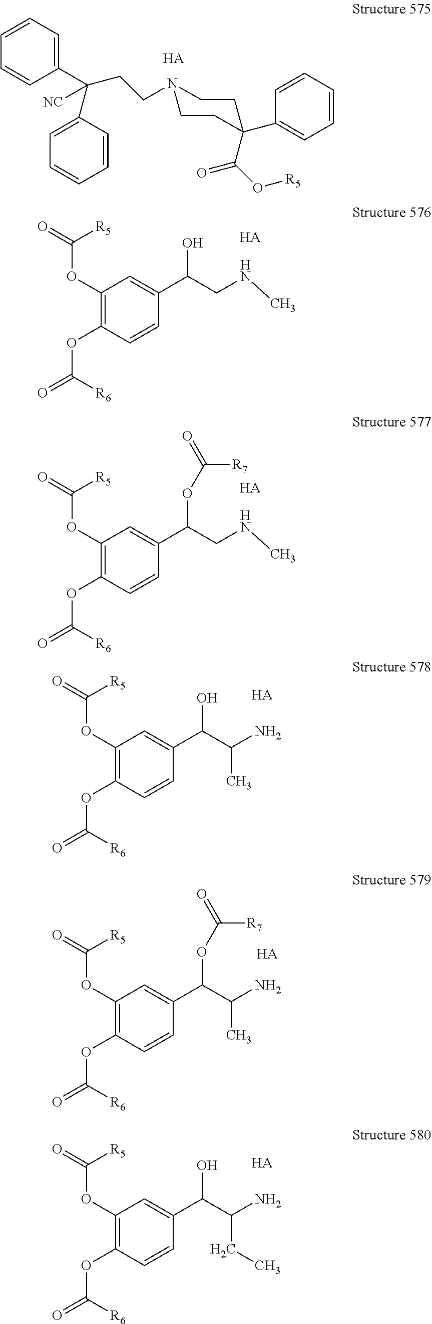

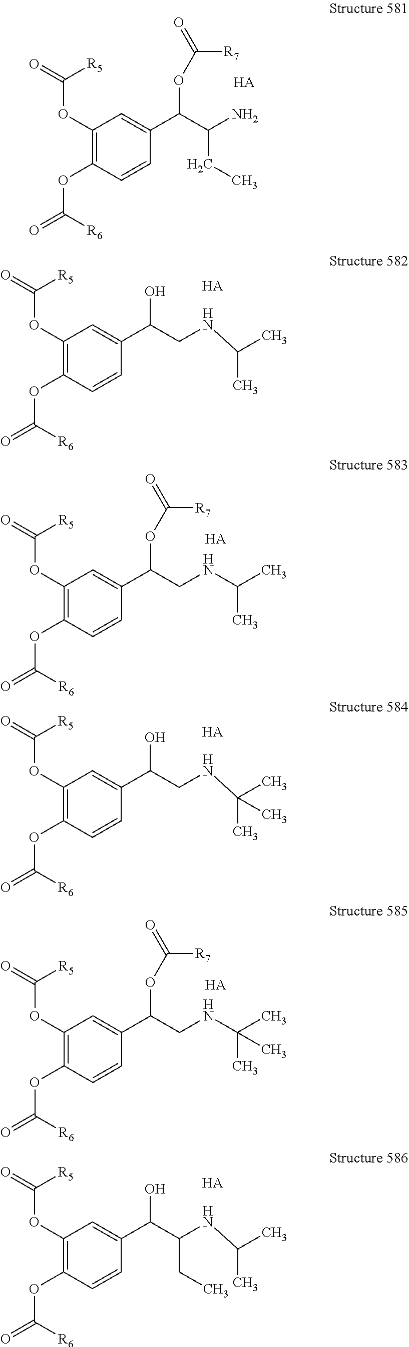

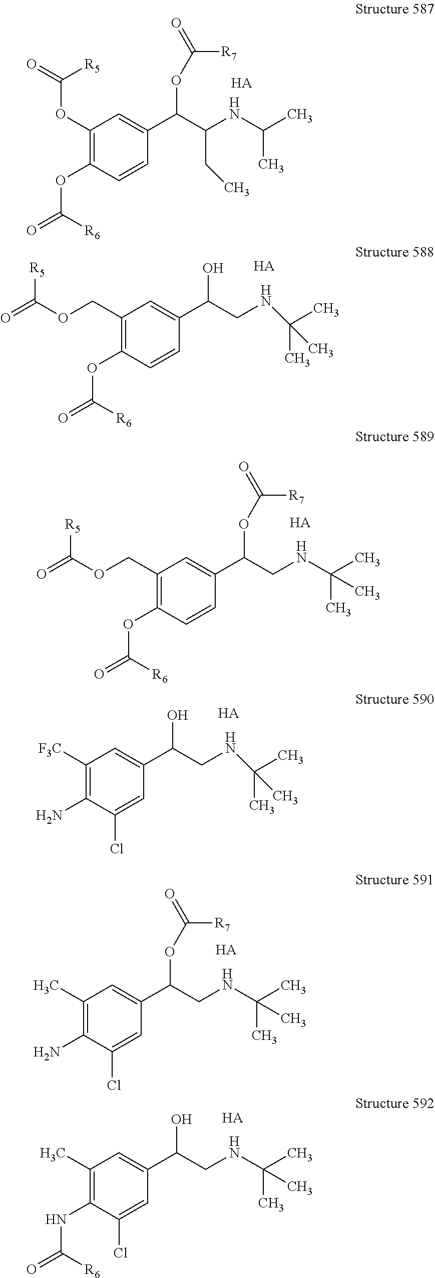

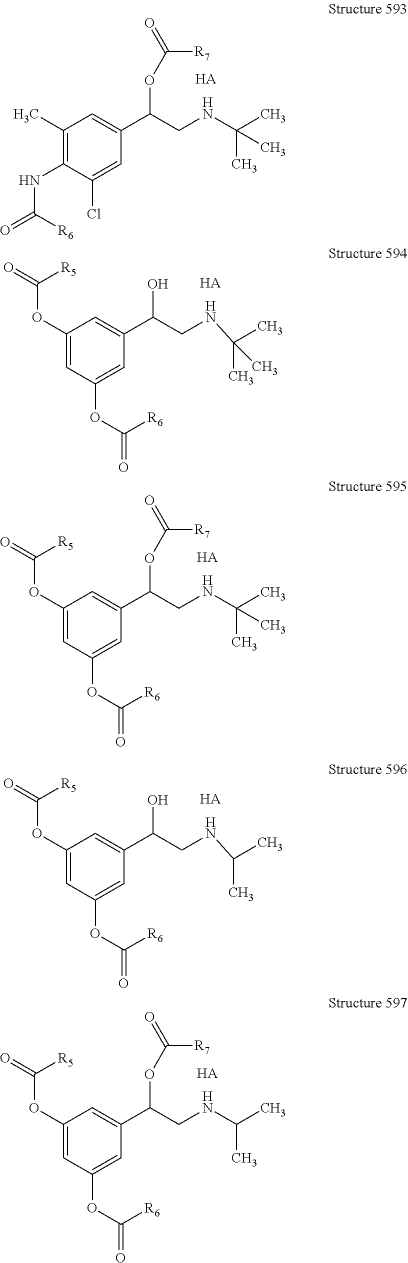

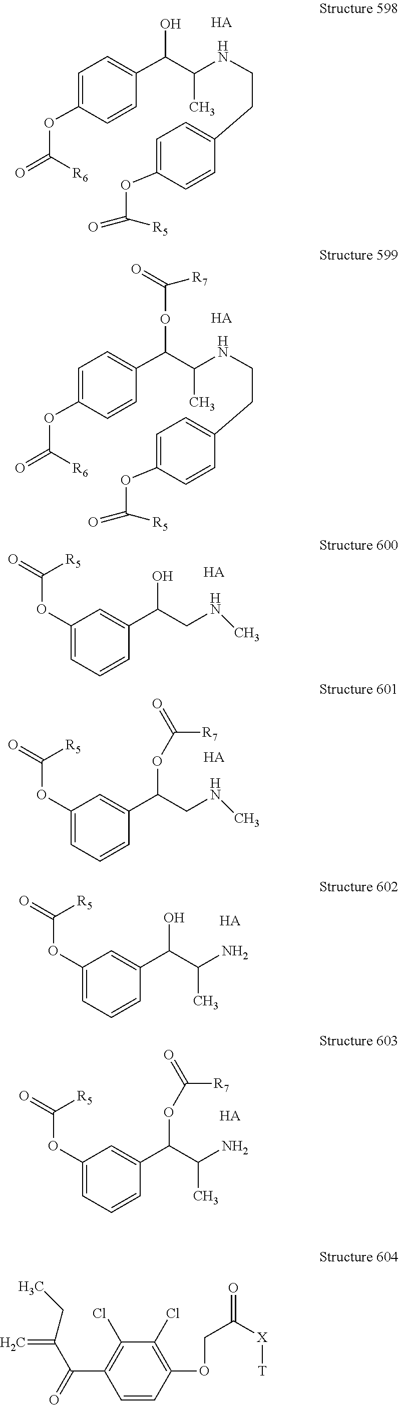

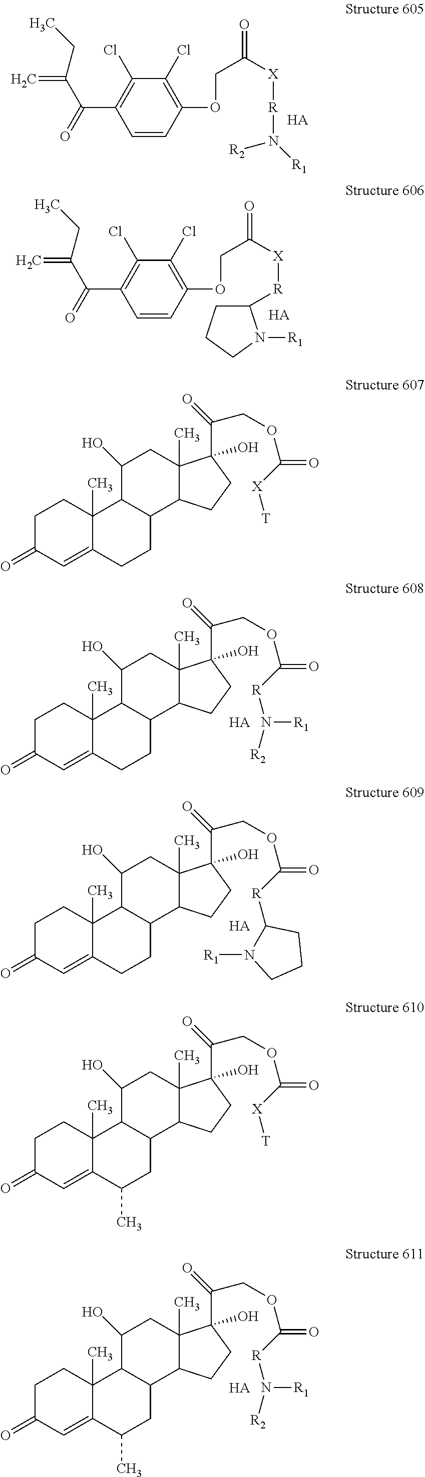

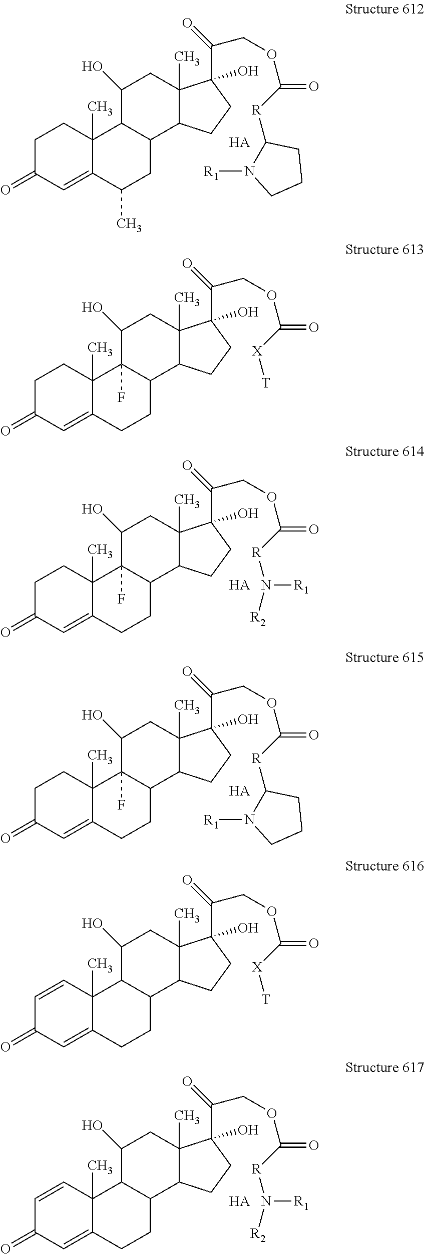

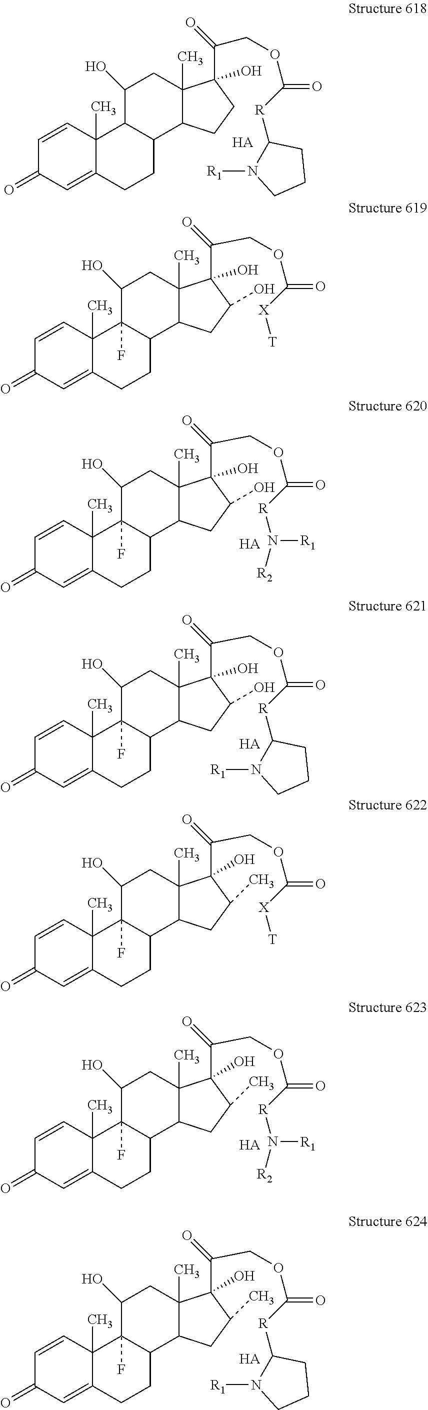

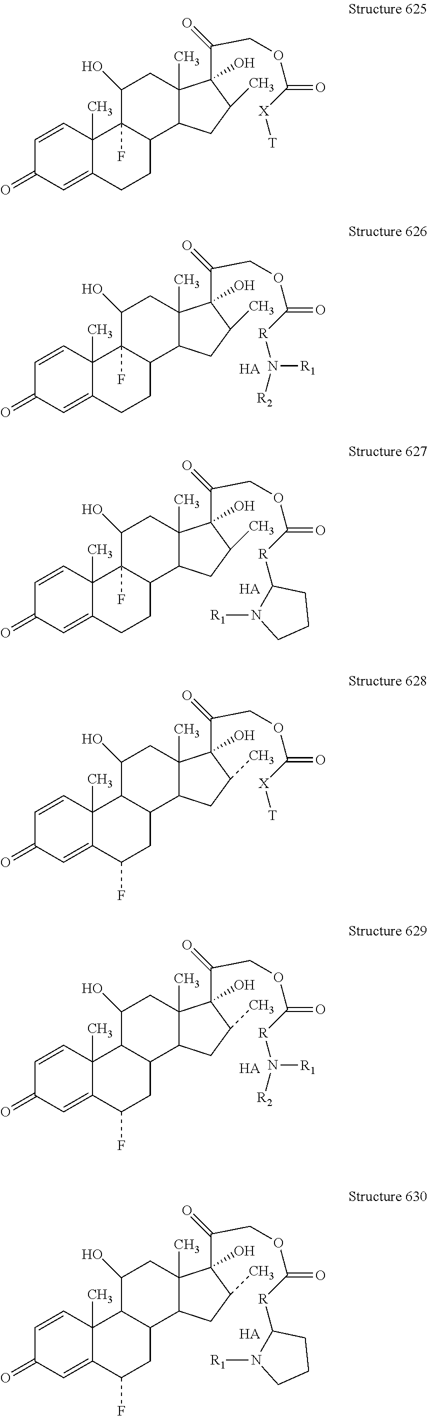

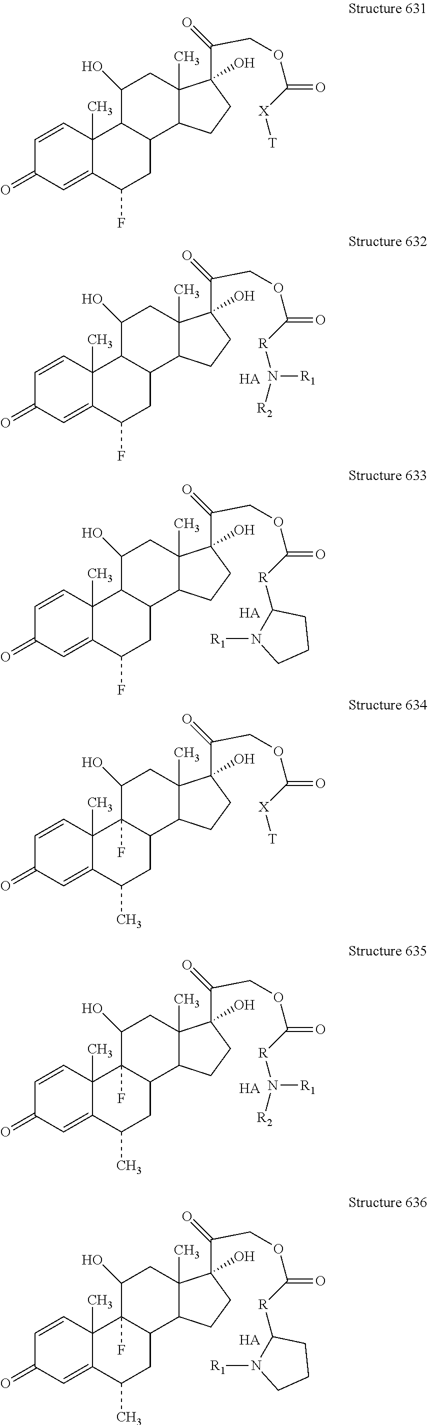

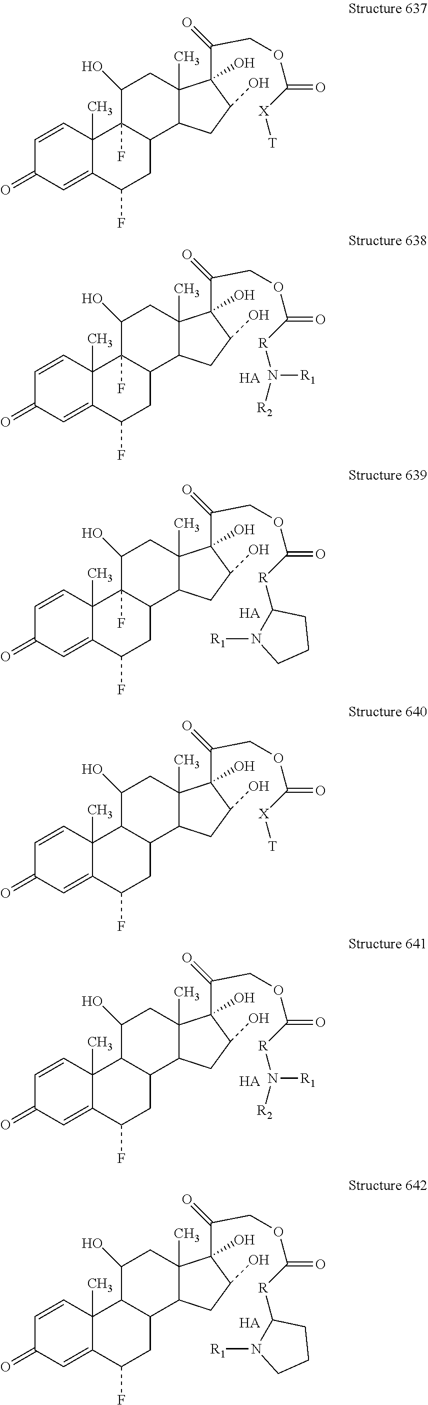

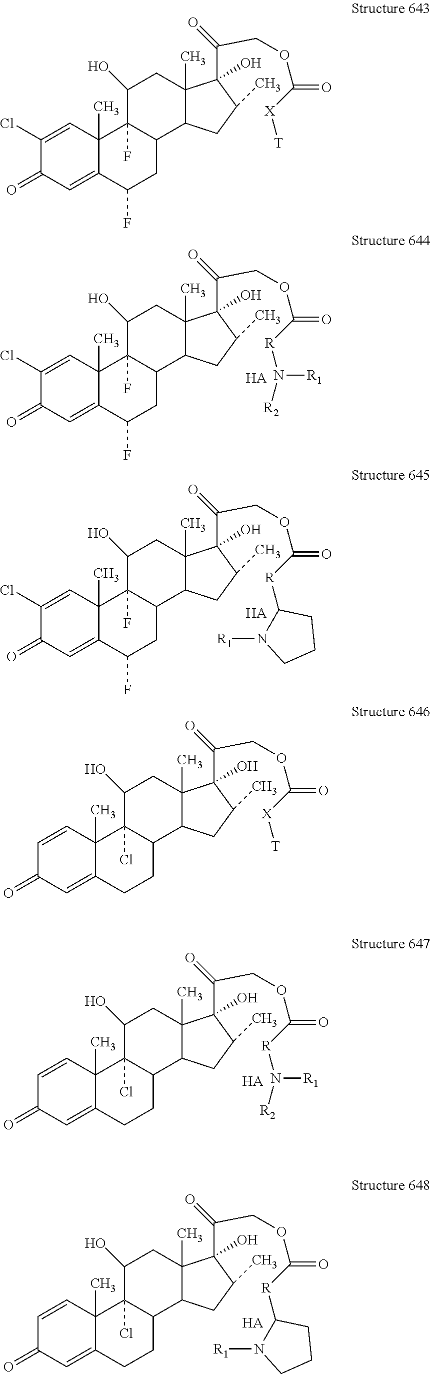

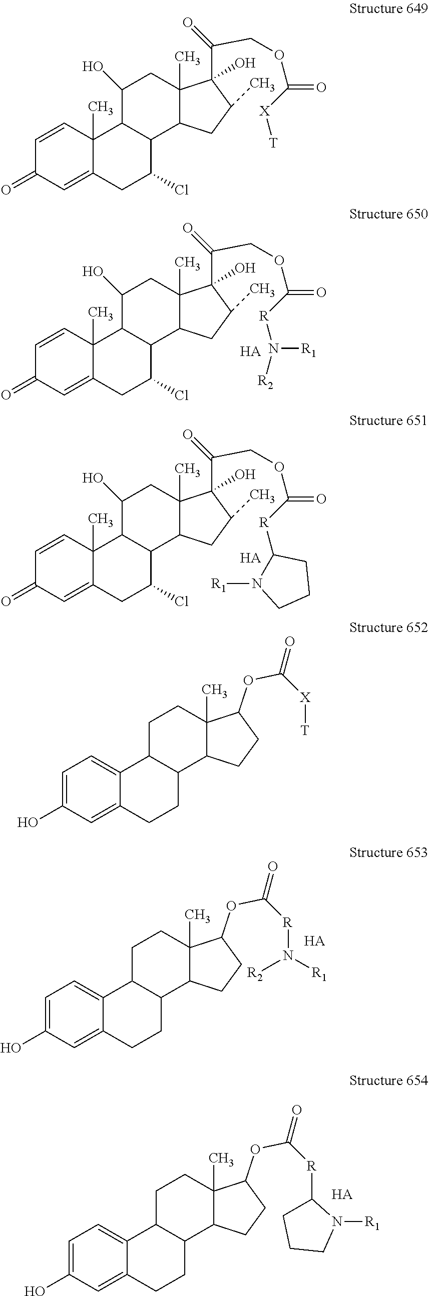

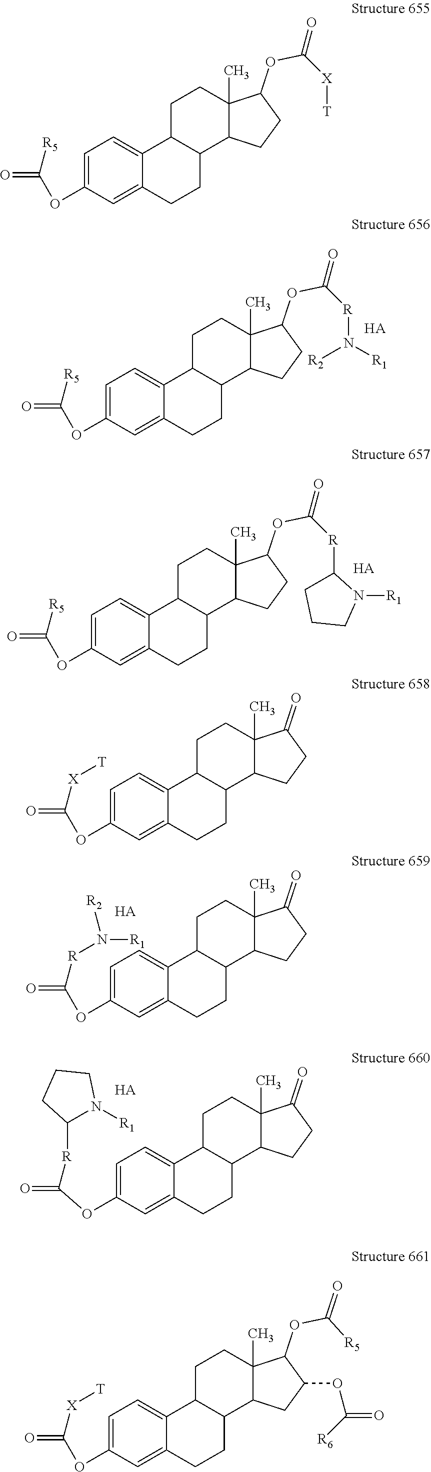

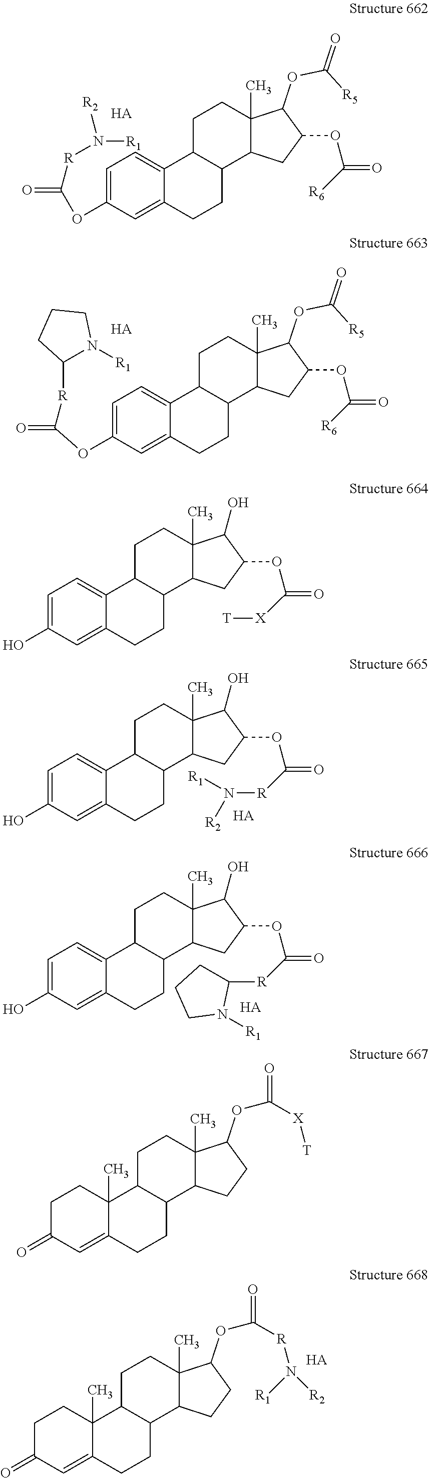

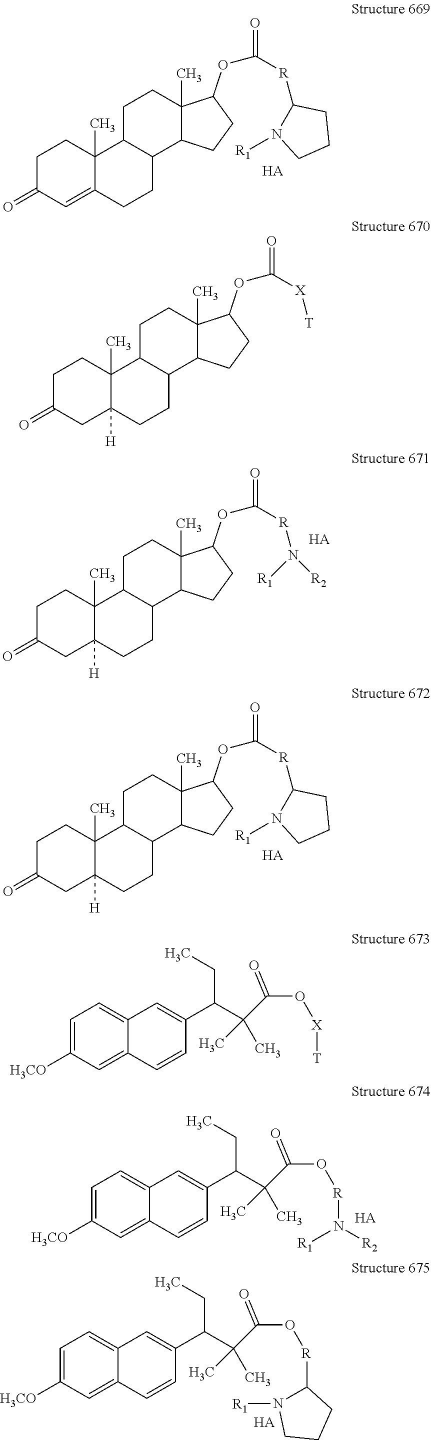

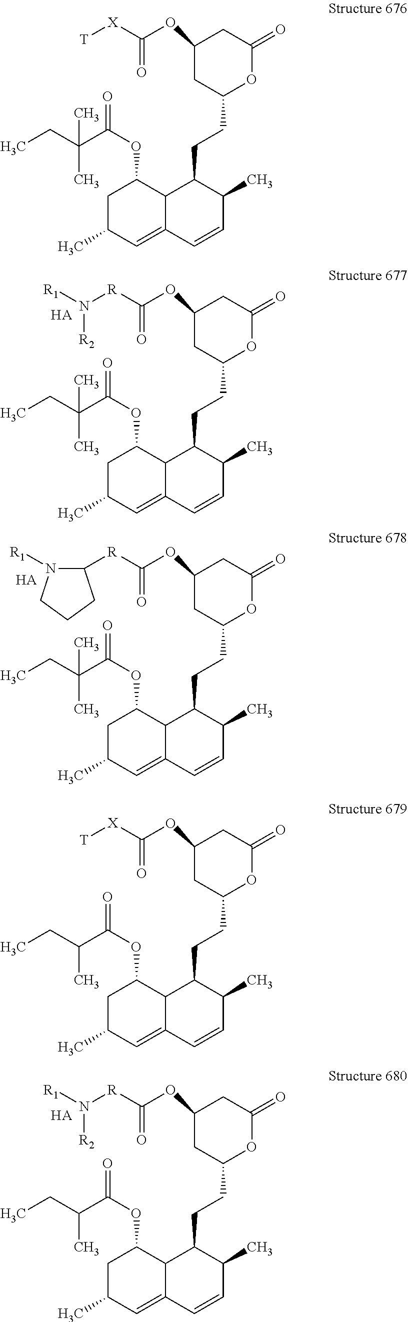

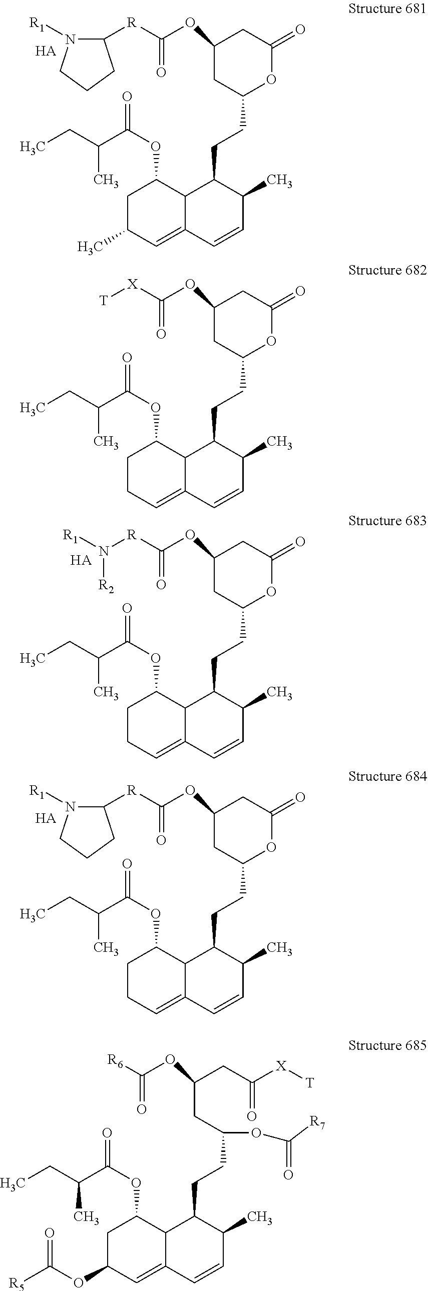

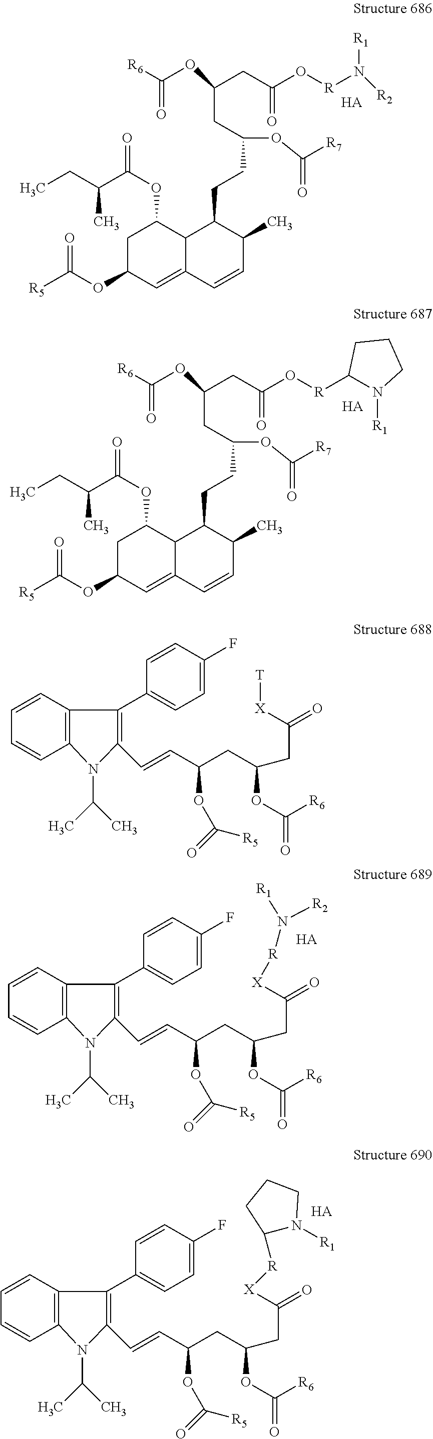

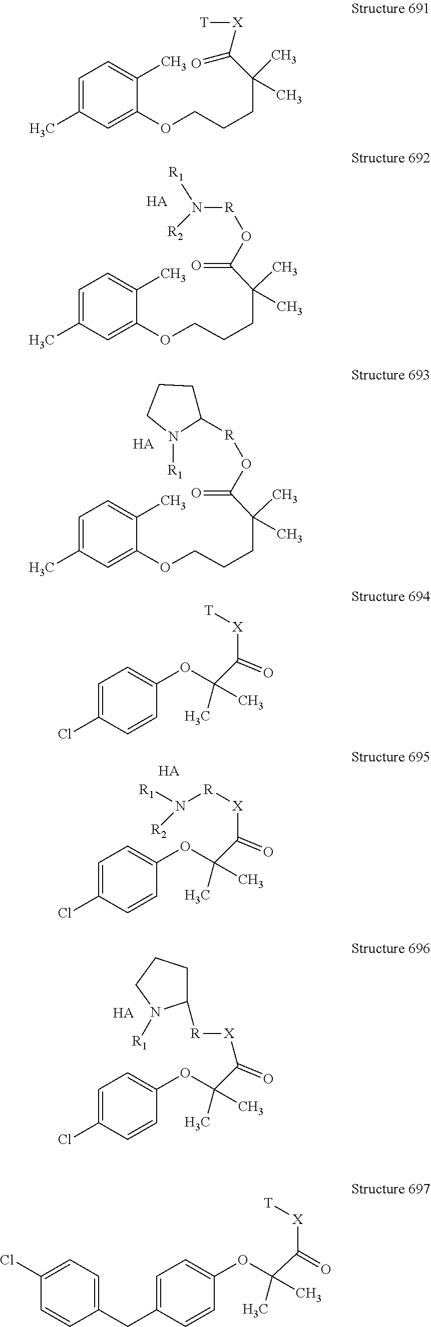

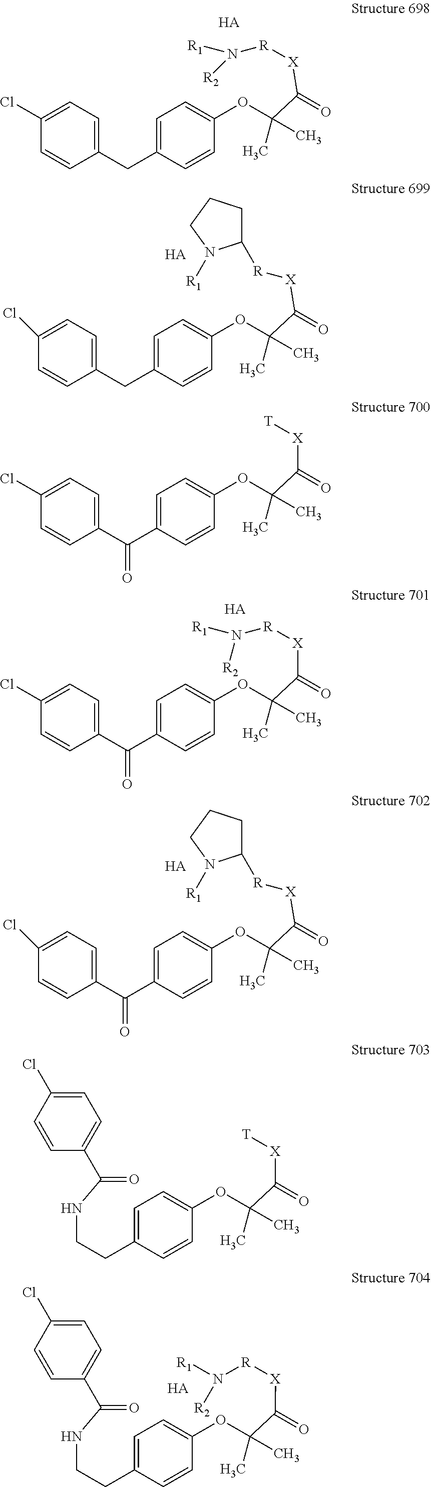

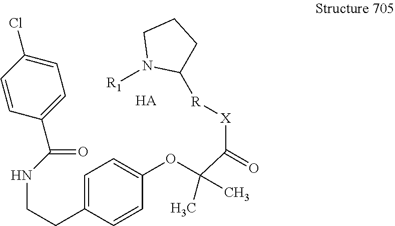

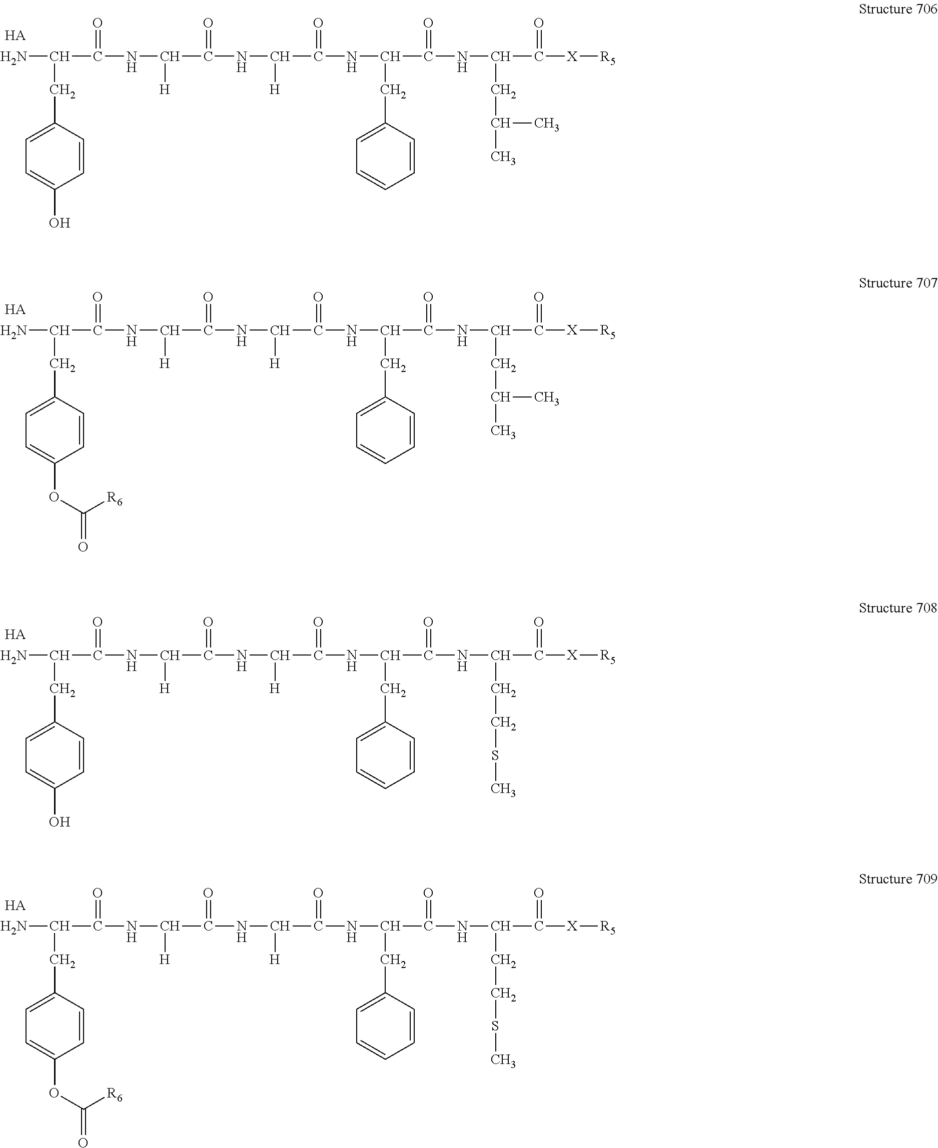

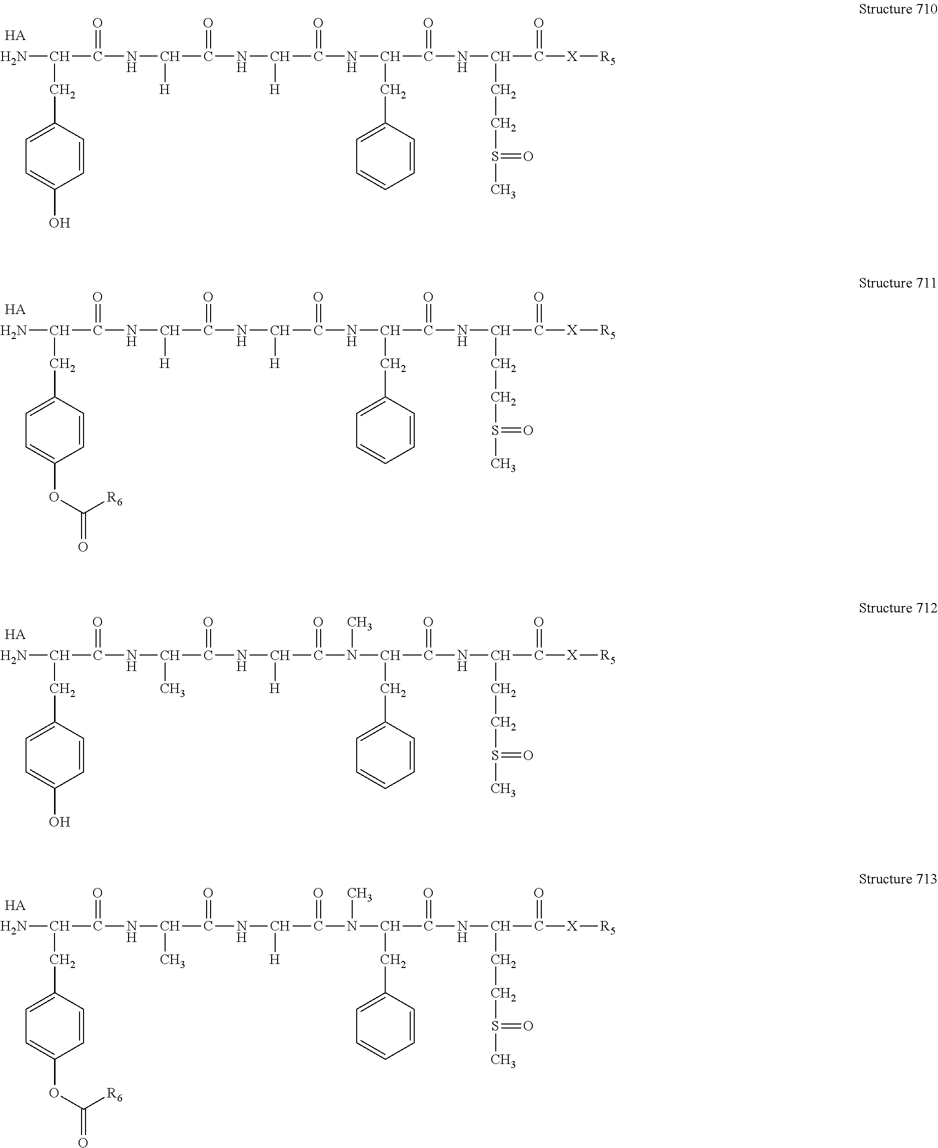

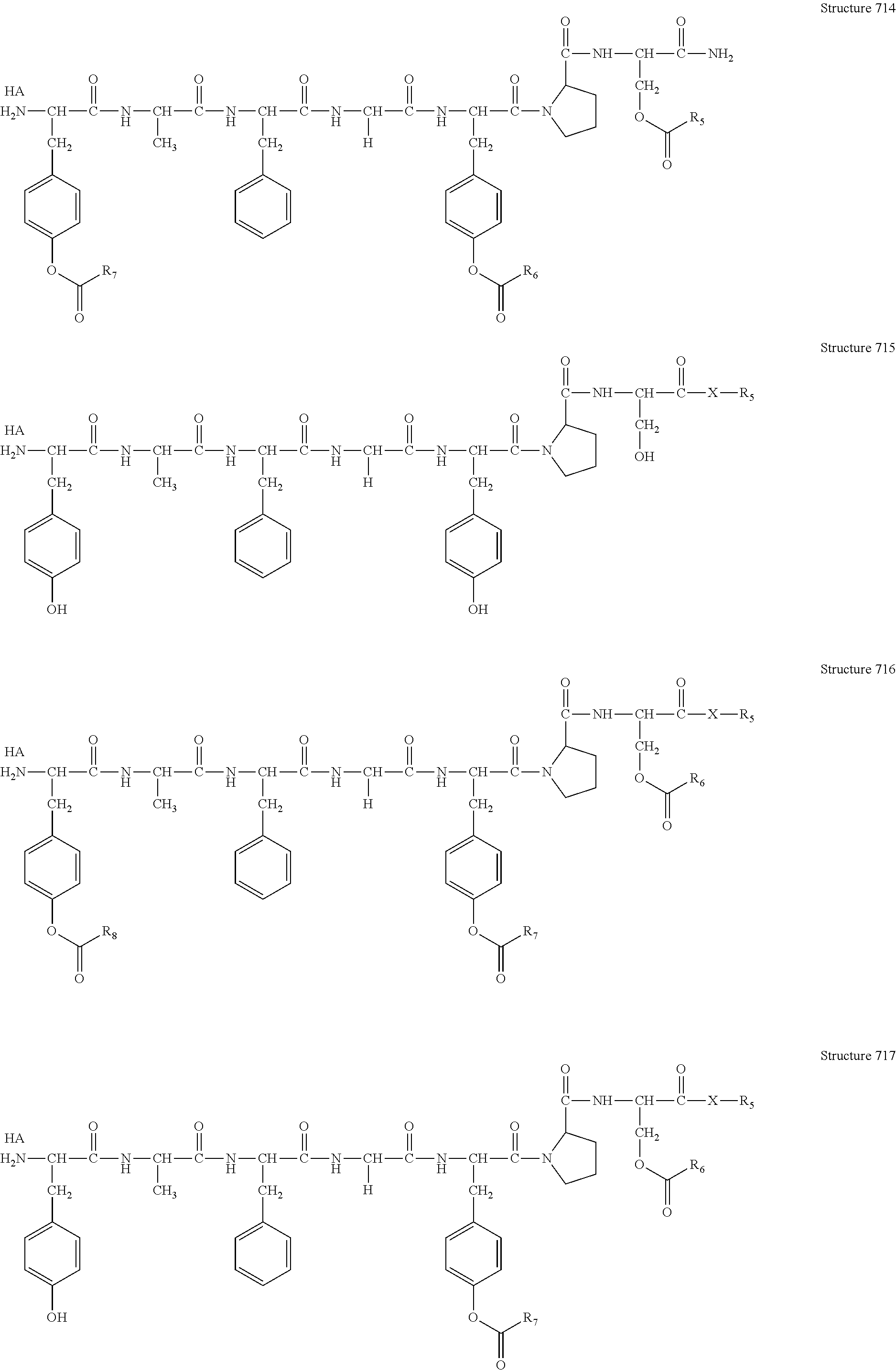

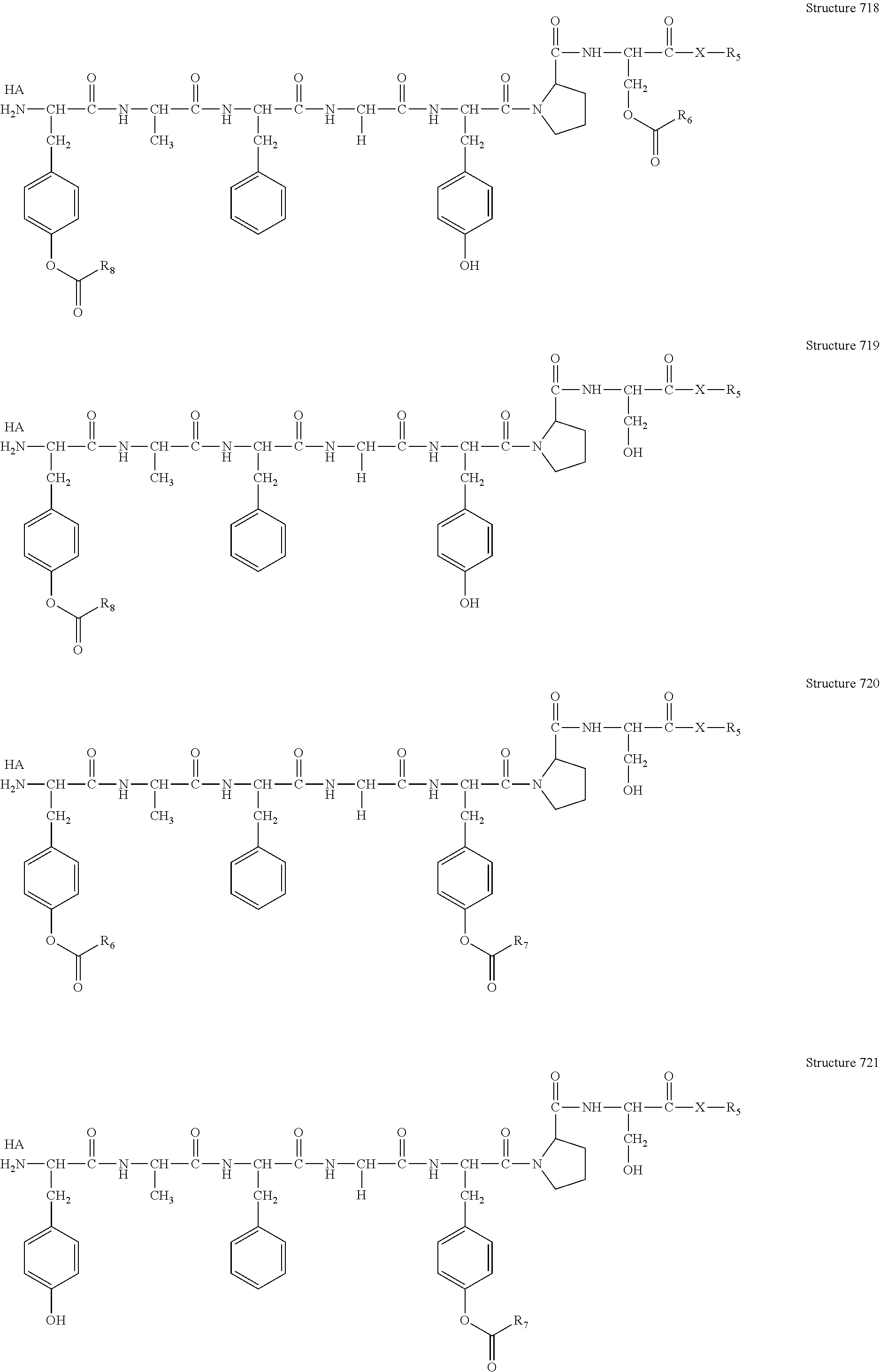

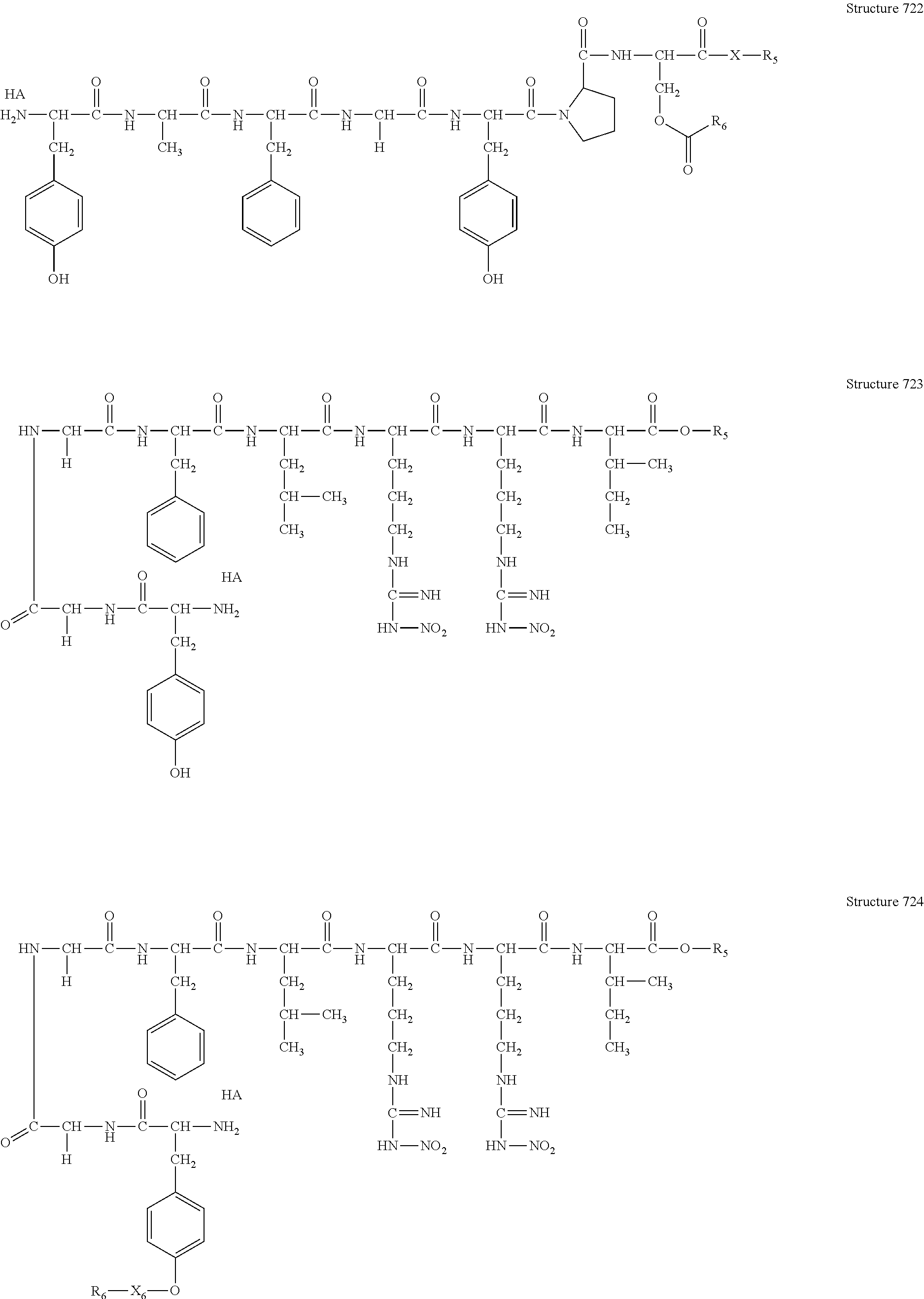

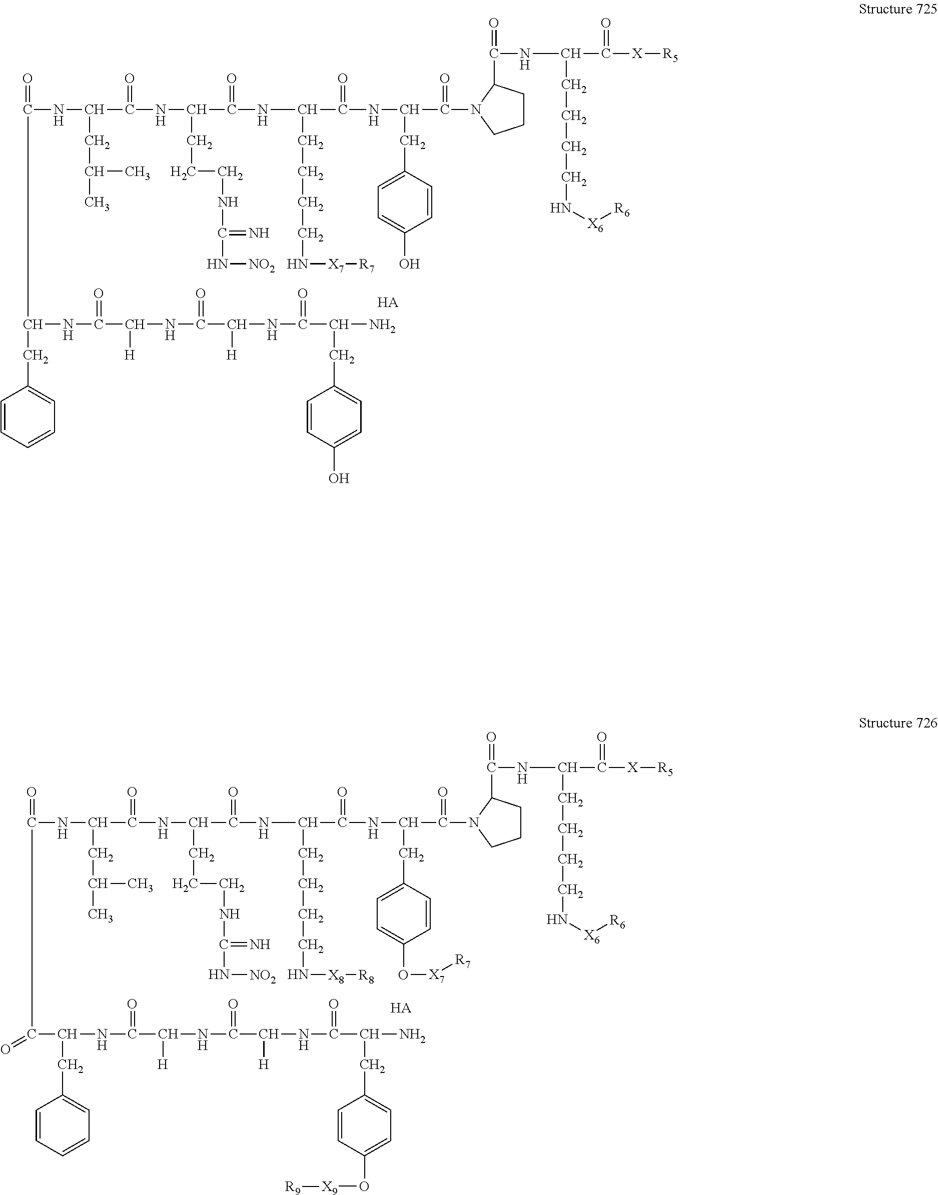

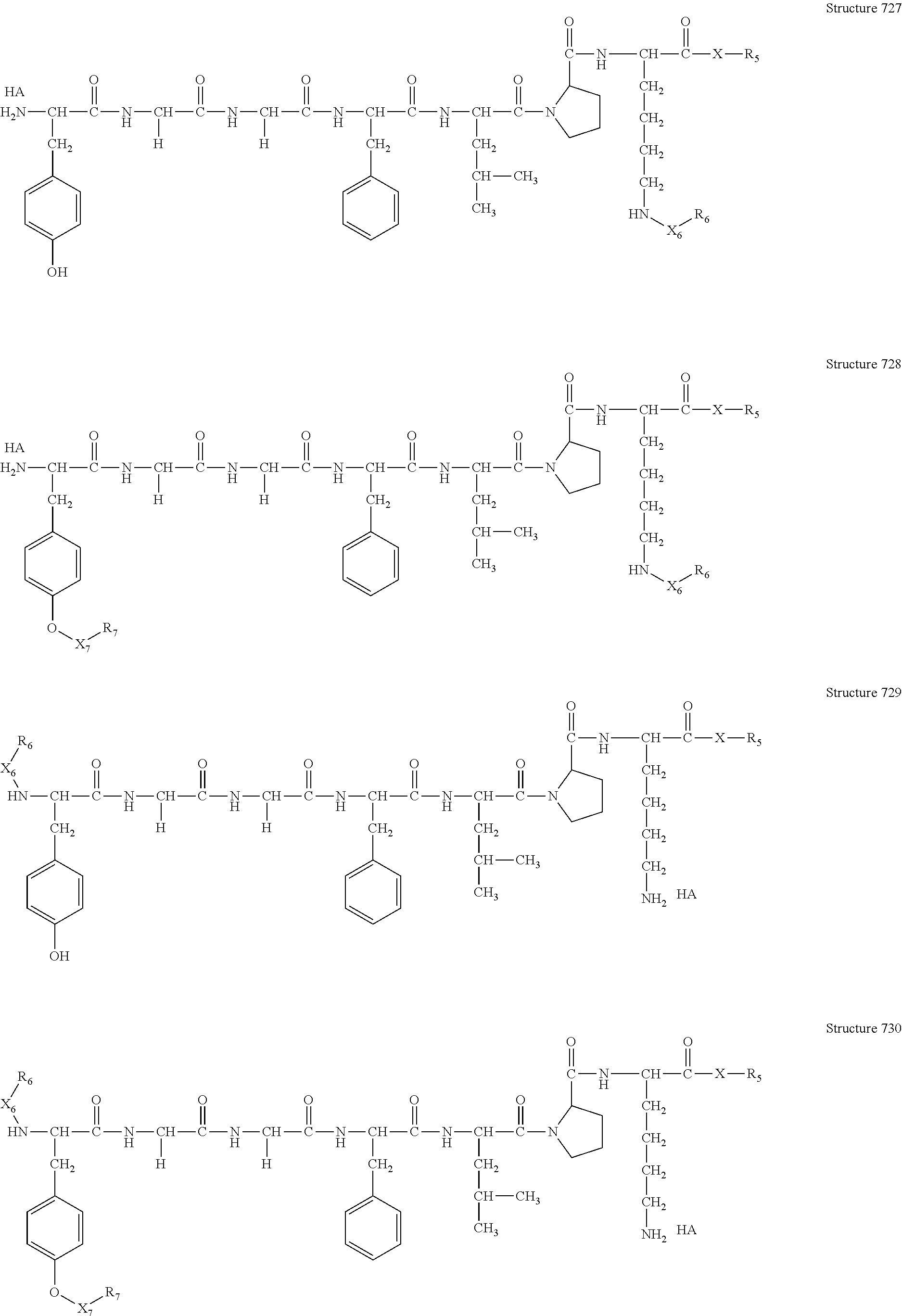

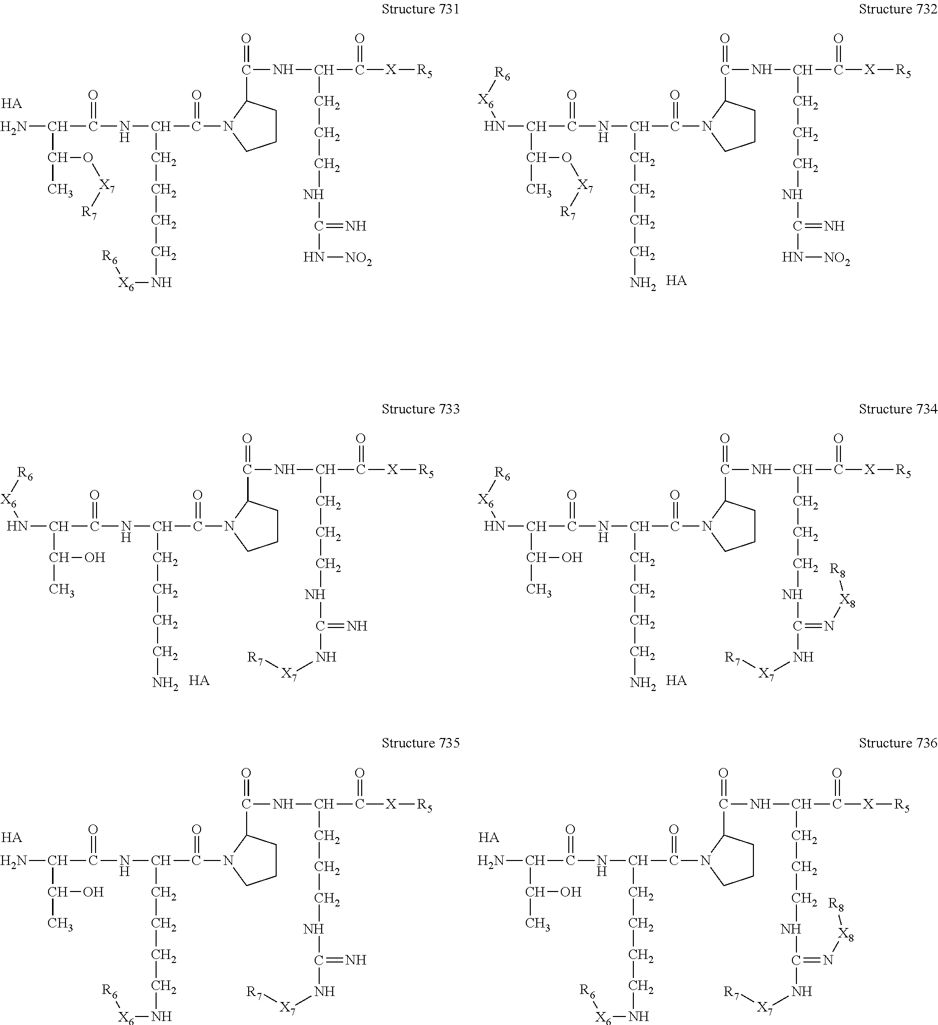

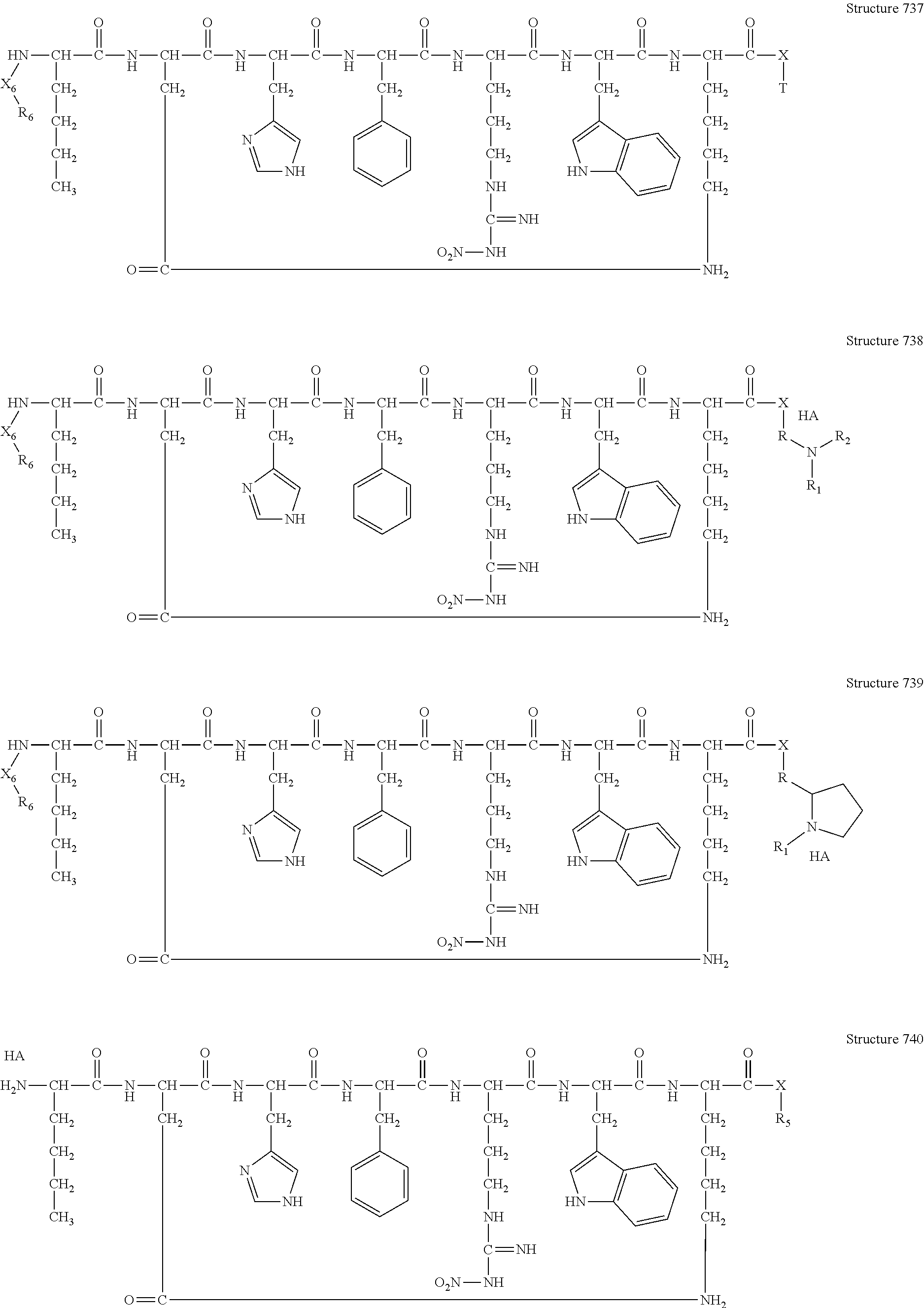

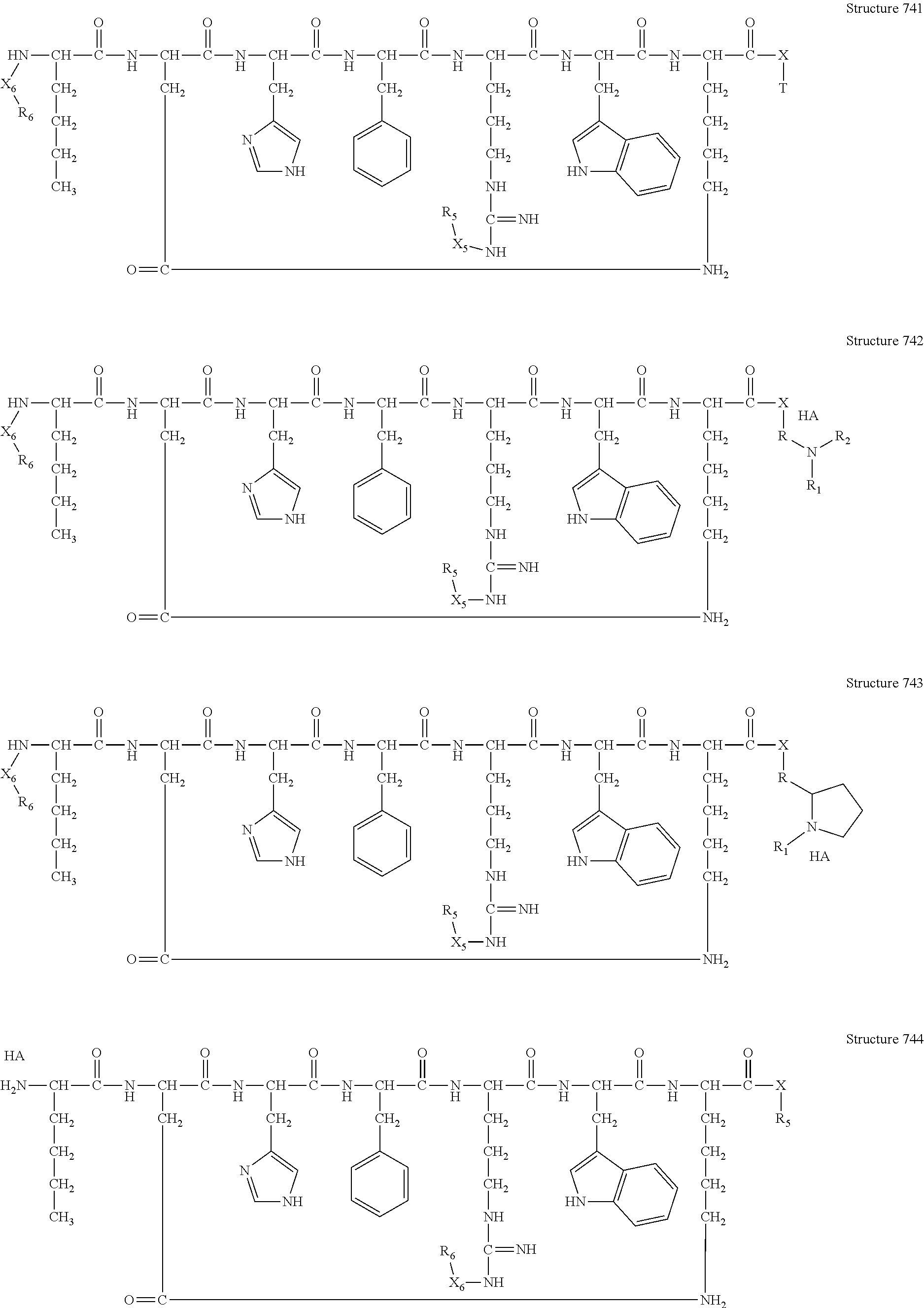

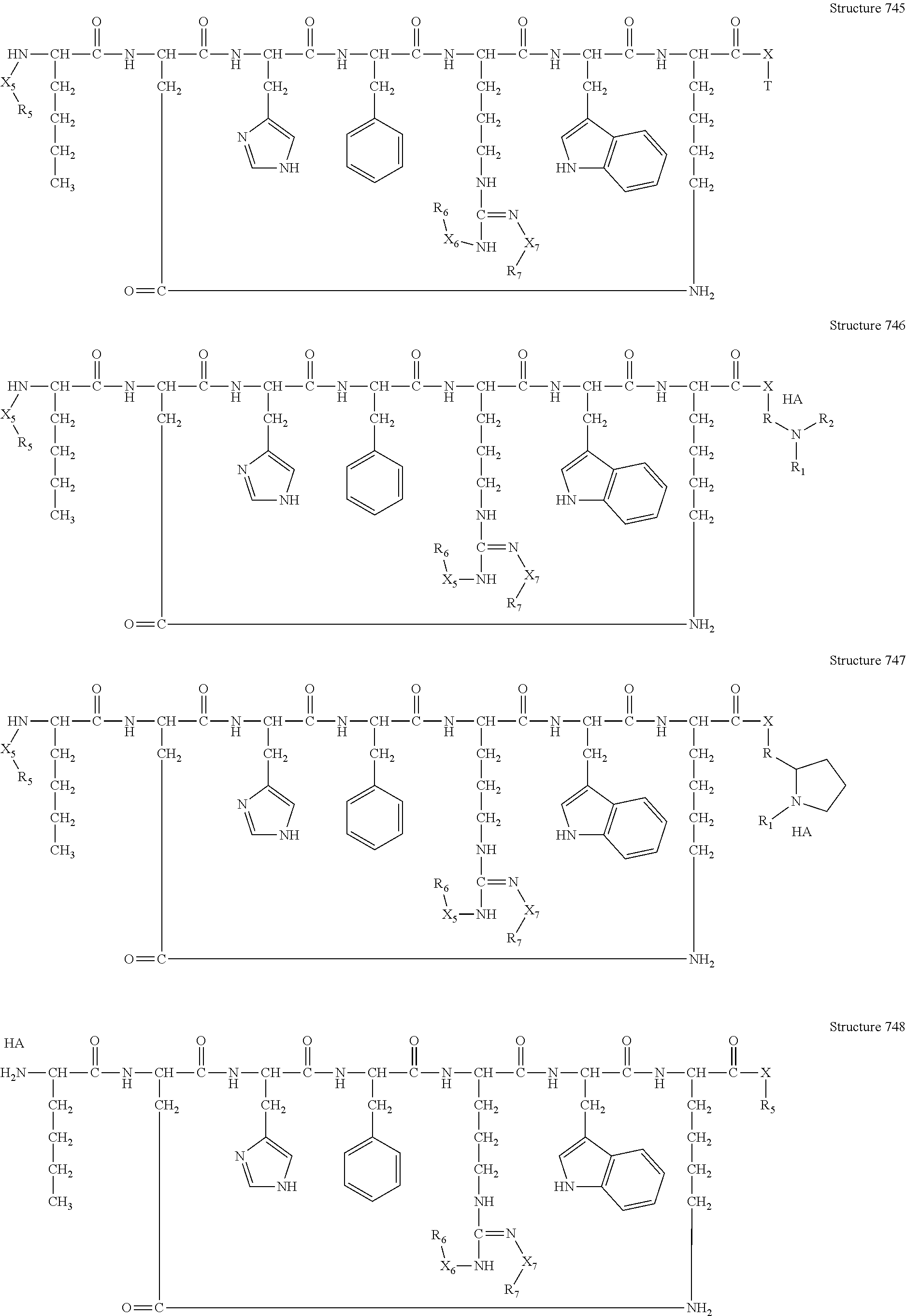

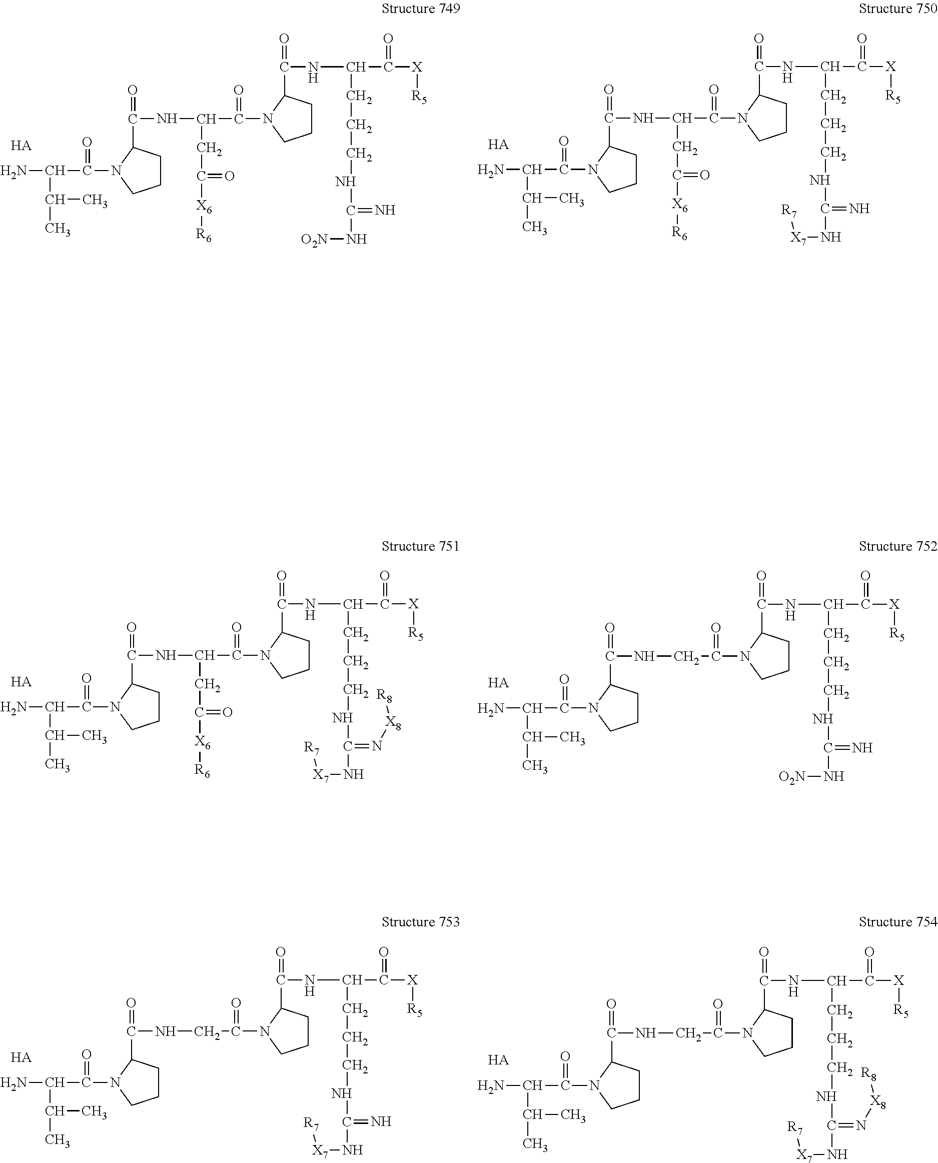

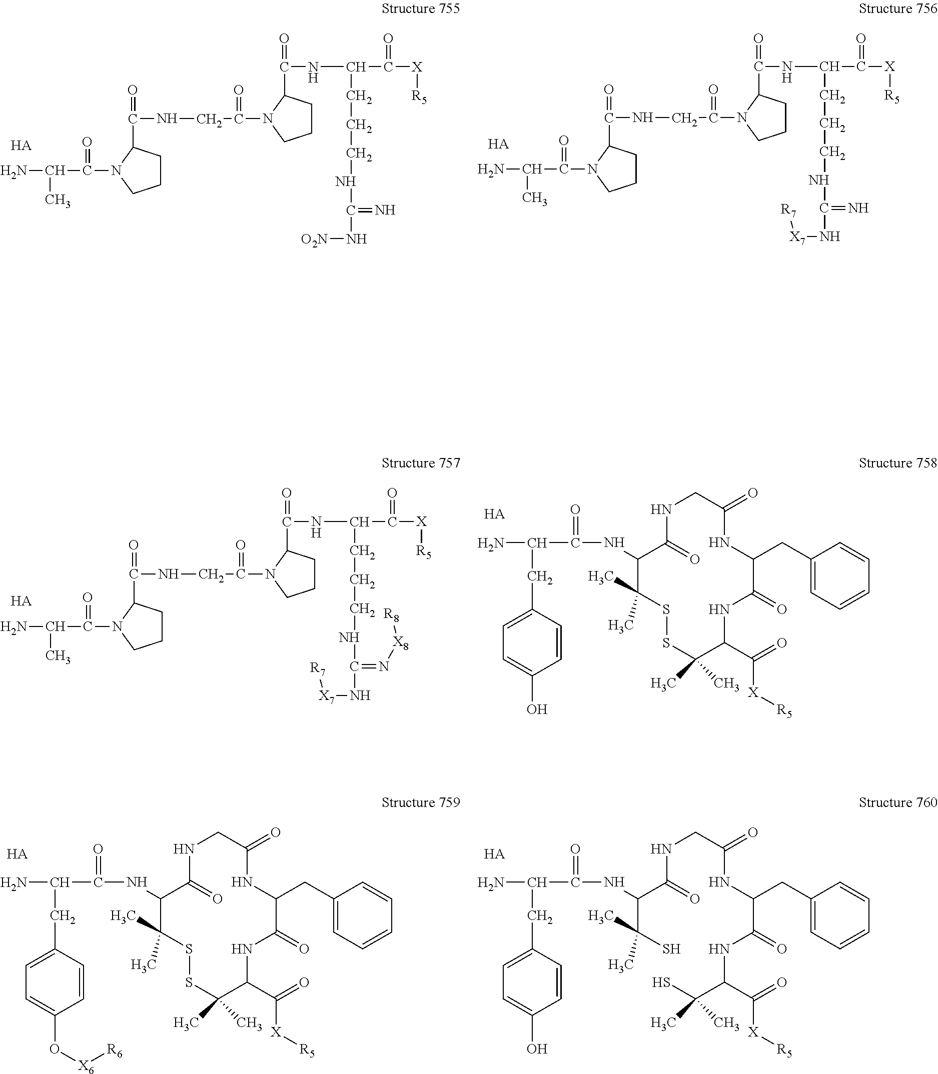

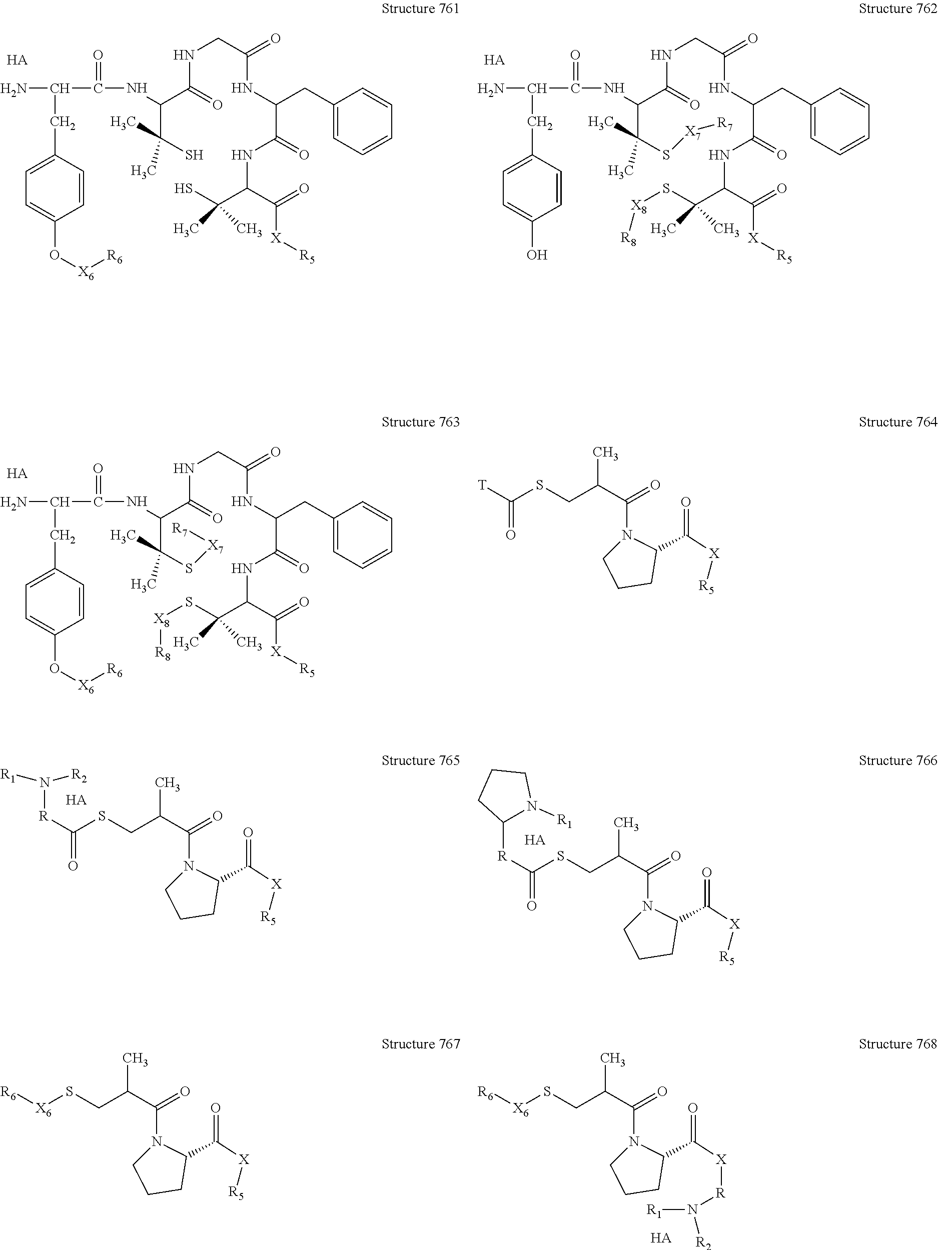

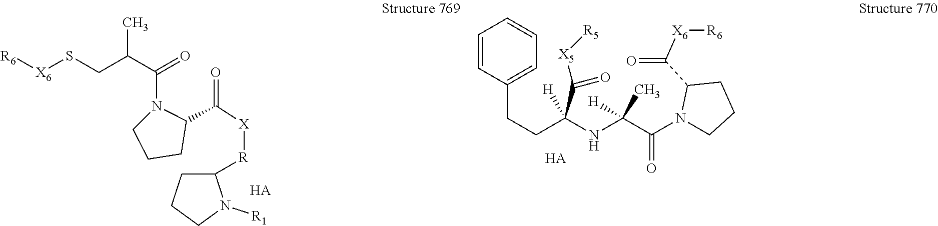

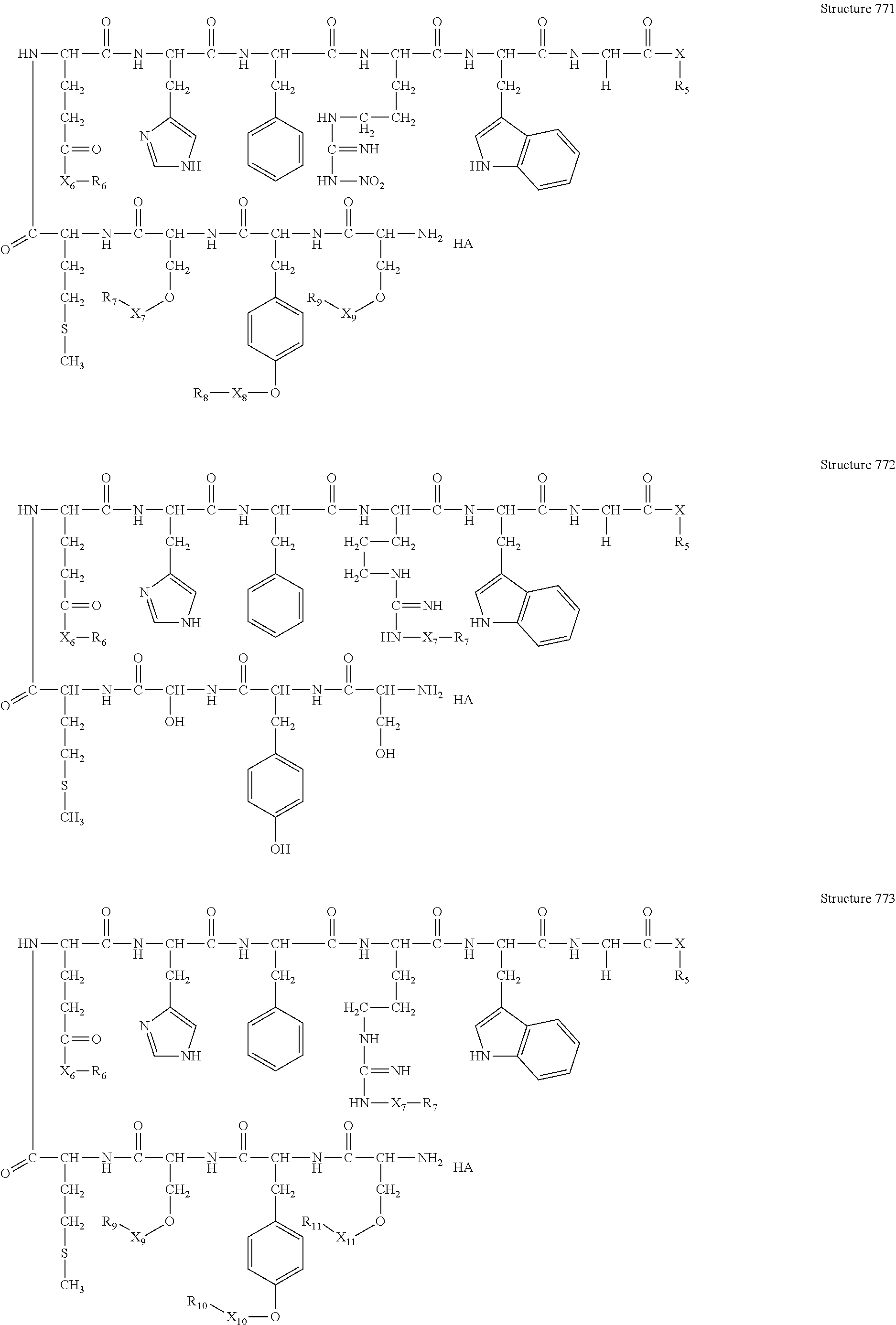

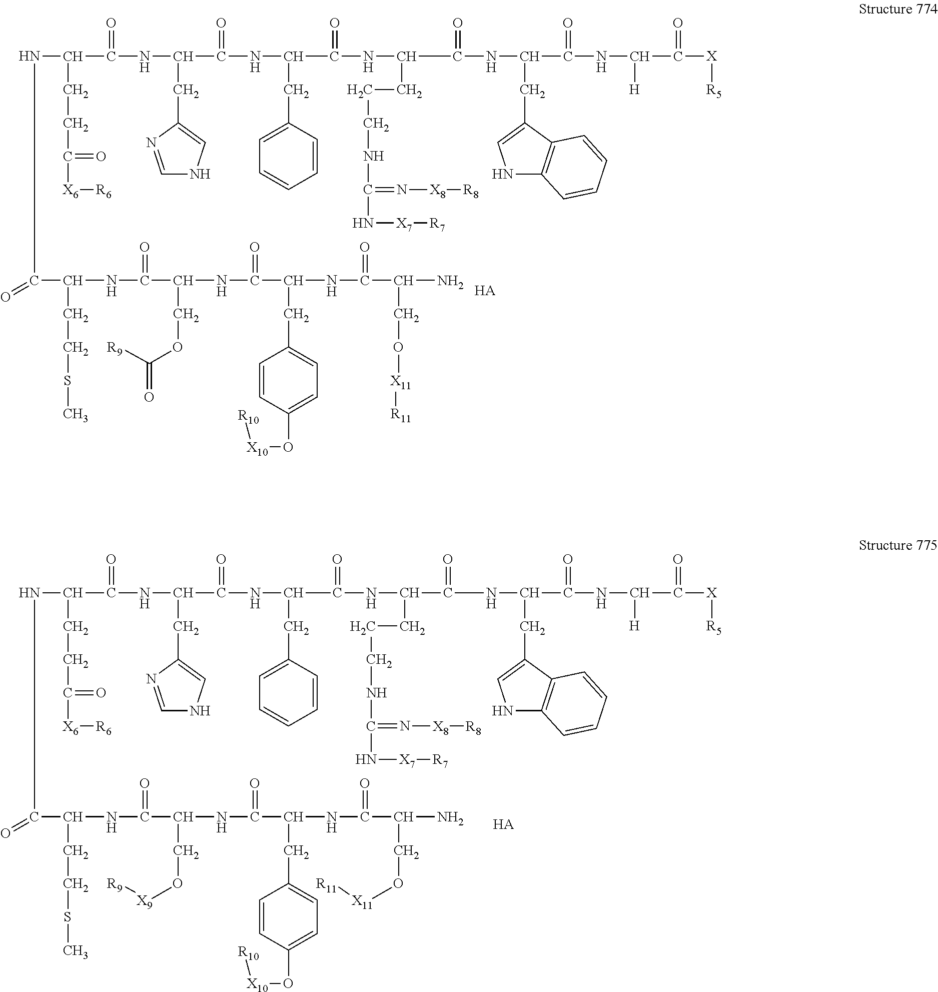

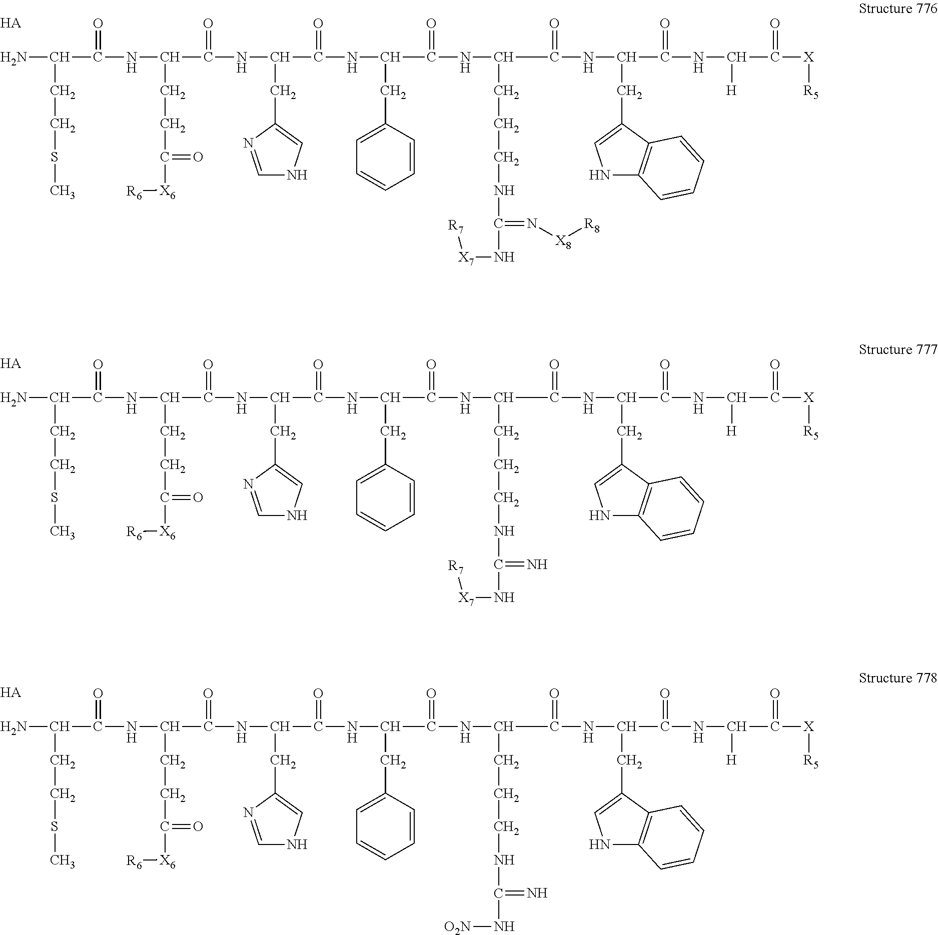

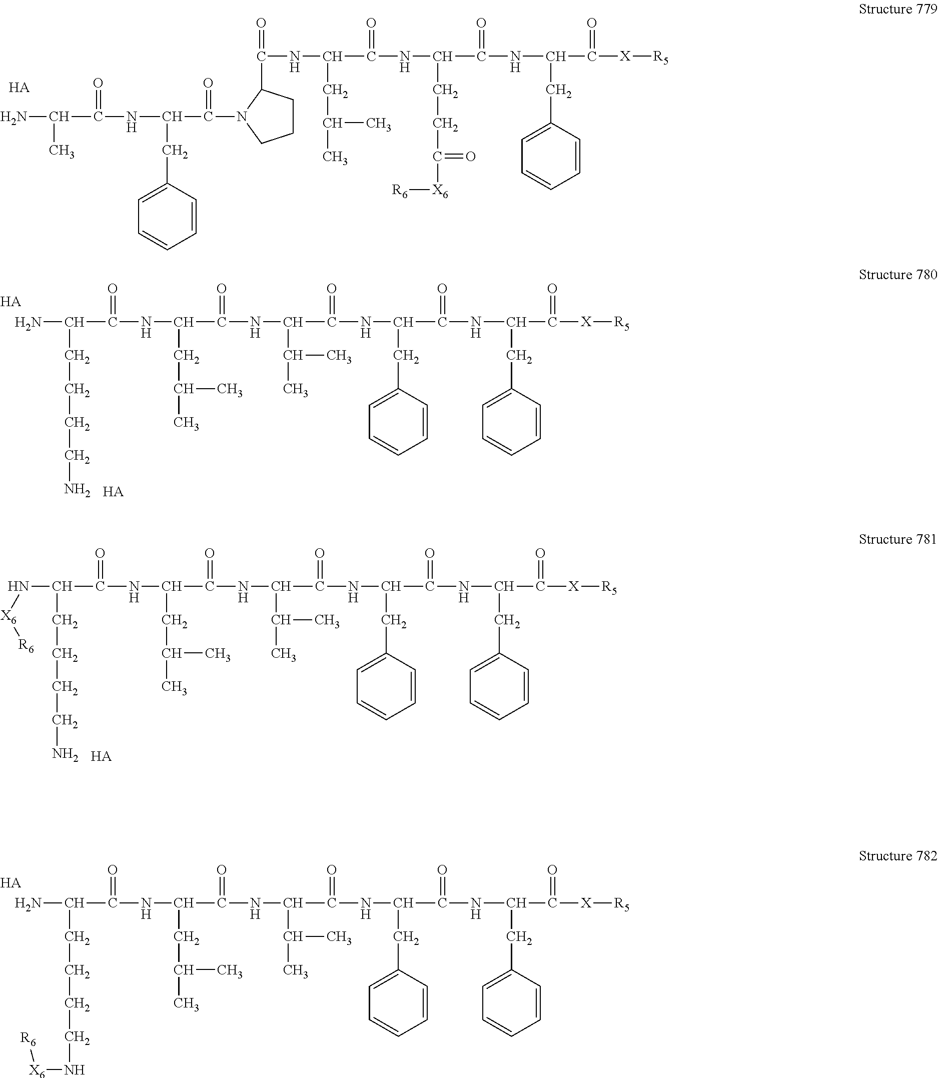

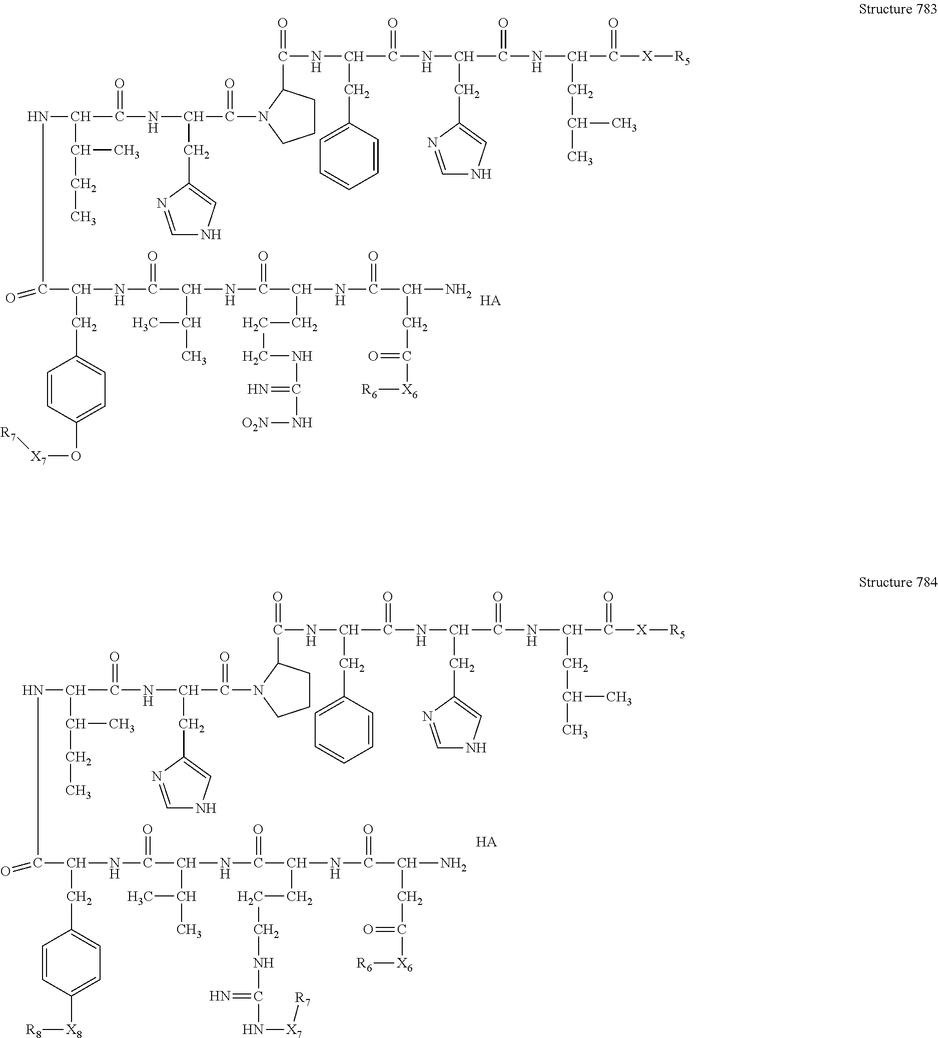

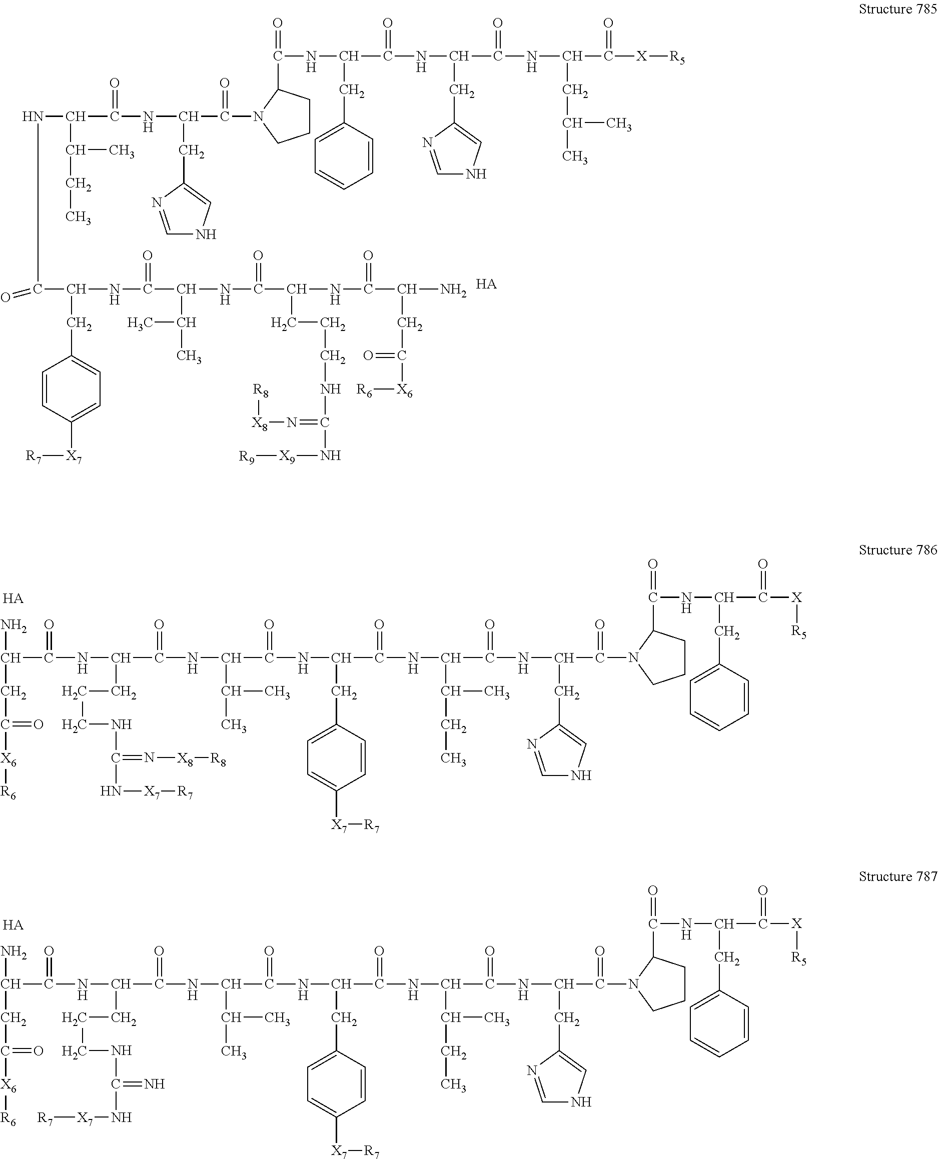

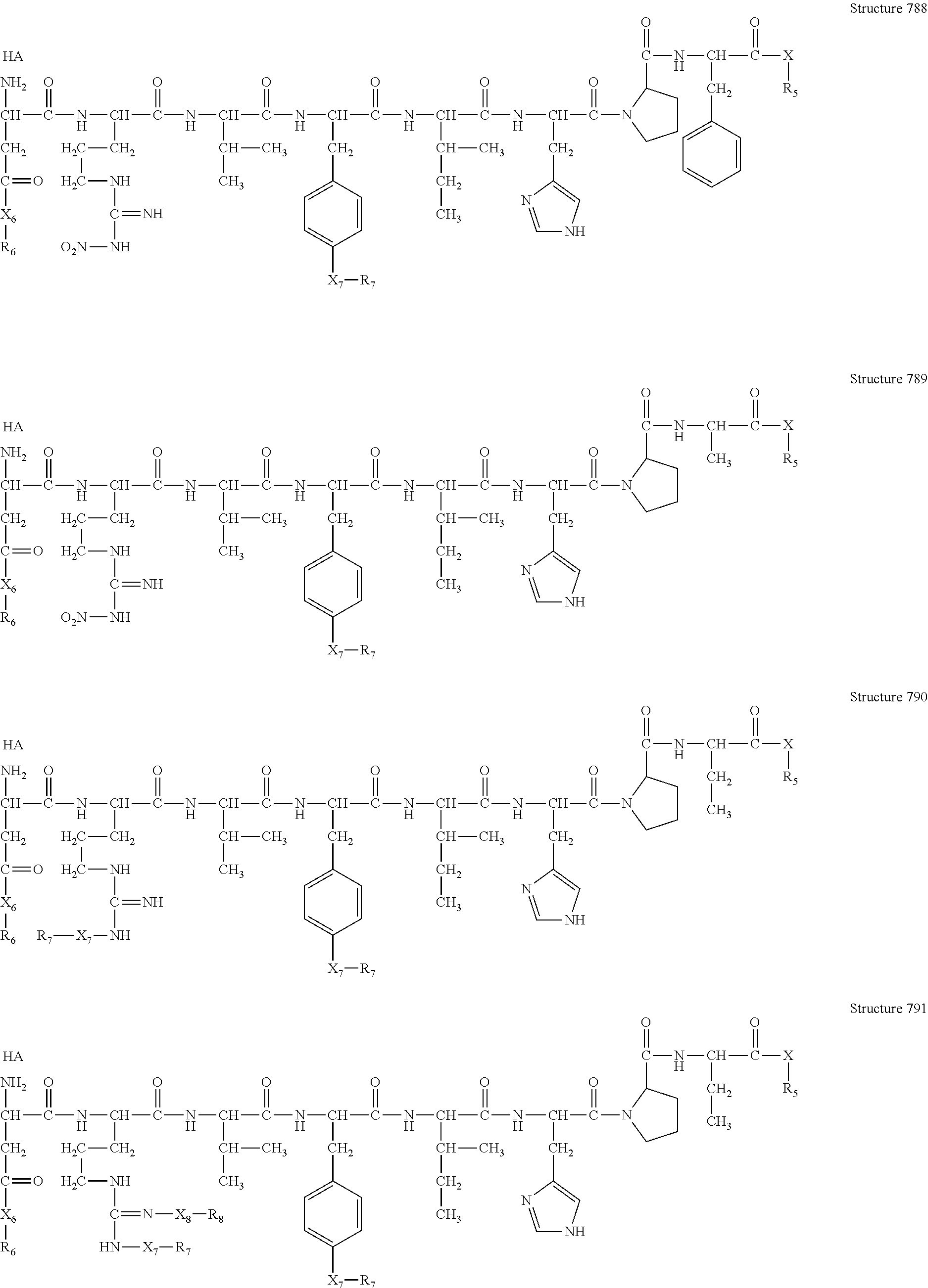

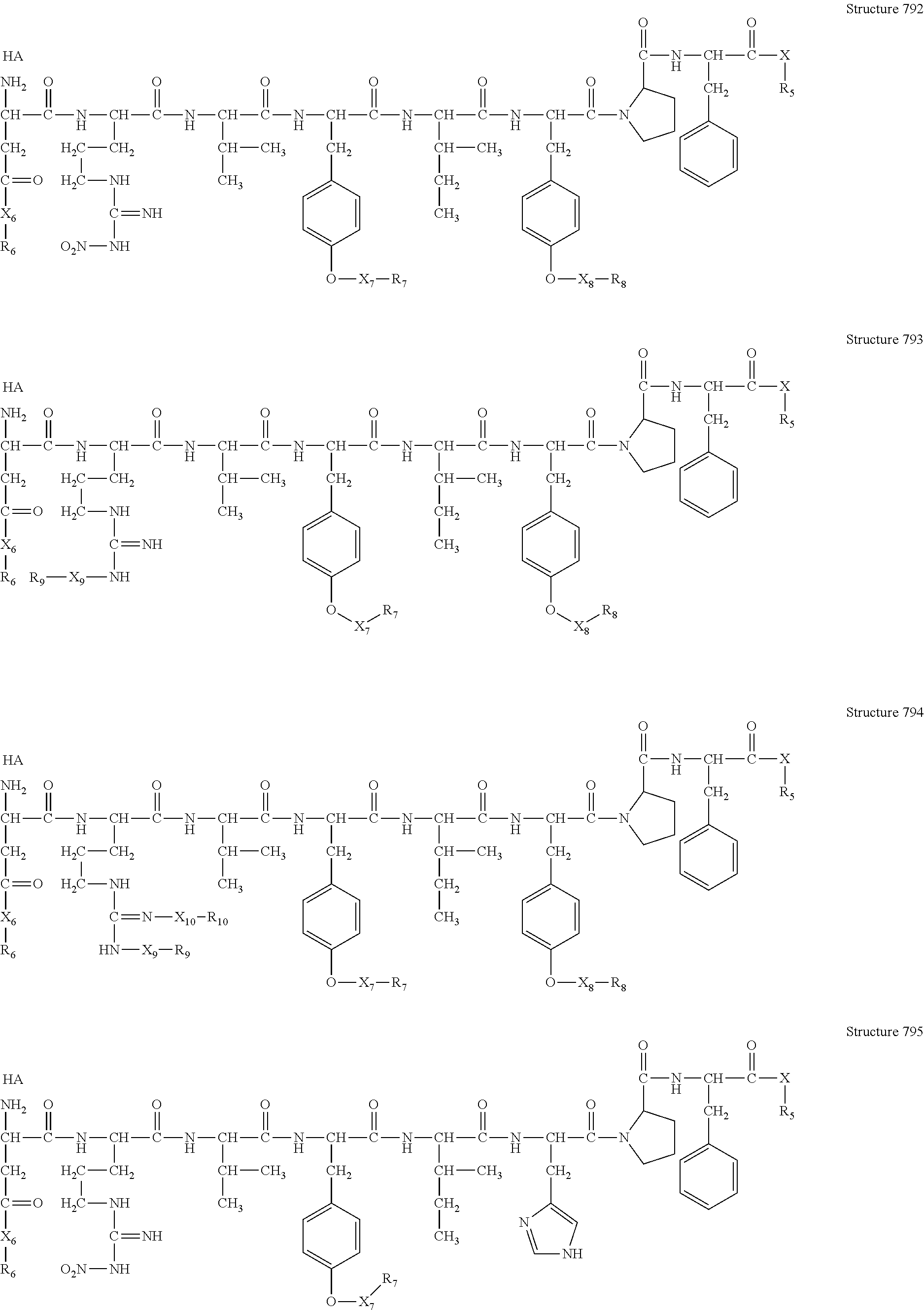

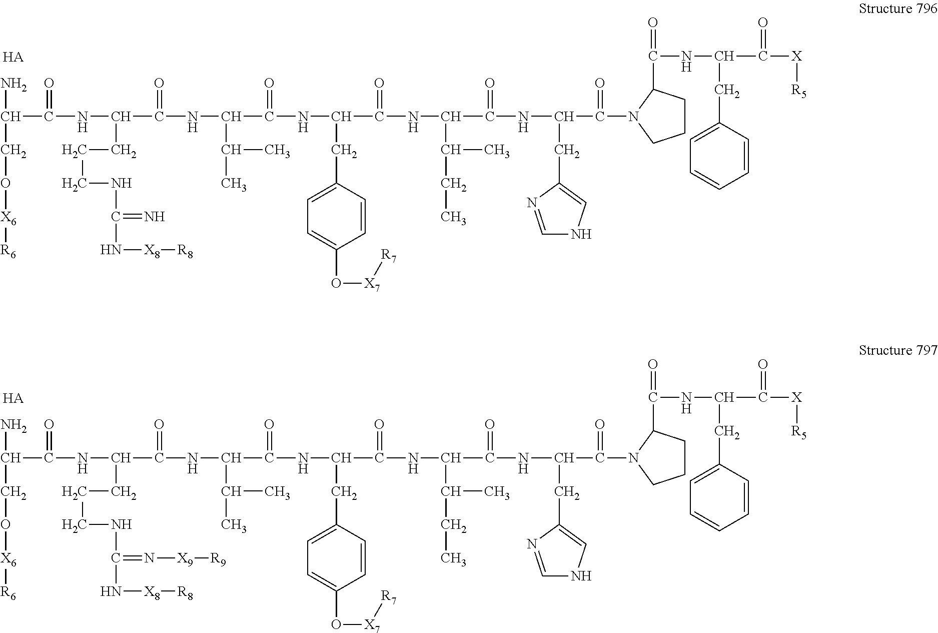

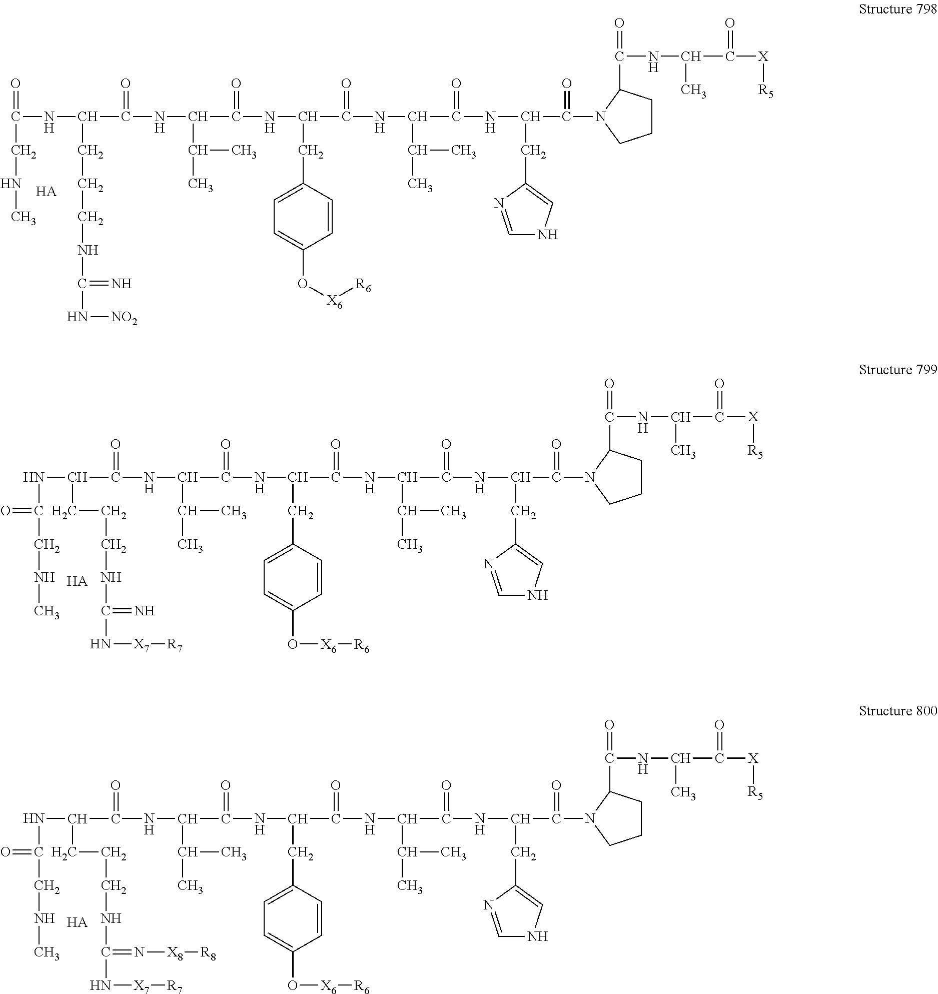

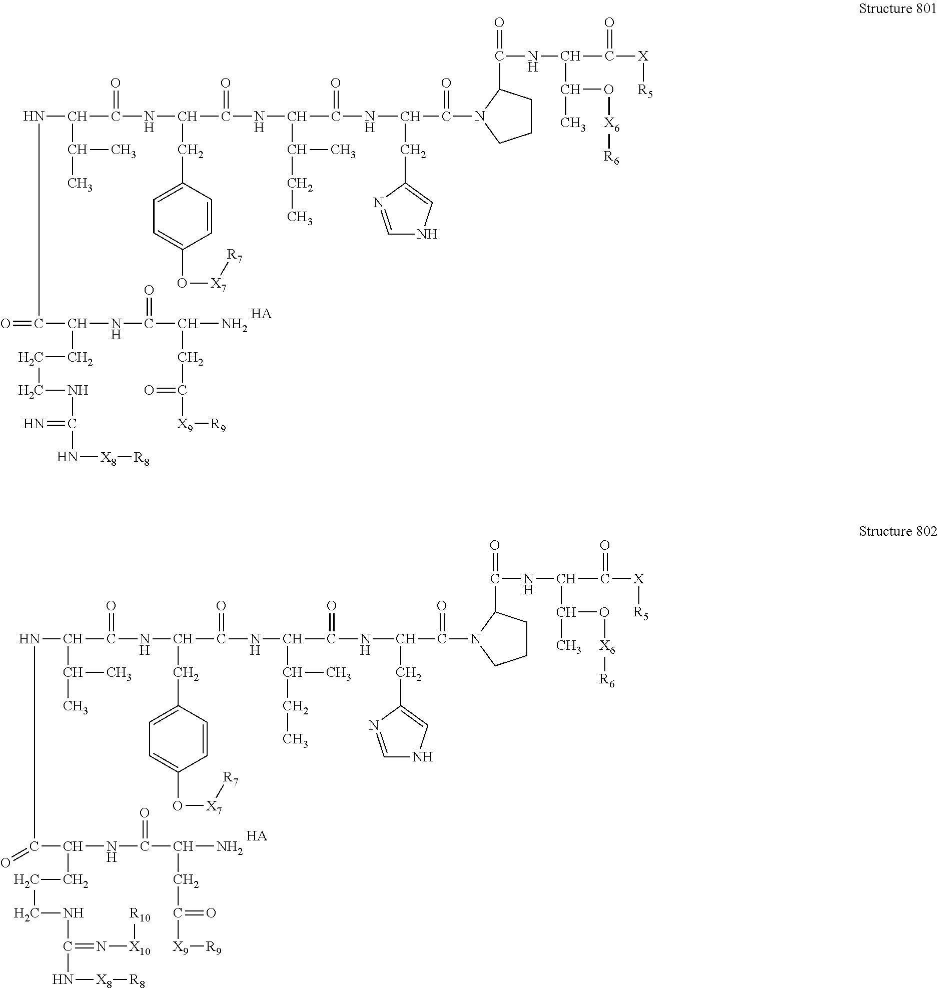

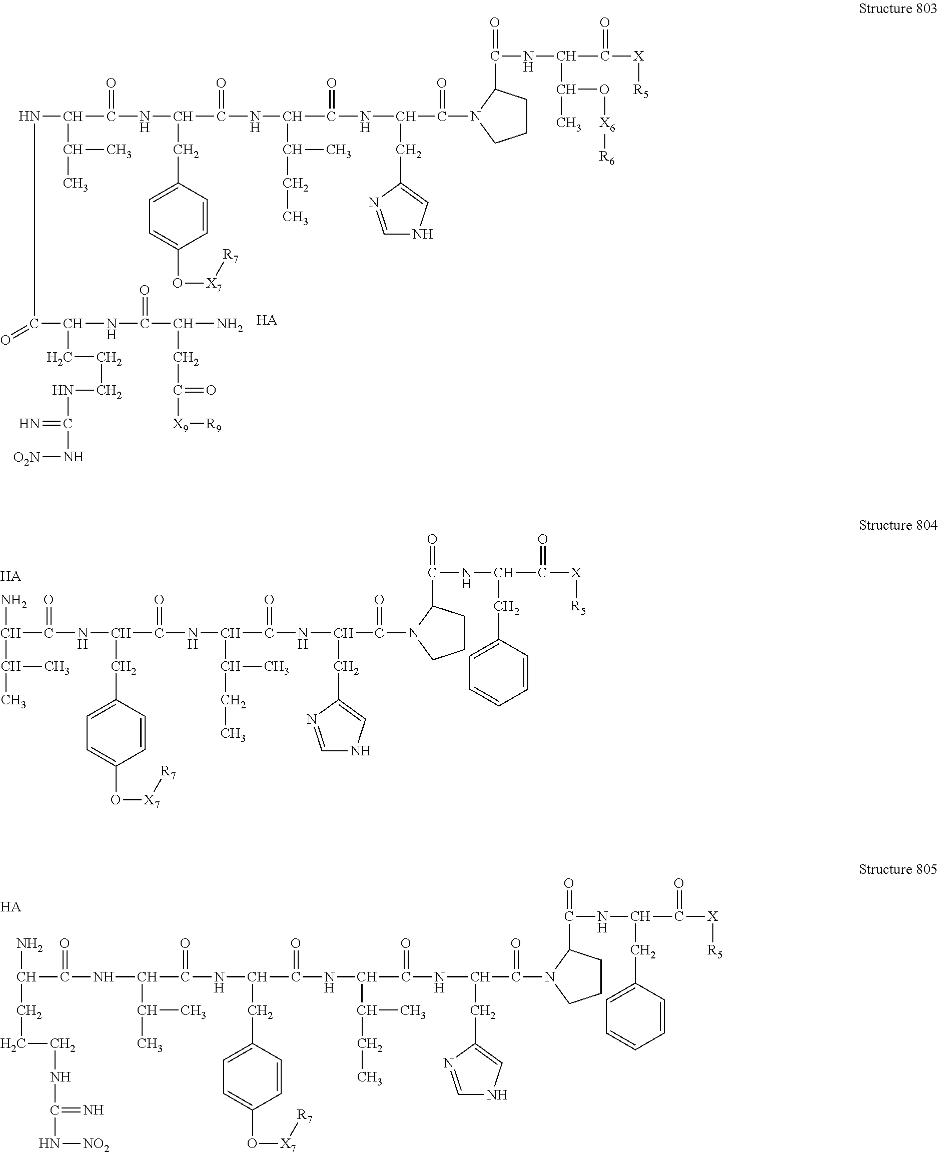

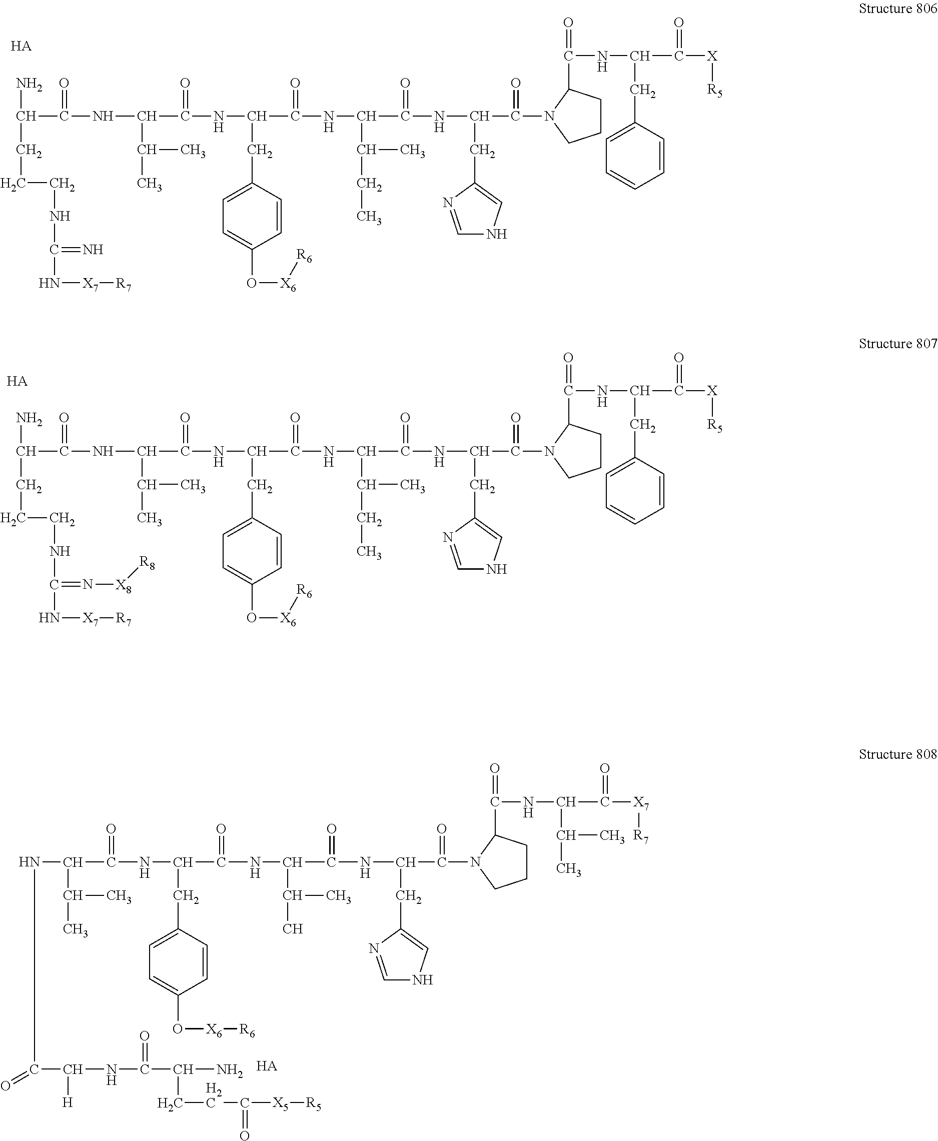

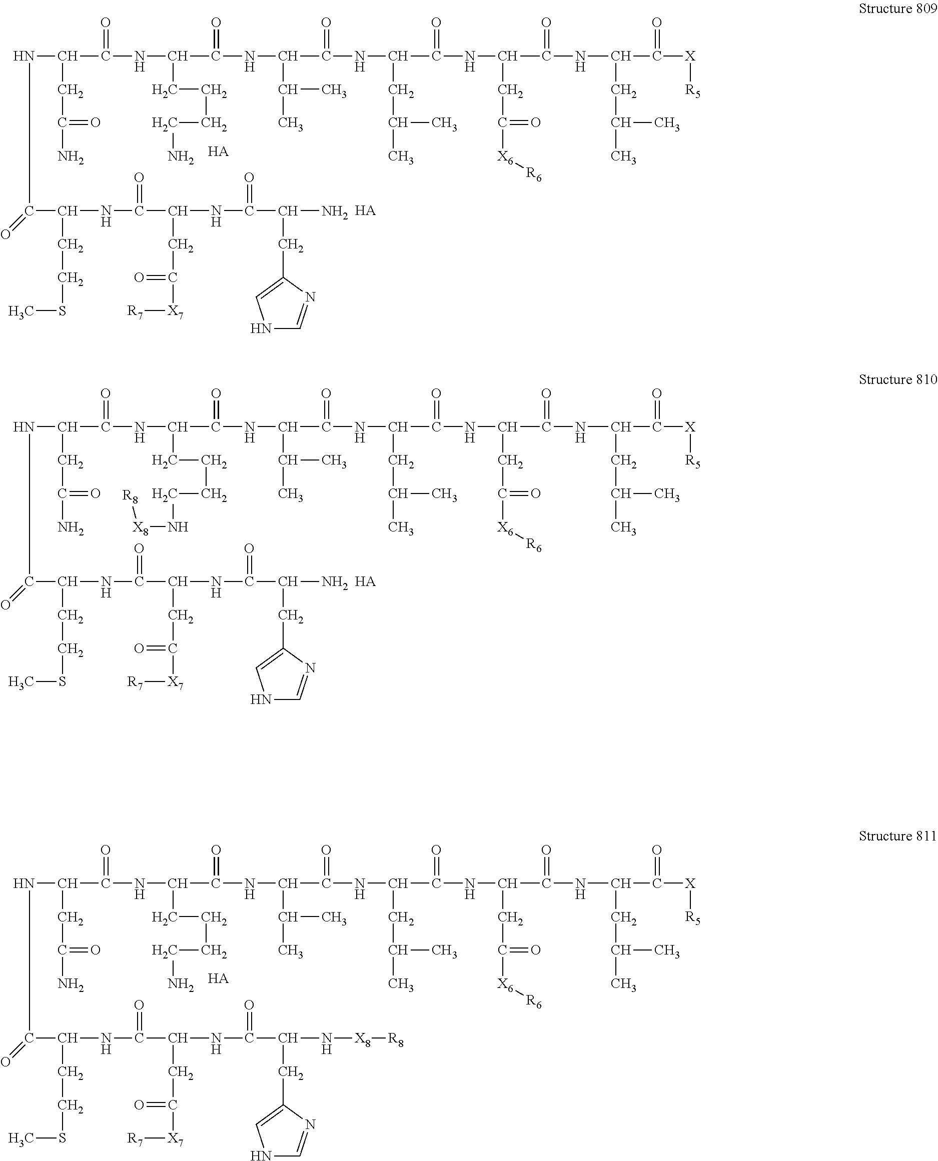

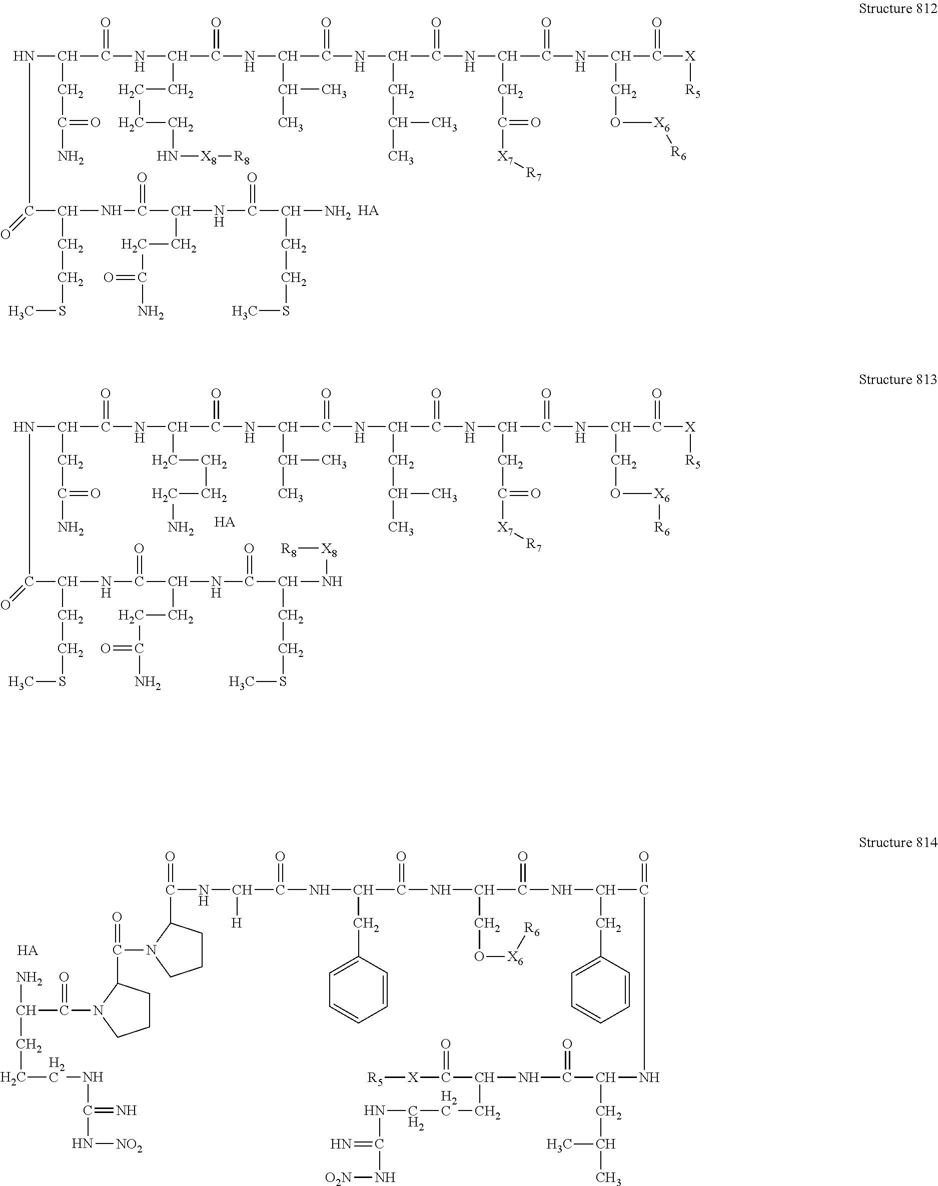

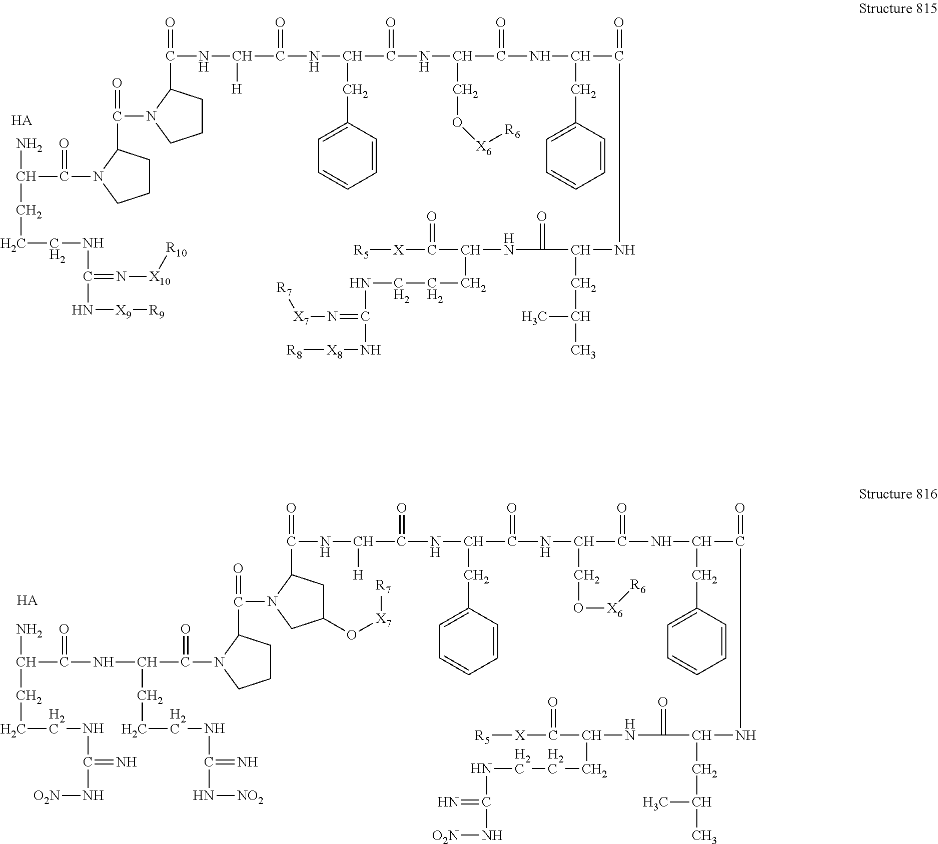

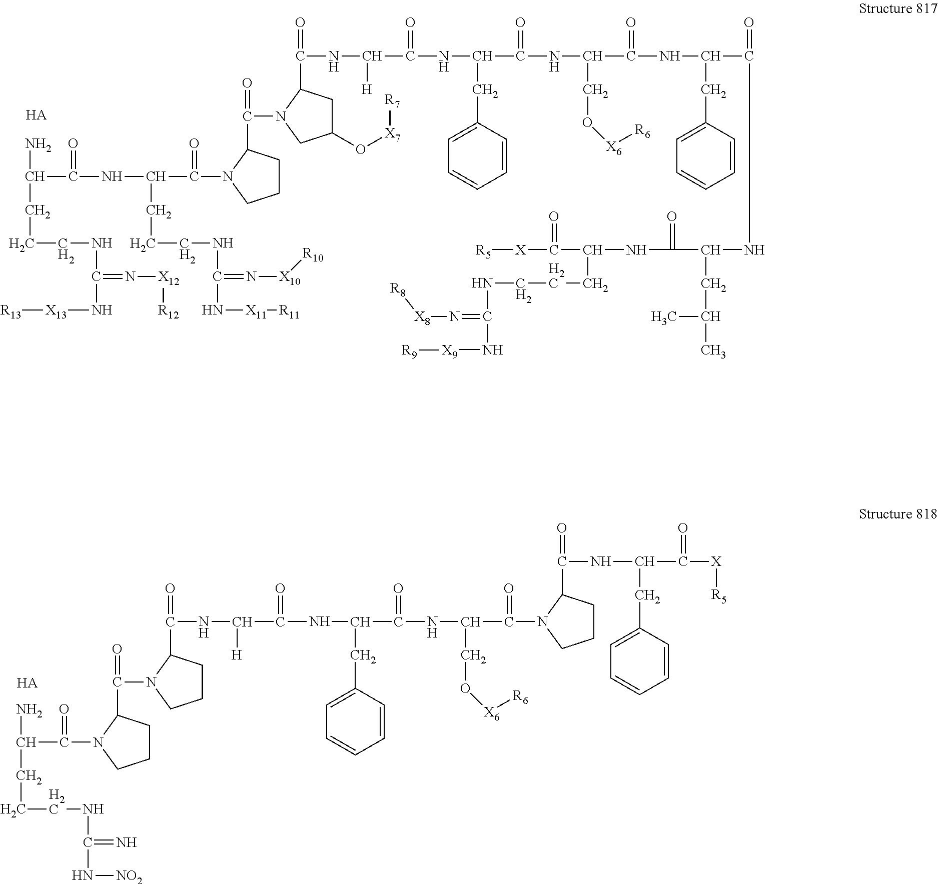

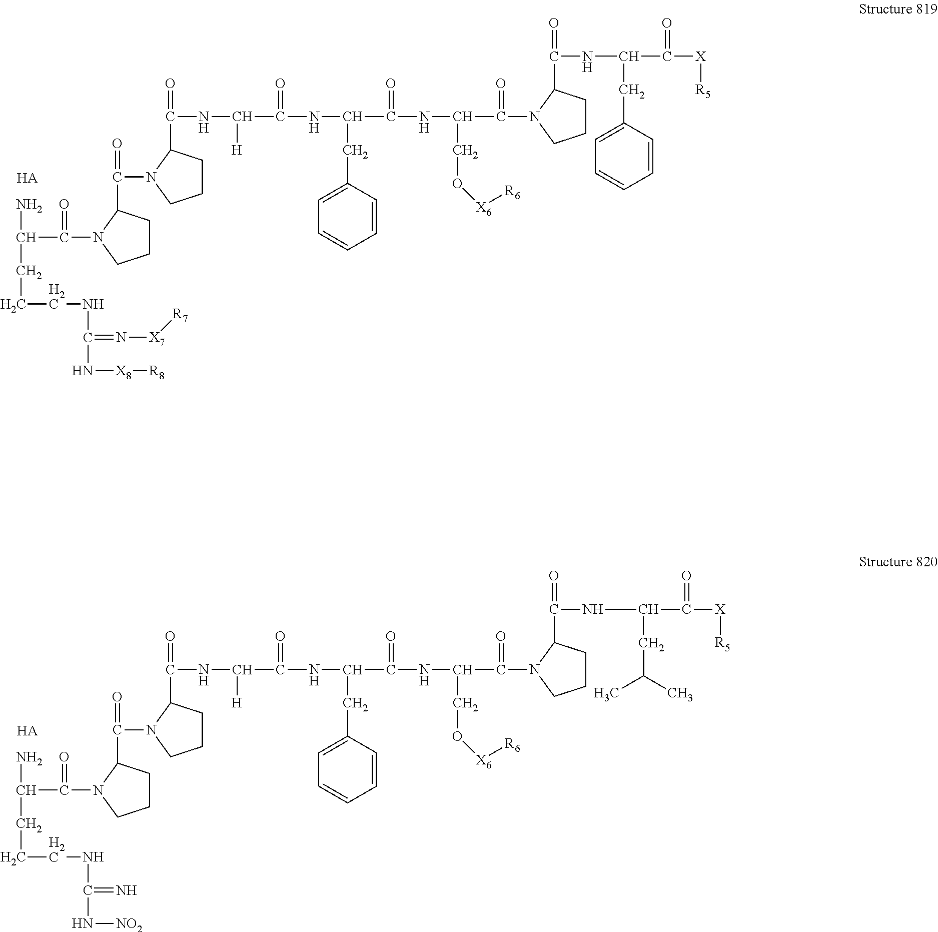

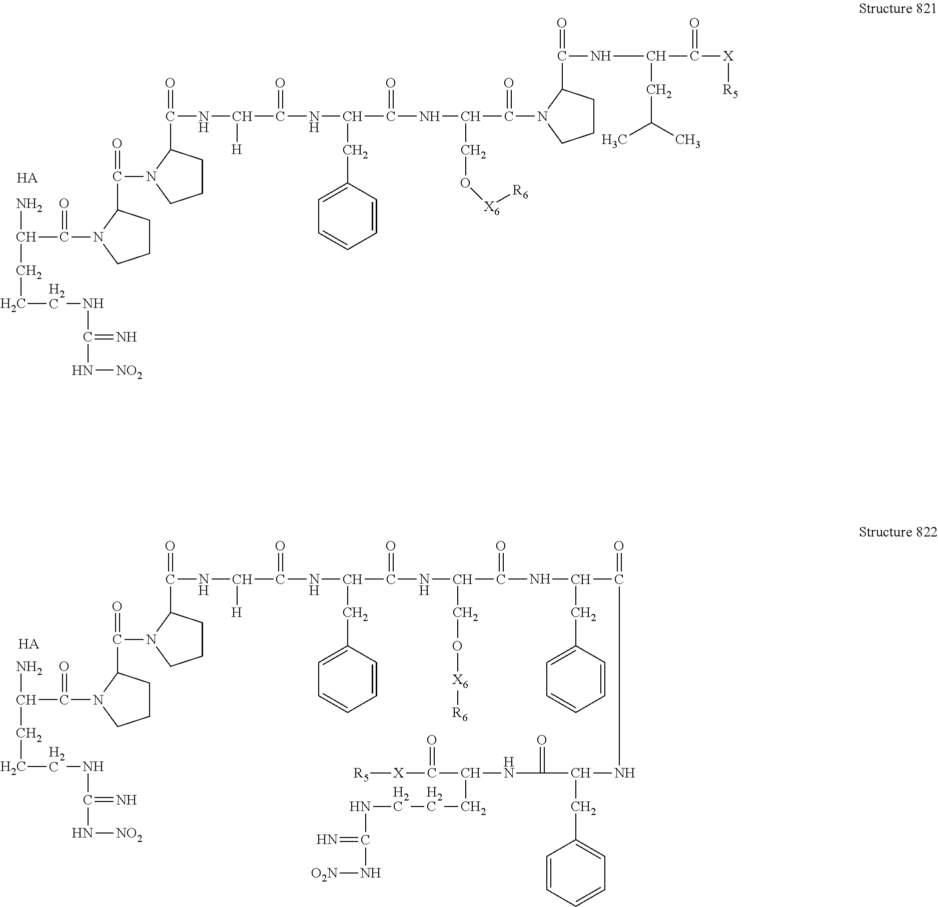

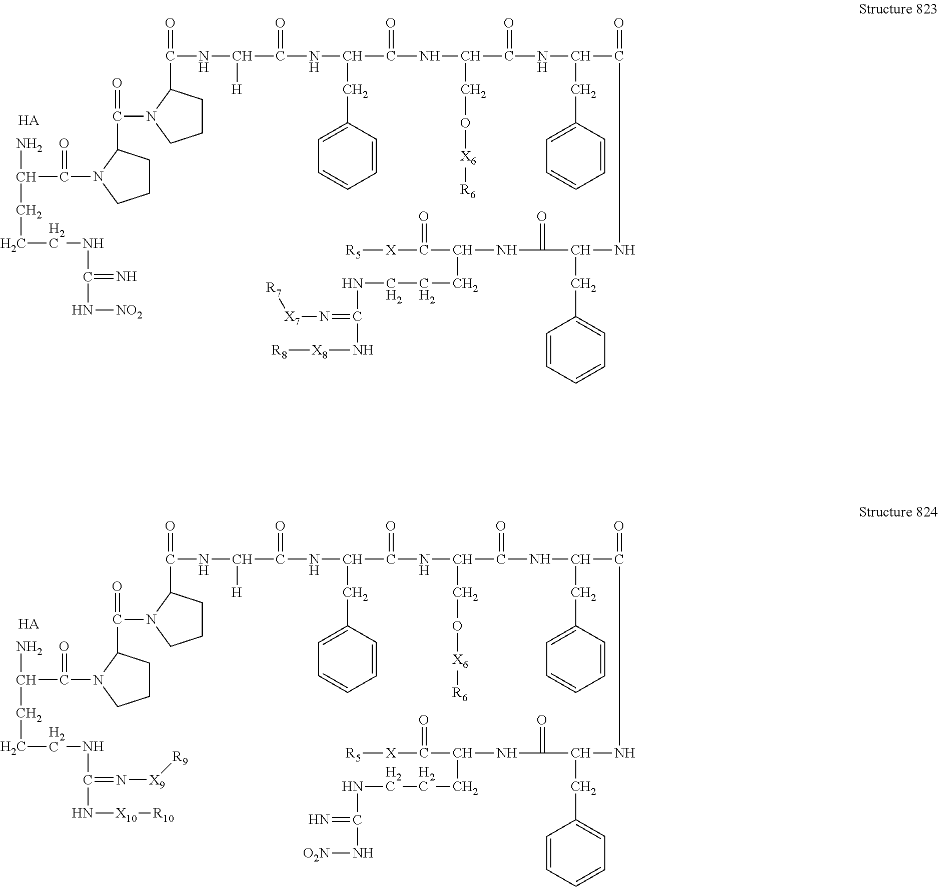

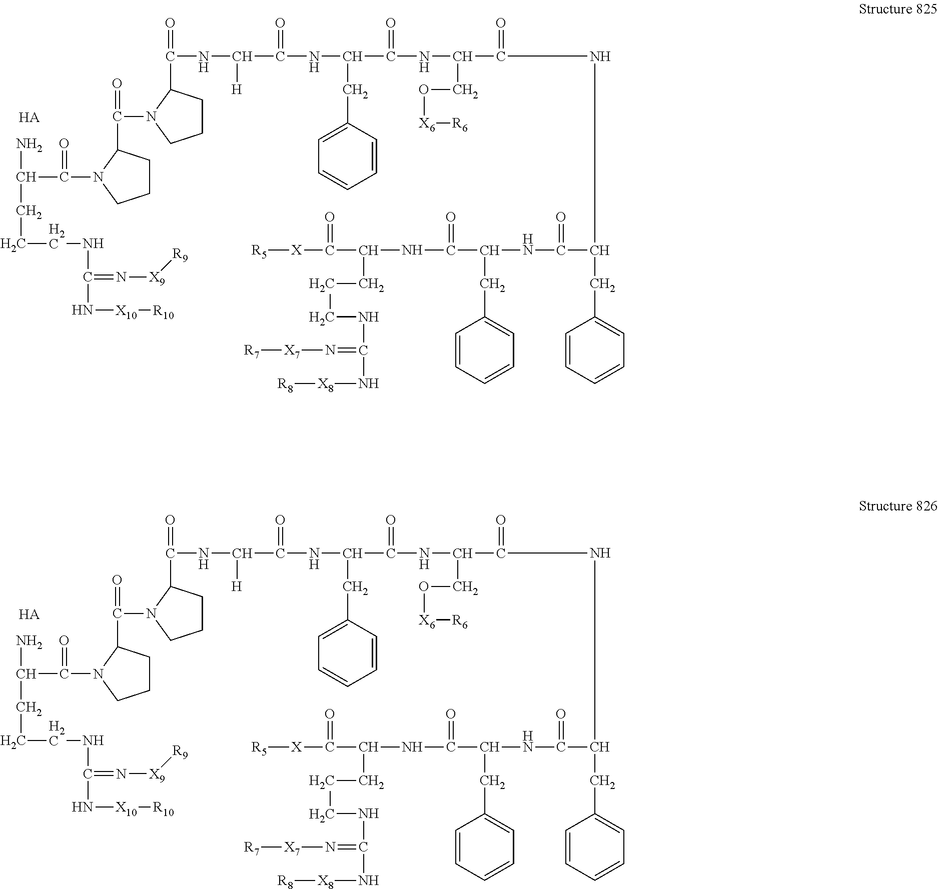

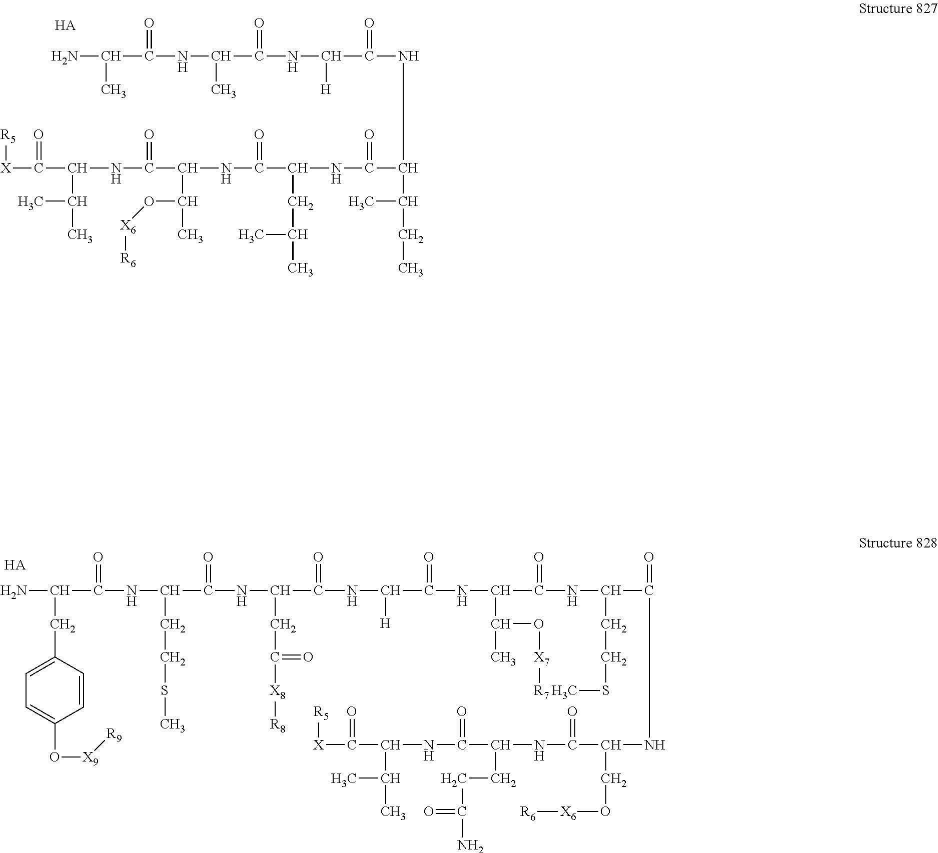

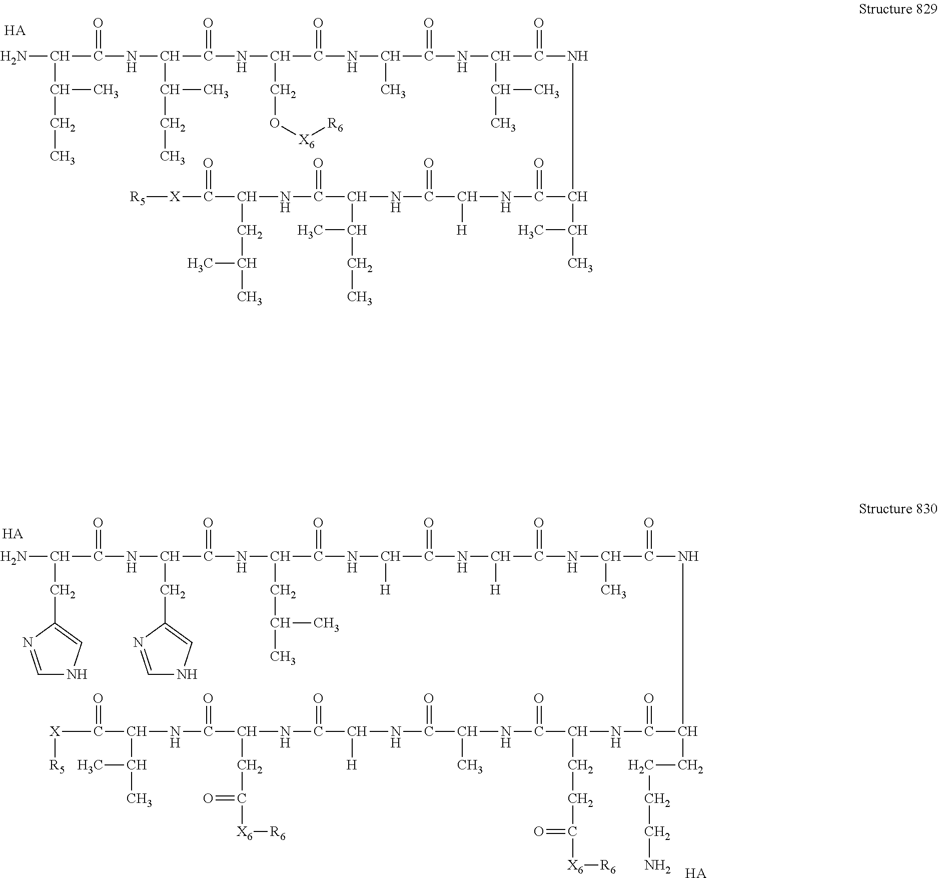

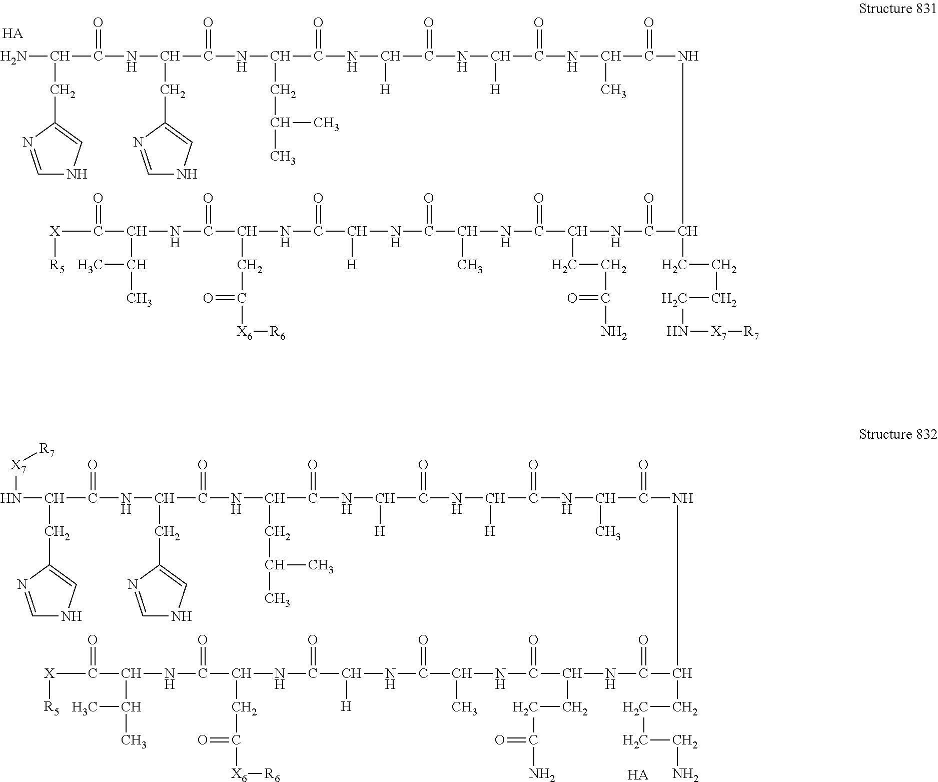

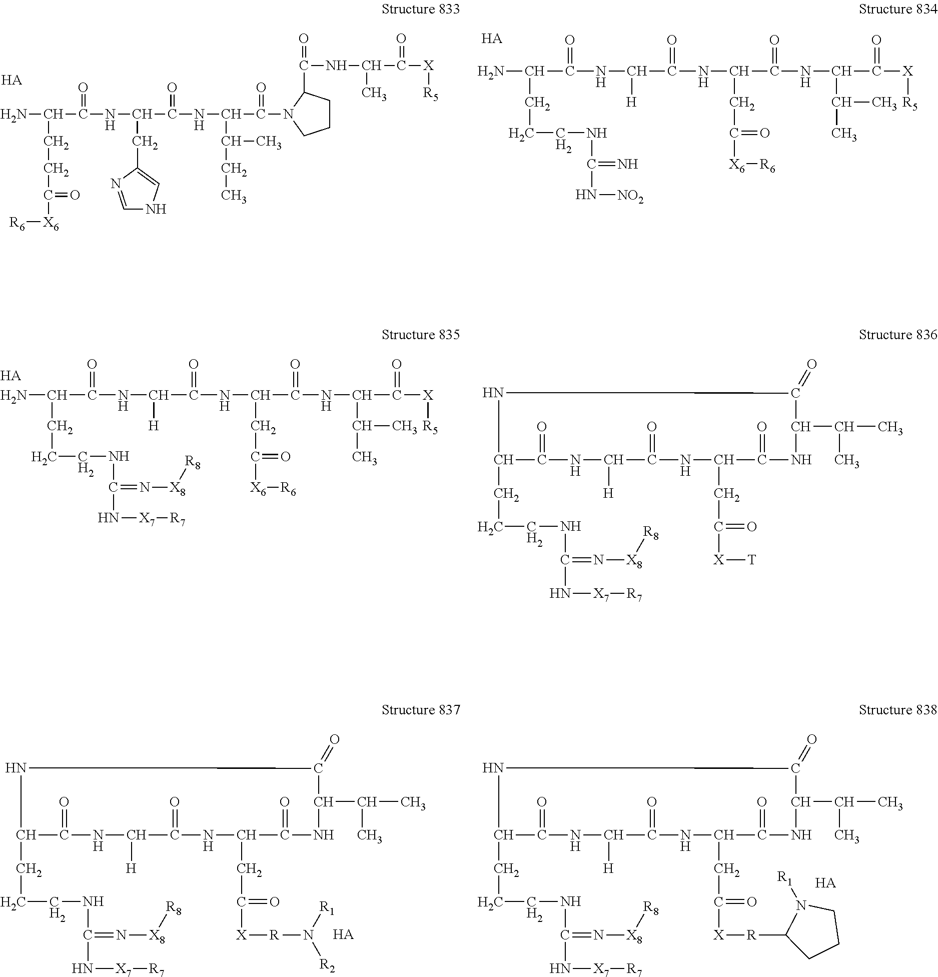

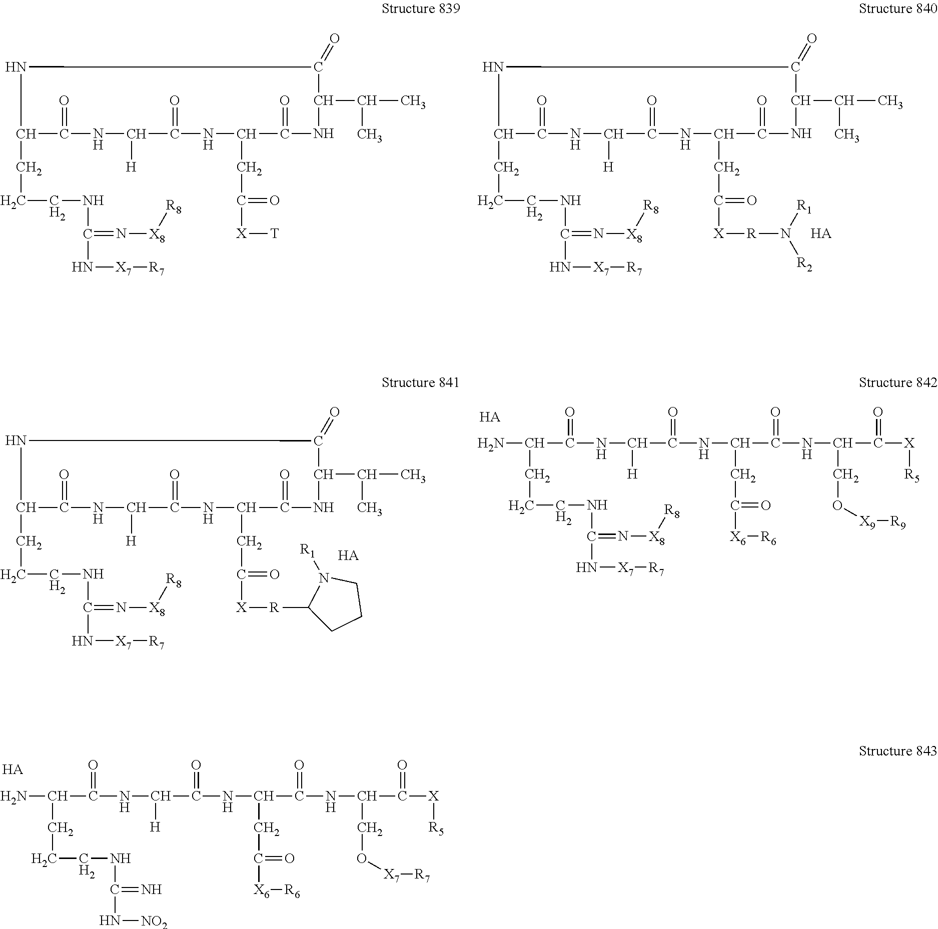

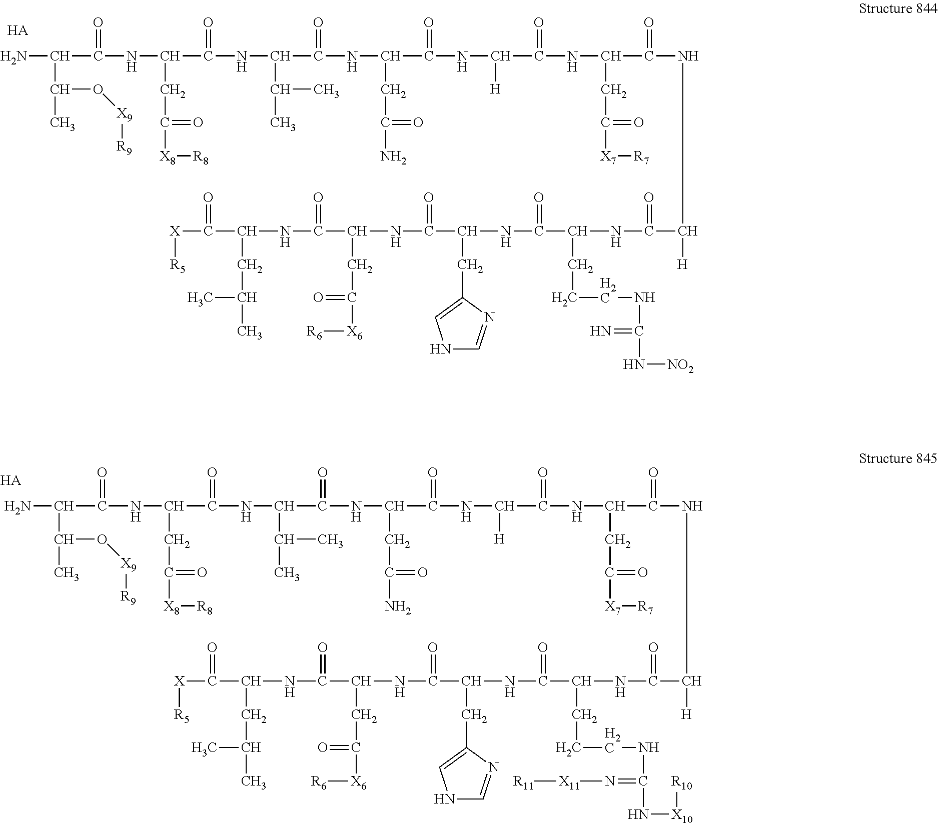

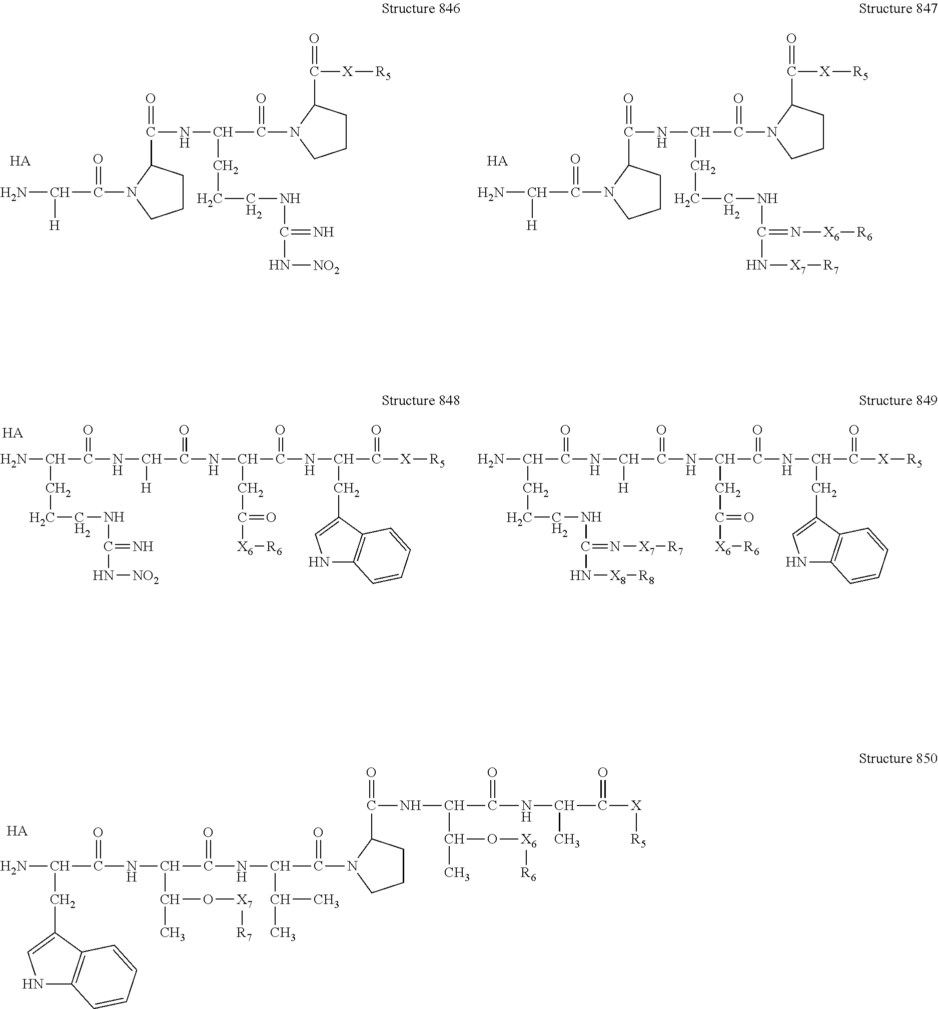

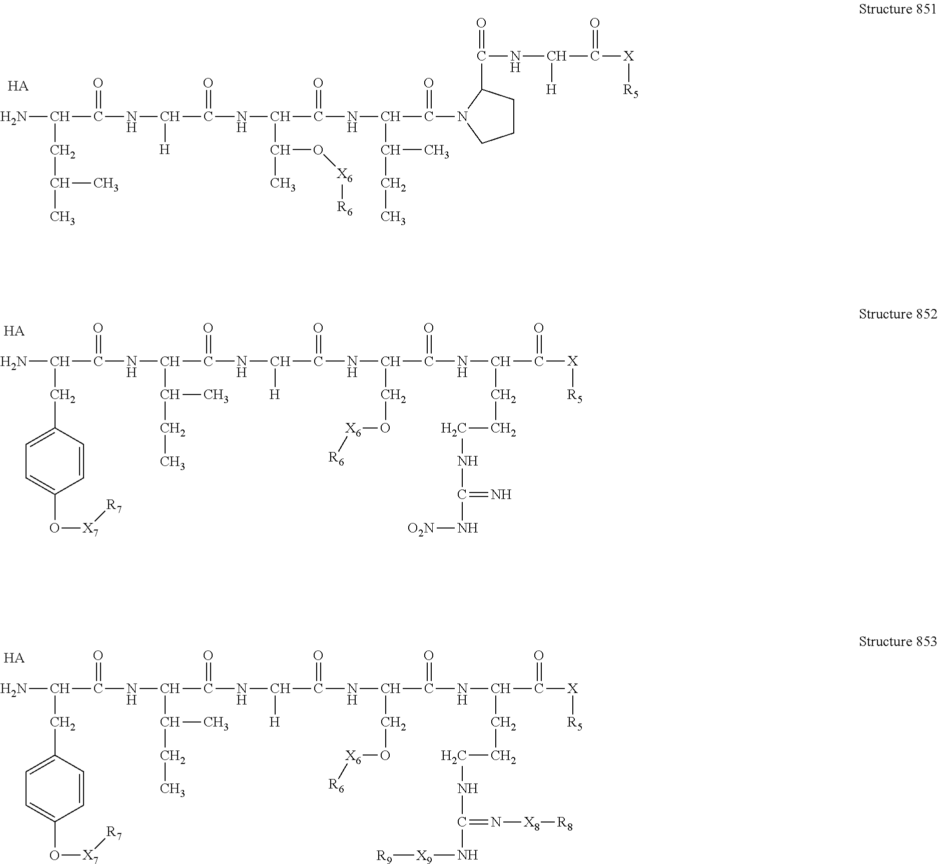

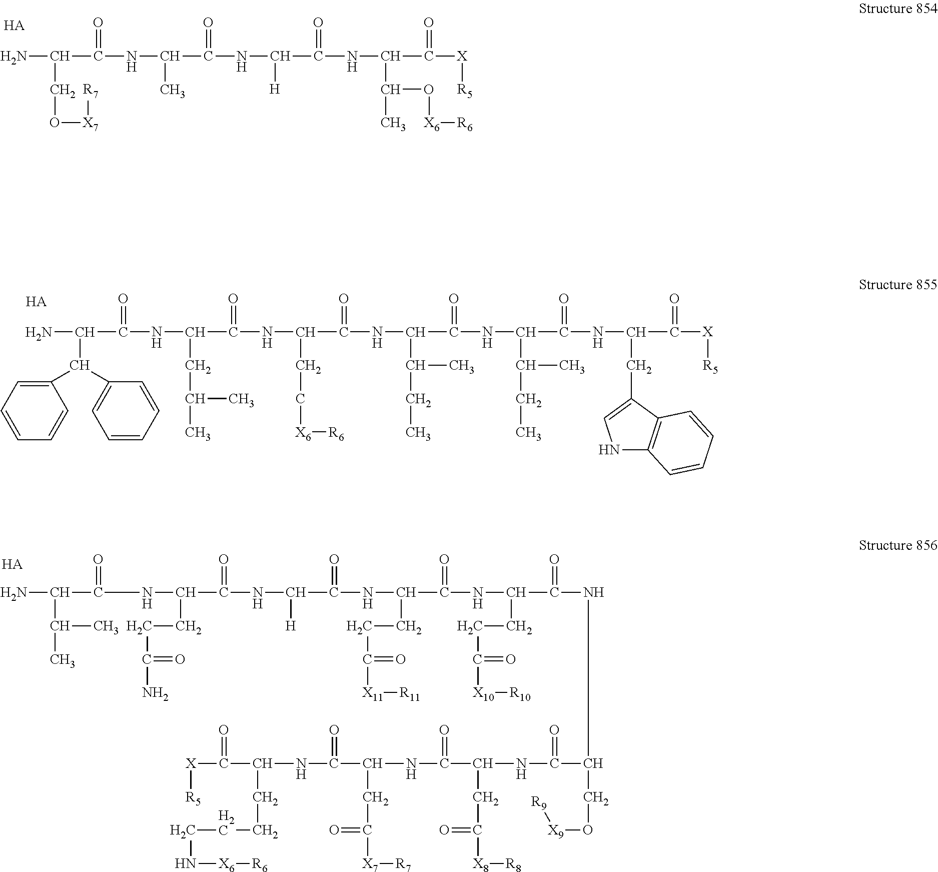

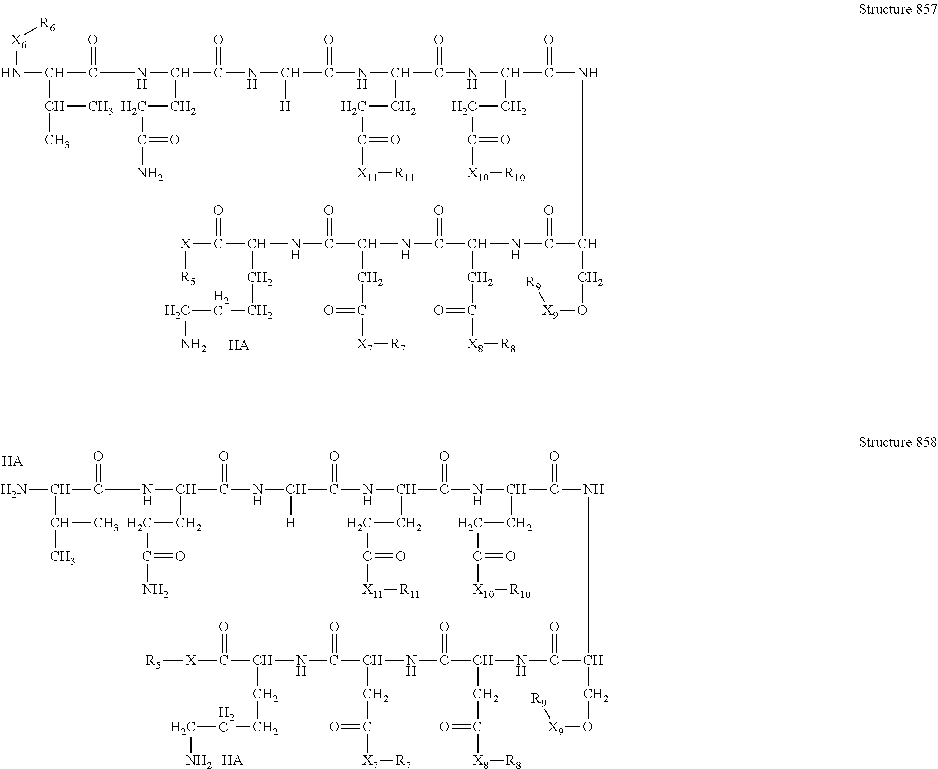

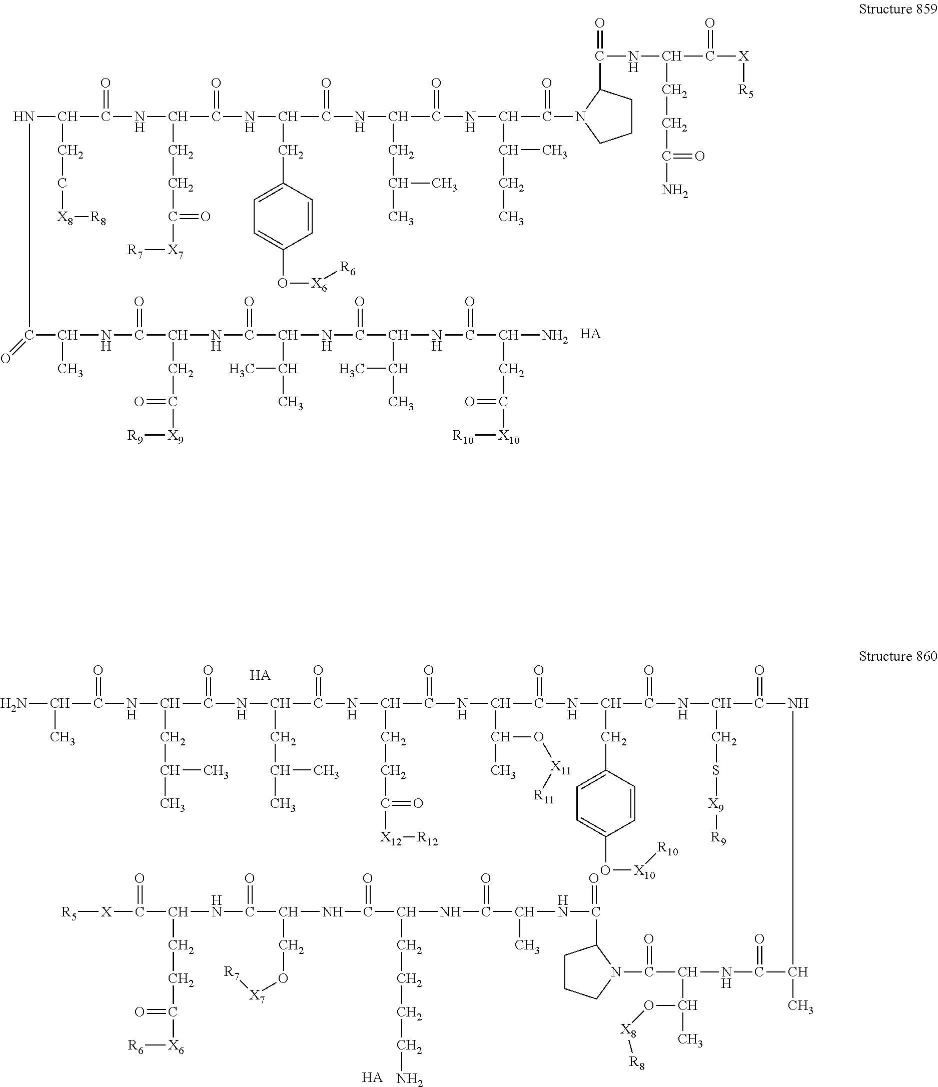

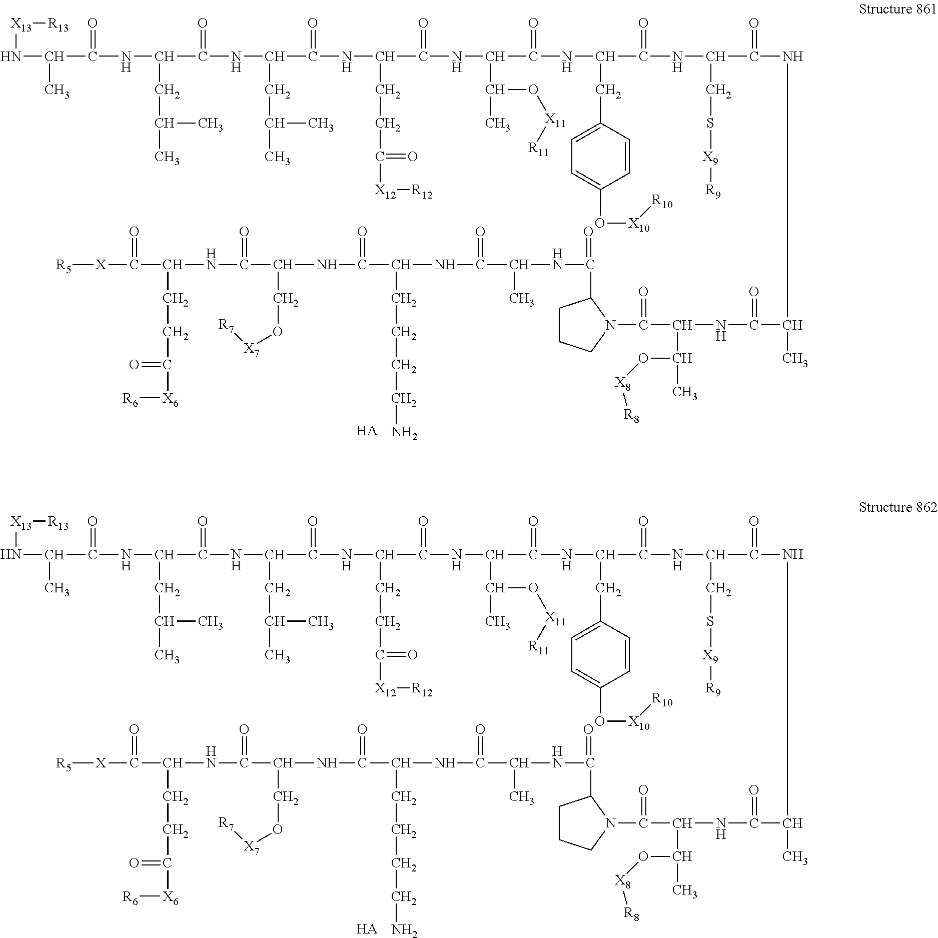

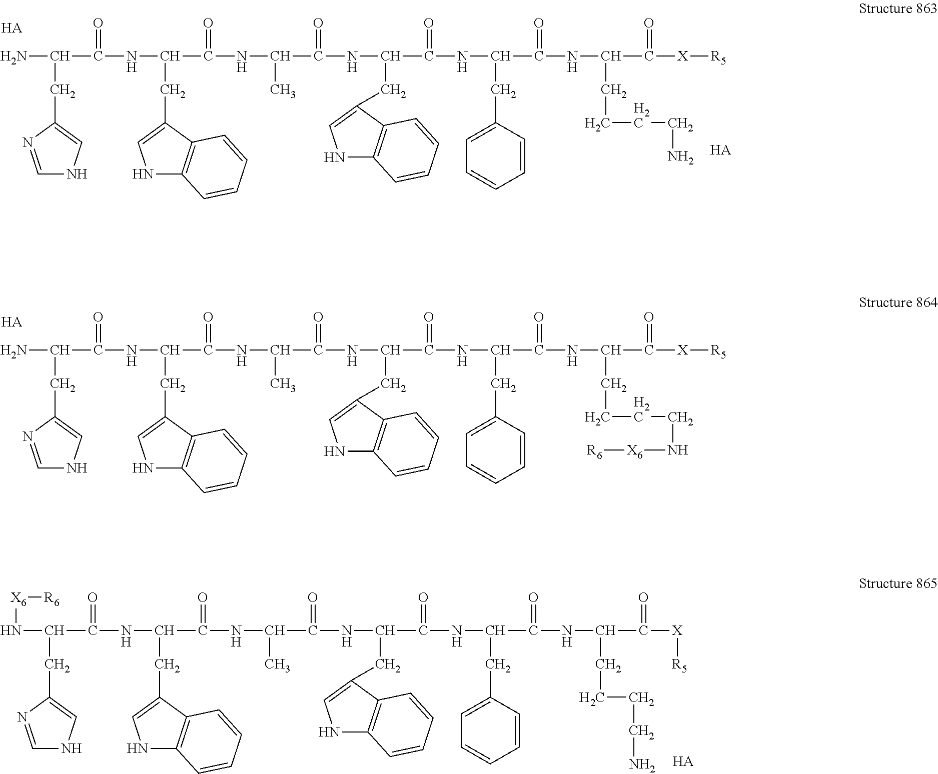

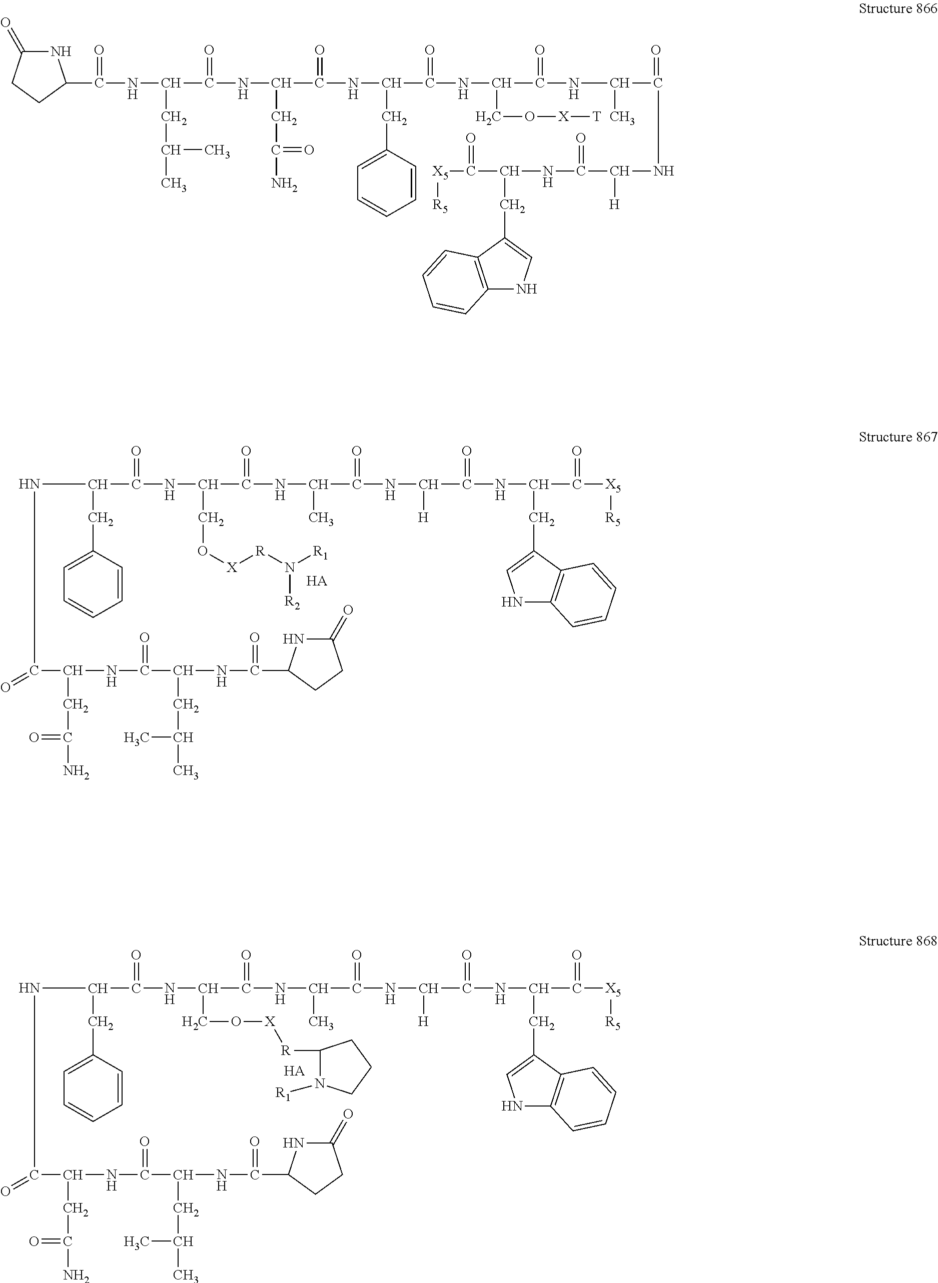

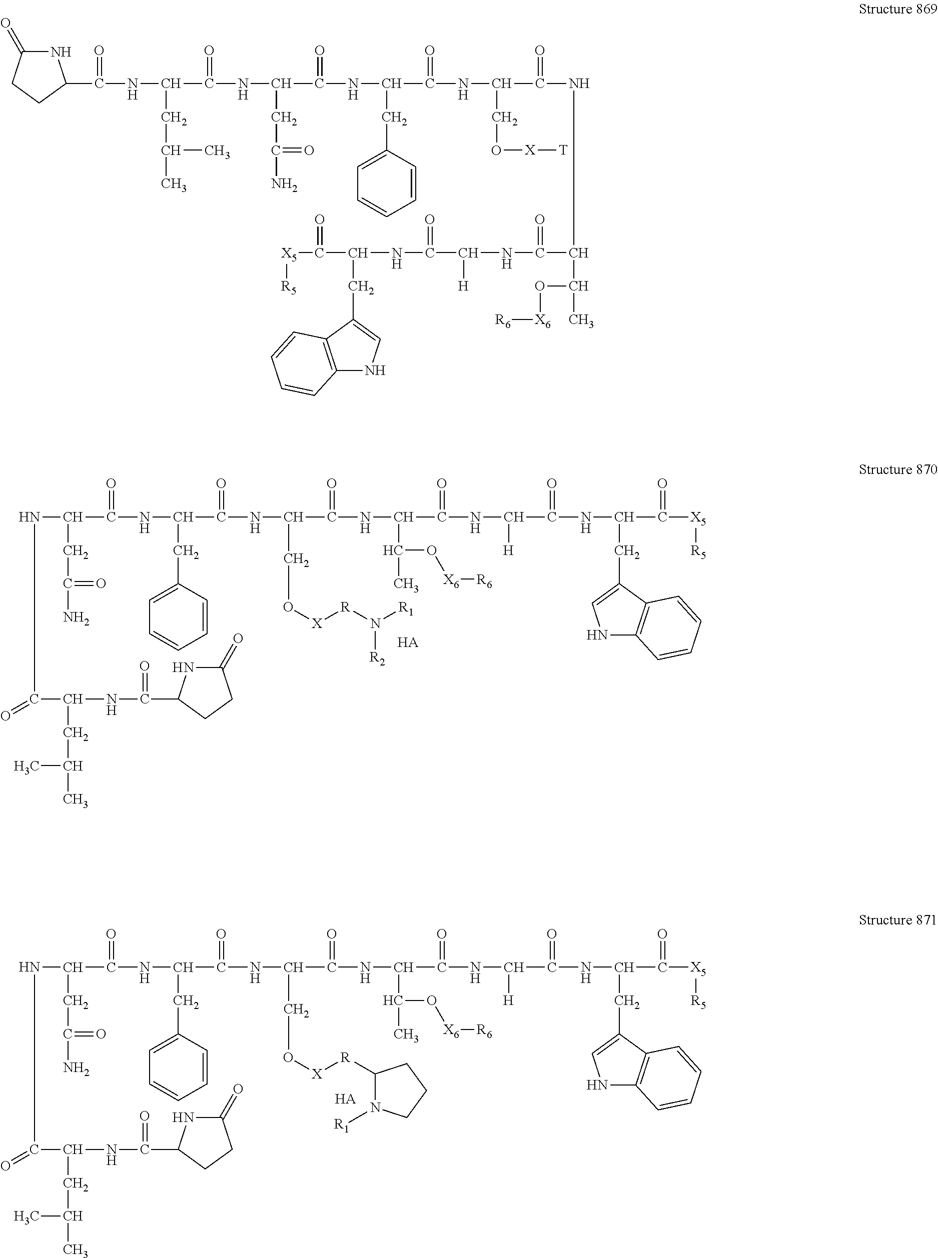

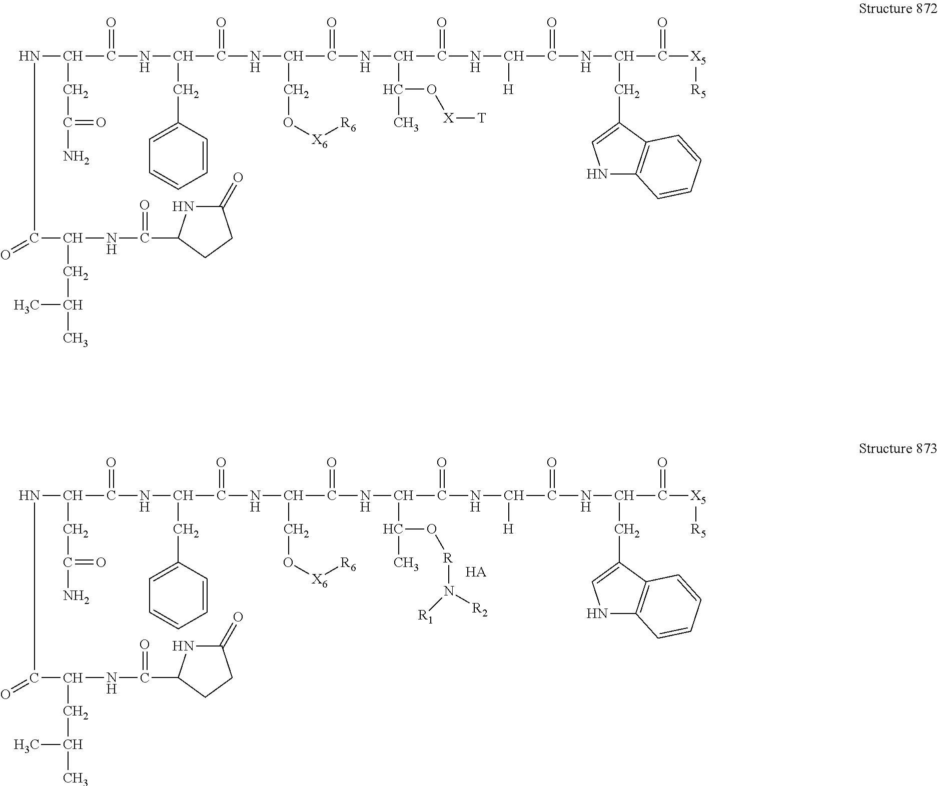

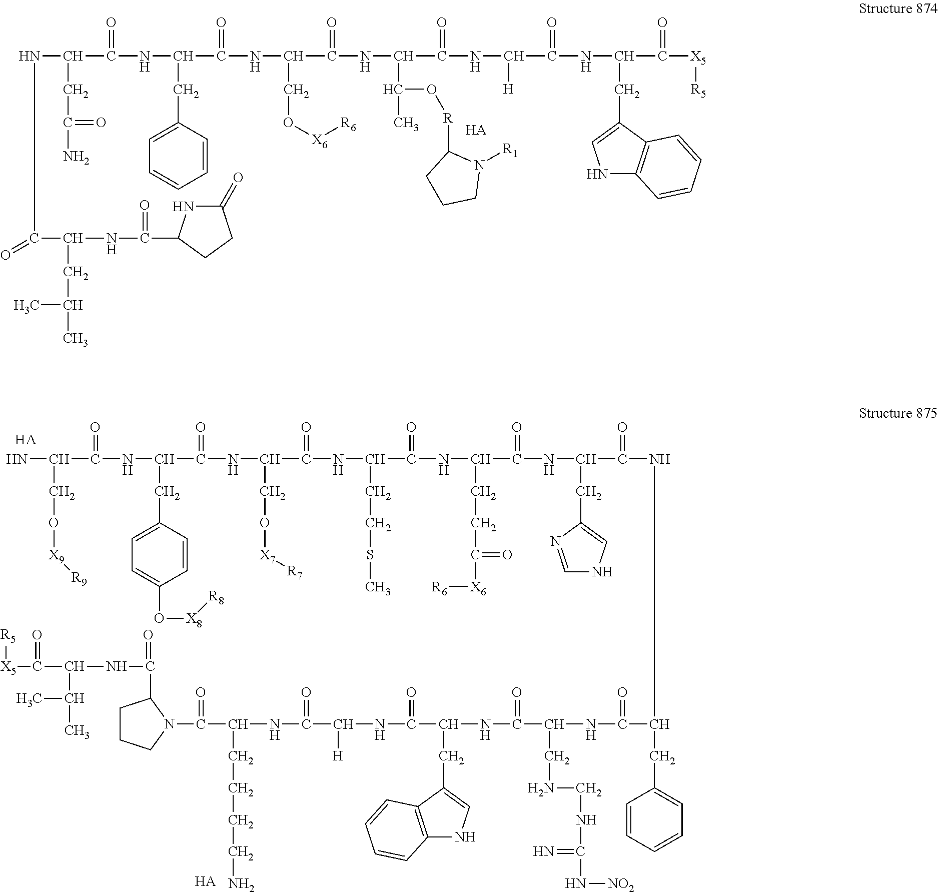

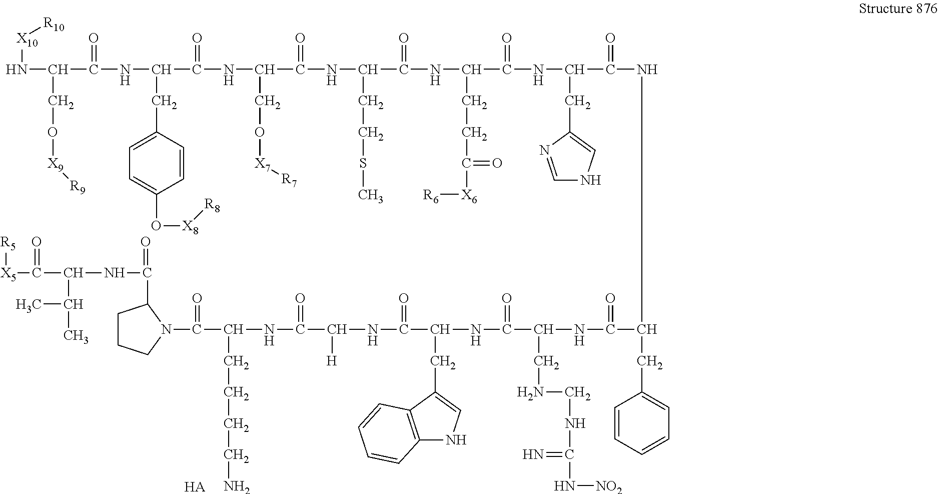

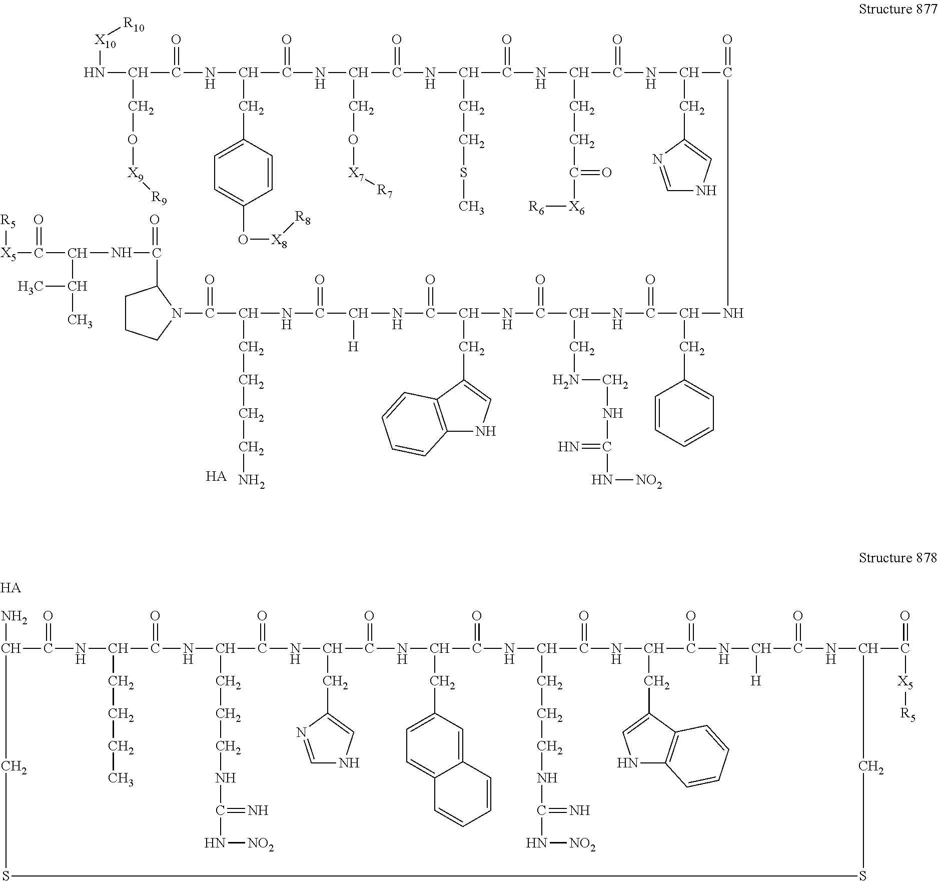

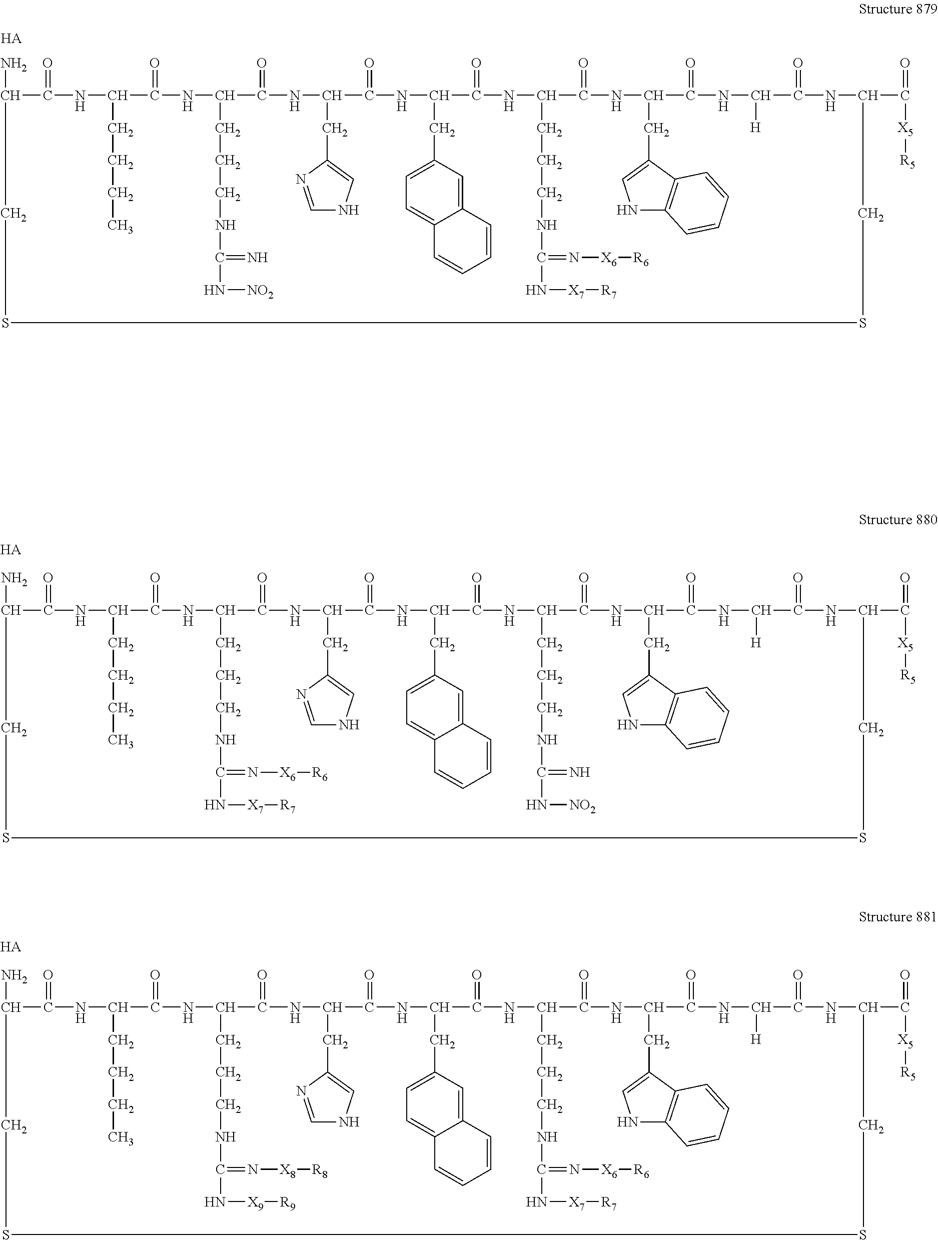

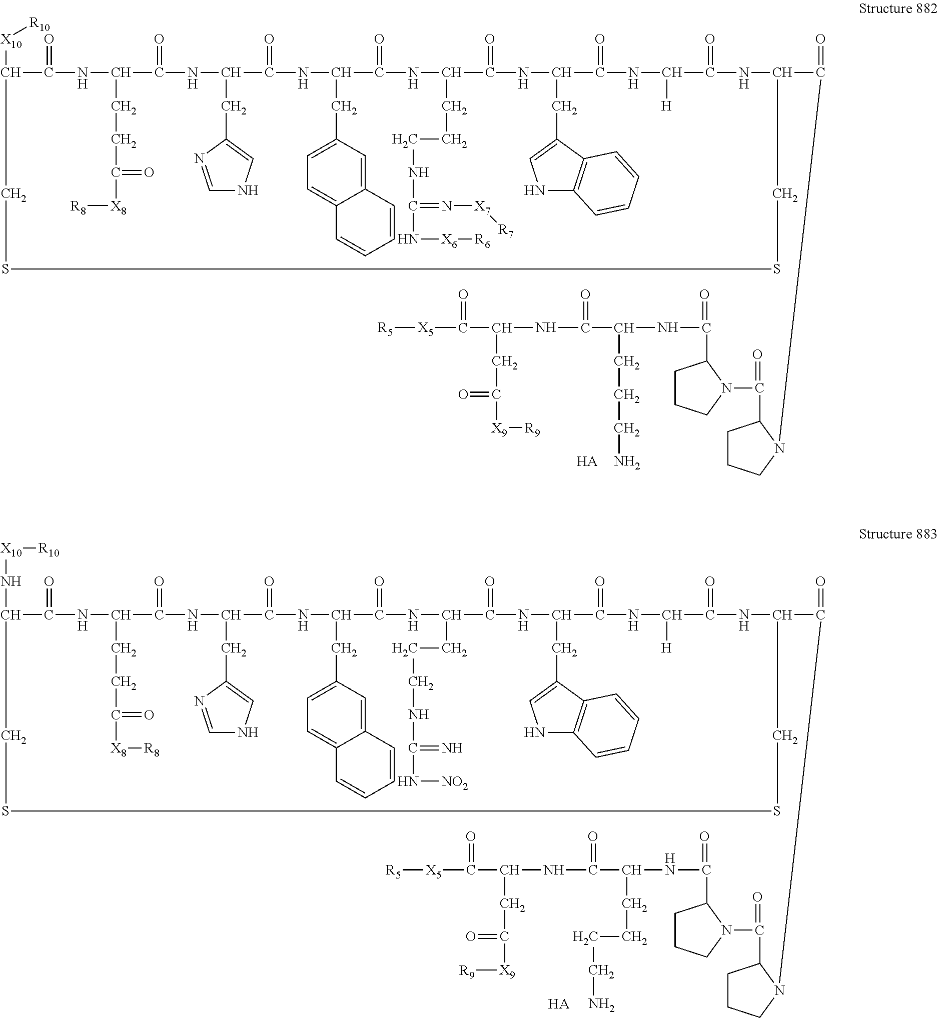

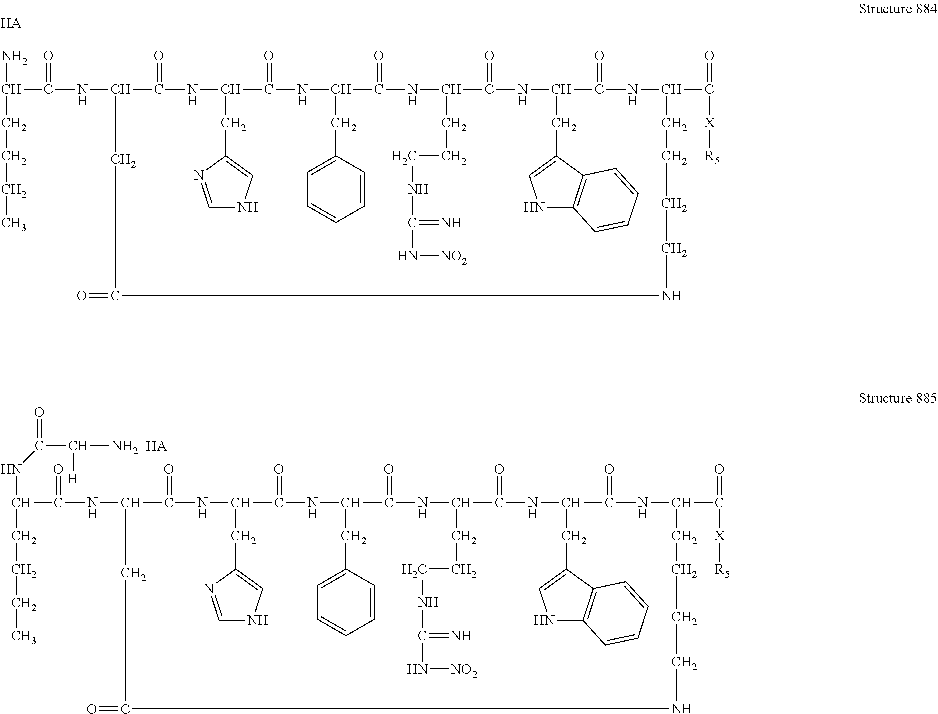

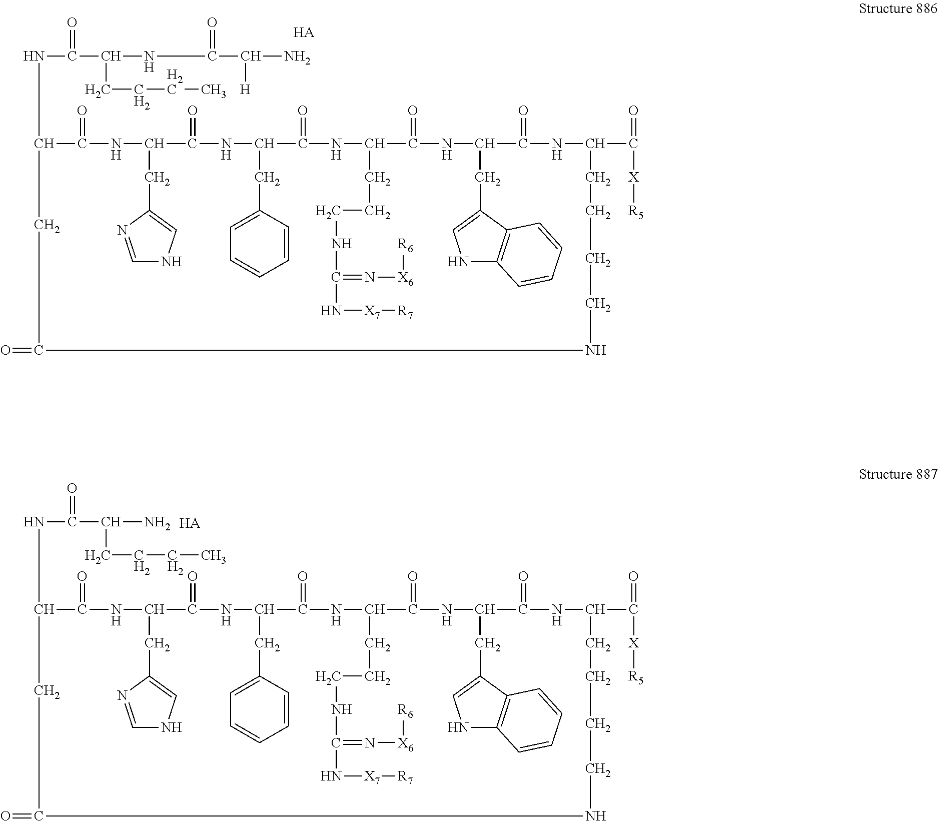

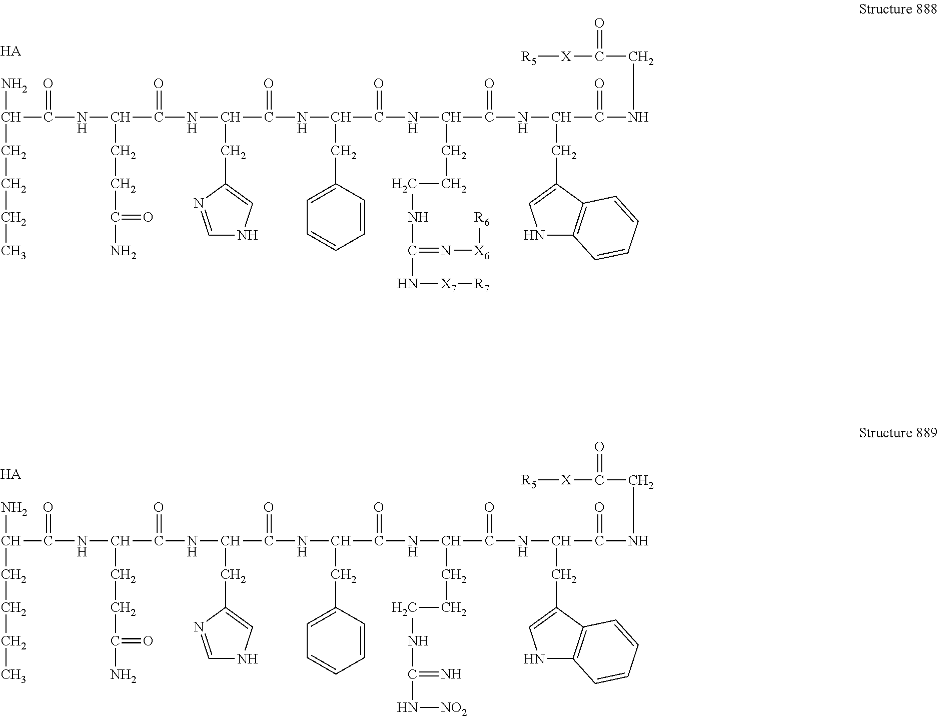

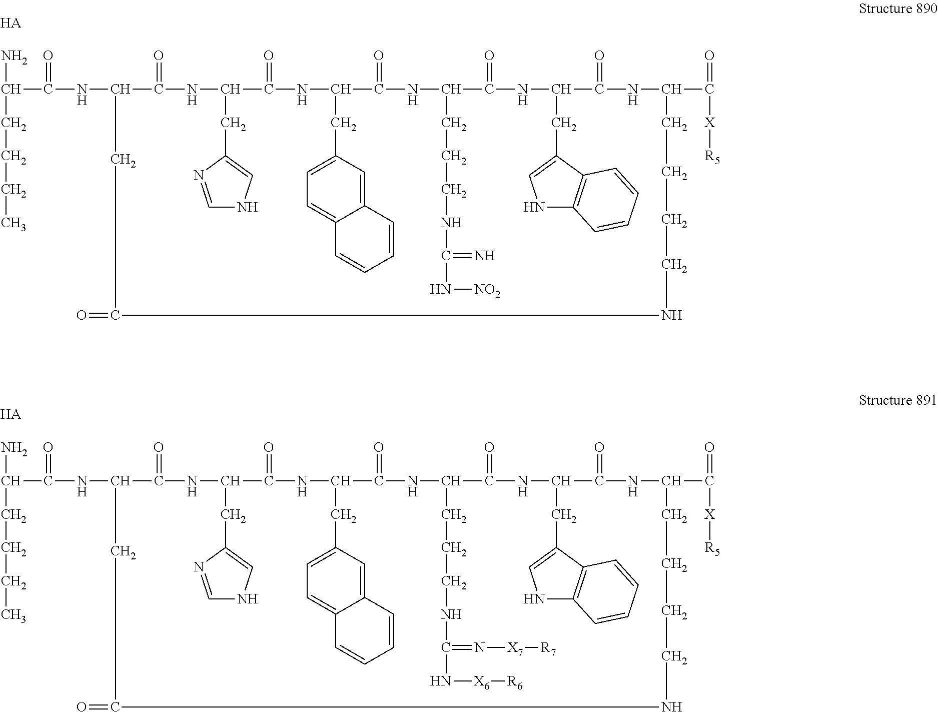

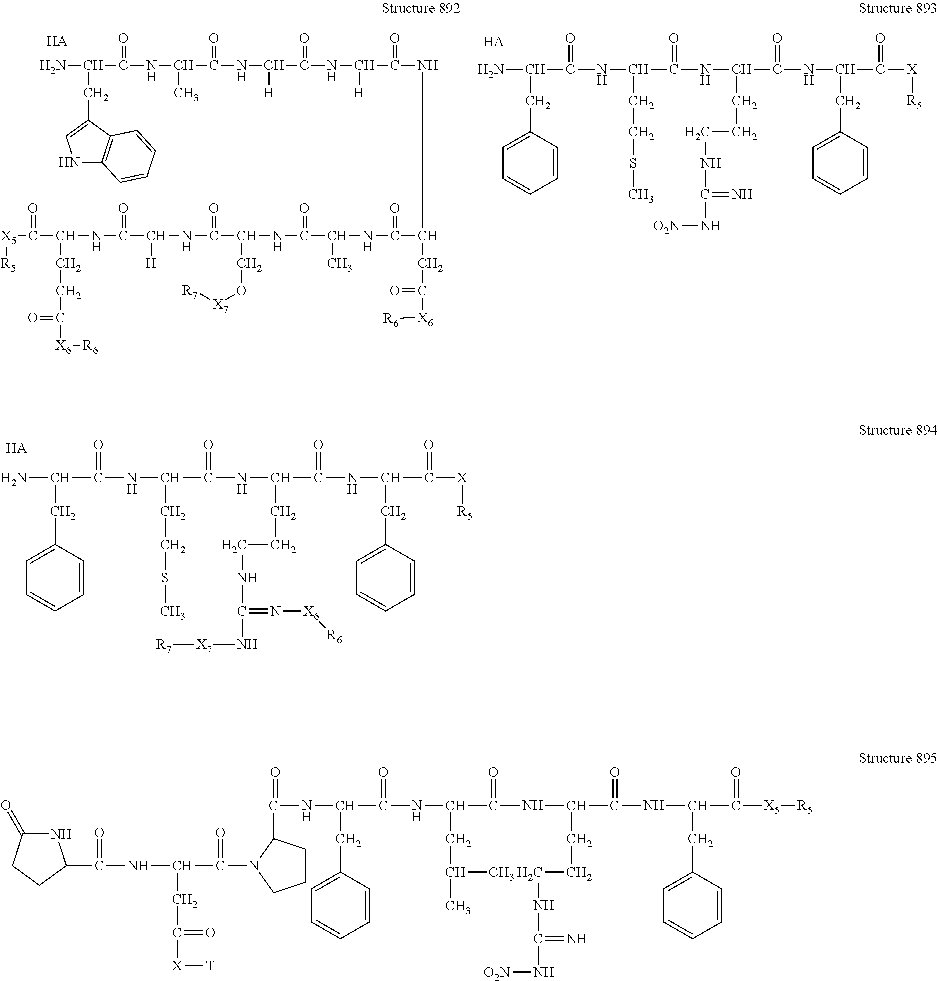

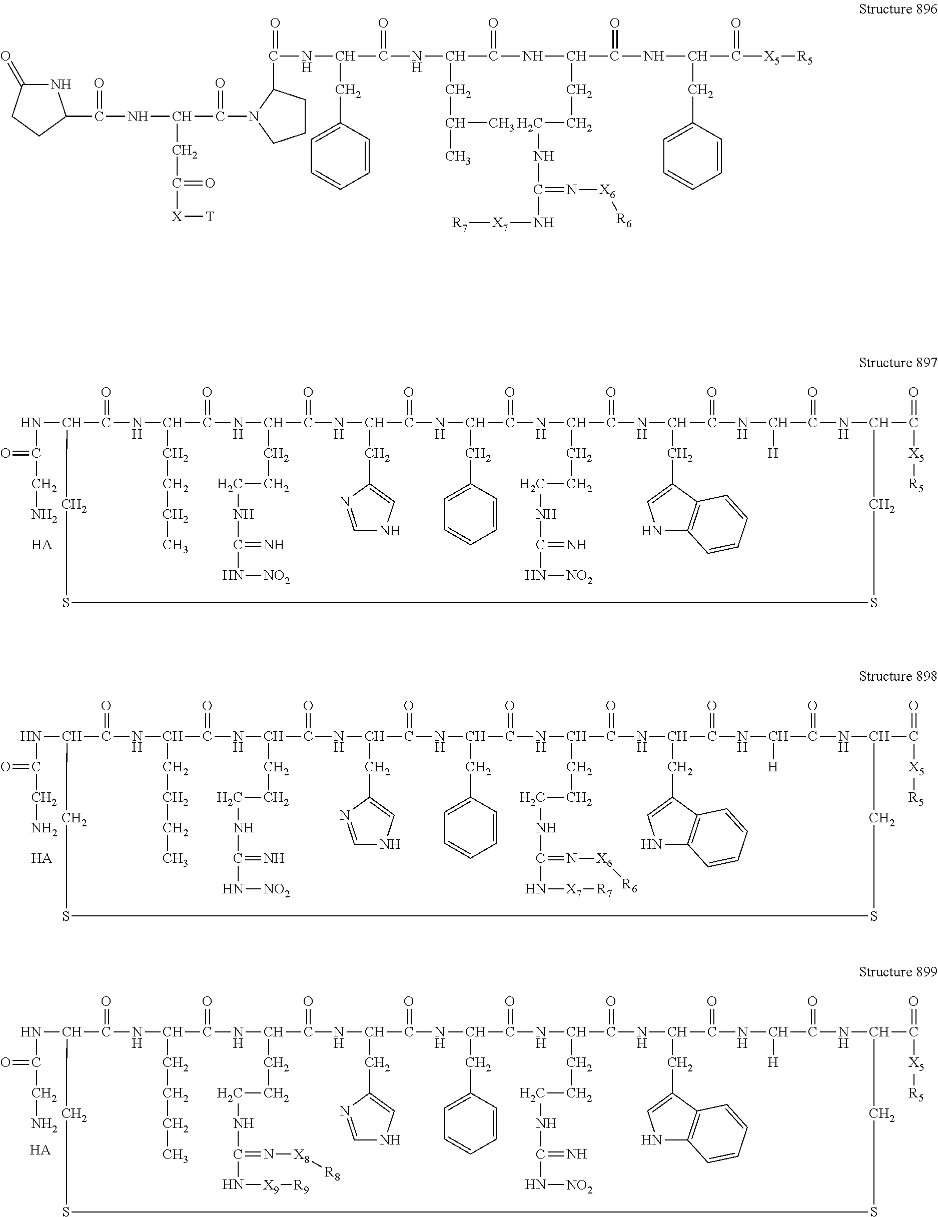

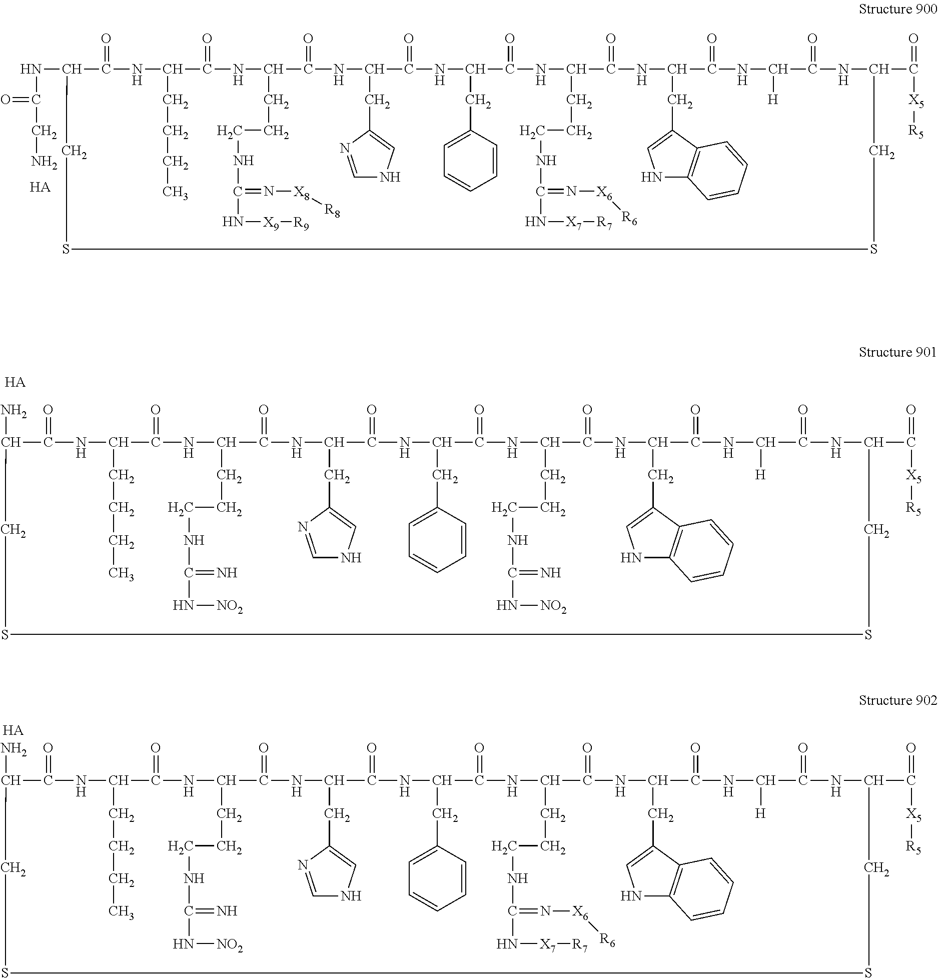

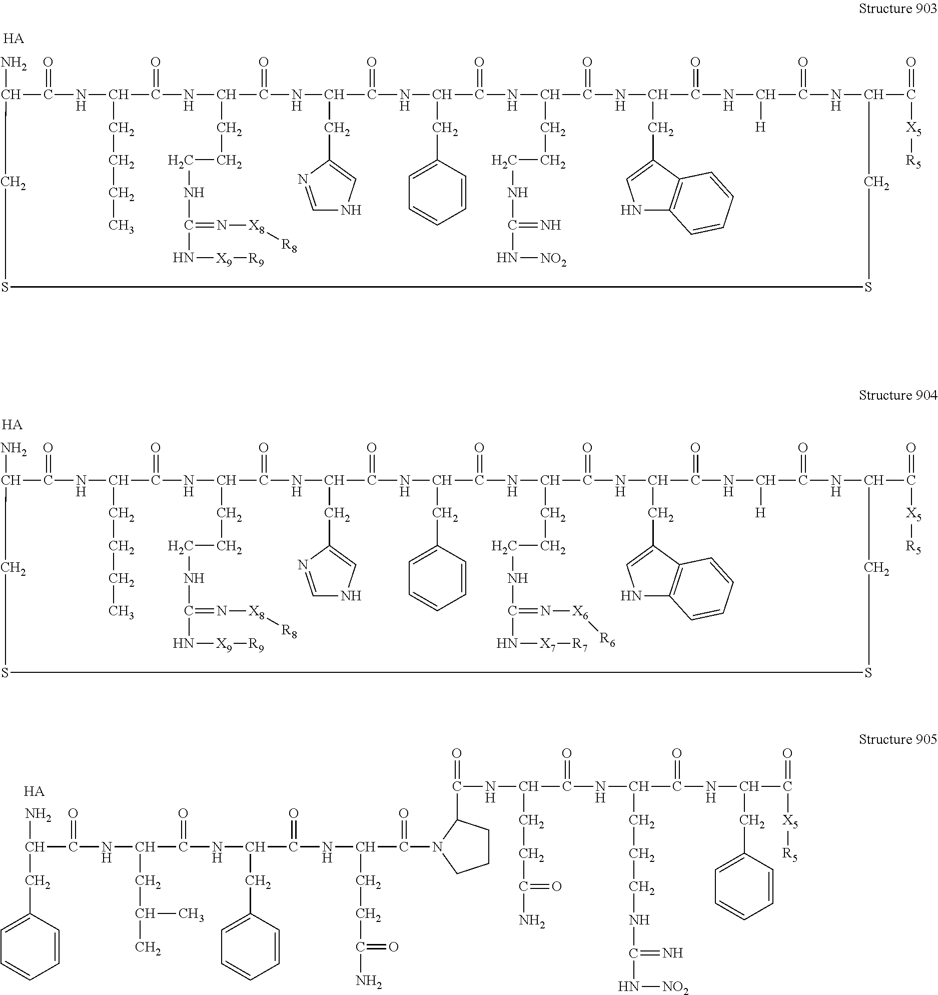

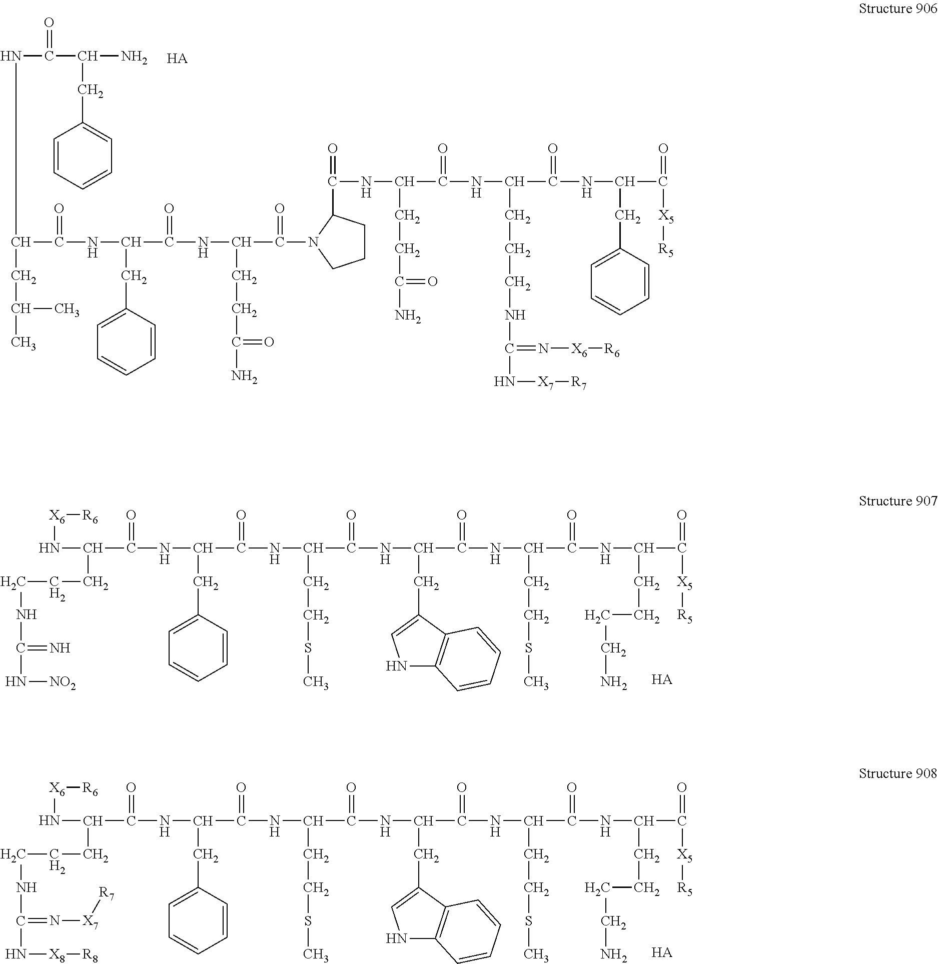

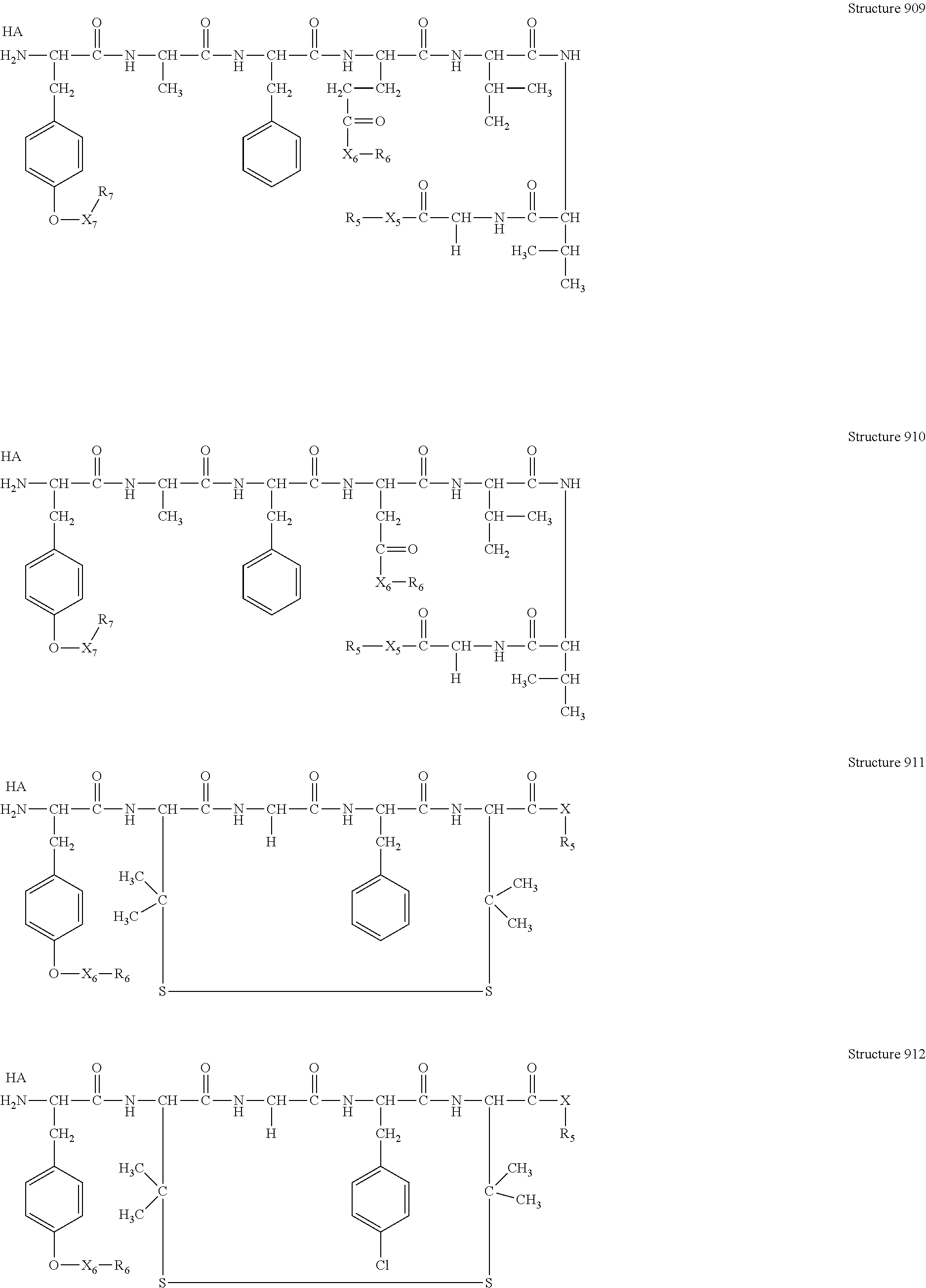

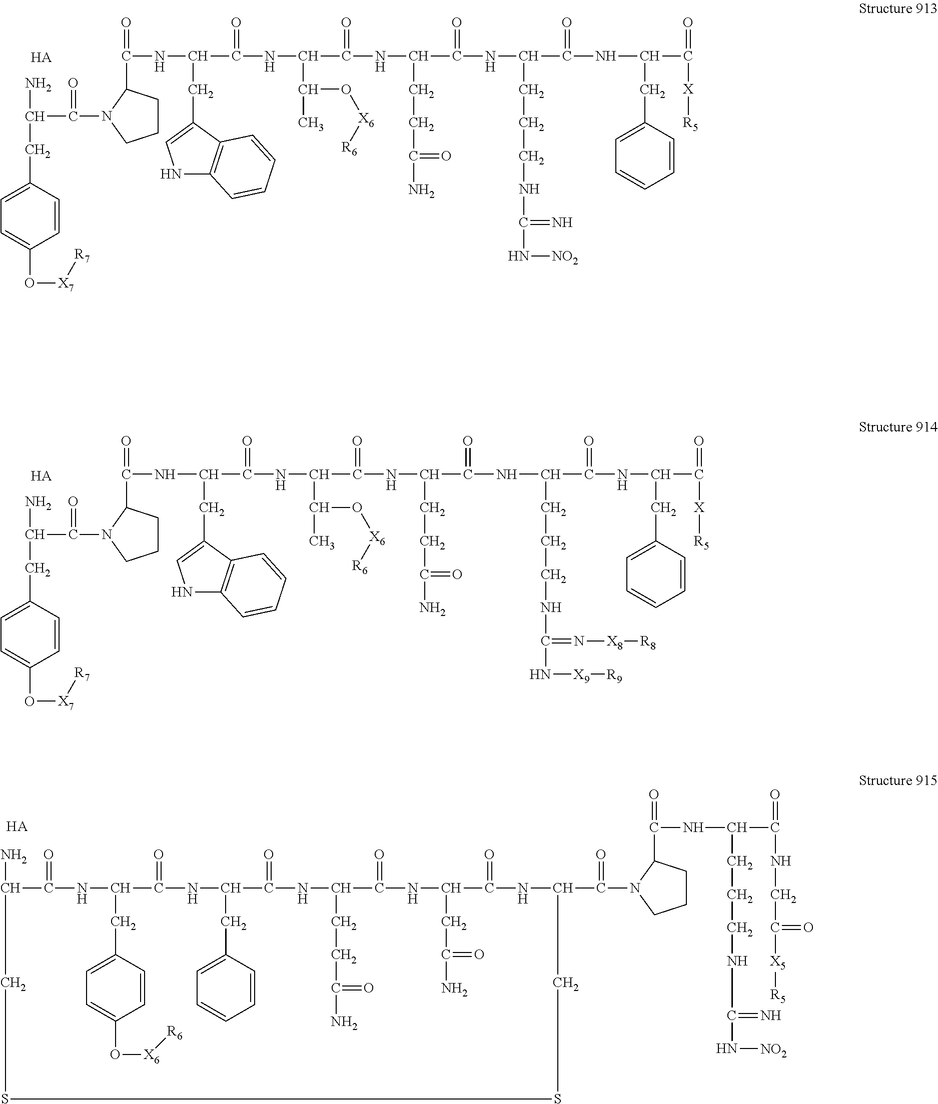

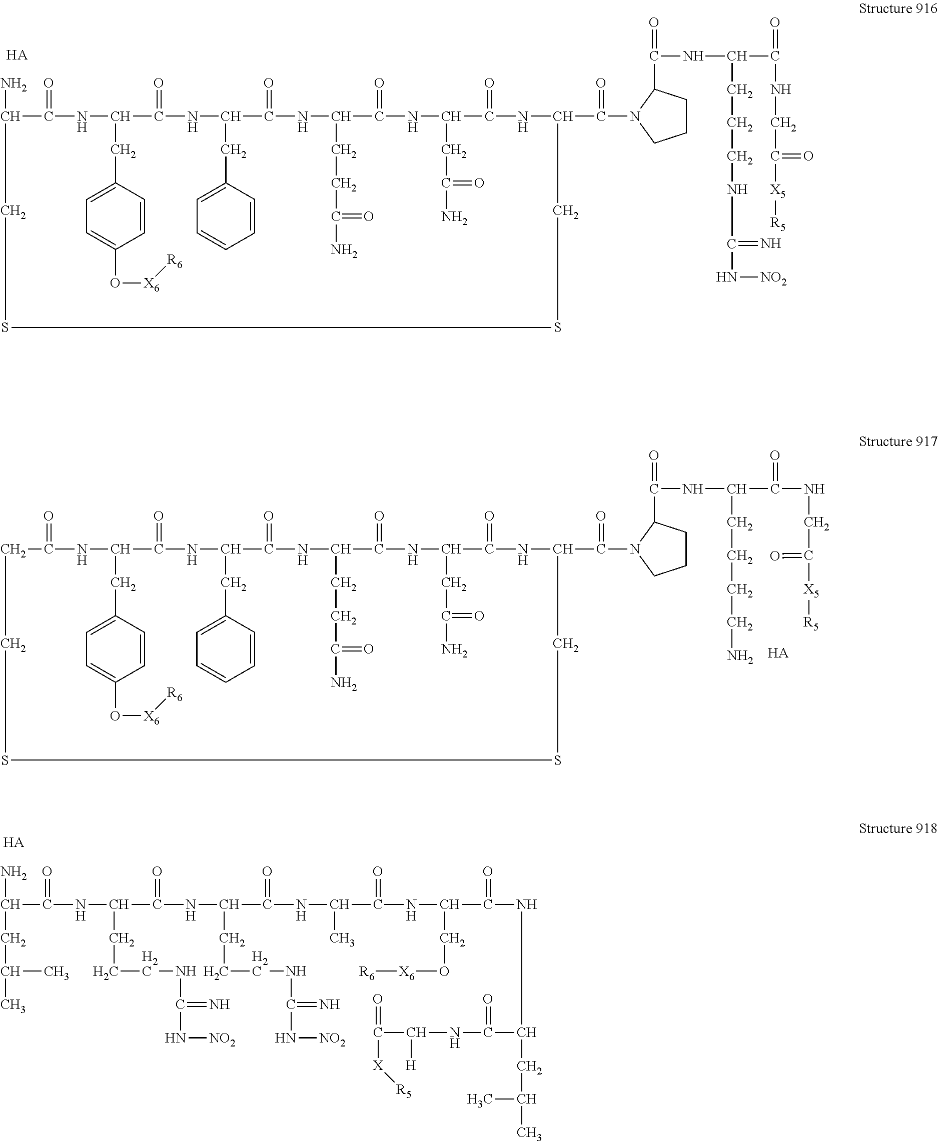

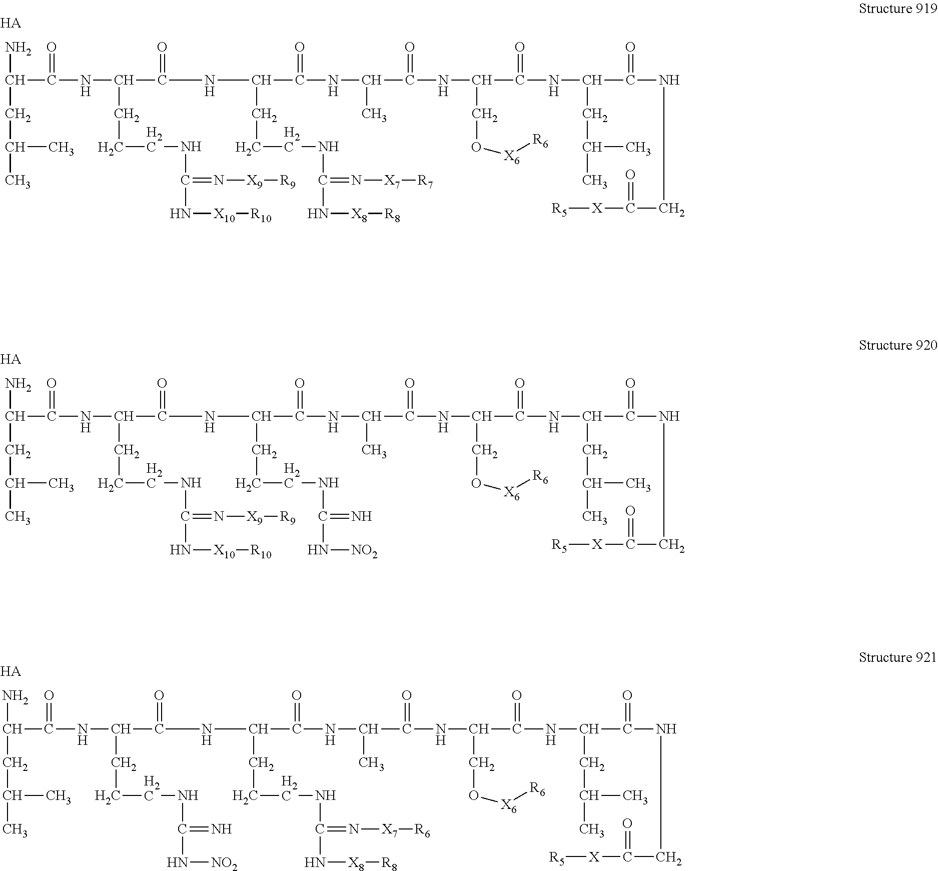

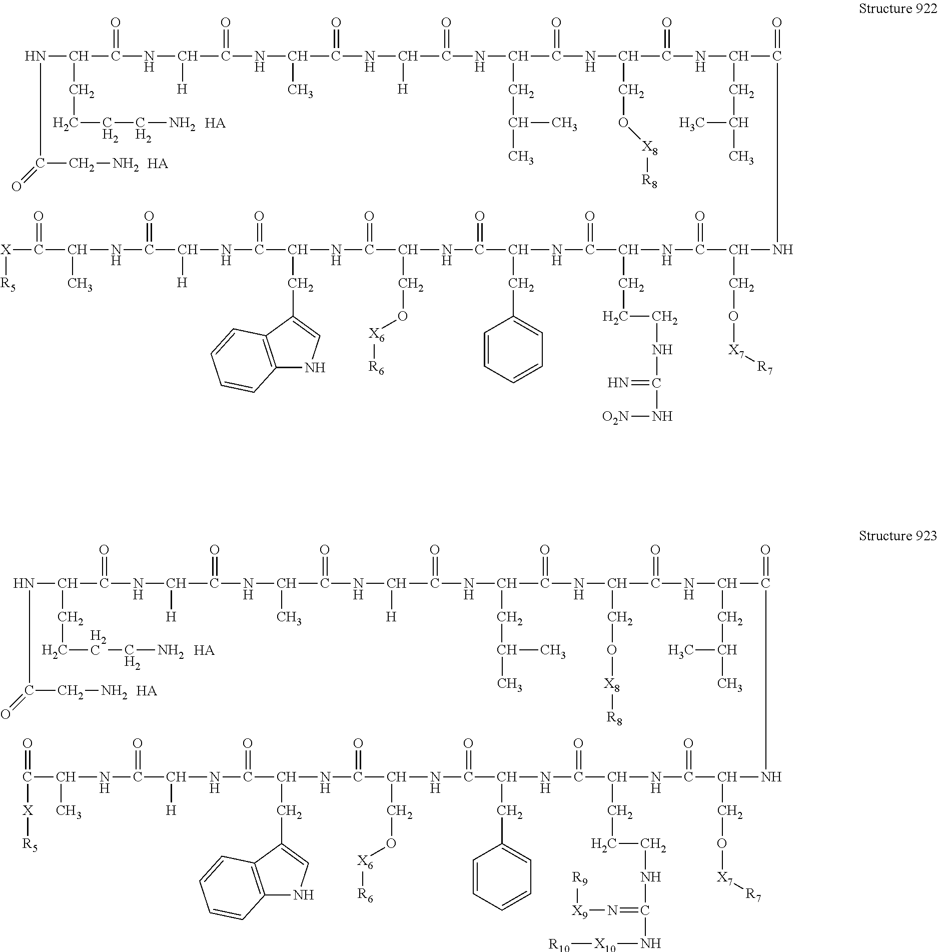

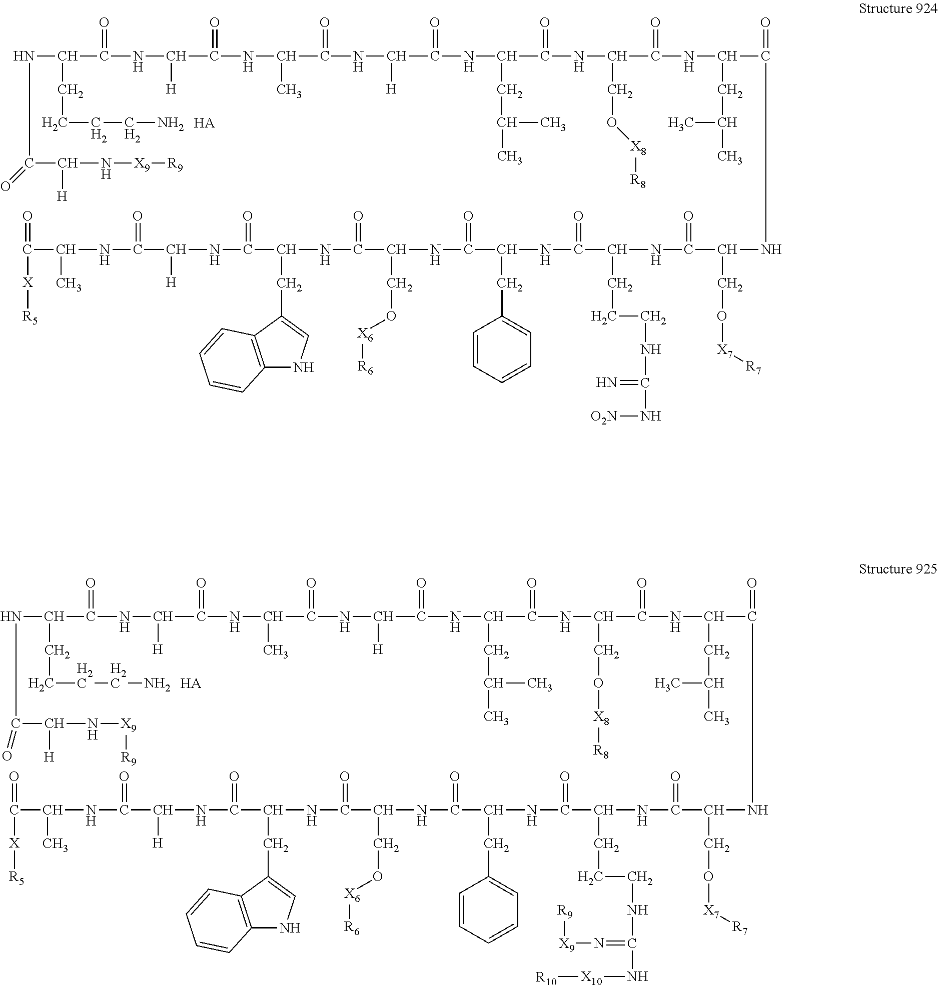

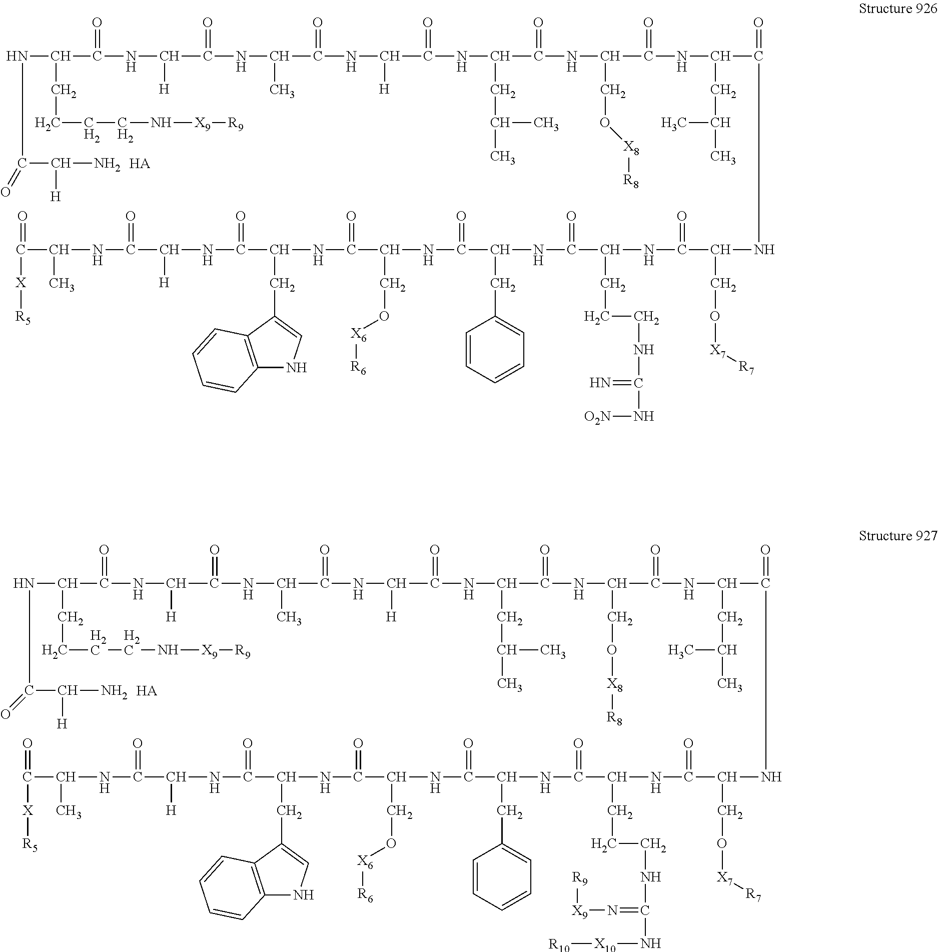

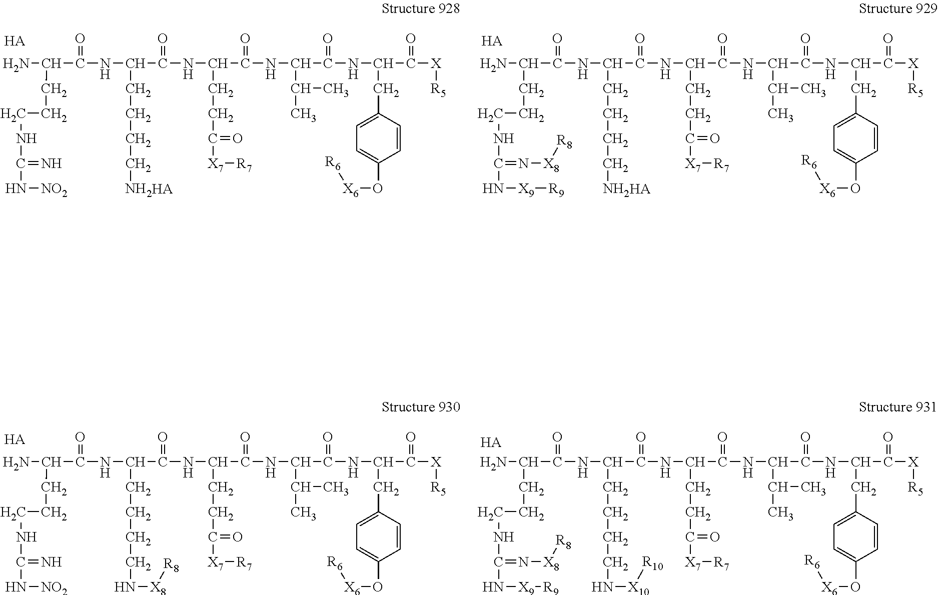

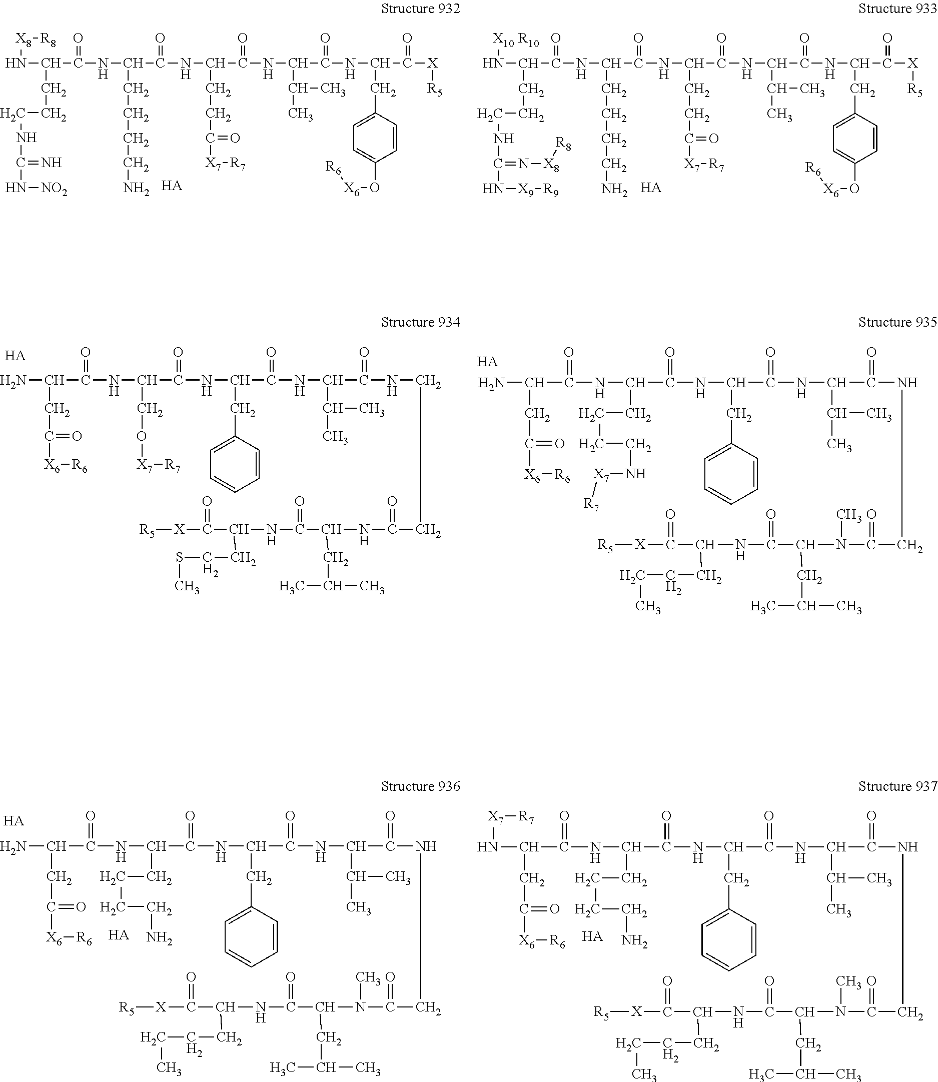

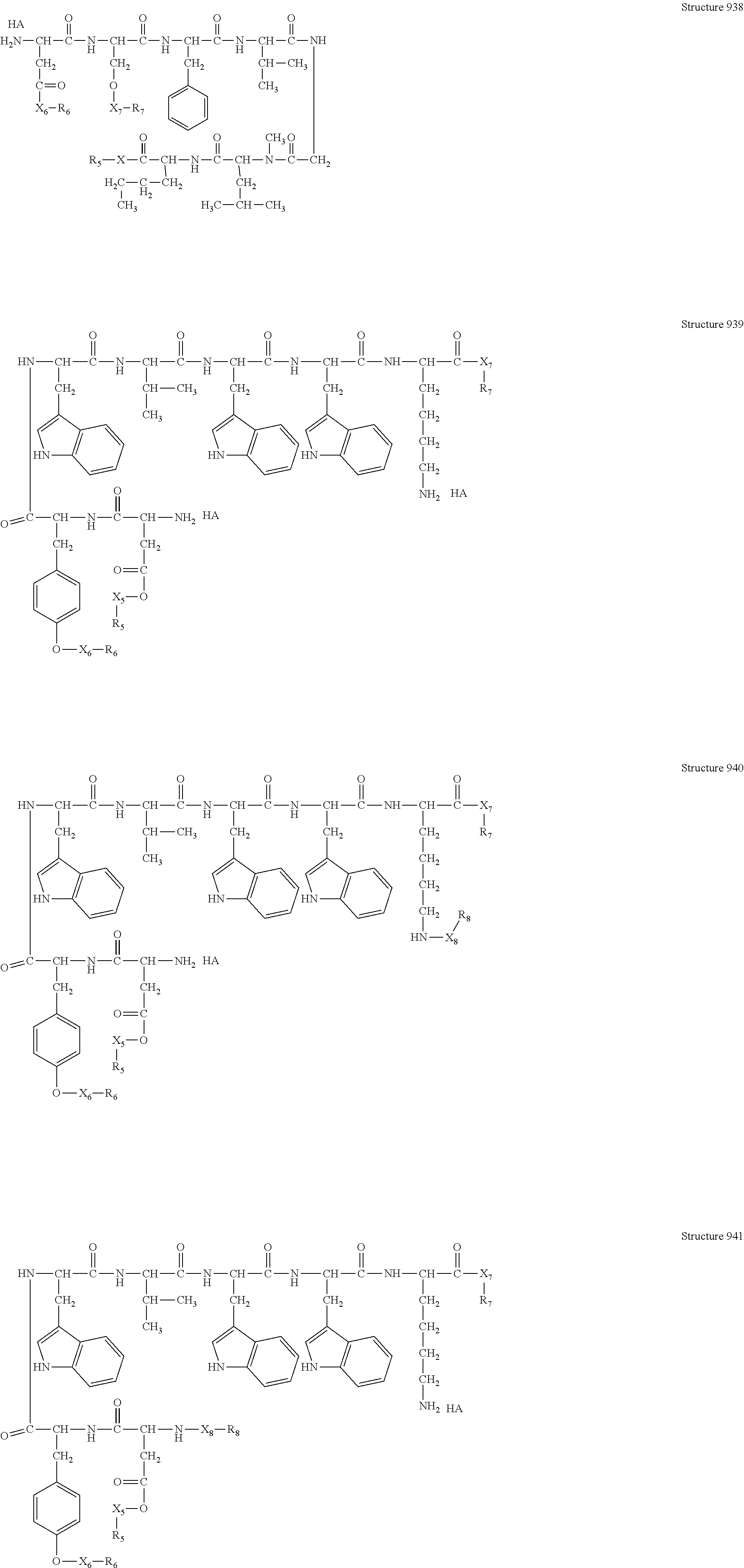

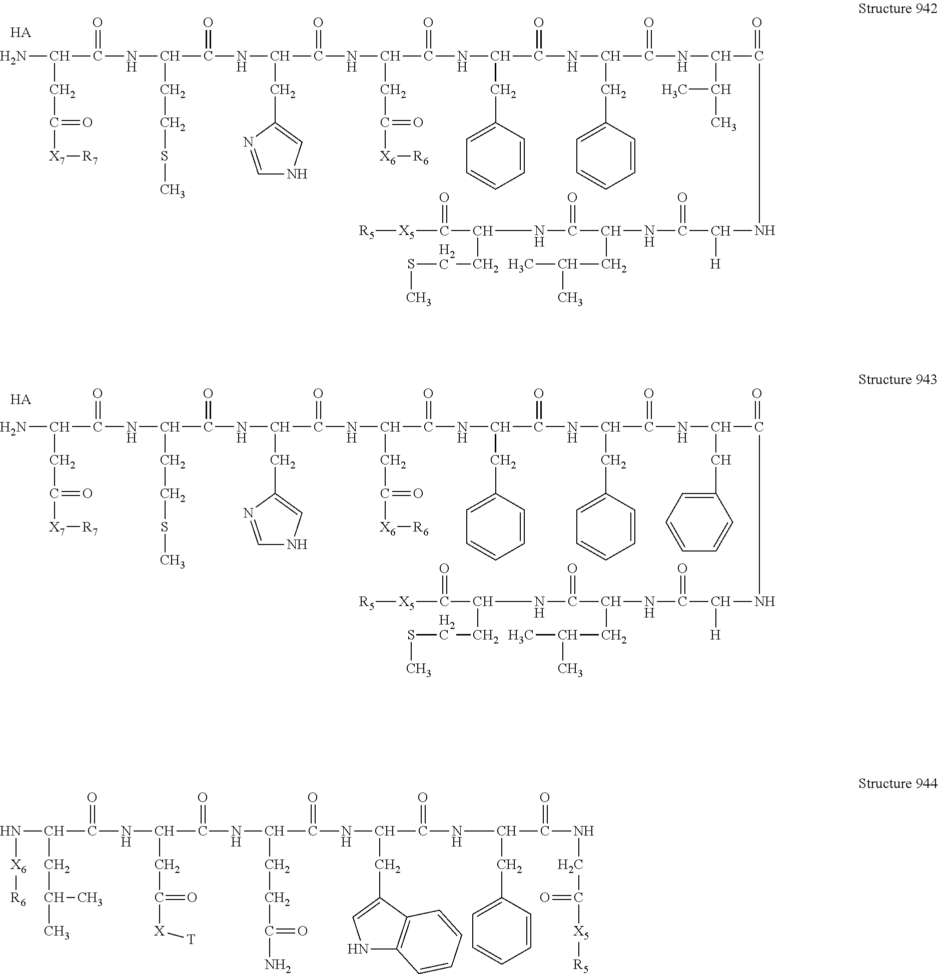

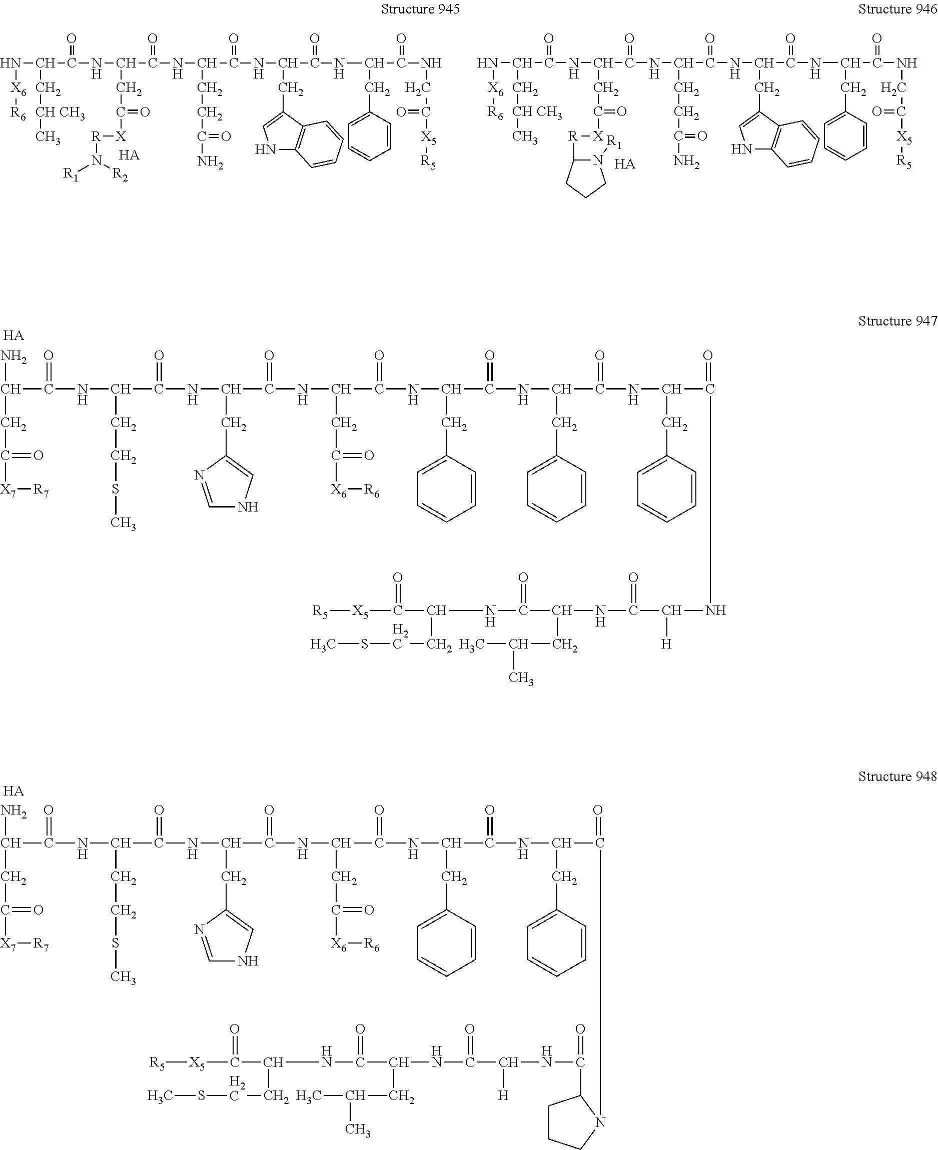

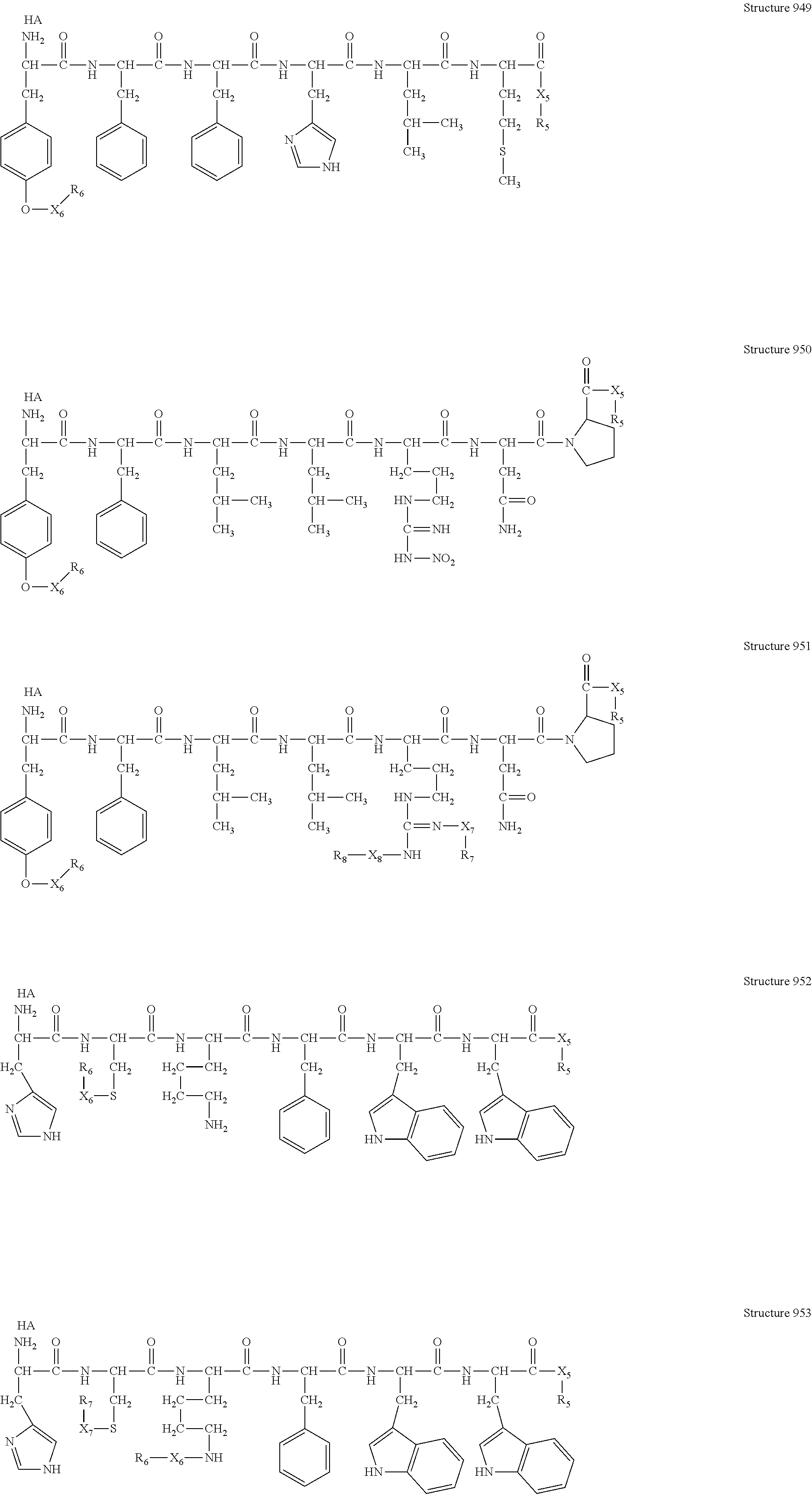

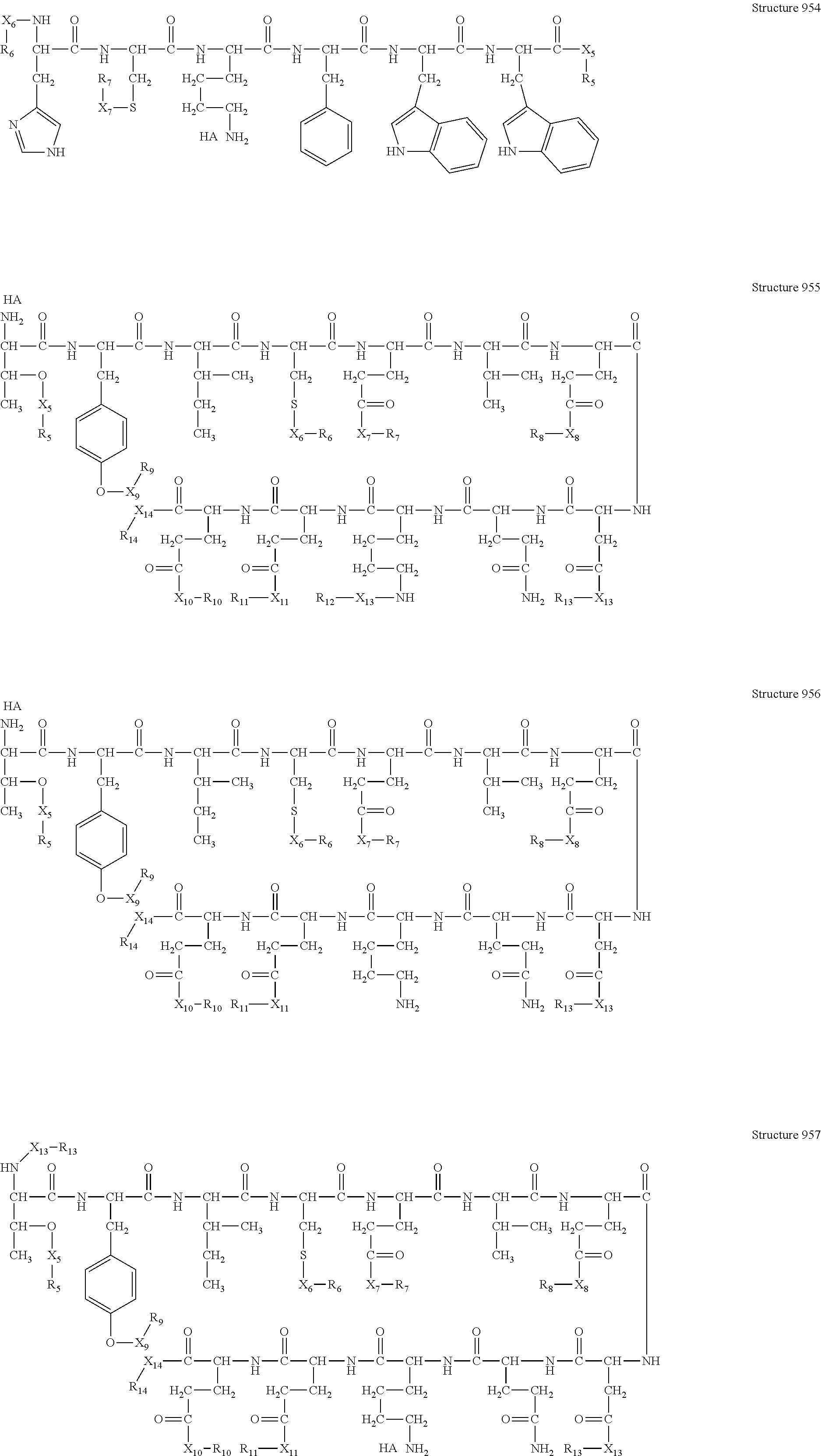

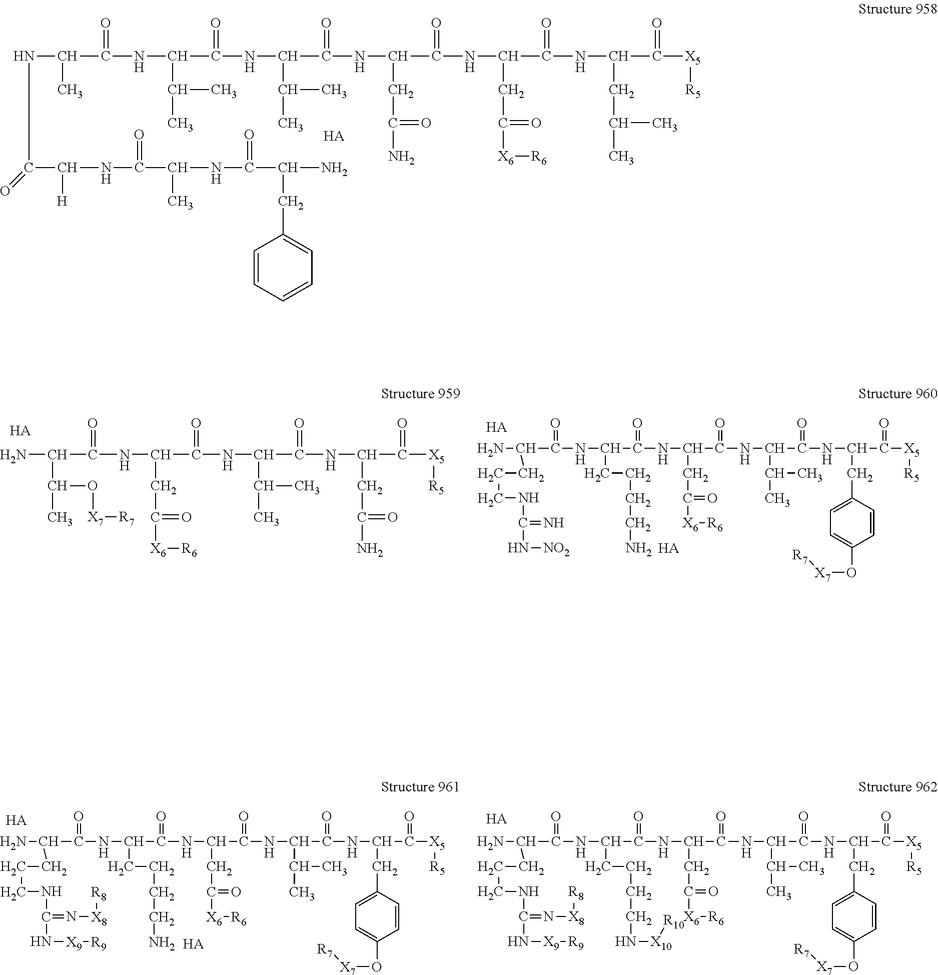

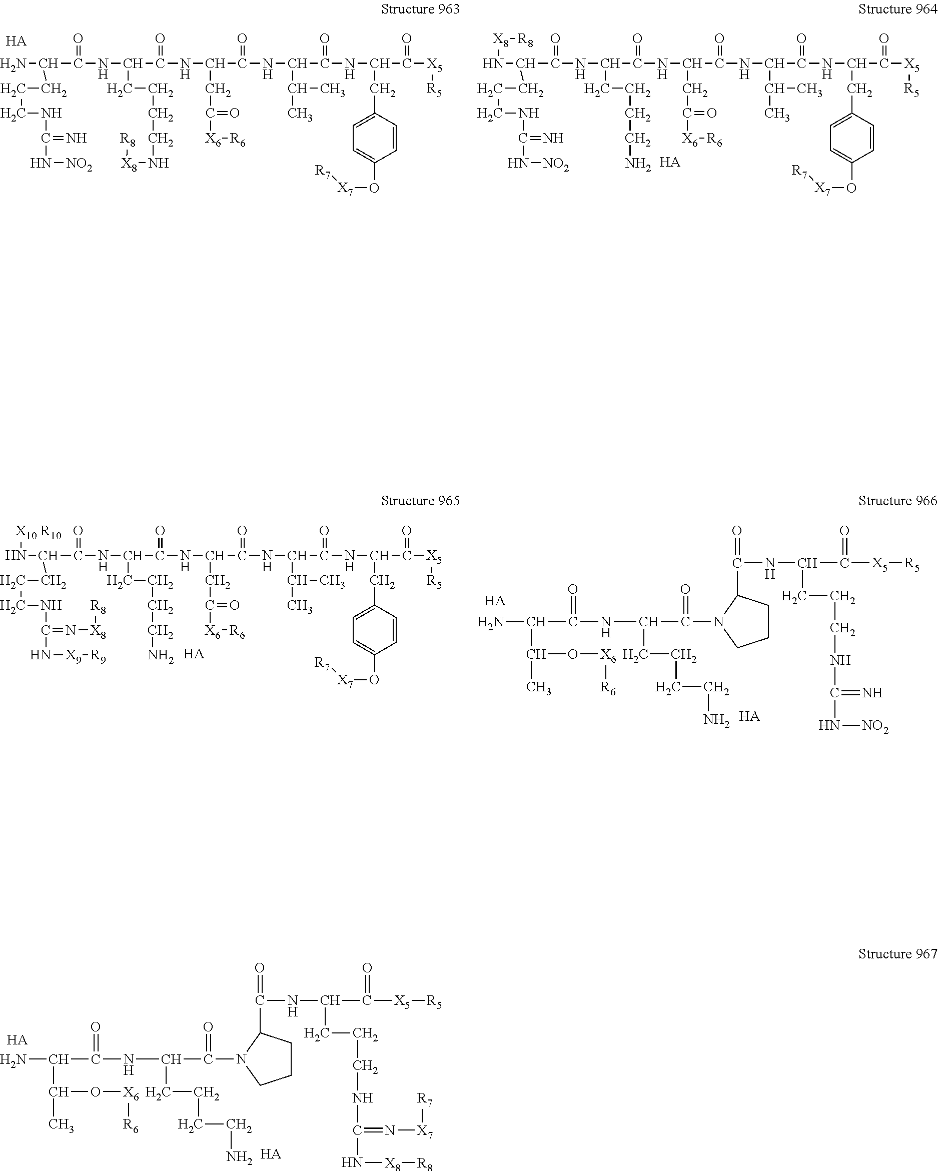

wherein

X is selected from the group consisting of nothing, O, C═O, OC(═O), C(═O)O, OC(═O)OCHR₁O, OC(═O)OCHR₁S, S, SC(═O), C(═O)S, OC(═O)SCHR₁O, SC(═O)OCHR₁O, NH, NR₆, and NR₆—C(═O)O;

X₁, X₂, X₃, X₄, X₅, X₆, X₇, X₈, X₉, X₁₀, X₁₁, X₁₂, X₁₃, X₁₄ and X₁₅ are independently selected from the group consisting of nothing, O, C═O, OC(═O), C(═O)O, OC(═O)OCHR₁O, OC(═O)OCHR₁S, S, SC(═O), C(═O)S, OC(═O)SCHR₁O, SC(═O)OCHR₁O, NH, NR₆, NR₆—C(═O)O, H, CH₃, CH₃CH₂, CH₃CH₂CH₂, (CH₃)₂CH, CH₃CH₂CH₂CH₂, CH₃CH₂CH(CH₃), CH₃CO, R₅CO, CH₃CS, R₅CS, CH₃OCO, R₅OCO, CH₃OCS, CH₃O, CH₃S, CH₃NH, R₅OCS, substituted and unsubstituted alkyl residues having 1 to 12 carbon atoms, substituted and unsubstituted cycloalkyl residues having 1 to 12 carbon atoms, substituted and unsubstituted heterocycloalkyl residues having 1 to 12 carbon atoms, substituted and unsubstituted alkenyl residues having 1 to 12 carbon atoms, substituted and unsubstituted alkynyl residues having 1 to 12 carbon atoms, substituted and unsubstituted alkyloxyl residues having 1 to 12 carbon atoms, substituted and unsubstituted cycloalkyloxyl residues having 1 to 12 carbon atoms, substituted and unsubstituted aryl residues having 1 to 12 carbon atoms, substituted and unsubstituted heteroaryl residues having 1 to 12 carbon atoms;

Y₁ is selected from the group consisting of H, F, Br, Cl, I, CH₃, CH₃O, CF₃, OR₇, CF₃O, and R₅O;

Y₂ is selected from the group consisting of H, phenyl, 4-chlorophenyl, 4-fluorophenyl, 4-bromophenyl, and 4-iodophenyl;

Y₃ is selected from the group consisting of H, phenyl, 4-chlorophenyl, 4-fluorophenyl, 4-bromophenyl, and 4-iodophenyl;

Y₄ is selected from the group consisting of H, F, Br, Cl, I, CH₃, CF₃, OR₇, and CH₃O;

Y₅ is selected from the group consisting of H, CH₃CO, C₂HSCO, and C₃H₇CO;

Y₆ is selected from the group consisting of H, F, Br, Cl, I, CH₃, CF₃, OR₇, and CH₃O;

Y₇ is selected from the group consisting of H, F, Br, Cl, I, CH₃, CF₃, OR₇, and CH₃O;

HA is a pharmaceutically acceptable acid, and can be selected from the group consisting of hydrofluoride, hydrochloride, hydrobromide, hydroiodide, nitric acid, sulfic acid, bisulfic acid, phosphoric acid, phosphorous acid, phosphonic acid, isonicotinic acid, acetic acid, propionic acid, butyric acid, valeric acid, caproic acid, heptanoic acid, octanoic acid, nonanoic acid, decanoic acid, dodecanoic acid, palmitic acid, stearic acid, lactic acid, salicylic acid, citric acid, ascorbic acid, tartaric acid, uric acid, pantothenic acid, bitartaric acid, succinic acid, maleic acid, gentisinic acid, fumaric acid, gluconic acid, glucaronic acid, saccharic acid, formic acid, benzoic acid, glutamic acid, methanesulfonic acid, ethanesulfonic acid, benzensulfonic acid, p-toluenesulfonic acid and pamoic acid;

R is selected from the group consisting of nothing, substituted and unsubstituted alkyl residues having 1 to 12 carbon atoms, substituted and unsubstituted alkenyl residues having 1 to 12 carbon atoms, substituted and unsubstituted alkynyl residues having 1 to 12 carbon atoms, substituted and unsubstituted cycloalkyl, cycloalkenyl or cycloalkynyl residues having 1 to 12 carbon atoms, substituted and unsubstituted heterocycloalkyl or heterocycloalkenyl residues having 1 to 12 carbon atoms, substituted and unsubstituted alkoxyl or alkenyloxyl residues having 1 to 12 carbon atoms, substituted and unsubstituted perfluoroalkyl residues having 1 to 12 carbon atoms, substituted and unsubstituted haloalkyl residues having 1 to 12 carbon atoms, substituted and unsubstituted aryl residues having 1 to 12 carbon atoms, and substituted and unsubstituted heteroaryl residues having 1 to 12 carbon atoms, wherein any CH₂ in R may be further replaced with O, S, P, NR₆, or any other pharmaceutically acceptable groups, and any combination thereof, Examples of R are CH₂, CHR₅, CHRSCH₂, CH₂CH₂CH₂, CH₂CH₂CH₂CH₂, CH₂CH₂CH₂CH₂CH₂;

R₁, R₂, R₃, R₄, R₆, R₆, R₇, R₇, R₈, R_{8′}, R₉, R_{9′}, R₁₀, R_{10′}, R₁₁, R₁₂, R₁₃, R₁₄, and R₁₅ are independently selected from the group consisting of H, CH₃CO, R₅CO, CH₃CS, R₅CS, CH₃OCO, R₅OCO, CH₃OCS, CH₃O, CH₃S, CH₃NH, R₅OCS, substituted and unsubstituted alkyl having 1 to 12 carbon atoms, substituted and unsubstituted alkenyl having 1 to 12 carbon atoms, substituted and unsubstituted alkynyl having 1 to 12 carbon atoms, substituted and unsubstituted cycloalkyl, cycloalkenyl or cycloalkynyl having 1 to 12 carbon atoms, substituted and unsubstituted heterocycloalkyl or heterocycloalkenyl having 1 to 12 carbon atoms, substituted and unsubstituted alkoxyl or alkenoxyl having 1 to 12 carbon atoms, substituted and unsubstituted cycloalkyloxyl or cycloalkenyloxyl having 1 to 12 carbon atoms, substituted and unsubstituted aryl having 1 to 12 carbon atoms, substituted and unsubstituted heteroaryl having 1 to 12 carbon atoms, and any combination thereof,

R₅ is selected from the group consisting of substituted and unsubstituted alkyl having 1 to 12 carbon atoms, substituted and unsubstituted alkenyl having 1 to 12 carbon atoms, substituted and unsubstituted alkynyl having 1 to 12 carbon atoms, substituted and unsubstituted cycloalkyl having 1 to 12 carbon atoms, substituted and unsubstituted heterocycloalkyl having 1 to 12 carbon atoms, substituted and unsubstituted alkoxyl having 1 to 12 carbon atoms, substituted and unsubstituted cycloalkyloxyl having 1 to 12 carbon atoms, substituted and unsubstituted aryl having 1 to 12 carbon atoms, substituted and unsubstituted heteroaryl having 1 to 12 carbon atoms, and residues thereof;

Z represents CH₂═C, CH═CH, C—C, CONH, CSNH, COO, OCO, COS, COCH₂, or CH₂CO;

Every hydrogen in parent drugs or transportational units can be replaced with a deuterium without significant changes in pharmaceutical properties, chemical properties and physical properties;

T is a transportational unit, for example, selected from the group consisting of protonated amine groups, especially pharmaceutically acceptable substituted and unsubstituted primary amine groups, pharmaceutically acceptable substituted and unsubstituted secondary amine groups, and pharmaceutically acceptable substituted and unsubstituted tertiary amine groups in protonated form. Examples of T are Structure T-1, Structure T-2, Structure T-3, Structure T-4, Structure T-5, Structure T-6, Structure T-7, Structure T-8, Structure T-9, Structure T-10, Structure T-11, and Structure T-12:

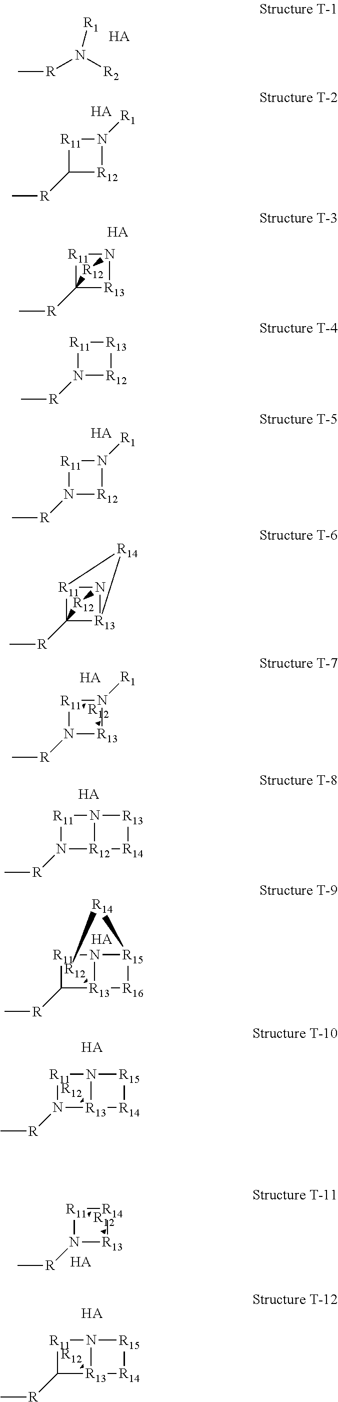

wherein R₁ and R₂ are defined as above; R₁₁, R₁₂, R₁₃, R₁₄, R₁₅ and R₁₆ are selected from the group consisting of nothing, substituted and unsubstituted alkyl residues having 1 to 12 carbon atoms, substituted and unsubstituted alkenyl residues having 1 to 12 carbon atoms, substituted and unsubstituted alkynyl residues having 1 to 12 carbon atoms, substituted and unsubstituted cycloalkyl, cycloalkenyl or cycloalkynyl residues having 1 to 12 carbon atoms, substituted and unsubstituted heterocycloalkyl or heterocycloalkenyl residues having 1 to 12 carbon atoms, substituted and unsubstituted alkoxyl or alkenoxyl residues having 1 to 12 carbon atoms, substituted and unsubstituted perfluoroalkyl residues having 1 to 12 carbon atoms, substituted and unsubstituted haloalkyl residues having 1 to 12 carbon atoms, substituted and unsubstituted aryl residues having 1 to 12 carbon atoms, and substituted and unsubstituted heteroaryl residues having 1 to 12 carbon atoms, wherein any CH₂ in R may be further replaced with O, S, P, NR₆, or any other pharmaceutically acceptable groups, and any combination thereof, every hydrogen in parent drugs or transportational units can be replaced with a deuterium without significant changes in pharmaceutical properties, chemical properties and physical properties.

As used herein, the term “pharmaceutically acceptable salt” means those salts of compounds of the invention that are safe for application in a subject. Pharmaceutically acceptable salts include salts of acidic or basic groups present in compounds of the invention. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,11-methylene-bis-(2-hydroxy-3-naphthoate)) salts. Certain compounds of the invention can form pharmaceutically acceptable salts with various amino acids. Suitable base salts include, but are not limited to, aluminum, calcium, lithium, magnesium, potassium, sodium, zinc, and diethanolamine salts. For a review on pharmaceutically acceptable salts see BERGE ET AL., 66 J. PHARM. SCI. 1-19 (1977), incorporated herein by reference.

As a person of skill in the art would understand, the structures defined above encompass only those stable compounds without violation of covalent bond forming principles.

### II. Methods for Improving the Stability of the Reconstitution Solution of the Pharmaceutical Composition

Unexpectedly, it is discovered that, unlike common ester or ammonium compounds, the stability of the HPDs in a solution varies significantly with the pH value, concentration and temperature of the solution, whereas the acid which forms salt with the amine group, and the substituents on the amine group, only have a slight effect on the stability. The results are illustrated in the following.

### 1. Effect of Concentration on the Stability

TABLE 1
Effect of Concentration of H-Val-Pro-Gly-Pro-Arg(NO₂)—OCH₂CH₃•HCl
salt with 1 equivalent sodium acetate in 50% ethanol at 25° C. on the stability.

| Concentration (wt %) | 0.01% | 0.1% | 1% | 3% | 5% | 7% | 8% | 10% | 15% | 20% | 30% | 50% |
|---|---|---|---|---|---|---|---|---|---|---|---|---|
| Purity (%) at day 0 | 98.1 | 98.2 | 98.5 | 98.6 | 98.7 | 98.7 | 98.6 | 98.6 | 98.8 | 98.6 | 98.7 | 98.7 |
| Purity(%) at Day 30 | 55.5 | 65.7 | 91.1 | 96.8 | 97.3 | 97.6 | 97.7 | 97.5 | 97.7 | 97.5 | 97.5 | 97.7 |
| Purity (%) at Day 90 | 22.2 | 31.3 | 77.3 | 93.2 | 94.9 | 95.2 | 95.2 | 95.3 | 95.3 | 95.1 | 95.1 | 95.3 |
| Purity (%) at Day 180 | 0 | 9.6 | 58.6 | 87.8 | 90.5 | 91.7 | 91.6 | 91.7 | 91.8 | 91.5 | 91.6 | 91.8 |

The concentration of H-Val-Pro-Gly-Pro-Arg(NO₂)—OCH₂CH₃.HCl salt affects the stability, and it is not stable when the concentration is 0.1% or lower.

TABLE 2
Effect of Concentration of H-Val-Pro-Gly-Pro-Arg(NO₂)—OCH(CH₃)₂•HCl
salt with 1 equivalent sodium acetate in 50% ethanol at 25° C. on the stability.

| Concentration (wt %) | 0.01% | 0.1% | 1% | 3% | 5% | 7% | 8% | 10% | 15% | 20% | 30% | 50% |
|---|---|---|---|---|---|---|---|---|---|---|---|---|
| Purity (%) at day 0 | 98.1 | 98.2 | 98.5 | 98.6 | 98.7 | 98.7 | 98.6 | 98.6 | 98.8 | 98.6 | 98.7 | 98.7 |
| Purity(%) at Day 30 | 63.6 | 78.7 | 93.3 | 97.5 | 98.2 | 98.3 | 98.3 | 98.3 | 98.3 | 98.4 | 98.3 | 98.3 |
| Purity (%) at Day 90 | 31.2 | 49.3 | 81.9 | 95.4 | 97.6 | 97.7 | 97.6 | 97.7 | 97.6 | 97.8 | 97.7 | 97.6 |
| Purity (%) at Day 180 | 10.3 | 25.6 | 68.6 | 92.2 | 95.2 | 95.5 | 95.4 | 95.6 | 95.3 | 95.7 | 95.5 | 95.5 |
| Purity(%) at Day 360 | 0 | 5.1 | 47.9 | 84.5 | 91.9 | 92.3 | 92.1 | 92.3 | 92.0 | 92.4 | 92.3 | 92.2 |

The concentration of H-Val-Pro-Gly-Pro-Arg(NO₂)—OCH(CH₃)₂.HBr salt affects the stability, and it is not stable when the concentration is 0.1% or lower.

TABLE 3
Effect of Concentration of H-Val-Pro-Gly-Pro-Arg(NO₂)—OCH(CH₃)₂•HBr
salt with 1 equivalent sodium acetate in 50% ethanol at 25° C. on the stability.

| Concentration (wt %) | 0.01% | 0.1% | 1% | 3% | 5% | 7% | 8% | 10% | 15% | 20% | 30% | 50% |
|---|---|---|---|---|---|---|---|---|---|---|---|---|
| Purity (%) at day 0 | 98.0 | 98.1 | 98.3 | 98.4 | 98.5 | 98.5 | 98.5 | 98.5 | 98.4 | 98.5 | 98.5 | 98.5 |
| Purity(%) at Day 30 | 63.9 | 78.9 | 93.4 | 97.5 | 98.2 | 98.3 | 98.2 | 98.3 | 98.3 | 98.2 | 98.2 | 98.2 |
| Purity (%) at Day 90 | 33.9 | 50.3 | 82.8 | 95.6 | 97.9 | 98.0 | 97.9 | 98.0 | 97.8 | 97.8 | 97.8 | 98.0 |
| Purity (%) at Day 180 | 12.3 | 28.6 | 69.6 | 93.5 | 95.8 | 95.9 | 95.7 | 95.9 | 95.6 | 95.8 | 95.8 | 95.9 |
| Purity(%) at Day 360 | 0 | 8.1 | 49.5 | 86.3 | 92.5 | 93.1 | 92.3 | 92.7 | 92.8 | 92.6 | 92.5 | 92.6 |

The concentration of H-Val-Pro-Gly-Pro-Arg(NO₂)-)OCH(CH₃)₂.HBr salt affects the stability, and it is not stable when the concentration is 0.1% or lower.

TABLE 4
Effect of Concentration of H-Val-Pro-Gly-Pro-Arg(NO₂)—OCH(CH₃)₂•citric
acid salt with 1 equivalent sodium acetate in 50% ethanol at 25° C. on the stability.

| Concentration (wt %) | 0.01% | 0.1% | 1% | 3% | 5% | 7% | 8% | 10% | 15% | 20% | 30% | 50% |
|---|---|---|---|---|---|---|---|---|---|---|---|---|
| Purity (%) at day 0 | 98.1 | 98.2 | 98.3 | 98.4 | 98.5 | 98.6 | 98.5 | 98.6 | 98.6 | 98.5 | 98.5 | 98.6 |
| Purity(%) at Day 30 | 63.1 | 78.5 | 93.3 | 97.6 | 98.1 | 98.3 | 98.2 | 98.3 | 98.4 | 98.3 | 98.3 | 98.4 |
| Purity (%) at Day 90 | 31.2 | 49.9 | 81.1 | 95.3 | 97.7 | 97.9 | 97.8 | 98.0 | 98.0 | 98.1 | 98.0 | 98.0 |
| Purity (%) at Day 180 | 11.0 | 28.2 | 67.8 | 93.2 | 95.3 | 95.7 | 95.6 | 95.9 | 95.7 | 95.6 | 95.5 | 95.9 |
| Purity(%) at Day 360 | 0 | 7.8 | 47.9 | 85.6 | 92.2 | 92.8 | 92.7 | 92.9 | 92.8 | 92.7 | 92.6 | 92.8 |

The concentration of H-Val-Pro-Gly-Pro-Arg(NO₂)—OCH(CH₃)₂.citric acid salt affects the stability, and it is not stable when the concentration is 0.1% or lower.

TABLE 5
Effect of Concentration of H-Ala-Pro-Gly-Pro-Arg(NO₂)—OCH₂CH₃•HCl
salt with 1 equivalent sodium acetate in 50% ethanol at 25° C. on the stability.

| Concentration (wt %) | 0.01% | 0.1% | 1% | 3% | 5% | 7% | 8% | 10% | 15% | 20% | 30% | 50% |
|---|---|---|---|---|---|---|---|---|---|---|---|---|
| Purity (%) at day 0 | 98.3 | 98.4 | 98.5 | 98.8 | 98.9 | 98.9 | 98.8 | 98.9 | 98.8 | 98.9 | 98.9 | 98.8 |
| Purity(%) at Day 30 | 55.6 | 65.7 | 91.3 | 96.8 | 97.1 | 97.5 | 97.6 | 97.6 | 97.6 | 97.5 | 97.6 | 97.5 |
| Purity (%) at Day 90 | 21.3 | 30.2 | 75.9 | 92.1 | 94.4 | 95.0 | 95.1 | 95.2 | 95.0 | 95.1 | 95.1 | 95.2 |
| Purity (%) at Day 180 | 0 | 8.6 | 56.6 | 85.8 | 89.7 | 91.3 | 91.4 | 91.5 | 91.4 | 91.5 | 91.7 | 91.6 |

The concentration of H-Ala-Pro-Gly-Pro-Arg(NO₂)—OCH₂CH₃.HCl salt affects the stability, and it is not stable when the concentration is 0.1% or lower.

TABLE 6
Effect of Concentration of 2-(diethylamino)ethyl
(R,S)-2-(6-methoxy-2-naphthyl)propionate•HCl
salt, in water at 25° C. on stability.

| Concentration (wt %) | 0.01% | 0.1% | 1% | 3% | 5% | 8% | 10% | 15% | 20% |
|---|---|---|---|---|---|---|---|---|---|
| Purity (%) at hr 0 | 98.3 | 98.6 | 98.6 | 98.8 | 98.9 | 98.9 | 98.8 | 98.8 | 98.8 |
| Purity (%) at hr 24 | 91.7 | 98.4 | 98.7 | 98.7 | 98.8 | 98.8 | 98.8 | 98.8 | 98.8 |
| Purity (%) at Day 3 | 79.6 | 97.9 | 98.2 | 98.5 | 98.7 | 98.8 | 98.8 | 98.8 | 98.8 |
| Purity (%) at Day 7 | 73.4 | 97.2 | 97.8 | 98.3 | 98.5 | 98.6 | 98.6 | 98.7 | 98.7 |
| Purity(%) at Day 14 | 68.7 | 96.1 | 97.4 | 97.9 | 98.2 | 98.2 | 98.3 | 98.3 | 98.3 |
| Purity(%) at Day 21 | 64.5 | 95.4 | 96.9 | 97.6 | 97.7 | 97.5 | 97.4 | 97.5 | 97.4 |
| Purity(%) at Day 28 | 61.4 | 94.1 | 96.4 | 97.1 | 97.5 | 97.6 | 97.6 | 97.6 | 97.6 |

Note:
in many cases, (R,S)-is omitted before racemic chemical name; 2-(diethylamino)ethyl 2-(6-methoxy-2-naphthyl)propionate is same as 2-(diethylamino)ethyl (R,S)-2-(6-methoxy-2-naphthyl) propionate.

The concentration of 2-(diethylamino)ethyl 2-(6-methoxy-2-naphthyl) propionate.HCl salt affects the stability, and it is not stable when the concentration is 0.1% or lower.

TABLE 7
Effect of Concentration of 2-(diethylamino)ethyl 1-(4-chlorobenzoyl)-5-methoxy-
2-methyl-1H-indole-3-acetate•HCl salt, in water at 25° C. on stability.

| Concentration (wt %) | 0.01% | 0.1% | 1% | 3% | 5% | 7% | 8% | 10% | 15% | 20% | 30% | 50% |
|---|---|---|---|---|---|---|---|---|---|---|---|---|
| Purity (%) at hr 0 | 98.0 | 98.2 | 98.3 | 98.4 | 98.5 | 98.5 | 98.6 | 98.5 | 98.5 | 98.6 | 98.5 | 98.6 |
| Purity (%) at Day 3 | 84.2 | 96.3 | 97.2 | 97.8 | 98.3 | 98.4 | 98.4 | 98.4 | 98.4 | 98.4 | 98.5 | 98.5 |
| Purity (%) at Day 7 | 72.9 | 93.6 | 96.1 | 97.2 | 98.0 | 98.2 | 98.3 | 98.3 | 98.2 | 98.5 | 98.3 | 98.4 |
| Purity(%) at Day 14 | 61.3 | 88.9 | 94.0 | 96.0 | 97.7 | 98.0 | 98.0 | 98.2 | 98.1 | 98.2 | 98.0 | 98.1 |
| Purity(%) at Day 21 | 53.4 | 84.7 | 92.4 | 95.0 | 97.1 | 97.7 | 97.8 | 98.0 | 98.0 | 98.1 | 98.1 | 97.9 |
| Purity(%) at Day 28 | 45.5 | 78.7 | 91.2 | 94.1 | 96.5 | 97.4 | 97.4 | 97.6 | 97.5 | 97.7 | 97.5 | 97.6 |

The concentration of 2-(diethylamino)ethyl 1-(4-chlorobenzoyl)-5-methoxy-2-methyl-1H-indole-3-acetate.HCl salt affects the stability, and it is not stable when the concentration is 0.1% or lower.

TABLE 8
The effect of concentration of 2-(diethylamino)ethyl acetylsalicylate•HCl
salt on its stability at 5° C. in 15% ethanol.

| Concentration (wt %) | 0.01% | 0.1% | 1% | 3% | 5% | 7% | 8% | 10% | 15% | 20% | 30% | 50% |
|---|---|---|---|---|---|---|---|---|---|---|---|---|
| Purity (%) at day 0 | 99.1 | 99.2 | 99.3 | 99.5 | 99.5 | 99.6 | 99.7 | 99.6 | 99.5 | 99.5 | 99.5 | 99.7 |
| Purity (%) at day 3 | 93.1 | 95.8 | 97.6 | 98.6 | 99.3 | 99.4 | 99.5 | 99.7 | 99.5 | 99.6 | 99.5 | 99.6 |
| Purity (%) at day 7 | 88.2 | 93.1 | 96.9 | 97.7 | 99.1 | 99.3 | 99.3 | 99.4 | 99.3 | 99.3 | 99.4 | 99.5 |
| Purity (%) at day 14 | 81.4 | 89.5 | 95.2 | 96.4 | 98.9 | 99.1 | 99.3 | 99.1 | 99.2 | 99.2 | 99.4 | 99.3 |
| Purity (%) at day 21 | 74.9 | 86.6 | 93.7 | 94.5 | 98.5 | 98.9 | 98.8 | 98.7 | 98.9 | 99.0 | 99.0 | 99.2 |
| Purity (%) at day 28 | 68.3 | 83.7 | 91.5 | 92.7 | 98.0 | 98.7 | 98.6 | 98.7 | 98.7 | 98.8 | 98.9 | 99.0 |
| Purity (%) at day 60 | 47.1 | 71.8 | 82.7 | 86.1 | 96.7 | 98.0 | 98.0 | 98.3 | 98.1 | 98.1 | 98.2 | 98.2 |
| Purity (%) at day 90 | 30.1 | 59.9 | 74.1 | 80.2 | 96.1 | 97.2 | 97.4 | 97.2 | 97.3 | 97.3 | 97.4 | 97.4 |

The concentration of 2-(diethylamino)ethyl acetylsalicylate.HCl salt affects the stability, and it is not stable when the concentration is 0.1% or lower.

TABLE 9
The effect of concentration of 2-(diethylamino)ethyl 5-(2,4-difluorophenyl)-
2-acetoxybenzoate•HCl salt on its stability at 5° C. in 15% acetone.

| Concentration (wt %) | 0.01% | 0.1% | 1% | 3% | 5% | 7% | 8% | 10% | 15% | 20% | 30% | 50% |
|---|---|---|---|---|---|---|---|---|---|---|---|---|
| Purity (%) at day 0 | 98.2 | 98.3 | 98.4 | 98.5 | 98.6 | 98.7 | 98.6 | 98.7 | 98.6 | 98.7 | 98.5 | 98.7 |
| Purity (%) at day 3 | 93.9 | 95.1 | 97.0 | 97.7 | 98.2 | 98.4 | 98.5 | 98.5 | 98.4 | 98.5 | 98.5 | 98.7 |
| Purity (%) at day 7 | 88.1 | 93.1 | 95.9 | 96.6 | 97.8 | 98.4 | 98.3 | 98.5 | 98.4 | 98.5 | 98.4 | 98.5 |
| Purity (%) at day 15 | 80.3 | 89.0 | 94.0 | 94.9 | 97.3 | 98.0 | 98.1 | 98.2 | 98.3 | 98.3 | 98.1 | 98.3 |
| Purity (%) at day 22 | 73.7 | 85.3 | 92.2 | 92.1 | 96.1 | 97.5 | 97.4 | 97.7 | 97.5 | 98.0 | 97.9 | 97.8 |
| Purity (%) at day 28 | 67.9 | 82.3 | 89.7 | 91.2 | 95.5 | 97.2 | 97.1 | 97.3 | 97.2 | 97.1 | 97.3 | 97.2 |
| Purity (%) at day 60 | 48.1 | 70.5 | 79.4 | 84.4 | 94.5 | 96.2 | 96.0 | 96.7 | 96.5 | 96.6 | 96.7 | 96.9 |
| Purity (%) at day 90 | 30.0 | 61.2 | 71.9 | 79.2 | 93.9 | 95.8 | 95.9 | 96.1 | 96.2 | 96.0 | 96.2 | 96.3 |

The concentration of 2-(diethylamino)ethyl 5-(2,4-difluorophenyl)-2-acetoxybenzoate.HCl salt affects the stability, and it is not stable when the concentration is 0.1% or lower.

TABLE 10
Effect of Concentration of 2-(diethylamino)ethyl 1-(4-chlorobenzoyl)-5-methoxy-
2-methyl-1H-indole-3-acetate•HCl salt, in water at 25° C. on stability.

| Concentration (wt %) | 0.01% | 0.1% | 1% | 3% | 5% | 7% | 8% | 10% | 15% | 20% | 30% | 50% |
|---|---|---|---|---|---|---|---|---|---|---|---|---|
| Purity (%) at hr 0 | 98.0 | 98.2 | 98.3 | 98.4 | 98.5 | 98.5 | 98.6 | 98.5 | 98.5 | 98.6 | 98.5 | 98.6 |
| Purity (%) at Day 3 | 84.2 | 96.3 | 97.2 | 97.8 | 98.3 | 98.4 | 98.4 | 98.4 | 98.4 | 98.4 | 98.5 | 98.5 |
| Purity (%) at Day 7 | 72.9 | 93.6 | 96.1 | 97.2 | 98.0 | 98.2 | 98.3 | 98.3 | 98.2 | 98.5 | 98.3 | 98.4 |
| Purity(%) at Day 14 | 61.3 | 88.9 | 94.0 | 96.0 | 97.7 | 98.0 | 98.0 | 98.2 | 98.1 | 98.2 | 98.0 | 98.1 |
| Purity(%) at Day 21 | 53.4 | 84.7 | 92.4 | 95.0 | 97.1 | 97.7 | 97.8 | 98.0 | 98.0 | 98.1 | 98.1 | 97.9 |
| Purity(%) at Day 28 | 45.5 | 78.7 | 91.2 | 94.1 | 96.5 | 97.4 | 97.4 | 97.6 | 97.5 | 97.7 | 97.5 | 97.6 |

The concentration of 2-(diethylamino)ethyl 1-(4-chlorobenzoyl)-5-methoxy-2-methyl-1H-indole-3-acetate.HCl salt affects the stability, and it is not stable when the concentration is 0.1% or lower.

TABLE 11
Effect of Concentration of 2-(diethylamino)ethyl 5-fluoro-2-methyl-1-[[4-(methylsulfinyl)phenyl]methylene]-1H-
indene-3-acetate•HCl salt, in water at 25° C. on stability.

| Concentration (wt %) | 0.01% | 0.1% | 1% | 3% | 5% | 7% | 8% | 10% | 15% | 20% | 30% | 50% |
|---|---|---|---|---|---|---|---|---|---|---|---|---|
| Purity (%) at hr 0 | 97.1 | 97.2 | 97.3 | 97.3 | 97.5 | 97.6 | 97.5 | 97.5 | 97.6 | 97.6 | 97.7 | 97.7 |
| Purity (%) at Day 3 | 82.3 | 95.7 | 96.2 | 96.7 | 97.3 | 97.4 | 97.4 | 97.5 | 97.4 | 97.4 | 97.5 | 97.5 |
| Purity (%) at Day 7 | 71.2 | 93.6 | 95.2 | 96.3 | 97.1 | 97.3 | 97.2 | 97.3 | 97.4 | 97.6 | 97.5 | 97.5 |
| Purity(%) at Day 14 | 59.4 | 89.8 | 93.1 | 95.0 | 96.7 | 97.1 | 96.9 | 97.0 | 97.1 | 97.2 | 97.3 | 97.2 |
| Purity(%) at Day 21 | 50.4 | 84.4 | 91.5 | 94.1 | 96.2 | 96.6 | 96.8 | 97.0 | 97.1 | 97.1 | 97.0 | 96.8 |
| Purity(%) at Day 28 | 43.7 | 78.9 | 90.1 | 93.1 | 95.4 | 96.4 | 96.5 | 96.7 | 96.8 | 96.8 | 96.7 | 96.8 |

The concentration of 2-(diethylamino)ethyl 5-fluoro-2-methyl-1-[[4-(methylsulfinyl)phenyl]methylene]-1H-indene-3-acetate.HCl salt affects the stability, and it is not stable when the concentration is 0.1% or lower.

TABLE 12
Effect of Concentration of 2-(diethylamino)ethyl 1-methyl-5-(4-methylbenzoyl)-
1H-pyrrole-2-acetate•HCl salt, in water at 25° C. on stability.

| Concentration (wt %) | 0.01% | 0.1% | 1% | 3% | 5% | 7% | 8% | 10% | 15% | 20% | 30% | 50% |
|---|---|---|---|---|---|---|---|---|---|---|---|---|
| Purity (%) at hr 0 | 97.4 | 97.5 | 97.7 | 97.9 | 97.9 | 98.1 | 98.1 | 98.2 | 98.1 | 98.0 | 98.1 | 98.2 |
| Purity (%) at Day 3 | 82.3 | 95.6 | 95.7 | 96.5 | 97.2 | 97.5 | 97.7 | 97.9 | 97.7 | 97.6 | 97.7 | 97.8 |
| Purity (%) at Day 7 | 72.2 | 93.3 | 94.6 | 96.2 | 97.0 | 97.2 | 97.3 | 97.5 | 97.6 | 97.5 | 97.5 | 97.5 |
| Purity(%) at Day 14 | 57.4 | 88.3 | 92.3 | 94.8 | 96.8 | 97.2 | 96.9 | 97.3 | 97.4 | 97.2 | 97.3 | 97.2 |
| Purity(%) at Day 21 | 45.4 | 83.5 | 90.1 | 94.0 | 95.6 | 96.7 | 96.9 | 97.1 | 97.1 | 97.1 | 97.0 | 96.9 |
| Purity(%) at Day 28 | 35.7 | 78.1 | 88.6 | 92.7 | 94.4 | 96.5 | 96.6 | 96.5 | 96.8 | 96.8 | 96.7 | 96.8 |

The concentration of 2-(diethylamino)ethyl 1-methyl-5-(4-methylbenzoyl)-1H-pyrrole-2-acetate.HCl salt affects the stability, and it is not stable when the concentration is 0.1% or lower.

TABLE 13
Effect of Concentration of 2-(diethylamino)ethyl 5-(4-chlorobenzoyl)-1,4-
dimethyl-1H-pyrrole-2-acetate•HCl salt, in water at 25° C. on stability.

| Concentration (wt %) | 0.01% | 0.1% | 1% | 3% | 5% | 7% | 8% | 10% | 15% | 20% | 30% | 50% |
|---|---|---|---|---|---|---|---|---|---|---|---|---|
| Purity (%) at hr 0 | 97.5 | 97.5 | 97.7 | 97.9 | 98.0 | 98.2 | 98.1 | 98.2 | 98.1 | 98.0 | 98.1 | 98.1 |
| Purity (%) at Day 3 | 85.3 | 95.3 | 95.6 | 96.5 | 97.2 | 97.7 | 97.7 | 97.8 | 97.7 | 97.6 | 97.8 | 97.7 |
| Purity (%) at Day 7 | 72.9 | 93.3 | 94.2 | 96.3 | 97.0 | 97.2 | 97.3 | 97.4 | 97.7 | 97.5 | 97.5 | 97.5 |
| Purity(%) at Day 14 | 60.1 | 88.5 | 92.5 | 94.7 | 96.5 | 97.2 | 97.0 | 97.3 | 97.6 | 97.2 | 97.3 | 97.2 |
| Purity(%) at Day 21 | 47.4 | 83.2 | 90.7 | 93.8 | 95.2 | 96.8 | 96.9 | 97.2 | 97.3 | 97.3 | 97.1 | 97.2 |
| Purity(%) at Day 28 | 37.7 | 77.6 | 88.1 | 92.5 | 94.7 | 96.6 | 96.5 | 96.7 | 96.6 | 96.8 | 96.9 | 96.8 |

The concentration of 2-(diethylamino)ethyl S-(4-chlorobenzoyl)-1,4-dimethyl-1H-pyrrole-2-acetate.HCl salt affects the stability, and it is not stable when the concentration is 0.1% or lower.

TABLE 14
Effect of Concentration of 2-(diethylamino)ethyl 3-(6-methoxy-2-naphthyl)propionate•HCl
salt, in water at 25° C. on stability.

| Concentration (wt %) | 0.01% | 0.1% | 1% | 3% | 5% | 7% | 8% | 10% | 15% | 20% | 30% | 50% |
|---|---|---|---|---|---|---|---|---|---|---|---|---|
| Purity (%) at hr 0 | 97.7 | 97.9 | 98.1 | 98.3 | 98.4 | 98.5 | 98.4 | 98.5 | 98.5 | 98.4 | 98.5 | 98.5 |
| Purity (%) at Day 3 | 84.7 | 96.7 | 97.0 | 97.7 | 98.2 | 98.4 | 98.4 | 98.4 | 98.4 | 98.4 | 98.4 | 98.5 |
| Purity (%) at Day 7 | 73.9 | 95.5 | 96.0 | 97.0 | 98.0 | 98.2 | 98.3 | 98.3 | 98.2 | 98.2 | 98.3 | 98.3 |
| Purity(%) at Day 14 | 62.3 | 91.1 | 94.0 | 96.0 | 97.6 | 98.0 | 98.0 | 98.2 | 98.1 | 98.2 | 98.1 | 98.1 |
| Purity(%) at Day 21 | 53.9 | 87.2 | 92.3 | 94.8 | 97.0 | 97.8 | 97.9 | 98.0 | 98.0 | 98.1 | 98.1 | 97.8 |
| Purity(%) at Day 28 | 45.9 | 83.8 | 90.9 | 93.8 | 96.3 | 97.4 | 97.5 | 97.5 | 97.6 | 97.5 | 97.5 | 97.6 |

The concentration of 2-(diethylamino)ethyl 3-(6-methoxy-2-naphthyl)propionate.HCl salt affects the stability, and it is not stable when the concentration is 0.1% or lower.

TABLE 15
Effect of Concentration of 2-(diethylamino)ethyl 4-(4-chlorophenyl)-2-phenyl-
5-thiazoleacetate•HCl salt, in water at 25° C. on stability.

| Concentration (wt %) | 0.01% | 0.1% | 1% | 3% | 5% | 7% | 8% | 10% | 15% | 20% | 30% | 50% |
|---|---|---|---|---|---|---|---|---|---|---|---|---|
| Purity (%) at hr 0 | 98.4 | 98.5 | 98.7 | 98.9 | 98.9 | 99.1 | 99.1 | 99.2 | 99.1 | 99.0 | 99.1 | 99.1 |
| Purity (%) at Day 3 | 83.4 | 96.7 | 96.6 | 96.5 | 98.4 | 98.6 | 98.7 | 98.8 | 98.7 | 98.8 | 98.7 | 98.8 |
| Purity (%) at Day 7 | 74.2 | 95.6 | 95.5 | 96.2 | 98.0 | 98.2 | 98.3 | 98.5 | 98.6 | 98.5 | 98.5 | 98.5 |
| Purity(%) at Day 14 | 58.4 | 91.5 | 92.9 | 94.8 | 97.8 | 98.2 | 97.9 | 98.3 | 98.4 | 98.2 | 98.3 | 98.2 |
| Purity(%) at Day 21 | 46.5 | 84.7 | 90.9 | 94.0 | 96.6 | 97.7 | 97.9 | 98.1 | 98.1 | 98.1 | 98.0 | 98.9 |
| Purity(%) at Day 28 | 37.5 | 80.5 | 89.1 | 92.7 | 95.4 | 97.5 | 97.6 | 97.5 | 97.8 | 97.7 | 97.8 | 97.8 |

The concentration of 2-(diethylamino)ethyl 4-(4-chlorophenyl)-2-phenyl-5-thiazoleacetate.HCl salt affects the stability, and it is not stable when the concentration is 0.1% or lower.

TABLE 16
Effect of Concentration of 2-(diethylamino)ethyl [(1-benzyl-1H-indazol-3-
yl)oxy]acetate•HCl salt, in water at 25° C. on stability.

| Concentration (wt %) | 0.01% | 0.1% | 1% | 3% | 5% | 7% | 8% | 10% | 15% | 20% | 30% | 50% |
|---|---|---|---|---|---|---|---|---|---|---|---|---|
| Purity (%) at hr 0 | 97.4 | 97.5 | 97.7 | 97.9 | 97.9 | 98.1 | 98.1 | 98.2 | 98.1 | 98.0 | 98.1 | 98.2 |
| Purity (%) at Day 3 | 83.8 | 95.7 | 95.6 | 95.5 | 97.4 | 97.6 | 97.7 | 97.8 | 97.7 | 97.8 | 97.7 | 97.8 |
| Purity (%) at Day 7 | 73.2 | 94.6 | 94.5 | 95.2 | 97.0 | 97.2 | 97.3 | 97.5 | 97.6 | 97.5 | 97.5 | 97.5 |
| Purity(%) at Day 14 | 58.4 | 88.5 | 91.9 | 93.8 | 96.8 | 97.2 | 96.9 | 97.3 | 97.4 | 97.2 | 97.3 | 97.2 |
| Purity(%) at Day 21 | 45.5 | 82.7 | 90.1 | 93.0 | 95.6 | 96.7 | 96.9 | 97.1 | 97.1 | 97.1 | 97.0 | 96.9 |
| Purity(%) at Day 28 | 36.7 | 79.5 | 88.1 | 91.7 | 94.4 | 96.5 | 96.6 | 96.5 | 96.8 | 96.7 | 96.8 | 96.7 |

The concentration of 2-(diethylamino)ethyl [(1-benzyl-1H-indazol-3-yl)oxy]acetate.HCl salt affects the stability, and it is not stable when the concentration is 0.1% or lower.

TABLE 17
Effect of Concentration of 2-(diethylamino)ethyl 2-[(4-chlorophenyl)-5-benzoxazole]propionate•HCl
salt, in water at 25° C. on stability.

| Concentration (wt %) | 0.01% | 0.1% | 1% | 3% | 5% | 7% | 8% | 10% | 15% | 20% | 30% | 50% |
|---|---|---|---|---|---|---|---|---|---|---|---|---|
| Purity (%) at hr 0 | 97.8 | 97.9 | 98.1 | 98.3 | 98.5 | 98.6 | 98.6 | 98.5 | 98.6 | 98.5 | 98.6 | 98.5 |
| Purity (%) at Day 3 | 84.7 | 96.7 | 97.0 | 97.5 | 98.0 | 98.4 | 98.5 | 98.4 | 98.5 | 98.4 | 98.5 | 98.5 |
| Purity (%) at Day 7 | 73.9 | 95.5 | 96.0 | 97.0 | 97.9 | 98.2 | 98.3 | 98.3 | 98.3 | 98.3 | 98.3 | 98.3 |
| Purity(%) at Day 14 | 62.3 | 91.1 | 94.0 | 95.7 | 97.6 | 98.0 | 98.1 | 98.2 | 98.1 | 98.2 | 98.0 | 98.0 |
| Purity(%) at Day 21 | 52.9 | 87.2 | 92.3 | 94.3 | 97.0 | 97.8 | 97.8 | 97.7 | 97.7 | 97.8 | 97.7 | 97.9 |
| Purity(%) at Day 28 | 42.9 | 83.0 | 89.9 | 92.8 | 96.3 | 97.4 | 97.4 | 97.3 | 97.5 | 97.4 | 97.3 | 97.5 |

The concentration of 2-(diethylamino)ethyl 2-(diethylamino)ethyl 2-[(4-chlorophenyl)-5-benzoxazole]propionate.HCl salt affects the stability, and it is not stable when the concentration is 0.10% or lower.

TABLE 18
Effect of Concentration of 2-(diethylamino)ethyl 4,5-diphenyl-2-oxazolepropionate•HCl
salt, in water at 25° C. on stability.

| Concentration (wt %) | 0.01% | 0.1% | 1% | 3% | 5% | 7% | 8% | 10% | 15% | 20% | 30% | 50% |
|---|---|---|---|---|---|---|---|---|---|---|---|---|
| Purity (%) at hr 0 | 97.8 | 97.9 | 98.0 | 98.4 | 98.5 | 98.8 | 98.7 | 98.8 | 98.8 | 98.7 | 98.8 | 98.8 |
| Purity (%) at Day 3 | 83.8 | 94.7 | 95.3 | 96.3 | 98.2 | 98.6 | 98.7 | 98.6 | 98.7 | 98.7 | 98.7 | 98.7 |
| Purity (%) at Day 7 | 73.9 | 90.6 | 92.9 | 95.1 | 97.8 | 98.2 | 98.3 | 98.5 | 98.4 | 98.5 | 98.5 | 98.5 |
| Purity (%) at Day 14 | 58.9 | 83.5 | 88.9 | 92.5 | 97.0 | 98.0 | 97.9 | 98.2 | 98.1 | 98.2 | 98.3 | 98.2 |
| Purity (%) at Day 21 | 47.6 | 75.7 | 84.6 | 89.0 | 96.3 | 97.7 | 97.7 | 97.8 | 97.9 | 97.8 | 97.9 | 97.8 |
| Purity (%) at Day 28 | 38.9 | 65.5 | 79.2 | 85.7 | 95.0 | 97.4 | 97.5 | 97.4 | 97.5 | 97.5 | 97.6 | 97.5 |

The concentration of 2-(diethyl amino)ethyl 4,5-diphenyl-2-oxazolepropionate.HCl salt affects the stability, and it is not stable when the concentration is 0.1% or lower.

TABLE 19
Effect of Concentration of 2-(diethylamino)ethyl 4-[bis(2-chloroethyl)amino]benzenebutyrate•HCl
salt, in water at 25° C. on stability.

| Concentration (wt %) | 0.01% | 0.1% | 1% | 3% | 5% | 7% | 8% | 10% | 15% | 20% | 30% | 50% |
|---|---|---|---|---|---|---|---|---|---|---|---|---|
| Purity (%) at hr 0 | 97.8 | 97.9 | 98.2 | 98.4 | 98.5 | 98.5 | 98.5 | 98.6 | 98.6 | 98.6 | 98.5 | 98.6 |
| Purity (%) at Day 3 | 83.1 | 94.1 | 95.1 | 96.2 | 98.2 | 98.4 | 98. | 98.5 | 98.5 | 98.4 | 98.4 | 98.4 |
| Purity (%) at Day 7 | 72.9 | 89.6 | 91.9 | 95.0 | 97.8 | 98.2 | 98.3 | 98.3 | 98.4 | 98.3 | 98.4 | 98.3 |
| Purity(%) at Day 14 | 56.9 | 81.5 | 85.9 | 91.0 | 97.0 | 98.0 | 97.9 | 98.2 | 98.1 | 98.2 | 98.3 | 98.2 |
| Purity(%) at Day 21 | 43.2 | 73.7 | 81.9 | 87.3 | 96.3 | 97.6 | 97.7 | 97.7 | 97.7 | 97.8 | 97.7 | 97.8 |
| Purity(%) at Day 28 | 34.3 | 62.5 | 76.9 | 83.7 | 95.0 | 97.4 | 97.3 | 97.4 | 97.3 | 97.3 | 97.4 | 97.4 |

The concentration of 2-(diethylamino)ethyl 4-[bis(2-chloroethyl)amino]benzenebutyrate.HCl salt affects the stability, and it is not stable when the concentration is 0.1% or lower.

TABLE 20
Effect of Concentration of 2-(diethylamino)ethyl 4-[bis(2-methylsulfonylethyl)amino]benzenebutyrate•HCl
salt, in water at 25° C. on stability.

| Concentration (wt %) | 0.01% | 0.1% | 1% | 3% | 5% | 7% | 8% | 10% | 15% | 20% | 30% | 50% |
|---|---|---|---|---|---|---|---|---|---|---|---|---|
| Purity (%) at hr 0 | 97.6 | 97.7 | 98.0 | 98.1 | 98.2 | 98.4 | 98.3 | 98.4 | 98.4 | 98.4 | 98.5 | 98.4 |
| Purity (%) al Day 3 | 83.4 | 93.7 | 95.1 | 96.0 | 97.8 | 98.3 | 98.2 | 98.3 | 98.4 | 98.4 | 98.4 | 98.3 |
| Purity (%) at Day 7 | 73.2 | 88.3 | 90.8 | 94.3 | 97.2 | 98.1 | 98.2 | 98.3 | 98.2 | 98.3 | 98.2 | 98.2 |
| Purity(%) at Day 14 | 55.2 | 80.6 | 85.1 | 89.7 | 95.3 | 98.0 | 98.1 | 98.0 | 98.1 | 98.2 | 98.1 | 98.2 |
| Purity(%) at Day 21 | 43.1 | 71.5 | 80.3 | 84.8 | 93.0 | 97.5 | 97.6 | 97.5 | 97.7 | 97.6 | 97.8 | 97.7 |
| Purity(%) at Day 28 | 32.0 | 60.7 | 73.8 | 80.6 | 90.3 | 97.0 | 97.0 | 97.1 | 97.2 | 97.2 | 97.3 | 97.3 |

The concentration of 2-(diethylamino)ethyl 4-[bis(2-methylsulfonylethyl)amino]benzenebutyrate.HCl salt affects the stability, and it is not stable when the concentration is 0.1% or lower.

It can be seen that the concentration of HPD affects the stability of pharmaceutical composition significantly. Less than 1% by weight of HPD in aqueous solution is not stable, whereas 1% by weight or a higher concentration of the HPD is desirable. Advantageously, the concentration of the HPD in the composition may be 1-30% by weight, preferably 1-20% by weight, more preferably 3-15%, and most preferably 5-10%. The substituent groups and the type of the salt have shown little effect on the stability.

In contrast, the concentration of common esters does not affect the stability significantly.

TABLE 21
Effect of Concentration of ethyl benzoate
in 50% ethanol at 25° C. on stability.

| Concentration (wt %) | 0.01% | 0.1% | 1% | 3% | 5% | 10% |
|---|---|---|---|---|---|---|
| Purity (%) at hr 0 | 97.8 | 97.9 | 98.0 | 97.9 | 98.0 | 97.9 |
| Purity (%) at hr 24 | 97.6 | 97.7 | 97.8 | 97.8 | 97.8 | 97.8 |
| Purity (%) at Day 3 | 97.4 | 97.6 | 97.7 | 97.8 | 97.8 | 97.8 |
| Purity (%) at Day 7 | 96.9 | 97.5 | 97.6 | 97.7 | 97.7 | 97.8 |
| Purity(%) at Day 14 | 96.1 | 97.2 | 97.5 | 97.6 | 97.6 | 97.7 |
| Purity(%) at Day 28 | 95.3 | 95.9 | 97.3 | 97.5 | 97.6 | 97.6 |

Ethyl benzoate is very stable at 0.01% to 10% or a higher concentration. The concentration affects the stability slightly.

TABLE 22
Effect of Concentration of isopropyl benzoate
in 50% ethanol at 25° C. on stability.

| Concentration (wt %) | 0.01% | 0.1% | 1% | 3% | 5% | 10% |
|---|---|---|---|---|---|---|
| Purity (%) at hr 0 | 98.4 | 98.4 | 98.5 | 98.4 | 98.5 | 98.5 |
| Purity (%) at hr 24 | 98.3 | 98.5 | 98.4 | 98.6 | 98.4 | 98.8 |
| Purity (%) at Day 3 | 98.1 | 98.4 | 98.5 | 98.4 | 98.7 | 98.4 |
| Purity (%) at Day 7 | 98.3 | 98.4 | 98.4 | 98.4 | 98.4 | 98.4 |
| Purity(%) at Day 14 | 98.2 | 98.3 | 98.3 | 98.4 | 98.4 | 98.4 |
| Purity(%) at Day 28 | 98.2 | 98.3 | 98.3 | 98.4 | 98.4 | 98.4 |

Isopropyl benzoate is very stable at 0.01% to 10% or a higher concentration and more stable than ethyl benzoate. The concentration does not affect the stability.

TABLE 23
Effect of Concentration of t-butyl benzoate (a normal
ester) in 50% ethanol at 25° C. on stability.

| Concentration (wt %) | 0.01% | 0.1% | 1% | 3% | 5% | 10% |
|---|---|---|---|---|---|---|
| Purity (%) at hr 0 | 97.5 | 97.4 | 97.5 | 97.4 | 97.4 | 97.8 |
| Purity (%) at hr 2 | 25.2 | 31.2 | 41.3 | 52.3 | 56.3 | 65.5 |
| Purity (%) at hr 24 | 0.0 | 0.0 | 1.6 | 15.4 | 20.3 | 25.1 |
| Purity (%) at Day 3 | 0.0 | 0.0 | 0.0 | 1.1 | 3.6 | 5.1 |
| Purity(%) at Day 7 | 0.0 | 0.0 | 0.0 | 0.0 | 0.0 | 0.0 |
| Purity(%) at Day 14 | 0.0 | 0.0 | 0.0 | 0.0 | 0.0 | 0.0 |

t-Butyl benzoate is very unstable at any concentration and much less stable than ethyl benzoate and isopropyl benzoate.

TABLE 24
Effect of Concentration of isopropyl 2-amino-3-phenylpropanoate
in 50% ethanol at 25° C. on stability.

| Concentration (wt %) | 0.01% | 0.1% | 1% | 3% | 5% | 10% |
|---|---|---|---|---|---|---|
| Purity (%) at hr 0 | 98.4 | 98.5 | 98.7 | 98.7 | 98.7 | 98.8 |
| Purity (%) at hr 24 | 98.3 | 98.4 | 98.5 | 98.6 | 98.6 | 98.7 |
| Purity (%) at Day 3 | 97.4 | 97.6 | 97.9 | 98.2 | 98.5 | 98.6 |
| Purity (%) at Day 7 | 97.1 | 97.3 | 97.6 | 97.8 | 98.4 | 98.5 |
| Purity(%) at Day 14 | 96.5 | 96.8 | 97.2 | 97.4 | 98.2 | 98.3 |
| Purity(%) at Day 21 | 95.9 | 96.5 | 96.8 | 97.0 | 97.8 | 97.9 |
| Purity(%) at Day 28 | 95.1 | 95.7 | 96.1 | 96.5 | 97.1 | 97.3 |

Isopropyl 2-amino-3-phenylpropanoate is quite stable at 0.01% to 10% and much more stable than ethyl 2-amino-3-phenylpropanoate, perhaps because isopropyl group is more sterically hindered than ethyl group.

TABLE 25
Effect of Concentration of t-butyl 2-amino-3-phenylpropanoate
in 50% ethanol at 25° C. on stability.

| Concentration (wt %) | 0.01% | 0.1% | 1% | 3% | 5% | 10% |
|---|---|---|---|---|---|---|
| Purity (%) at hr 0 | 98.0 | 98.0 | 98.1 | 98.2 | 98.1 | 98.2 |
| Purity (%) at hr 24 | 96.1 | 96.4 | 96.7 | 96.9 | 97.0 | 97.6 |
| Purity (%) at Day 3 | 92.1 | 93.1 | 93.9 | 94.6 | 94.8 | 95.9 |
| Purity (%) at Day 7 | 89.4 | 91.4 | 92.2 | 92.4 | 93.1 | 93.9 |
| Purity(%) at Day 14 | 83.0 | 86.1 | 88.9 | 89.5 | 90.0 | 91.0 |
| Purity(%) at Day 21 | 60.2 | 64.1 | 70.9 | 72.4 | 73.0 | 74.9 |
| Purity(%) at Day 28 | 35.2 | 41.0 | 48.9 | 50.4 | 56.9 | 59.2 |

t-Butyl 2-amino-3-phenylpropanoate is not stable at 0.01% to 10%.

### 2. Effect of pH Value on the Stability

TABLE 26
Stabilities of 5% solution of H-Val-Pro-Gly-Pro-Arg(NO₂)—OCH(CH₃)₂•HCl
salt in 25% ethanol at different pH for 30 days at 25° C.

| | | | 25% | | 25% | 25% | 25% | | |
|---|---|---|---|---|---|---|---|---|---|
| | | | ethanol | | ethanol | ethanol | ethanol | | |
| | 25% | 25% | (after | | with 0.5 | with 1 | with 1.5 | 25% | 25% |
| | ethanol | ethanol | multiple | | equivalent | equivalent | equivalent | ethanol | ethanol |
| | with | with | recrystal- | 25% | sodium | sodium | sodium | with | with |
| | HCl | HCl | ization | ethanol | acetate | acetate | acetate | NaOH | NaOH |
| | pH 1.0 | pH 2.0 | pH 3.7 | pH 3.0 | pH 3.7 | pH 4.6 | pH 5.6 | pH 7 | pH 8 |
| Purity(%) | 98.3 ± 0.2 | 98.4 ± 0.2 | 98.6 ± 0.2 | 98.5 ± 0.1 | 98.6 ± 0.2 | 98.6 ± 0.2 | 98.5 ± 0.2 | 98.3 ± 0.3 | 98.0 ± 0.3 |
| at day 1 | | | | | | | | | |
| Purity(%) | 85.0 ± 0.2 | 91.7 ± 0.2 | 98.1 ± 0.2 | 97.4 ± 0.2 | 98.2 ± 0.1 | 98.2 ± 0.2 | 97.0 ± 0.2 | 78.1 ± 0.4 | 56.3 ± 0.3 |
| at day 30 | | | | | | | | | |
| Purity(%) | 59.5 ± 0.2 | 84.7 ± 0.3 | 97.2 ± 0.1 | 94.7 ± 0.1 | 97.1 ± 0.2 | 97.0 ± 0.1 | 93.8 ± 0.1 | 44.1 ± 0.4 | 32.3 ± 0.4 |
| at day 90 | | | | | | | | | |
| Purity(%) | 43.5 ± 0.3 | 71.7 ± 0.3 | 95.4 ± 0.1 | 90.7 ± 0.2 | 95.6 ± 0.2 | 95.6 ± 0.2 | 88.1 ± 0.2 | 25.1 ± 0.4 | 3.9 ± 0.4 |
| at day 180 | | | | | | | | | |
| Purity(%) | 17.6 ± 0.3 | 52.7 ± 0.3 | 92.1 ± 0.2 | 82.7 ± 0.3 | 92.0 ± 0.2 | 92.1 ± 0.2 | 80.1 ± 0.3 | 0 | 0 |
| at day 360 | | | | | | | | | |

The solution of H-Val-Pro-Gly-Pro-Arg(NO₂)—OCH(CH₃)₂.HCl salt is only stable at pH 3-6 and can be stored for about 1 year at room temperature.

TABLE 27
Stabilities of 5% solution of H-Val-Pro-Asp[OCH(CH₃)₂]-Pro-Arg(NO₂)—OCH(CH₃)₂•HCl
salt in 25% ethanol at different pH for 30 days at 25° C.

| | | 25% | | 25% | 25% | 25% | | |
|---|---|---|---|---|---|---|---|---|
| | | ethanol | | ethanol | ethanol | ethanol | | |
| | 25% | (after | | with 0.5 | with 1 | with 1.5 | 25% | 25% |
| | ethanol | multiple | | equivalent | equivalent | equivalent | ethanol | ethanol |
| | with | recrystal- | 25% | sodium | sodium | sodium | with | with |
| | HCl | ization | ethanol | acetate | acetate | acetate | NaOH | NaOH |
| | pH 2.0 | pH 3.7 | pH 3.0 | pH 3.7 | pH 4.6 | pH 5.6 | pH 7 | pH 8 |
| Purity(%) | 98.7 ± 0.1 | 98.9 ± 0.2 | 98.8 ± 0.2 | 98.9 ± 0.1 | 98.9 ± 0.2 | 98.8 ± 0.2 | 98.6 ± 0.3 | 98.4 ± 0.3 |
| at day 1 | | | | | | | | |
| Purity(%) | 91.2 ± 0.2 | 97.6 ± 0.2 | 97.1 ± 0.2 | 97.9 ± 0.1 | 97.8 ± 0.2 | 97.0 ± 0.2 | 77.0 ± 0.2 | 52.3 ± 0.2 |
| at day 30 | | | | | | | | |
| Purity(%) | 83.9 ± 0.2 | 96.9 ± 0.1 | 94.1 ± 0.1 | 96.9 ± 0.2 | 97.0 ± 0.1 | 93.4 ± 0.1 | 41.1 ± 0.3 | 29.3 ± 0.3 |
| at day 90 | | | | | | | | |
| Purity(%) | 69.8 ± 0.2 | 95.0 ± 0.1 | 89.3 ± 0.2 | 95.0 ± 0.2 | 95.0 ± 0.2 | 86.9 ± 0.2 | 21.1 ± 0.3 | 2.9 ± 0.3 |
| at day 180 | | | | | | | | |
| Purity(%) | 48.7 ± 0.3 | 91.9 ± 0.2 | 81.3 ± 0.3 | 92.2 ± 0.2 | 92.0 ± 0.2 | 79.8 ± 0.3 | 0 | 0 |
| at day 360 | | | | | | | | |

The solution of H-Val-Pro-Asp[OCH(CH₃)₂]-Pro-Arg(NO₂)—OCH(CH₃)₂.HC salt is stable at pH 3-6 and can be stored for about 1 year at room temperature.

TABLE 28
Stabilities of 5% solution of H-Tyr-Gly-Gly-Phe-Leu-OCH₂CH₃•HCl
salt in 25% ethanol at different pH for 30 days at 25° C.

| | | | 25% | | 25% | 25% | 25% | | |
|---|---|---|---|---|---|---|---|---|---|
| | | | ethanol | | ethanol | ethanol | ethanol | | |
| | 25% | 25% | (after | | with 0.5 | with 1 | with 1.5 | 25% | 25% |
| | ethanol | ethanol | multiple | | equivalent | equivalent | equivalent | ethanol | ethanol |
| | with | with | recrystal- | 25% | sodium | sodium | sodium | with | with |
| | HCl | HCl | ization | ethanol | acetate | acetate | acetate | NaOH | NaOH |
| | pH 1.0 | pH 2.0 | pH 3.7 | pH 3.0 | pH 3.7 | pH 4.6 | pH 5.6 | pH 7 | pH 8 |
| Purity(%) | 98.3 ± 0.2 | 98.4 ± 0.2 | 98.5 ± 0.1 | 98.4 ± 0.1 | 98.5 ± 0.1 | 98.5 ± 0.1 | 98.5 ± 0.2 | 98.2 ± 0.3 | 98.2 ± 0.2 |
| at day 1 | | | | | | | | | |
| Purity(%) | 68.3 ± 0.1 | 85.6 ± 0.2 | 97.8 ± 0.2 | 96.1 ± 0.2 | 97.7 ± 0.2 | 97.7 ± 0.2 | 95.2 ± 0.2 | 57.3 ± 0.3 | 25.1 ± 0.3 |
| at day 30 | | | | | | | | | |
| Purity(%) | 37.3 ± 0.2 | 71.9 ± 0.2 | 95.4 ± 0.3 | 93.3 ± 0.3 | 95.3 ± 0.2 | 95.3 ± 0.3 | 92.3 ± 0.3 | 33.9 ± 0.4 | 0 |
| at day 90 | | | | | | | | | |
| Purity(%) | 14.5 ± 0.2 | 44.5 ± 0.3 | 91.9 ± 0.2 | 86.9 ± 0.2 | 91.8 ± 0.3 | 91.7 ± 0.3 | 85.1 ± 0.3 | 0 | 0 |
| at day 180 | | | | | | | | | |

The solution of H-Tyr-Gly-Gly-Phe-Leu-OCH₂CH₃.HCl salt is stable only at pH 3-6 and can be stored for only about 3 months at room temperature.

TABLE 29
Stabilities of 5% solution of H-Ala-Pro-Gly-Pro-Arg(NO₂)—OCH₂CH₃•HCl
salt in 25% ethanol at different pH for 30 days at 25° C.

| | | 25% | | 25% | 25% | 25% | | |
|---|---|---|---|---|---|---|---|---|
| | | ethanol | | ethanol | ethanol | ethanol | | |
| | | (after | | with 0.5 | with 1 | with 1.5 | 25% | 25% |
| | 25% | multiple | | equivalent | equivalent | equivalent | ethanol | ethanol |
| | ethanol | recrystal- | 25% | sodium | sodium | sodium | with | with |
| | with HCl | ization | ethanol | acetate | acetate | acetate | NaOH | NaOH |
| | pH 2.0 | pH 3.7 | pH 3.0 | pH 3.7 | pH 4.6 | pH 5.6 | pH 7 | pH 8 |
| Purity(%) | 97.8 ± 0.1 | 98.1 ± 0.2 | 98.1 ± 0.2 | 98.2 ± 0.1 | 98.2 ± 0.2 | 98.1 ± 0.2 | 97.9 ± 0.2 | 97.5 ± 0.1 |
| at day 1 | | | | | | | | |
| Purity(%) | 84.2 ± 0.2 | 97.3 ± 0.2 | 95.7 ± 0.3 | 97.4 ± 0.2 | 97.3 ± 0.2 | 94.7 ± 0.2 | 55.2 ± 0.3 | 22.6 ± 0.2 |
| at day 30 | | | | | | | | |
| Purity(%) | 69.6 ± 0.2 | 95.0 ± 0.1 | 92.8 ± 0.3 | 95.1 ± 0.3 | 95.0 ± 0.3 | 91.5 ± 0.3 | 31.3 ± 0.3 | 0 |
| at day 90 | | | | | | | | |
| Purity(%) | 39.9 ± 0.3 | 91.2 ± 0.3 | 86.0 ± 0.3 | 91.2 ± 0.3 | 91.1 ± 0.3 | 83.7 ± 0.3 | 0 | 0 |
| at day 180 | | | | | | | | |

The solution of H-Ala-Pro-Gly-Pro-Arg(NO₂)—OCH₂CH₃.HCl salt is stable only at pH 3-6 and can be stored for only about 3 months at room temperature.

TABLE 30
Stabilities of 5% solution of H-Tyr-Gly-Gly-Phe-Met-OCH(CH₃)₂•HCl
salt in 25% ethanol at different pH for 30 days at 25° C.

| | | 25% | | 25% | 25% | 25% | | |
|---|---|---|---|---|---|---|---|---|
| | | ethanol | | ethanol | ethanol | ethanol | | |
| | | (after | | with 0.5 | with 1 | with 1.5 | 25% | 25% |
| | 25% | multiple | | equivalent | equivalent | equivalent | ethanol | ethanol |
| | ethanol | recrystal- | 25% | sodium | sodium | sodium | with | with |
| | with HCl | ization | ethanol | acetate | acetate | acetate | NaOH | NaOH |
| | pH 2.0 | pH 3.7 | pH 3.0 | pH 3.7 | pH 4.6 | pH 5.6 | pH 7 | pH 8 |
| Purity(%) | 98.5 ± 0.1 | 98.9 ± 0.2 | 98.7 ± 0.2 | 98.9 ± 0.1 | 98.9 ± 0.2 | 98.8 ± 0.2 | 98.5 ± 0.3 | 98.2 ± 0.2 |
| at day 1 | | | | | | | | |
| Purity(%) | 92.1 ± 0.2 | 98.3 ± 0.2 | 97.9 ± 0.2 | 98.4 ± 0.1 | 98.4 ± 0.2 | 97.2 ± 0.2 | 77.9 ± 0.2 | 56.2 ± 0.2 |
| at day 30 | | | | | | | | |
| Purity(%) | 85.1 ± 0.2 | 97.2 ± 0.1 | 95.1 ± 0.1 | 97.3 ± 0.2 | 97.4 ± 0.2 | 94.1 ± 0.1 | 43.6 ± 0.2 | 33.3 ± 0.3 |
| at day 90 | | | | | | | | |
| Purity(%) | 71.9 ± 0.2 | 95.4 ± 0.1 | 90.9 ± 0.2 | 95.5 ± 0.2 | 95.5 ± 0.2 | 88.7 ± 0.2 | 24.3 ± 0.3 | 5.8 ± 0.3 |
| at day 180 | | | | | | | | |
| Purity(%) | 51.8 ± 0.2 | 92.3 ± 0.2 | 82.8 ± 0.3 | 92.5 ± 0.2 | 92.4 ± 0.2 | 81.1 ± 0.3 | 0 | 0 |
| at day 360 | | | | | | | | |

The solution of H-Tyr-Gly-Gly-Phe-Met-OCH(CH₃)₂.HCl salt is only stable at pH 3-6 and can be stored for about 1 year at room temperature.

The results show that the reconstitution solution is stable only at pH 3-6, preferably at pH 3-5, more preferably at pH 3.5-4.5. The pH of the solution can be adjusted with any acid or base, such as HCl or NaOH, preferably with weak base. The pH adjusting and buffering agent can be sodium, potassium, calcium, lithium, or magnesium salt of an organic acid, for example, sodium, potassium or lithium salt of acetic acid, propionic acid, butyric acid, valeric acid, benzoic acid, lactic acid, salicylic acid, citric acid, ascorbic acid, succinic acid, or maleic acid.

TABLE 31
Stabilities of 7% solution of 2-(diethylamino)ethyl (R,S)-2-(6-methoxy-2-naphthyl)
propionate•HCl salt (C-1), 2-(diethylamino)ethyl (R,S)-2-(6-methoxy-2-naphthyl)
propionate•HBr salt (C-2), and 2-(diethylamino)ethyl (R,S)-2-(6-methoxy-
2-naphthyl) propionate•citric acid salt (C-3) at various pH values in water
(pH was adjusted with 3N HCl or 3N NaOH) at 25° C. for 28 days.

| | | Purity(%) from | Purity(%) from | Purity(%) from |
|---|---|---|---|---|
| pH | solvent | day 0-28 (C-1) | day 0-28 (C-2) | day 0-28 (C-3) |
| 1.0 | water | 98.5 ± 0.3→68.9 ± 0.3 | 98.3 ± 0.3→69.7 ± 0.3 | 98.0 ± 0.3→68.2 ± 0.4 |
| 2.0 | water | 98.6 ± 0.2→80.9 ± 0.4 | 98.5 ± 0.3→80.1 ± 0.3 | 98.2 ± 0.4→80.1 ± 0.3 |
| 3.0 | water | 98.6 ± 0.2→90.3 ± 0.3 | 98.6 ± 0.4→90.7 ± 0.3 | 98.2 ± 0.2→90.3 ± 0.3 |
| 3.7 | water | 98.8 ± 0.3→97.2 ± 0.2 | 98.6 ± 0.3→97.1 ± 0.3 | 98.3 ± 0.4→96.8 ± 0.2 |
| 4.0 | water | 98.8 ± 0.2→97.8 ± 0.2 | 98.7 ± 0.4→97.7 ± 0.2 | 98.4 ± 0.3→97.1 ± 0.2 |
| 4.3 | water | 98.8 ± 0.3→97.9 ± 0.2 | 98.8 ± 0.2→97.8 ± 0.2 | 98.3 ± 0.4→97.1 ± 0.3 |
| 4.6 | water | 98.8 ± 0.2→97.9 ± 0.1 | 98.7 ± 0.3→97.8 ± 0.2 | 98.3 ± 0.2→97.0 ± 0.3 |
| 5.0 | water | 98.7 ± 0.3→96.3 ± 0.2 | 98.7 ± 0.2→96.5 ± 0.3 | 98.1 ± 0.3→96.0 ± 0.3 |
| 6.0 | water | 98.6 ± 0.2→89.9 ± 0.3 | 98.7 ± 0.3→89.0 ± 0.4 | 98.1 ± 0.3→88.4 ± 0.3 |
| 7.0 | water | 98.5 ± 0.3→65.8 ± 0.3 | 98.6 ± 0.3→65.5 ± 0.3 | 98.0 ± 0.3→55.8 ± 0.4 |
| 8.0 | water | 98.3 ± 0.3→45.8 ± 0.4 | 98.5 ± 0.3→45.5 ± 0.5 | 98.0 ± 0.2→35.8 ± 0.5 |

The results show that only pH values, but not the acid, such as HCl, HBr or citric acid, which formed the salt with 2-(diethyl amino)ethyl 2-(6-methoxy-2-naphthyl) propionate, affect the stability of the 2-(diethylamino)ethyl 2-(6-methoxy-2-naphthyl) propionate salt significantly.

TABLE 32
Stabilities of 7% of 2-(diethylamino)ethyl (R,S)-2-(p-isobutylphenyl)propionate•HCl
salt (C-4), 2-(dimethylamino)ethyl (R,S)-2-(p-isobutylphenyl)propionate•HCl salt (C-5), and
2-(dibutylamino)ethyl (R,S)-2-(p-isobutylphenyl)propionate•HCl salt (C-6) at various pH
values in 25% ethanol (pH was adjusted with 3N HCl or 3N NaOH) at 25° C. for 28 days.

| | | Purity(%) from | Purity(%) from | Purity(%) from |
|---|---|---|---|---|
| pH | solvent | day 0-28 (C-4) | day 0-28 (C-5) | day 0-28 (C-6) |
| 1.0 | 25% ethanol | 98.2 ± 0.3→69.1 ± 0.2 | 98.0 ± 0.3→65.3 ± 0.3 | 97.9 ± 0.3→68.4 ± 0.3 |
| 2.0 | 25% ethanol | 98.3 ± 0.3→80.8 ± 0.4 | 98.2 ± 0.4→77.4 ± 0.3 | 98.0 ± 0.3→79.2 ± 0.4 |
| 3.0 | 25% ethanol | 98.5 ± 0.2→92.5 ± 0.3 | 98.3 ± 0.3→88.4 ± 0.2 | 98.2 ± 0.4→91.2 ± 0.3 |
| 3.7 | 25% ethanol | 98.6 ± 0.3→97.2 ± 0.4 | 98.5 ± 0.4→95.7 ± 0.3 | 98.3 ± 0.3→96.9 ± 0.2 |
| 4.0 | 25% ethanol | 98.6 ± 0.2→97.8 ± 0.3 | 98.5 ± 0.3→96.6 ± 0.4 | 98.4 ± 0.3→97.3 ± 0.2 |
| 4.3 | 25% ethanol | 98.7 ± 0.3→97.7 ± 0.2 | 98.6 ± 0.3→96.8 ± 0.3 | 98.4 ± 0.3→97.3 ± 0.3 |
| 4.6 | 25% ethanol | 98.6 ± 0.2→97.3 ± 0.3 | 98.5 ± 0.2→96.8 ± 0.2 | 98.3 ± 0.3→97.1 ± 0.3 |
| 5.0 | 25% ethanol | 98.5 ± 0.3→94.3 ± 0.2 | 98.5 ± 0.3→93.1 ± 0.4 | 98.3 ± 0.3→94.5 ± 0.3 |
| 6.0 | 25% ethanol | 98.4 ± 0.3→89.9 ± 0.3 | 98.4 ± 0.3→85.3 ± 0.2 | 98.2 ± 0.4→89.2 ± 0.4 |
| 7.0 | 25% ethanol | 98.2 ± 0.3→52.8 ± 0.4 | 98.2 ± 0.3→47.1 ± 0.3 | 98.0 ± 0.3→51.5 ± 0.3 |
| 8.0 | 25% ethanol | 98.0 ± 0.4→35.3 ± 0.3 | 98.0 ± 0.3→34.9 ± 0.5 | 97.8 ± 0.4→35.1 ± 0.3 |

The results show that the size of R₁, R₂ and R on the amino group does not affect the stability of aminoalkyl (R,S)-2-(p-isobutylphenyl)propionate significantly.

TABLE 33
Stabilities of 7% of 2-pyrrolidinemethyl (R,S)-2-(p-isobutylphenyl)propionate•HCl salt
(C-7), 4-piperidineethyl (R,S)-2-(p-isobutylphenyl)propionate•HCl salt (C-8), 1-pyrrolidineethyl
(R,S)-2-(p-isobutylphenyl)propionate•HCl salt (C-9), and 1-piperidineethyl (R,S)-2-(p-
isobutylphenyl)propionate•HCl salt (C-10) at various pH values and temperature in 25% ethanol
(pH was adjusted with 3N HCl or 3N NaOH) at 25° C. for 28 days.

| | | Purity(%) from | Purity(%) from | Purity(%) from | Purity(%) from |
|---|---|---|---|---|---|
| pH | solvent | day 0-28 (C-7) | day 0-28 (C-8) | day 0-28 (C-9) | day 0-28 (C-10) |
| 1.0 | 25% ethanol | 97.8 ± 0.4→66.8 ± 0.3 | 97.6 ± 0.4→68.4 ± 0.4 | 98.0 ± 0.3→68.3 ± 0.2 | 97.6 ± 0.3→68.1 ± 0.3 |
| 2.0 | 25% ethanol | 98.1 ± 0.3→86.0 ± 0.4 | 98.0 ± 0.3→87.3 ± 0.3 | 98.2 ± 0.4→87.7 ± 0.3 | 97.9 ± 0.4→87.0 ± 0.3 |
| 3.0 | 25% ethanol | 98.3 ± 0.3→86.0 ± 0.3 | 98.1 ± 0.2→87.3 ± 0.3 | 98.5 ± 0.3→87.7 ± 0.2 | 98.0 ± 0.3→87.0 ± 0.2 |
| 3.7 | 25% ethanol | 98.5 ± 0.2→94.3 ± 0.3 | 98.3 ± 0.3→95.0 ± 0.2 | 98.6 ± 0.4→95.5 ± 0.3 | 98.2 ± 0.3→95.1 ± 0.3 |
| 4.0 | 25% ethanol | 98.6 ± 0.3→95.8 ± 0.3 | 98.4 ± 0.3→96.1 ± 0.3 | 98.6 ± 0.3→96.3 ± 0.3 | 98.2 ± 0.2→95.8 ± 0.3 |
| 4.3 | 25% ethanol | 98.5 ± 0.3→96.5 ± 0.4 | 98.4 ± 0.2→96.3 ± 0.3 | 98.7 ± 0.2→96.4 ± 0.2 | 98.3 ± 0.3→95.9 ± 0.2 |
| 4.6 | 25% ethanol | 98.5 ± 0.2→96.2 ± 0.3 | 98.3 ± 0.2→96.3 ± 0.2 | 98.6 ± 0.3→96.4 ± 0.3 | 98.2 ± 0.4→95.8 ± 0.3 |
| 5.0 | 25% ethanol | 98.4 ± 0.3→89.3 ± 0.2 | 98.3 ± 0.4→89.7 ± 0.3 | 98.5 ± 0.4→88.6 ± 0.3 | 98.2 ± 0.3→88.5 ± 0.4 |
| 6.0 | 25% ethanol | 98.4 ± 0.4→86.2 ± 0.3 | 98.2 ± 0.3→87.3 ± 0.3 | 98.4 ± 0.5→89.0 ± 0.4 | 98.1 ± 0.3→87.9 ± 0.3 |
| 7.0 | 25% ethanol | 98.2 ± 0.3→48.0 ± 0.3 | 97.9 ± 0.3→48.3 ± 0.4 | 98.1 ± 0.5→48.1 ± 0.3 | 97.8 ± 0.3→47.8 ± 0.3 |
| 8.0 | 25% ethanol | 97.7 ± 0.3→34.3 ± 0.4 | 97.6 ± 0.5→34.2 ± 0.3 | 97.9 ± 0.5→34.4 ± 0.4 | 97.5 ± 0.5→34.1 ± 0.4 |

The results show that the size of R₁, R₂ and R on the amino group does not affect the stability of aminoalkyl (R,S′)-2-(p-isobutylphenyl)propionate significantly.

TABLE 34
Stabilities of 7% of 2-(diethylamino)ethyl acetylsalicylate•maleic acid salt (A-1), 2-
(diethylamino)ethyl acetylsalicylate•benzoic acid salt (A-2), 2-(diethylamino)ethyl
acetylsalicylate•lactic acid salt (A-3), and 2-(diethylamino)ethyl acetylsalicylate•valeric acid (A-4) salt
at various pH values in water (pH was adjusted with 3N HCl or 3N NaOH) at 25° C. for 14 days.

| | | Purity(%) from | Purity(%) from | Purity(%) from | Purity (%) |
|---|---|---|---|---|---|
| pH | solvent | day 0-14 (A-1) | day 0-14 (A-2) | day 0-14 (A-3) | from day 0-14 (A-4) |
| 1.0 | water | 98.1 ± 0.3→59.1 ± 0.4 | 98.2 ± 0.2→60.5 ± 0.3 | 97.8 ± 0.3→57.9 ± 0.4 | 97.8 ± 0.2→58.1 ± 0.3 |
| 2.0 | water | 98.2 ± 0.3→78.8 ± 0.3 | 98.4 ± 0.2→79.7 ± 0.4 | 98.0 ± 0.2→77.0 ± 0.4 | 98.1 ± 0.2→77.3 ± 0.2 |
| 3.0 | water | 98.4 ± 0.1→90.9 ± 0.2 | 98.5 ± 0.1→91.0 ± 0.2 | 98.2 ± 0.1→88.7 ± 0.2 | 98.3 ± 04→87.5 ± 0.3 |
| 3.7 | water | 98.5 ± 0.1→94.0 ± 0.3 | 98.6 ± 0.1→94.1 ± 0.2 | 98.2 ± 0.2→93.1 ± 0.3 | 98.4 ± 0.1→93.2 ± 0.3 |
| 4.0 | water | 98.6 ± 0.1→94.5 ± 0.2 | 98.6 ± 0.2→95.0 ± 0.3 | 98.3 ± 0.1→94.1 ± 0.4 | 98.4 ± 0.2→94.2 ± 0.4 |
| 4.3 | water | 98.5 ± 0.2→94.6 ± 0.3 | 98.7 ± 0.2→95.0 ± 0.4 | 98.2 ± 0.1→94.4 ± 0.2 | 98.5 ± 0.1→94.7 ± 0.2 |
| 4.6 | water | 98.5 ± 0.3→94.3 ± 0.2 | 98.6 ± 0.3→94.4 ± 0.3 | 98.2 ± 0.3→94.1 ± 0.2 | 98.4 ± 0.3→94.0 ± 0.2 |
| 5.0 | water | 98.4 ± 0.1→86.9 ± 0.3 | 98.5 ± 0.2→87.3 ± 0.2 | 98.2 ± 0.2→85.6 ± 0.4 | 98.3 ± 0.2→85.5 ± 0.4 |
| 6.0 | water | 98.3 ± 0.2→78.9 ± 0.3 | 98.4 ± 0.3→78.3 ± 0.3 | 98.0 ± 0.1→78.3 ± 0.3 | 98.2 ± 0.2→77.3 ± 0.3 |
| 7.0 | water | 98.2 ± 0.2→57.8 ± 0.4 | 98.2 ± 0.2→56.0 ± 0.4 | 98.0 ± 0.2→55.1 ± 0.3 | 98.0 ± 0.3→52.5 ± 0.4 |
| 8.0 | water | 98.0 ± 0.3→16.1 ± 0.4 | 98.0 ± 0.3→17.7 ± 0.4 | 97.8 ± 0.2→15.0 ± 0.4 | 97.9 ± 0.2→15.2 ± 0.4 |

The results show that only pH values, but not the acid, such as maleic acid, benzoic acid, lactic acid, or valeric acid, which formed the salt with 2-(diethylamino)ethyl acetylsalicylate, affect the stability of the 2-(diethylamino)ethyl acetylsalicylate salt significantly.

The results further show that only pH values, but not the acid, such as HCl, HBr, citric acid, maleic acid, benzoic acid, or lactic acid, which forms the protonated amine groups, affect the stability of the solution significantly. In addition, the size of the groups on the amino group, such as R₁, R₂ and R, does not affect the stability significantly.

### 3. Effect of Temperature on the Stability

TABLE 35
Stabilities of 5% solution of H-Val-Pro-Gly-Pro-Arg(NO₂)—OCH(CH₃)₂•HCl salt with
1 equivalent sodium acetate (T-1), H-Ala-Pro-Gly-Pro-Arg(NO₂)—OCH(CH₃)₂•HCl salt with 1 equivalent
sodium acetate (T-2), and H-Val-Pro-Asp[OCH(CH₃)₂]-Pro-Arg(NO₂)—OCH(CH₃)₂•HCl
salt with 1 equivalent sodium acetate (T-3) at various temperature in 25% ethanol.

| | | | Purity(%) at | Purity(%) at | Purity(%) at | Purity(%) at | Purity (%) at |
|---|---|---|---|---|---|---|---|
| | pH | solvent | day 0 | day 30 (5° C.) | day 30 (25° C.) | day 30 (40° C.) | day 30 (60° C.) |
| T-1 | 4.50 | 25% ethanol | 98.7 ± 0.2 | 98.5 ± 0.3 | 97.7 ± 0.2 | 71.6 ± 0.2 | 32.4 ± 0.4 |
| T-2 | 4.51 | 25% ethanol | 98.9 ± 0.2 | 98.8 ± 0.2 | 97.8 ± 0.3 | 71.3 ± 0.2 | 32.8 ± 0.2 |
| T-3 | 4.49 | 25% ethanol | 99.1 ± 0.1 | 98.9 ± 0.2 | 97.9 ± 0.3 | 71.9 ± 0.2 | 33.2 ± 0.3 |
| | | | Purity(%) at | Purity(%) at | Purity(%) at | Purity(%) at | Purity (%) at |
| | pH | solvent | day 0 | day 90 (5° C.) | day 90 (25° C.) | day 90 (40° C.) | day 90 (60° C.) |
| T-1 | 4.50 | 25% ethanol | 98.7 ± 0.2 | 98.4 ± 0.3 | 97.0 ± 0.3 | 32.7 ± 0.3 | 0 |
| T-2 | 4.51 | 25% ethanol | 98.9 ± 0.2 | 98.5 ± 0.3 | 97.0 ± 0.2 | 33.6 ± 0.2 | 0 |
| T-3 | 4.49 | 25% ethanol | 99.1 ± 0.1 | 98.6 ± 0.3 | 97.2 ± 0.3 | 34.2 ± 0.3 | 0 |
| | | | Purity(%) at | Purity(%) at | Purity(%) at | Purity(%) at | Purity (%) at |
| | pH | solvent | day 0 | day 180 (5° C.) | day 180 (25° C.) | day 180 (40° C.) | day 180 (60° C.) |
| T-1 | 4.50 | 25% ethanol | 98.7 ± 0.2 | 97.8 ± 0.3 | 95.1 ± 0.3 | 12.2 ± 0.3 | 0 |
| T-2 | 4.51 | 25% ethanol | 98.9 ± 0.2 | 97.8 ± 0.2 | 95.2 ± 0.3 | 13.6 ± 0.2 | 0 |
| T-3 | 4.49 | 25% ethanol | 99.1 ± 0.1 | 97.9 ± 0.2 | 95.2 ± 0.3 | 13.5 ± 0.3 | 0 |
| | | | Purity(%) at | Purity(%) at | Purity(%) at | Purity(%) at | Purity (%) at |
| | pH | solvent | day 0 | day 360 (5° C.) | day 360 (25° C.) | day 360 (40° C.) | day 360 (60° C.) |
| T-1 | 4.50 | 25% ethanol | 98.7 ± 0.2 | 97.3 ± 0.3 | 92.2 ± 0.2 | 0 | 0 |
| T-2 | 4.51 | 25% ethanol | 98.9 ± 0.2 | 97.4 ± 0.3 | 92.3 ± 0.3 | 0 | 0 |
| T-3 | 4.49 | 25% ethanol | 99.1 ± 0.1 | 97.4 ± 0.3 | 92.5 ± 0.4 | 0 | 0 |

The solutions of H-Val-Pro-Gly-Pro-Arg(NO₂)—OCH(CH₃)₂.HCl salt(T-1), H-Ala-Pro-Gly-Pro-Arg(NO₂)—OCH(CH₃)₂.HCl salt (T-2), and H-Val-Pro-Asp[OCH(CH₃)₂]-Pro-Arg(NO₂)—OCH(CH₃)₂.HCl salt (T-3) are more stable at lower temperature and can be stored for more than 1 years at 25° C. and 5° C.

TABLE 36
Stabilities of 7% solution of (R,S)-2-(diethylamino)ethyl 2-(6-
methoxy-2-naphthyl) propionate•HCl salt at various pH values
and temperature in water (pH was adjusted with 3N HCl or 3N NaOH).

| | | Purity(%) at | Purity(%) at | Purity(%) at | Purity(%) at | Purity (%) at |
|---|---|---|---|---|---|---|
| pH | solvent | day 0 | day 21 (5° C.) | day 21 (25° C.) | day 21 (40° C.) | day 21 (60° C.) |
| 1.02 | water | 98.6 ± 0.2 | 93.8 ± 0.2 | 72.2 ± 0.2 | 18.1 ± 0.3 | 0 |
| 2.09 | water | 98.6 ± 0.3 | 98.1 ± 0.2 | 86.3 ± 0.2 | 30.2 ± 0.4 | 0 |
| 3.00 | water | 98.7 ± 0.2 | 98.4 ± 0.2 | 96.3 ± 0.2 | 56.7 ± 0.2 | 7.9 ± 0.2 |
| 3.68 | water | 98.8 ± 0.3 | 98.6 ± 0.1 | 98.0 ± 0.1 | 63.7 ± 0.2 | 15.3 ± 0.4 |
| 4.07 | water | 98.7 ± 0.4 | 98.7 ± 0.1 | 98.3 ± 0.2 | 72.5 ± 0.3 | 21.5 ± 0.3 |
| 4.35 | water | 98.8 ± 0.3 | 98.7 ± 0.1 | 98.2 ± 0.1 | 72.3 ± 0.2 | 18.7 ± 0.2 |
| 4.67 | water | 98.8 ± 0.3 | 98.7 ± 0.1 | 98.2 ± 0.2 | 58.5 ± 0.2 | 10.6 ± 0.2 |
| 4.95 | water | 98.7 ± 0.2 | 97.9 ± 0.2 | 95.5 ± 0.3 | 50.6 ± 0.2 | 3.1 ± 0.3 |
| 5.98 | water | 98.8 ± 0.3 | 96.1 ± 0.2 | 90.7 ± 0.3 | 30.3 ± 0.3 | 0 |
| 6.98 | water | 98. ± 50.2 | 90.9 ± 0.2 | 70.8 ± 0.2 | 19.9 ± 0.3 | 0 |
| 8.01 | water | 98.3 ± 0.3 | 81.9 ± 0.2 | 58.8 ± 0.2 | 0 | 0 |

The results show that the solution of 2-(diethylamino)ethyl 2-(6-methoxy-2-naphthyl) propionate.HCl salt in water is not stable at a temperature higher than 40° C. and a pH lower than 3 or greater than 6.

TABLE 37
Stabilities of 7% solution of 2-(diethylamino)ethyl (R,S)-2-(p-
isobutylphenyl)propionate•HCl salt at various pH values and
temperature in water (pH was adjusted with 3N HCl or 3N NaOH).

| | | Purity(%) at | Purity(%) at | Purity(%) at | Purity(%) at | Purity (%) at |
|---|---|---|---|---|---|---|
| pH | solvent | day 0 | day 21 (5° C.) | day 21 (25° C.) | day 21 (40° C.) | day 21 (60° C.) |
| 1.02 | water | 98.0 ± 0.4 | 93.7 ± 0.3 | 73.0 ± 0.4 | 18.6 ± 0.4 | 0 |
| 2.09 | water | 98.2 ± 0.4 | 97.6 ± 0.4 | 86.1 ± 0.3 | 30.4 ± 0.3 | 0 |
| 3.00 | water | 98.5 ± 0.3 | 98.0 ± 0.4 | 96.5 ± 0.4 | 56.3 ± 0.4 | 8.8 ± 0.3 |
| 3.68 | water | 98.5 ± 0.2 | 98.4 ± 0.3 | 98.1 ± 0.4 | 64.3 ± 0.4 | 16.4 ± 0.4 |
| 4.07 | water | 98.6 ± 0.3 | 98.5 ± 0.3 | 98.2 ± 0.2 | 72.7 ± 0.3 | 22.6 ± 0.4 |
| 4.35 | water | 98.6 ± 0.2 | 98.6 ± 0.4 | 98.2 ± 0.3 | 72.6 ± 0.3 | 18.8 ± 0.2 |
| 4.67 | water | 98.5 ± 0.1 | 98.5 ± 0.4 | 98.1 ± 0.4 | 58.2 ± 0.4 | 10.3 ± 0.3 |
| 4.95 | water | 98.5 ± 0.3 | 97.5 ± 0.4 | 95.4 ± 0.4 | 50.2 ± 0.4 | 3.9 ± 0.3 |
| 5.98 | water | 98.4 ± 0.3 | 96.1 ± 0.3 | 90.7 ± 0.3 | 29.9 ± 0.2 | 0 |
| 6.98 | water | 98.3 ± 0.4 | 90.7 ± 0.4 | 78.6 ± 0.4 | 19.7 ± 0.4 | 0 |
| 8.01 | water | 98.0 ± 0.4 | 81.6 ± 0.5 | 52.5 ± 0.5 | 0 | 0 |

The results show that the solution of 2-(diethylamino)ethyl (R,S)-2-(p-isobutylphenyl)propionate.HCl salt in water is not stable at a temperature higher than 40C and a pH lower than 3 or greater than 6.

TABLE 38
Stabilities of 7% solution of 2-(diethylamino)ethyl (R)-2-(p-
isobutylphenyl)propionate•HCl salt at various pH values and
temperature in water (pH was adjusted with 3N HCl or 3N NaOH).

| | | Purity(%) at | Purity(%) at | Purity(%) at | Purity(%) at | Purity (%) at |
|---|---|---|---|---|---|---|
| pH | solvent | day 0 | day 21 (5° C.) | day 21 (25° C.) | day 21 (40° C.) | day 21 (60° C.) |
| 1.02 | water | 97.8 ± 0.4 | 93.1 ± 0.3 | 70.7 ± 0.4 | 18.5 ± 0.3 | 0 |
| 2.09 | water | 98.0 ± 0.4 | 97.0 ± 0.4 | 86.0 ± 0.4 | 30.6 ± 0.4 | 0 |
| 3.01 | water | 98.1 ± 0.3 | 97.8 ± 0.4 | 96.1 ± 0.3 | 56.1 ± 0.4 | 8.4 ± 0.3 |
| 3.68 | water | 98.2 ± 0.2 | 98.1 ± 0.3 | 97.7 ± 0.3 | 64.1 ± 0.5 | 16.0 ± 0.4 |
| 4.07 | water | 98.2 ± 0.3 | 98.0 ± 0.4 | 97.8 ± 0.4 | 72.6 ± 0.4 | 22.7 ± 0.4 |
| 4.35 | water | 98.3 ± 0.2 | 98.1 ± 0.4 | 97.8 ± 0.3 | 72.6 ± 0.5 | 18.3 ± 0.3 |
| 4.67 | water | 98.2 ± 0.3 | 98.1 ± 0.2 | 97.5 ± 0.4 | 58.3 ± 0.4 | 10.4 ± 0.5 |
| 4.96 | water | 98.1 ± 0.3 | 97.0 ± 0.4 | 95.1 ± 0.4 | 50.5 ± 0.5 | 4.0 ± 0.3 |
| 5.98 | water | 98.0 ± 0.4 | 95.8 ± 0.4 | 90.2 ± 0.4 | 29.9 ± 0.4 | 0 |
| 6.99 | water | 97.9 ± 0.4 | 90.0 ± 0.5 | 77.1 ± 0.4 | 19.9 ± 0.4 | 0 |
| 8.01 | water | 97.6 ± 0.5 | 81.1 ± 0.5 | 52.3 ± 0.4 | 0 | 0 |

The results show that the solution of 2-(diethylamino)ethyl (R)-2-(p-isobutylphenyl)propionate.HCl salt in water is not stable at a temperature higher than 40° C. and a pH lower than 3 or greater than 6, and there is no significant difference between (R,S)- and (R)-isomer.

TABLE 39
Stabilities of 7% solution of 2-(diethylamino)ethyl 2-(2,4-dichlorophenoxy)benzeneacetate•HCl
salt at various pH values and temperature in water (pH was adjusted
with 3N HCl or 3N NaOH).

| | | Purity(%) at | Purity(%) at | Purity(%) at | Purity(%) at | Purity (%) at |
|---|---|---|---|---|---|---|
| pH | solvent | day 0 | day 21 (5° C.) | day 21 (25° C.) | day 21 (40° C.) | day 21 (60° C.) |
| 1.0 | water | 97.9 ± 0.3 | 91.3 ± 0.3 | 67.3 ± 0.3 | 15.1 ± 0.3 | 0 |
| 2.0 | water | 98.0 ± 0.2 | 95.1 ± 0.3 | 82.1 ± 0.3 | 27.0 ± 0.3 | 0 |
| 3.0 | water | 98.3 ± 0.2 | 97.0 ± 0.2 | 93.5 ± 0.2 | 48.3 ± 0.3 | 0 |
| 3.7 | water | 98.4 ± 0.1 | 97.6 ± 0.2 | 96.9 ± 0.3 | 57.2 ± 0.2 | 13.3 ± 0.5 |
| 4.0 | water | 98.5 ± 0.1 | 98.0 ± 0.1 | 97.0 ± 0.2 | 68.1 ± 0.3 | 18.2 ± 0.3 |
| 4.3 | water | 98.5 ± 0.2 | 98.0 ± 0.1 | 97.0 ± 0.3 | 68.1 ± 0.3 | 17.2 ± 0.2 |
| 4.6 | water | 98.6 ± 0.1 | 98.1 ± 0.2 | 96.1 ± 0.2 | 54.5 ± 0.2 | 14.3 ± 0.3 |
| 5.0 | water | 98.4 ± 0.2 | 96.9 ± 0.2 | 93.1 ± 0.3 | 40.8 ± 0.3 | 1.2 ± 0.2 |
| 6.0 | water | 98.2 ± 0.2 | 95.0 ± 0.2 | 86.7 ± 0.2 | 27.8 ± 0.2 | 0 |
| 7.0 | water | 98.0 ± 0.3 | 90.0 ± 0.3 | 59.3 ± 0.3 | 11.0 ± 0.4 | 0 |
| 8.0 | water | 97.8 ± 0.3 | 78.3 ± 0.3 | 47.1 ± 0.3 | 0 | 0 |

The results show that 2-(diethylamino)ethyl 2-(2,4-dichlorophenoxy)benzeneacetate.HCl salt is not stable at a temperature higher than 40° C. and a pH lower than 3 or greater than 6.

TABLE 40
Stabilities of 7% solution of 2-(diethylamino)ethyl (R,S)-2-(2-
fluoro-4-biphenyl)propionate•HCl salt at various pH values
and temperature in water (pH was adjusted with 3N HCl or 3N NaOH).

| | | Purity(%) at | Purity(%) at | Purity(%) at | Purity(%) at | Purity (%) at |
|---|---|---|---|---|---|---|
| pH | solvent | day 0 | day 21 (5° C.) | day 21 (25° C.) | day 21 (40° C.) | day 21 (60° C.) |
| 1.0 | water | 98.2 ± 0.3 | 94.0 ± 0.3 | 72.0 ± 0.3 | 18.5 ± 0.3 | 0 |
| 2.0 | water | 98.3 ± 0.3 | 97.8 ± 0.2 | 86.1 ± 0.3 | 30.1 ± 0.4 | 0 |
| 3.0 | water | 98.4 ± 0.2 | 98.0 ± 0.2 | 96.0 ± 0.2 | 56.5 ± 0.3 | 8.7 ± 0.3 |
| 3.7 | water | 98.5 ± 0.3 | 98.2 ± 0.1 | 97.5 ± 0.1 | 64.5 ± 0.3 | 16.2 ± 0.3 |
| 4.0 | water | 98.5 ± 0.2 | 98.3 ± 0.1 | 97.7 ± 0.2 | 72.3 ± 0.3 | 22.4 ± 0.3 |
| 4.3 | water | 98.5 ± 0.3 | 98.3 ± 0.1 | 97.8 ± 0.1 | 72.2 ± 0.3 | 18.3 ± 0.2 |
| 4.6 | water | 98.4 ± 0.2 | 98.3 ± 0.1 | 97.7 ± 0.1 | 58.1 ± 0.4 | 10.4 ± 0.3 |
| 5.0 | water | 98.5 ± 0.3 | 97.6 ± 0.2 | 96.7 ± 0.2 | 50.1 ± 0.3 | 3.0 ± 0.3 |
| 6.0 | water | 98.3 ± 0.3 | 95.4 ± 0.2 | 90.5 ± 0.3 | 29.7 ± 0.4 | 0 |
| 7.0 | water | 98.1 ± 0.4 | 89.7 ± 0.3 | 75.1 ± 0.3 | 19.8 ± 0.3 | 0 |
| 8.0 | water | 98.0 ± 0.4 | 81.9 ± 0.3 | 52.8 ± 0.3 | 0 | 0 |

The results show that the solution of 2-(diethylamino)ethyl (R,S′)-2-(2-fluoro-4-biphenyl)propionate.HCl salt in water is not stable at a temperature higher than 40PC and a pH lower than 3 or greater than 6.

TABLE 41
Stabilities of 7% solution of 2-(diethylamino)ethyl 1-(4-chlorobenzoyl)-
5-methoxy-2-methyl-1H-indole-3-acetate•HCl salt at various pH values
and temperature in water (pH was adjusted with 3N HCl or 3N NaOH).

| | | Purity(%) at | Purity(%) at | Purity(%) at | Purity(%) at | Purity (%) at |
|---|---|---|---|---|---|---|
| pH | solvent | day 0 | day 21 (5° C.) | day 21 (25° C.) | day 21 (40° C.) | day 21 (60° C.) |
| 1.0 | water | 98.1 ± 0.3 | 92.3 ± 0.3 | 69.0 ± 0.3 | 15.3 ± 0.3 | 0 |
| 2.0 | water | 98.3 ± 0.4 | 95.9 ± 0.3 | 83.1 ± 0.3 | 27.5 ± 0.3 | 0 |
| 3.0 | water | 98.5 ± 0.2 | 97.3 ± 0.3 | 94.5 ± 0.2 | 49.3 ± 0.3 | 0 |
| 3.7 | water | 98.5 ± 0.2 | 97.7 ± 0.2 | 97.3 ± 0.3 | 57.9 ± 0.2 | 16.3 ± 0.5 |
| 4.0 | water | 98.7 ± 0.1 | 98.1 ± 0.3 | 97.5 ± 0.2 | 68.7 ± 0.3 | 19.6 ± 0.3 |
| 4.3 | water | 98.8 ± 0.2 | 98.3 ± 0.3 | 97.3 ± 0.3 | 70.1 ± 0.3 | 17.7 ± 0.2 |
| 4.6 | water | 98.7 ± 0.1 | 98.2 ± 0.2 | 96.4 ± 0.2 | 56.3 ± 0.2 | 15.3 ± 0.3 |
| 5.0 | water | 98.6 ± 0.3 | 97.0 ± 0.2 | 94.1 ± 0.3 | 42.8 ± 0.3 | 1.9 ± 0.2 |
| 6.0 | water | 98.5 ± 0.4 | 95.3 ± 0.3 | 87.8 ± 0.2 | 27.6 ± 0.2 | 0 |
| 7.0 | water | 98.4 ± 0.3 | 90.2 ± 0.3 | 59.9 ± 0.3 | 11.4 ± 0.4 | 0 |
| 8.0 | water | 98.0 ± 0.5 | 79.3 ± 0.4 | 47.6 ± 0.3 | 0 | 0 |

The results show that the solution of 2-(diethylamino)ethyl 1-(4-chlorobenzoyl)-5-methoxy-2-methyl-1H-indole-3-acetate.HCl salt in water is not stable at a temperature higher than 40° C. and a pH lower than 3 or greater than 6.

TABLE 42
Stabilities of 7% solution of 2-(diethylamino)ethyl 5-fluoro-2-methyl-1-[[4-
(methylsulfinyl)phenyl]methylene]-1H-indene-3-acetate•HCl salt at various
pH values and temperature in water (pH was adjusted with 3N HCl or 3N NaOH).

| | | Purity(%) at | Purity(%) at | Purity(%) at | Purity(%) at | Purity (%) at |
|---|---|---|---|---|---|---|
| pH | solvent | day 0 | day 21 (5° C.) | day 21 (25° C.) | day 21 (40° C.) | day 21 (60° C.) |
| 1.0 | water | 97.2 ± 0.3 | 91.1 ± 0.4 | 67.5 ± 0.4 | 10.4 ± 0.4 | 0 |
| 2.0 | water | 97.3 ± 0.3 | 94.7 ± 0.3 | 81.1 ± 0.3 | 23.6 ± 0.3 | 0 |
| 3.0 | water | 97.6 ± 0.2 | 96.3 ± 0.2 | 92.2 ± 0.2 | 45.7 ± 0.3 | 0 |
| 3.7 | water | 97.6 ± 0.2 | 97.3 ± 0.2 | 96.8 ± 0.2 | 55.5 ± 0.2 | 13.5 ± 0.3 |
| 4.0 | water | 97.9 ± 0.1 | 97.4 ± 0.1 | 96.9 ± 0.1 | 60.3 ± 0.2 | 19.7 ± 0.3 |
| 4.3 | water | 97.7 ± 0.2 | 97.3 ± 0.1 | 96.7 ± 0.1 | 63.1 ± 0.1 | 16.8 ± 0.2 |
| 4.6 | water | 97.8 ± 0.1 | 97.3 ± 0.2 | 95.9 ± 0.2 | 57.3 ± 0.2 | 15.5 ± 0.3 |
| 5.0 | water | 97.7 ± 0.3 | 96.1 ± 0.2 | 93.9 ± 0.3 | 38.9 ± 0.3 | 1.5 ± 0.3 |
| 6.0 | water | 97.5 ± 0.3 | 94.4 ± 0.3 | 88.9 ± 0.2 | 21.6 ± 0.2 | 0 |
| 7.0 | water | 97.3 ± 0.3 | 89.0 ± 0.3 | 59.8 ± 0.3 | 5.4 ± 0.3 | 0 |
| 8.0 | water | 96.9 ± 0.4 | 77.1 ± 0.3 | 44.5 ± 0.4 | 0 | 0 |

The results show that the solution of 2-(diethylamino)ethyl 5-fluoro-2-methyl-1-[[4-(methyl sulfinyl)phenyl]methyl ene]-1H-indene-3-acetate.HCl salt in water is not stable at a temperature higher than 40C and a pH lower than 3 or greater than 6.

TABLE 43
Stabilities of 7% solution of 2-(diethylamino)ethyl 1-methyl-5-(4-
methylbenzoyl)-1H-pyrrole-2-acetate•HCl salt at various pH values
and temperature in water (pH was adjusted with 3N HCl or 3N NaOH).

| | | Purity(%) at | Purity(%) at | Purity(%) at | Purity(%) at | Purity (%) at |
|---|---|---|---|---|---|---|
| pH | solvent | day 0 | day 21 (5° C.) | day 21 (25° C.) | day 21 (40° C.) | day 21 (60° C.) |
| 1.0 | water | 97.4 ± 0.4 | 90.3 ± 0.3 | 65.5 ± 0.3 | 9.4 ± 0.3 | 0 |
| 2.0 | water | 97.6 ± 0.3 | 93.5 ± 0.3 | 78.1 ± 0.3 | 20.6 ± 0.3 | 0 |
| 3.0 | water | 97.8 ± 0.2 | 95.3 ± 0.3 | 91.2 ± 0.2 | 41.7 ± 0.2 | 0 |
| 3.7 | water | 97.9 ± 0.2 | 96.4 ± 0.2 | 95.6 ± 0.2 | 56.6 ± 0.2 | 11.5 ± 0.3 |
| 4.0 | water | 98.1 ± 0.1 | 96.8 ± 0.1 | 95.7 ± 0.2 | 58.3 ± 0.2 | 23.0 ± 0.3 |
| 4.3 | water | 98.1 ± 0.2 | 96.9 ± 0.1 | 95.4 ± 0.1 | 60.1 ± 0.1 | 25.8 ± 0.2 |
| 4.6 | water | 97.9 ± 0.1 | 96.3 ± 0.2 | 94.9 ± 0.2 | 55.3 ± 0.2 | 13.5 ± 0.3 |
| 5.0 | water | 97.8 ± 0.2 | 96.1 ± 0.2 | 92.1 ± 0.3 | 37.8 ± 0.3 | 1.7 ± 0.3 |
| 6.0 | water | 97.6 ± 0.3 | 94.4 ± 0.2 | 79.5 ± 0.3 | 19.5 ± 0.2 | 0 |
| 7.0 | water | 97.4 ± 0.3 | 90.0 ± 0.3 | 59.7 ± 0.3 | 5.7 ± 0.3 | 0 |
| 8.0 | water | 97.1 ± 0.3 | 78.1 ± 0.3 | 43.6 ± 0.4 | 0 | 0 |

The results show that the solution of 2-(diethylamino)ethyl 1-methyl-5-(4-methylbenzoyl)-1H-pyrrole-2-acetate.HCl salt in water is not stable at a temperature higher than 40PC and a pH lower than 3 or greater than 6.

TABLE 44
Stabilities of 7% solution of 2-(diethylamino)ethyl 3-(6-methoxy-
2-naphthyl)propionate•HCl salt at various pH values and temperature
in water (pH was adjusted with 3N HCl or 3N NaOH).

| | | Purity(%) at | Purity(%) at | Purity(%) at | Purity(%) at | Purity (%) at |
|---|---|---|---|---|---|---|
| pH | solvent | day 0 | day 21 (5° C.) | day 21 (25° C.) | day 21 (40° C.) | day 21 (60° C.) |
| 1.0 | water | 98.0 ± 0.3 | 92.7 ± 0.3 | 69.1 ± 0.3 | 16.3 ± 0.3 | 0 |
| 2.0 | water | 98.1 ± 0.3 | 95.9 ± 0.3 | 83.5 ± 0.3 | 27.5 ± 0.3 | 0 |
| 3.0 | water | 98.3 ± 0.2 | 97.4 ± 0.3 | 94.6 ± 0.2 | 49.3 ± 0.2 | 0 |
| 3.7 | water | 98.4 ± 0.2 | 97.7 ± 0.2 | 97.3 ± 0.2 | 59.9 ± 0.2 | 19.3 ± 0.3 |
| 4.0 | water | 98.5 ± 0.1 | 98.2 ± 0.2 | 97.5 ± 0.2 | 70.7 ± 0.2 | 23.6 ± 0.3 |
| 4.3 | water | 98.5 ± 0.2 | 98.2 ± 0.2 | 97.6 ± 0.1 | 70.1 ± 0.2 | 27.7 ± 0.2 |
| 4.6 | water | 98.5 ± 0.1 | 98.2 ± 0.2 | 97.5 ± 0.2 | 59.3 ± 0.2 | 22.3 ± 0.2 |
| 5.0 | water | 98.4 ± 0.2 | 97.5 ± 0.2 | 94.9 ± 0.2 | 45.8 ± 0.3 | 5.9 ± 0.2 |
| 6.0 | water | 98.2 ± 0.2 | 95.3 ± 0.2 | 88.8 ± 0.2 | 28.6 ± 0.2 | 0 |
| 7.0 | water | 98.0 ± 0.3 | 90.2 ± 0.3 | 60.9 ± 0.2 | 17.4 ± 0.3 | 0 |
| 8.0 | water | 97.7 ± 0.3 | 79.7 ± 0.3 | 49.9 ± 0.3 | 0 | 0 |

The results show that the solution of 2-(diethylamino)ethyl 3-(6-methoxy-2-naphthyl)propionate.HCl salt in water is not stable at a temperature higher than 40PC and a pH lower than 3 or greater than 6.

TABLE 45
Stabilities of 7% solution of 2-(diethylamino)ethyl 4-(4-chlorophenyl)-
2-phenyl-5-thiazoleacetate•HCl salt at various pH values and
temperature in water (pH was adjusted with 3N HCl or 3N NaOH).

| | | Purity(%) at | Purity(%) at | Purity(%) at | Purity(%) at | Purity (%) at |
|---|---|---|---|---|---|---|
| pH | solvent | day 0 | day 21 (5° C.) | day 21 (25° C.) | day 21 (40° C.) | day 21 (60° C.) |
| 1.0 | water | 98.5 ± 0.3 | 90.9 ± 0.3 | 66.5 ± 0.3 | 9.9 ± 0.3 | 0 |
| 2.0 | water | 98.7 ± 0.2 | 93.9 ± 0.2 | 78.9 ± 0.3 | 21.6 ± 0.3 | 0 |
| 3.0 | water | 98.8 ± 0.2 | 96.8 ± 0.2 | 92.3 ± 0.2 | 42.3 ± 0.2 | 0 |
| 3.7 | water | 98.9 ± 0.2 | 98.5 ± 0.2 | 96.6 ± 0.2 | 57.6 ± 0.2 | 10.5 ± 0.3 |
| 4.0 | water | 99.1 ± 0.1 | 98.6 ± 0.1 | 96.7 ± 0.2 | 60.2 ± 0.2 | 25.2 ± 0.2 |
| 4.3 | water | 99.1 ± 0.1 | 98.7 ± 0.2 | 95.4 ± 0.1 | 60.8 ± 0.1 | 26.8 ± 0.2 |
| 4.6 | water | 98.9 ± 0.1 | 98.5 ± 0.1 | 95.8 ± 0.1 | 55.9 ± 0.1 | 15.5 ± 0.1 |
| 5.0 | water | 98.8 ± 0.2 | 96.9 ± 0.2 | 92.8 ± 0.2 | 37.3 ± 0.2 | 4.6 ± 0.3 |
| 6.0 | water | 98.6 ± 0.2 | 94.7 ± 0.2 | 79.9 ± 0.3 | 21.5 ± 0.2 | 0 |
| 7.0 | water | 98.5 ± 0.3 | 90.3 ± 0.3 | 60.7 ± 0.3 | 7.7 ± 0.2 | 0 |
| 8.0 | water | 98.3 ± 0.3 | 78.4 ± 0.3 | 44.8 ± 0.3 | 0 | 0 |

The results show that the solution of 2-(diethylamino)ethyl 4-(4-chlorophenyl)-2-phenyl-5-thiazoleacetate.HCl salt in water is not stable at a temperature higher than 40° C. and a pH lower than 3 or greater than 6.

TABLE 46
Stabilities of 7% solution of 2-(diethylamino)ethyl 1-(4-chlorobenzoyl-
5-methoxy-2-methyl-1H-indole-3-acetoxyacetate•HCl salt at various pH
values and temperature in water (pH was adjusted with 3N HCl or 3N NaOH).

| | | Purity(%) at | Purity(%) at | Purity(%) at | Purity(%) at | Purity (%) at |
|---|---|---|---|---|---|---|
| pH | solvent | day 0 | day 21 (5° C.) | day 21 (25° C.) | day 21 (40° C.) | day 21 (60° C.) |
| 1.0 | water | 98.1 ± 0.3 | 91.9 ± 0.3 | 67.3 ± 0.3 | 15.6 ± 0.3 | 0 |
| 2.0 | water | 98.3 ± 0.3 | 95.7 ± 0.2 | 82.2 ± 0.2 | 26.7 ± 0.3 | 0 |
| 3.0 | water | 98.5 ± 0.2 | 98.0 ± 0.2 | 93.0 ± 0.2 | 47.7 ± 0.3 | 0 |
| 3.7 | water | 98.7 ± 0.1 | 98.2 ± 0.1 | 97.1 ± 0.1 | 57.6 ± 0.2 | 13.9 ± 0.3 |
| 4.0 | water | 98.7 ± 0.1 | 98.4 ± 0.1 | 97.4 ± 0.2 | 68.7 ± 0.3 | 19.2 ± 0.3 |
| 4.3 | water | 98.8 ± 0.2 | 98.5 ± 0.1 | 97.5 ± 0.1 | 68.6 ± 0.3 | 17.9 ± 0.2 |
| 4.6 | water | 98.8 ± 0.2 | 98.4 ± 0.2 | 96.5 ± 0.2 | 53.7 ± 0.2 | 14.8 ± 0.3 |
| 5.0 | water | 98.6 ± 0.2 | 97.1 ± 0.2 | 93.8 ± 0.2 | 39.8 ± 0.3 | 3.2 ± 0.2 |
| 6.0 | water | 98.4 ± 0.2 | 95.8 ± 0.2 | 86.9 ± 0.2 | 28.8 ± 0.2 | 0 |
| 7.0 | water | 98.1 ± 0.2 | 90.2 ± 0.3 | 59.7 ± 0.3 | 11.2 ± 0.4 | 0 |
| 8.0 | water | 97.7 ± 0.3 | 78.5 ± 0.2 | 47.9 ± 0.3 | 0 | 0 |

The results show that the solution of 2-(diethylamino)ethyl 1-(4-chlorobenzoyl-5-methoxy-2-methyl-1H-indole-3-acetoxyacetate.HCl salt in water is not stable at a temperature higher than 40° C. and a pH lower than 3 or greater than 6.

TABLE 47
Stabilities of 7% solution of 2-(diethylamino)ethyl [(1-benzyl-
1H-indazol-3-yl)oxy]acetate•HCl salt at various pH values
and temperature in water (pH was adjusted with 3N HCl or 3N NaOH).

| | | Purity(%) at | Purity(%) at | Purity(%) at | Purity(%) at | Purity (%) at |
|---|---|---|---|---|---|---|
| pH | solvent | day 0 | day 21 (5° C.) | day 21 (25° C.) | day 21 (40° C.) | day 21 (60° C.) |
| 1.0 | water | 97.5 ± 0.3 | 89.9 ± 0.3 | 63.5 ± 0.3 | 9.1 ± 0.3 | 0 |
| 2.0 | water | 97.7 ± 0.2 | 93.0 ± 0.2 | 77.8 ± 0.3 | 19.6 ± 0.3 | 0 |
| 3.0 | water | 97.8 ± 0.2 | 96.8 ± 0.2 | 91.3 ± 0.2 | 40.4 ± 0.2 | 0 |
| 3.7 | water | 97.9 ± 0.1 | 97.5 ± 0.1 | 95.6 ± 0.2 | 55.6 ± 0.2 | 8.5 ± 0.2 |
| 4.0 | water | 98.1 ± 0.1 | 97.6 ± 0.1 | 95.7 ± 0.1 | 57.2 ± 0.2 | 23.2 ± 0.2 |
| 4.3 | water | 98.1 ± 0.1 | 97.7 ± 0.2 | 95.5 ± 0.1 | 58.8 ± 0.3 | 25.8 ± 0.2 |
| 4.6 | water | 97.9 ± 0.1 | 97.6 ± 0.1 | 95.1 ± 0.1 | 54.7 ± 0.2 | 16.5 ± 0.2 |
| 5.0 | water | 97.8 ± 0.2 | 95.9 ± 0.2 | 91.8 ± 0.2 | 36.1 ± 0.2 | 4.2 ± 0.2 |
| 6.0 | water | 97.6 ± 0.3 | 93.7 ± 0.2 | 78.9 ± 0.2 | 18.5 ± 0.2 | 0 |
| 7.0 | water | 97.5 ± 0.3 | 89.3 ± 0.2 | 59.7 ± 0.3 | 7.1 ± 0.3 | 0 |
| 8.0 | water | 97.4 ± 0.4 | 77.5 ± 0.3 | 39.8 ± 0.3 | 0 | 0 |

The results show that the solution of 2-(diethylamino)ethyl [(1-benzyl-1H-indazol-3-yl)oxy]acetate.HCl salt in water is not stable at a temperature higher than 40C and a pH lower than 3 or greater than 6.

TABLE 48
Stabilities of 7% solution of 2-(diethylamino)ethyl 2-[(4-chlorophenyl)-
5-benzoxazole]propionate•HCl salt at various pH values and
temperature in water (pH was adjusted with 3N HCl or 3N NaOH).

| | | Purity(%) at | Purity(%) at | Purity(%) at | Purity(%) at | Purity (%) at |
|---|---|---|---|---|---|---|
| pH | solvent | day 0 | day 21 (5° C.) | day 21 (25° C.) | day 21 (40° C.) | day 21 (60° C.) |
| 1.0 | water | 98.1 ± 0.3 | 92.9 ± 0.3 | 69.2 ± 0.3 | 11.3 ± 0.3 | 0 |
| 2.0 | water | 98.2 ± 0.3 | 96.1 ± 0.3 | 83.0 ± 0.3 | 20.5 ± 0.3 | 0 |
| 3.0 | water | 98.3 ± 0.2 | 97.5 ± 0.2 | 94.0 ± 0.3 | 45.3 ± 0.2 | 0 |
| 3.7 | water | 98.5 ± 0.1 | 97.6 ± 0.2 | 96.6 ± 0.2 | 51.9 ± 0.2 | 11.3 ± 0.3 |
| 4.0 | water | 98.6 ± 0.1 | 98.0 ± 0.1 | 96.7 ± 0.2 | 62.7 ± 0.2 | 18.6 ± 0.2 |
| 4.3 | water | 98.6 ± 0.1 | 98.0 ± 0.1 | 96.7 ± 0.1 | 63.1 ± 0.2 | 17.7 ± 0.2 |
| 4.6 | water | 98.6 ± 0.1 | 98.0 ± 0.1 | 96.6 ± 0.1 | 50.3 ± 0.2 | 12.3 ± 0.2 |
| 5.0 | water | 98.4 ± 0.2 | 97.1 ± 0.2 | 92.9 ± 0.2 | 39.8 ± 0.2 | 2.9 ± 0.2 |
| 6.0 | water | 98.2 ± 0.2 | 94.8 ± 0.2 | 86.8 ± 0.2 | 20.6 ± 0.3 | 0 |
| 7.0 | water | 98.0 ± 0.2 | 89.1 ± 0.3 | 60.9 ± 0.2 | 7.4 ± 0.3 | 0 |
| 8.0 | water | 97.7 ± 0.3 | 78.7 ± 0.3 | 47.9 ± 0.3 | 0 | 0 |

The results show that the solution of 2-(diethylamino)ethyl 2-[(4-chlorophenyl)-5-benzoxazole]propionate.HCl salt in water is not stable at a temperature higher than 40° C. and a pH lower than 3 or greater than 6.

TABLE 49
Stabilities of 7% solution of 2-(diethylamino)ethyl 4,5-diphenyl-
2-oxazolepropionate•HCl salt at various pH values and temperature
in water (pH was adjusted with 3N HCl or 3N NaOH).

| | | Purity(%) at | Purity(%) at | Purity(%) at | Purity(%) at | Purity (%) at |
|---|---|---|---|---|---|---|
| pH | solvent | day 0 | day 21 (5° C.) | day 21 (25° C.) | day 21 (40° C.) | day 21 (60° C.) |
| 1.0 | water | 98.2 ± 0.3 | 90.1 ± 0.3 | 59.9 ± 0.3 | 7.9 ± 0.3 | 0 |
| 2.0 | water | 98.4 ± 0.3 | 93.0 ± 0.2 | 74.8 ± 0.2 | 17.6 ± 0.2 | 0 |
| 3.0 | water | 98.6 ± 0.1 | 96.9 ± 0.2 | 88.9 ± 0.2 | 36.4 ± 0.2 | 0 |
| 3.7 | water | 98.7 ± 0.2 | 97.8 ± 0.1 | 94.5 ± 0.2 | 50.6 ± 0.2 | 7.3 ± 0.3 |
| 4.0 | water | 98.8 ± 0.1 | 97.9 ± 0.1 | 95.1 ± 0.1 | 53.2 ± 0.2 | 20.2 ± 0.2 |
| 4.3 | water | 98.8 ± 0.2 | 97.9 ± 0.2 | 95.2 ± 0.2 | 54.8 ± 0.1 | 21.8 ± 0.3 |
| 4.6 | water | 98.7 ± 0.1 | 97.8 ± 0.2 | 95.0 ± 0.1 | 54.7 ± 0.2 | 15.5 ± 0.3 |
| 5.0 | water | 98.6 ± 0.3 | 96.3 ± 0.2 | 89.8 ± 0.1 | 30.1 ± 0.2 | 3.2 ± 0.2 |
| 6.0 | water | 98.4 ± 0.2 | 93.4 ± 0.2 | 73.9 ± 0.2 | 16.5 ± 0.2 | 0 |
| 7.0 | water | 98.2 ± 0.3 | 89.2 ± 0.3 | 59.7 ± 0.2 | 7.1 ± 0.3 | 0 |
| 8.0 | water | 98.0 ± 0.3 | 77.1 ± 0.3 | 40.8 ± 0.3 | 0 | 0 |

The results show that the solution of 2-(diethylamino)ethyl 4,5-diphenyl-2-oxazolepropionate.HCl salt in water is not stable at a temperature higher than 40° C. and a pH lower than 3 or greater than 6.

TABLE 50
Stabilities of 7% solution of 2-(diethylamino)ethyl 5-(2,4-difluorophenyl)-
2-acetoxybenzoate•HCl salt at various pH values and temperature
in water (pH was adjusted with 3N HCl or 3N NaOH).

| | | Purity(%) at | Purity(%) at | Purity(%) at | Purity(%) at | Purity (%) at |
|---|---|---|---|---|---|---|
| pH | solvent | day 0 | day 14 (5° C.) | day 14 (25° C.) | day 14 (40° C.) | day 14 (60° C.) |
| 1.0 | water | 99.1 | 90.2 | 61.2 | 0 | 0 |
| 2.0 | water | 99.1 | 95.8 | 82.7 | 0 | 0 |
| 3.0 | water | 99.1 | 97.5 | 87.7 | 2.3 | 0 |
| 3.7 | water | 99.1 | 98.2 | 95.5 | 14.1 | 0 |
| 4.0 | water | 99.1 | 98.4 | 95.9 | 14.3 | 0 |
| 4.3 | water | 99.1 | 98.4 | 95.8 | 12.8 | 0 |
| 4.6 | water | 99.1 | 98.3 | 95.6 | 3.4 | 0 |
| 5.0 | water | 99.1 | 97.0 | 91.0 | 0.9 | 0 |
| 6.0 | water | 99.1 | 91.6 | 84.1 | 0 | 0 |
| 7.0 | water | 99.1 | 87.3 | 60.3 | 0 | 0 |
| 8.0 | water | 99.1 | 57.3 | 21.2 | 0 | 0 |

The results show that the solution of 2-(diethylamino)ethyl 5-(2,4-difluorophenyl)-2-acetoxybenzoate.HCl salt in water is not stable at a temperature higher than 40° C. and a pH lower than 3 or greater than 6.

The results show that the solutions are more stable at a lower temperature and should be stored at temperatures no more than 25° C., preferably 2-8° C.

### 4. Effect of Solvent on the Stability

TABLE 51
Stabilities of 7% solution of 2-(diethylamino)ethyl acetylsalicylate•HCl salt
at various pH values and temperature in water (pH was adjusted with 3N HCl or 3N NaOH).

| | | Purity(%) at | Purity(%) at | Purity(%) at | Purity(%) at | Purity (%) at |
|---|---|---|---|---|---|---|
| pH | solvent | day 0 | day 14 (5° C.) | day 14 (25° C.) | day 14 (40° C.) | day 14 (60° C.) |
| 1.0 | water | 99.1 ± 0.3 | 90.8 ± 0.3 | 62.2 ± 0.4 | 0 | 0 |
| 2.0 | water | 99.3 ± 0.2 | 96.3 ± 0.2 | 83.3 ± 0.3 | 0 | 0 |
| 3.0 | water | 99.5 ± 0.2 | 98.1 ± 0.1 | 88.5 ± 0.2 | 2.5 ± 0.2 | 0 |
| 3.7 | water | 99.5 ± 0.1 | 98.7 ± 0.1 | 96.1 ± 0.1 | 14.5 ± 0.3 | 0 |
| 4.0 | water | 99.6 ± 0.1 | 98.8 ± 0.1 | 96.6 ± 0.2 | 14.5 ± 0.3 | 0 |
| 4.3 | water | 99.7 ± 0.1 | 98.9 ± 0.1 | 96.6 ± 0.1 | 12.9 ± 0.2 | 0 |
| 4.6 | water | 99.5 ± 0.2 | 98.5 ± 0.2 | 96.5 ± 0.2 | 3.6 ± 0.2 | 0 |
| 5.0 | water | 99.5 ± 0.1 | 97.3 ± 0.1 | 92.5 ± 0.2 | 1.0 | 0 |
| 6.0 | water | 99.4 ± 0.3 | 92.1 ± 0.2 | 84.7 ± 0.3 | 0 | 0 |
| 7.0 | water | 99.2 ± 0.3 | 87.9 ± 0.3 | 59.8 ± 0.3 | 0 | 0 |
| 8.0 | water | 99.0 ± 0.3 | 57.9 ± 0.3 | 21.7 ± 0.4 | 0 | 0 |

The results show that the solution of 2-(diethylamino)ethyl acetylsalicylate.HCl salt in water is not stable at a temperature higher than 40° C. and a pH lower than 3 or greater than 6.

TABLE 52
Stabilities of 7% solution of 2-(diethylamino)ethyl acetylsalicylate•HCl salt at
various pH values and temperature in 15% ethanol (pH was adjusted with 3N HCl or 3N NaOH).

| | | Purity(%) | Purity(%) at | Purity(%) at | Purity(%) at | Purity (%) at |
|---|---|---|---|---|---|---|
| pH | solvent | at day 0 | day 14 (5° C.) | day 14 (25° C.) | day 14 (40° C.) | day 14 (60° C.) |
| 1.0 | 15% ethanol | 99.1 ± 0.2 | 92.1 ± 0.3 | 62.7 ± 0.3 | 0 | 0 |
| 2.0 | 15% ethanol | 99.4 ± 0.2 | 96.5 ± 0.2 | 83.8 ± 0.3 | 0 | 0 |
| 3.0 | 15% ethanol | 99.5 ± 0.1 | 98.3 ± 0.3 | 88.8 ± 0.2 | 2.9 ± 0.3 | 0 |
| 3.7 | 15% ethanol | 99.6 ± 0.1 | 98.9 ± 0.2 | 96.2 ± 0.3 | 15.2 ± 0.3 | 0 |
| 4.0 | 15% ethanol | 99.7 ± 0.2 | 98.9 ± 0.1 | 96.7 ± 0.2 | 15.7 ± 0.3 | 0 |
| 4.3 | 15% ethanol | 99.7 ± 0.1 | 98.9 ± 0.1 | 96.7 ± 0.1 | 13.6 ± 0.3 | 0 |
| 4.6 | 15% ethanol | 99.6 ± 0.2 | 98.6 ± 0.2 | 96.5 ± 0.2 | 4.1 ± 0.3 | 0 |
| 5.0 | 15% ethanol | 99.5 ± 0.2 | 97.4 ± 0.2 | 92.9 ± 0.2 | 1.3 ± 0.3 | 0 |
| 6.0 | 15% ethanol | 99.4 ± 0.2 | 92.3 ± 0.3 | 84.9 ± 0.3 | 0 | 0 |
| 7.0 | 15% ethanol | 99.2 ± 0.1 | 87.9 ± 0.3 | 60.2 ± 0.3 | 0 | 0 |
| 8.0 | 15% ethanol | 99.1 ± 0.2 | 58.7 ± 0.4 | 22.7 ± 0.3 | 0 | 0 |

The results show that the solvent (15% ethanol) did not affect the stability of the solution of 2-(diethylamino)ethyl acetylsalicylate.HCl salt significantly, but somewhat improved the stability. Because 15% ethanol can inhibit bacteria growth, it is a good selection for 2-(diethylamino)ethyl acetylsalicylate.HCl medical uses.

TABLE 53
Stabilities of 7% solution of 2-(diethylamino)ethyl acetylsalicylate•HCl salt at
various pH values and temperature in 25% ethanol (pH was adjusted with 3N HCl or 3N NaOH).

| | | Purity(%) at | Purity(%) at | Purity(%) at | Purity(%) at | Purity (%) at |
|---|---|---|---|---|---|---|
| pH | solvent | day 0 | day 14 (5° C.) | day 14 (25° C.) | day 14 (40° C.) | day 14 (60° C.) |
| 1.0 | 25% ethanol | 99.1 ± 0.3 | 92.3 ± 0.3 | 63.5 ± 0.3 | 0 | 0 |
| 2.0 | 25% ethanol | 99.4 ± 0.3 | 96.9 ± 0.3 | 84.2 ± 0.3 | 0 | 0 |
| 3.0 | 25% ethanol | 99.5 ± 0.2 | 98.5 ± 0.2 | 88.9 ± 0.2 | 2.9 ± 0.2 | 0 |
| 3.7 | 25% ethanol | 99.6 ± 0.1 | 99.1 ± 0.2 | 96.3 ± 0.2 | 15.1 ± 0.3 | 0 |
| 4.0 | 25% ethanol | 99.8 ± 0.1 | 99.1 ± 0.1 | 96.9 ± 0.1 | 15.2 ± 0.3 | 0 |
| 4.3 | 25% ethanol | 99.7 ± 0.2 | 99.1 ± 0.2 | 96.8 ± 0.2 | 13.8 ± 0.3 | 0 |
| 4.6 | 25% ethanol | 99.7 ± 0.2 | 98.8 ± 0.1 | 96.5 ± 0.2 | 4.2 ± 0.1 | 0 |
| 5.0 | 25% ethanol | 99.6 ± 0.2 | 98.0 ± 0.2 | 92.8 ± 0.2 | 1.6 ± 0.1 | 0 |
| 6.0 | 25% ethanol | 99.5 ± 0.3 | 92.7 ± 0.2 | 85.0 ± 0.3 | 0 | 0 |
| 7.0 | 25% ethanol | 99.4 ± 0.3 | 88.0 ± 0.3 | 61.2 ± 0.3 | 0 | 0 |
| 8.0 | 25% ethanol | 99.1 ± 0.3 | 59.8 ± 0.3 | 23.1 ± 0.3 | 0 | 0 |

The results show that the solvent (25% ethanol) did not affect the stability of the solution of 2-(diethylamino)ethyl acetylsalicylate.HCl salt significantly.

TABLE 54
Stabilities of 7% solution of 2-(diethylamino)ethyl acetylsalicylate•HCl salt at
various pH values and temperature in 50% ethanol (pH was adjusted with 3N HCl or 3N NaOH).

| | | Purity(%) at | Purity(%) at | Purity(%) at | Purity(%) at | Purity (%) at |
|---|---|---|---|---|---|---|
| pH | solvent | day 0 | day 14 (5° C.) | day 14 (25° C.) | day 14 (40° C.) | day 14 (60° C.) |
| 1.0 | 50% ethanol | 99.2 ± 0.3 | 92.4 ± 0.3 | 63.8 ± 0.4 | 0 | 0 |
| 2.0 | 50% ethanol | 99.5 ± 0.2 | 96.9 ± 0.3 | 84.7 ± 0.3 | 0 | 0 |
| 3.0 | 50% ethanol | 99.6 ± 0.3 | 98.6 ± 0.3 | 89.5 ± 0.3 | 3.2 ± 0.3 | 0 |
| 3.7 | 50% ethanol | 99.7 ± 0.2 | 99.1 ± 0.3 | 96.6 ± 0.2 | 16.9 ± 0.3 | 0 |
| 4.0 | 50% ethanol | 99.8 ± 0.2 | 99.2 ± 0.3 | 97.2 ± 0.3 | 16.8 ± 0.4 | 0 |
| 4.3 | 50% ethanol | 99.7 ± 0.2 | 99.1 ± 0.2 | 97.2 ± 0.2 | 14.9 ± 0.3 | 0 |
| 4.6 | 50% ethanol | 99.7 ± 0.3 | 99.0 ± 0.3 | 96.9 ± 0.3 | 4.9 ± 0.3 | 0 |
| 5.0 | 50% ethanol | 99.6 ± 0.2 | 98.2 ± 0.3 | 93.1 ± 0.3 | 2.3 ± 0.2 | 0 |
| 6.0 | 50% ethanol | 99.5 ± 0.3 | 92.9 ± 0.4 | 85.5 ± 0.3 | 0 | 0 |
| 7.0 | 50% ethanol | 99.4 ± 0.3 | 88.2 ± 0.3 | 61.9 ± 0.4 | 0 | 0 |
| 8.0 | 50% ethanol | 99.2 ± 0.3 | 60.3 ± 0.4 | 23.9 ± 0.4 | 0 | 0 |

The results show that the solvent (50% ethanol) did not affect the stability of 2-(diethylamino)ethyl acetylsalicylate.HCl salt significantly.

From tables 51-54, it can be seen that the amount of ethanol did not affect the stability of 7% solution of 2-(diethylamino)ethyl acetylsalicylate.HCl salt significantly. Compared with pure water as the solvent, the solvents with different concentrations of ethanol make the solutions somewhat more stable. The concentration of ethanol may be 0-70% v/v, preferably 10-35% v/v, more preferably 15-25% v/v. For example, aqueous solution containing 15% ethanol, which can inhibit bacteria growth, is a good selection for medical uses.

Experiments with other solvents, such as aqueous solution containing different concentration of acetone or DMSO also gave similar results, i.e., the solvent did not affect the stability of the solution significantly.

Other HPDs also have very similar behavior. The other HPDs are, for example:

HPDs of peptides such as H-Val-Pro-Gly-Pro-Arg(NO₂)—OCH(CH₃)₂.HCl, H-Ala-Pro-Gly-Pro-Arg(NO₂)—OCH₂CH₃.HCl, H-Val-Pro-Asp[OCH(CH₃)₂]-Pro-Arg(NO₂)—OCH(CH₃)₂.HCl, H-Tyr-Gly-Gly-Phe-Leu-OCH(CH₃)₂.HCl, and H-Tyr-Gly-Gly-Phe-Met-OCH(CH₃)₂.HCl;

and other HPDs such as 2-(diethylamino)ethyl 2-(6-methoxy-2-naphthyl) propionate.HCl, 2-(diethylamino)ethyl (R,S)-2-(2-fluoro-4-biphenyl)propionate.HCl, 2-(diethylamino)ethyl 2-(p-isobutylphenyl)propionate.HCl, 2-(diethylamino)ethyl 1-(4-chlorobenzoyl)-5-methoxy-2-methyl-1H-indole-3-acetate.HCl, 2-(diethylamino)ethyl 5-fluoro-2-methyl-1-[[4-(methylsulfinyl)phenyl]methylene]-1H-indene-3-acetate.HCl, 2-(diethylamino)ethyl 1-methyl-5-(4-methylbenzoyl)-1H-pyrrole-2-acetate.HCl, 2-(diethylamino)ethyl 5-(4-chlorobenzoyl)-1,4-dimethyl-1H-pyrrole-2-acetate.HCl, 2-(diethylamino)ethyl 3-(6-methoxy-2-naphthyl)propionate.HCl, 2-(diethylamino)ethyl 4-(4-chlorophenyl)-2-phenyl-5-thiazoleacetate.HCl, 2-(diethylamino)ethyl 1-(4-chlorobenzoyl-5-methoxy-2-methyl-1H-indole-3-acetoxyacetate.HCl, 2-(diethylamino)ethyl [(1-benzyl-1H-indazol-3-yl)oxy]acetate.HCl, 2-(diethylamino)ethyl 2-[(4-chlorophenyl)-5-benzoxazole]propionate.HCl, 2-(diethylamino)ethyl 4,5-diphenyl-2-oxazolepropionate.HCl, 2-(diethylamino)ethyl 4-[bis(2-chloroethyl)amino]benzenebutyrate.HCl, 2-(diethylamino)ethyl 4-[bis(2-methylsulfonylethyl)amino]benzenebutyrate.HCl, and 2-(diethylamino)ethyl 5-(2,4-difluorophenyl)-2-acetoxybenzoate.HCl.

It should be understood that the above-mentioned general, preferable, or more preferable feature(s) in one aspect of the invention can be combined with other general, preferable, or more preferable feature(s) in another aspect of the invention. For example, the concentration of the HPD in the reconstitution solution is 3-10%, the pH is 3-5 and the pharmaceutically acceptable carrier is 15-35% ethanol in pure water.

### 5. Stability of the Pure HPDs

TABLE 55
Stabilities of H-Val-Pro-Gly-Pro-Arg(NO₂)—OCH(CH₃)₂•HCl salt(T-1),
H-Ala-Pro-Gly-Pro-Arg(NO₂)—OCH(CH₃)₂•HCl salt (T-2), and H-Val-Pro-
Asp[OCH(CH₃)₂]-Pro-Arg(NO₂)—OCH(CH₃)₂•HCl salt (T-3) at 25° C./RH 60%

| Time | Day 0 | 3 month | 6 month | 9 month | 12 month | 18 month | 24 month |
|---|---|---|---|---|---|---|---|
| Purity(T-1) | 98.7 ± 0.2 | 98.8 ± 0.2 | 98.6 ± 0.2 | 98.5 ± 0.2 | 98.4 ± 0.3 | 98.2 ± 0.3 | 98.2 ± 0.2 |
| Purity(T-2) | 98.9 ± 0.1 | 98.9 ± 0.3 | 98.6 ± 0.2 | 98.6 ± 0.3 | 98.5 ± 0.2 | 98.5 ± 0.2 | 98.4 ± 0.2 |
| Purity(T-3) | 99.1 ± 0.2 | 99.0 ± 0.2 | 99.0 ± 0.2 | 98.8 ± 0.2 | 98.7 ± 0.0 | 98.6 ± 0.0 | 98.5 ± 0.2 |

The results show that the pure powder of H-Val-Pro-Gly-Pro-Arg(NO₂)—OCH(CH₃)₂.HCl salt(T-1), H-Ala-Pro-Gly-Pro-Arg(NO₂)—OCH(CH₃)₂.HCl salt (T-2), and H-Val-Pro-Asp[OCH(CH₃)₂]-Pro-Arg(NO₂)—OCH(CH₃)₂.HCl salt (T-3) are very stable and can be stored for years at room temperature.

TABLE 56
Stabilities of H-Tyr-Gly-Gly-Phe-Leu-OCH(CH₃)₂•HCl (U-1) and H-Tyr-Gly-
Gly-Phe-Met-OCH(CH₃)₂•HCl salt (U-2) at 25° C./RH 60%

| Time | Day 0 | 3 month | 6 month | 9 month | 12 month | 18 month | 24 month |
|---|---|---|---|---|---|---|---|
| Purity(U-1) | 98.5 ± 0.2 | 98.5 ± 0.2 | 98.4 ± 0.2 | 98.3 ± 0.2 | 98.2 ± 0.3 | 98.2 ± 0.3 | 98.0 ± 0.2 |
| Purity(U-2) | 98.9 ± 0.1 | 98.9 ± 0.3 | 98.6 ± 0.2 | 98.6 ± 0.3 | 98.5 ± 0.2 | 98.5 ± 0.2 | 98.4 ± 0.2 |

The results show that the pure powder of H-Tyr-Gly-Gly-Phe-Leu-OCH(CH₃)₂.HCl (U-1) and H-Tyr-Gly-Gly-Phe-Met-OCH(CH₃)₂.HCl salt (U-2) are very stable and can be stored for years at room temperature.

TABLE 57
Stabilities of 2-(diethylamino)ethyl 2-(6-methoxy-2-naphthyl) propionate•HCl
salt (A-1-1), 2-(diethylamino)ethyl (S)-2-(6-methoxy-2-naphthyl) propionate•HCl
salt (A-1-2), 2-(diethylamino)ethyl (R)-2-(6-methoxy-2-naphthyl) propionate•HCl
salt (A-1-3), 2-(diethylamino)ethyl 2-(6-methoxy-2-naphthyl) propionate•HBr
salt (A-1-4), 2-(diethylamino)ethyl 2-(6-methoxy-2-naphthyl) propionate•citric
acid salt (A-1-5), 2-(dimethylamino)ethyl 2-(6-methoxy-2-naphthyl) propionate•HCl
salt (A-2), 2-(dibutylamino)ethyl 2-(6-methoxy-2-naphthyl) propionate•HCl
salt (A-3), 2-(dihexylamino)ethyl 2-(6-methoxy-2-naphthyl) propionate•HCl
salt (A-4), 2-(di-3-hexenylamino)ethyl 2-(6-methoxy-2-naphthyl) propionate•HCl
salt (A-5), 2-(di-3-hexynylamino)ethyl 2-(6-methoxy-2-naphthyl) propionate•HCl
salt (A-6), and 2-(di-2-(2-methoxyethoxy)ethylamino)ethyl 2-(6-methoxy-2-naphthyl)
propionate•HCl salt (A-7) at 25° C./RH 60%

| Time | Day 0 | 3 month | 6 month | 12 month | 18 month | 24 month |
|---|---|---|---|---|---|---|
| Purity(A-1-1) | 98.8 ± 0.2 | 98.6 ± 0.1 | 98.8 ± 0.1 | 98.5 ± 0.2 | 98.3 ± 0.2 | 98.2 ± 0.2 |
| Purity(A-1-2) | 98.6 ± 0.3 | 98.5 ± 0.3 | 98.4 ± 0.2 | 98.3 ± 0.2 | 98.2 ± 0.2 | 98.0 ± 0.1 |
| Purity(A-1-3) | 98.5 ± 0.2 | 98.4 ± 0.2 | 98.5 ± 0.2 | 98.2 ± 0.1 | 98.1 ± 0.2 | 97.9 ± 0.2 |
| Purity(A-1-4) | 98.6 ± 0.3 | 98.9 ± 0.1 | 98.6 ± 0.4 | 98.4 ± 0.2 | 98.2 ± 0.1 | 98.0 ± 0.4 |
| Purity(A-1-5) | 99.0 ± 0.2 | 99.0 ± 0.4 | 98.9 ± 0.1 | 98.7 ± 0.2 | 98.4 ± 0.2 | 98.2 ± 0.2 |
| Purity(A-2) | 98.5 ± 0.2 | 98.4 ± 0.1 | 98.2 ± 0.2 | 97.9 ± 0.1 | 97.6 ± 0.2 | 97.1 ± 0.2 |
| Purity(A-3) | 98.3 ± 0.1 | 98.4 ± 0.2 | 98.2 ± 0.2 | 97.9 ± 0.2 | 97.3 ± 0.1 | 96.8 ± 0.2 |
| Purity(A-4) | 98.6 ± 0.2 | 98.5 ± 0.1 | 98.6 ± 0.1 | 98.0 ± 0.2 | 97.9 ± 0.2 | 97.2 ± 0.1 |
| Purity(A-5) | 98.5 ± 0.3 | 98.3 ± 0.3 | 98.4 ± 0.2 | 97.9 ± 0.1 | 97.6 ± 0.2 | 97.1 ± 0.2 |
| Purity(A-6) | 98.3 ± 0.2 | 98.5 ± 0.2 | 98.0 ± 0.1 | 97.8 ± 0.2 | 97.4 ± 0.2 | 97.1 ± 0.2 |
| Purity(A-7) | 98.1 ± 0.2 | 98.0 ± 0.1 | 98.2 ± 0.2 | 97.4 ± 0.2 | 97.1 ± 0.2 | 96.7 ± 0.1 |

The solid of 2-(6-methoxy-2-naphthyl) propionate. HA salt is very stable and can be stored for more than 2 years are room temperature. The size and shape of alkyl group on amino group and A⁻ did not affect the stability significantly. The dry drug substances can be stored for 2 years or more at 25° C. without significant changes.

TABLE 58
Stabilities of 2-(diethylamino)ethyl 2-(2-fluoro-4-biphenyl)propionate•HCl
salt (B-1-1), 2-(diethylamino)ethyl (S)-2-(2-fluoro-4-biphenyl)propionate•HCl
salt (B-1-2), 2-(diethylamino)ethyl (R)-2-(2-fluoro-4-biphenyl)propionate•HCl
salt (B-1-3), 2-(diethylamino)ethyl 2-(2-fluoro-4-biphenyl)propionate•HBr
salt (B-1-4), 2-(diethylamino)ethyl 2-(2-fluoro-4-biphenyl)propionate•citric
acid salt (B-1-5), 2-(dimethylamino)ethyl 2-(2-fluoro-4-biphenyl)propionate•HCl
salt (B-2), 2-(dibutylamino)ethyl 2-(2-fluoro-4-biphenyl)propionate•HCl
salt (B-3), 2-(dihexylamino)ethyl 2-(2-fluoro-4-biphenyl)propionate•HCl
salt (B-4), 2-(di-3-hexenylamino)ethyl 2-(2-fluoro-4-biphenyl)propionate•HCl
salt (B-5), 2-(di-3-hexynylamino)ethyl 2-(2-fluoro-4-biphenyl)propionate•HCl
salt (B-6), and 2-(di-2-(2-methoxyethoxy)ethylamino)ethyl 2-(2-fluoro-4-
biphenyl)propionate•HCl salt (B-7) at 25° C./RH 60%

| Time | Day 0 | 3 month | 6 month | 12 month | 18 month | 24 month |
|---|---|---|---|---|---|---|
| Purity(B-1-1) | 98.5 ± 0.2 | 98.4 ± 0.1 | 98.1 ± 0.1 | 98.2 ± 0.1 | 98.1 ± 0.1 | 98.0 ± 0.1 |
| Purity(B-1-2) | 98.7 ± 0.2 | 98.6 ± 0.1 | 98.5 ± 0.2 | 98.4 ± 0.2 | 98.3 ± 0.1 | 98.2 ± 0.1 |
| Purity(B-1-3) | 98.4 ± 0.1 | 98.3 ± 0.1 | 98.3 ± 0.1 | 98.2 ± 0.1 | 98.1 ± 0.2 | 98.0 ± 0.1 |
| Purity(B-1-4) | 98.6 ± 0.2 | 98.5 ± 0.1 | 98.4 ± 0.1 | 98.3 ± 0.1 | 98.1 ± 0.1 | 97.9 ± 0.1 |
| Purity(B-1-5) | 98.3 ± 0.2 | 98.2 ± 0.1 | 98.2 ± 0.1 | 98.1 ± 0.1 | 98.0 ± 0.1 | 97.9 ± 0.1 |
| Purity(B-2) | 98.1 ± 0.3 | 98.0 ± 0.1 | 97.9 ± 0.1 | 98.0 ± 0.1 | 97.6 ± 0.1 | 97.4 ± 0.2 |
| Purity(B-3) | 98.3 ± 0.2 | 98.3 ± 0.1 | 98.1 ± 0.1 | 98.2 ± 0.1 | 97.6 ± 0.1 | 97.5 ± 0.1 |
| Purity(B-4) | 98.0 ± 0.2 | 98.0 ± 0.1 | 97.9 ± 0.1 | 97.8 ± 0.1 | 97.5 ± 0.3 | 97.2 ± 0.1 |
| Purity(B-5) | 98.2 ± 0.3 | 98.1 ± 0.1 | 98.1 ± 0.1 | 98.1 ± 0.1 | 97.8 ± 0.1 | 97.7 ± 0.2 |
| Purity(B-6) | 98.1 ± 0.2 | 98.1 ± 0.1 | 98.0 ± 0.1 | 97.9 ± 0.2 | 97.8 ± 0.2 | 97.6 ± 0.1 |
| Purity(B-7) | 98.3 ± 0.3 | 98.2 ± 0.1 | 98.2 ± 0.1 | 98.1 ± 0.1 | 98.0 ± 0.1 | 97.9 ± 0.1 |

The solid of 2-(2-fluoro-4-biphenyl)propionate.HCl salt is very stable and can be stored for more than 2 years are room temperature. The size of alkyl group on amino group and A⁻ did not affect the stability significantly. The dry drug substances can be stored for 2 years or more at 25° C. without significant changes.

TABLE 59
2-(diethylamino)ethyl (R,S)-2-(p-isobutylphenyl)propionate•HCl
salt (C-1-1), 2-(diethylamino)ethyl (S)-2-(p-isobutylphenyl)propionate•HCl
salt (C-1-2), 2-(diethylamino)ethyl (R)-2-(p-isobutylphenyl)propionate•HCl
salt (C-1-3), 2-(diethylamino)ethyl (R,S)-2-(p-isobutylphenyl)propionate•HBr
salt (C-1-4), 2-(diethylamino)ethyl (R,S)-2-(p-isobutylphenyl)propionate•citric
acid salt (C-1-5), 2-(dimethylamino)ethyl (R,S)-2-(p-isobutylphenyl)propionate•HCl
salt (C-2), 2-(dibutylamino)ethyl (R,S)-2-(p-isobutylphenyl)propionate•HCl
salt (C-3), 2-(dihexylamino)ethyl (R,S)-2-(p-isobutylphenyl)propionate•HCl
salt (C-4), 2-(di-3-hexenylamino)ethyl (R,S)-2-(p-isobutylphenyl)propionate•HCl
salt (C-5), 2-(di-3-hexynylamino)ethyl (R,S)-2-(p-isobutylphenyl)propionate•HCl
salt (C-6), and 2-(di-2-(2-methoxyethoxy)ethylamino)ethyl (R,S)-2-(p-
isobutylphenyl)propionate•HCl salt (C-7) at 25° C./RH 60%

| Time | Day 0 | 3 month | 6 month | 9 month | 12 month | 18 month | 24 month |
|---|---|---|---|---|---|---|---|
| Purity(C-1-1) | 99.8 ± 0.1 | 99.9 ± 0.2 | 99.8 ± 0.2 | 99.8 ± 0.3 | 99.5 ± 0.3 | 99.8 ± 0.3 | 99.7 ± 0.4 |
| Purity(C-1-2) | 98.5 ± 0.1 | 98.5 ± 0.3 | 98.4 ± 0.4 | 98.5 ± 0.3 | 98.3 ± 0.4 | 98.2 ± 0.1 | 98.0 ± 0.5 |
| Purity(C-1-3) | 98.2 ± 0.1 | 98.2 ± 0.2 | 98.1 ± 0.3 | 98.0 ± 0.1 | 98.0 ± 0.3 | 97.9 ± 0.3 | 97.8 ± 0.2 |
| Purity(C-1-4) | 98.5 ± 0.2 | 98.4 ± 0.1 | 98.5 ± 0.3 | 98.3 ± 0.1 | 98.3 ± 0.3 | 98.2 ± 0.4 | 98.0 ± 0.5 |
| Purity(C-1-5) | 98.8 ± 0.1 | 98.7 ± 0.2 | 98.8 ± 0.3 | 98.5 ± 0.1 | 98.7 ± 0.1 | 98.4 ± 0.4 | 98.3 ± 0.3 |
| Purity(C-2) | 98.5 ± 0.1 | 98.4 ± 0.2 | 98.4 ± 0.1 | 98.1 ± 0.1 | 98.2 ± 0.3 | 97.9 ± 0.3 | 97.7 ± 0.4 |
| Purity(C-3) | 98.4 ± 0.2 | 98.3 ± 0.1 | 98.2 ± 0.3 | 98.4 ± 0.3 | 98.1 ± 0.3 | 97.9 ± 0.4 | 97.8 ± 0.3 |
| Purity(C-4) | 98.3 ± 0.1 | 98.4 ± 0.2 | 98.2 ± 0.3 | 98.2 ± 0.1 | 98.1 ± 0.3 | 98.0 ± 0.1 | 97.9 ± 0.3 |
| Purity(C-5) | 98.2 ± 0.1 | 98.1 ± 0.2 | 98.1 ± 0.3 | 98.2 ± 0.1 | 98.0 ± 0.4 | 97.9 ± 0.1 | 97.7 ± 0.4 |
| Purity(C-6) | 98.5 ± 0.1 | 98.5 ± 0.3 | 98.4 ± 0.4 | 98.4 ± 0.1 | 98.3 ± 0.2 | 98.3 ± 0.3 | 98.1 ± 0.2 |
| Purity(C-7) | 98.3 ± 0.2 | 98.2 ± 0.1 | 98.2 ± 0.2 | 98.1 ± 0.1 | 98.2 ± 0.3 | 98.0 ± 0.3 | 97.9 ± 0.1 |

The solid of 2-(p-isobutylphenyl)propionate. HA salt is very stable and can be stored for more than 2 years are room temperature. The size and shape of alkyl group on amino group and A⁻ did not affect the stability significantly.

Other HPDs have very similar behavior. The other HPDs are, for example,

2-(diethylamino)ethyl 1-(4-chlorobenzoyl)-5-methoxy-2-methyl-1H-indole-3-acetate.HCl, 2-(diethylamino)ethyl 5-fluoro-2-methyl-1-[[4-(methylsulfinyl)phenyl]methylene]-1H-indene-3-acetate.HCl, 2-(diethylamino)ethyl 1-methyl-5-(4-methylbenzoyl)-1H-pyrrole-2-acetate.HCl, 2-(diethylamino)ethyl 5-(4-chlorobenzoyl)-1,4-dimethyl-1H-pyrrole-2-acetate.HCl, 2-(diethylamino)ethyl 3-(6-methoxy-2-naphthyl)propionate.HCl, 2-(diethylamino)ethyl 4-(4-chlorophenyl)-2-phenyl-5-thiazoleacetate.HCl, 2-(diethylamino)ethyl 1-(4-chlorobenzoyl-5-methoxy-2-methyl-1H-indole-3-acetoxyacetate.HCl, 2-(diethylamino)ethyl [(1-benzyl-1H-indazol-3-yl)oxy]acetate.HCl, 2-(diethylamino)ethyl 2-[(4-chlorophenyl)-5-benzoxazole]propionate.HCl, 2-(diethylamino)ethyl 4,5-diphenyl-2-oxazolepropionate.HCl, 2-(diethylamino)ethyl 4-[bis(2-chloroethyl)amino]benzenebutyrate.HCl, 2-(diethylamino)ethyl 4-[bis(2-methylsulfonylethyl)amino]benzenebutyrate.HCl, 2-(diethylamino)ethyl acetylsalicylate.HCl, and 2-(diethylamino)ethyl 5-(2,4-difluorophenyl)-2-acetoxybenzoate.HCl.

### III. Administering HPDs Via Penetrating a Biological Barrier

Another aspect of the invention relates to a method of using the pharmaceutical composition in penetrating one or more biological barriers in a biological subject. The method comprises a step of administering the pharmaceutical composition to a biological subject.

The term “biological barrier” as used herein refers to a biological layer that separates an environment into different spatial areas or compartments, which separation is capable of modulating (e.g., restricting, limiting, enhancing or taking no action in) the passing through, penetrating or translocation of substance or matter from one compartment/area to another. The different spatial areas or compartments as referred to herein may have the same or different chemical or biological environment(s). The biological layer as referred herein includes, but is not limited to, a biological membrane, a cell layer, a biological structure, an inner surface of subjects, organisms, organs or body cavities, an external surface of subjects, organisms, organs or body cavities, or any combination or plurality thereof.

Examples of a biological membrane include a lipid bilayer structure, eukaryotic cell membrane, prokaryotic cell membrane, and intracellular membrane (e.g., nucleus or organelle membrane, such as membrane or envelope of Golgi apparatus, rough and smooth endoplasmic reticulum (ER), ribosomes, vacuoles, vesicles, liposomes, mitochondria, lysosome, nucleus, chloroplasts, plastids, peroxisomes or microbodies).

The lipid bilayer referred to herein is a double layer of lipid-class molecules, including, but not limited to, phospholipids and cholesterol. In a particular embodiment, lipids for bilayer are amphiphilic molecules consisting of polar head groups and non-polar fatty acid tails. The bilayer is composed of two layers of lipids arranged so that their hydrocarbon tails face one another to form an oily core held together by the hydrophobic effect, while their charged heads face the aqueous solutions on either side of the membrane. In another particular embodiment, the lipid bilayer may contain one or more embedded protein and/or sugar molecule(s).

Examples of a cell layer include a lining of eukaryotic cells (e.g., epithelium, lamina propria and smooth muscle or muscularis mucosa (in gastrointestinal tract)), a lining of prokaryotic cells (e.g., surface layer or S-layer which refers to a two dimensional structure monomolecular layer composed of identical proteins or glycoproteins, specifically, an S-layer refers to a part of a cell envelope commonly found in bacteria and archaea), a biofilm (a structured community of microorganisms encapsulated within a self-developed polymeric matrix and adherent to a living or inert surface), and a plant cell layer (e.g., empidermis). The cells may be normal cells or pathological cells (e.g. disease cells, cancer cells).

Examples of biological structures include structures sealed by tight or occluding junctions which provide a barrier to the entry of toxins, bacteria and viruses, e.g. blood milk barrier, blood-cerebrospinal fluid (CSF) barrier, blood-synovial fluid (SF) barrier and blood brain barrier (BBB). In particular, BBB is composed of an impermeable class of endothelium, which presents both a physical barrier through tight junctions adjoining neighboring endothelial cells and a transport barrier comprised of efflux transporters. The biological structure may also include a mixture of cells, proteins and sugars (e.g. blood clots), for example, a myelin sheath, which is a layer around the axon of a neuron formed by a dielectric material, myelin.

Examples of the inner surface of subjects, organisms, organs or body cavities include buccal mucosa, esophageal mucosa, gastric mucosa, intestinal mucosa, olfactory mucosa, oral mucosa, bronchial mucosa, uterine mucosa and endometrium (the mucosa of the uterus, inner layer of the wall of a pollen grain or the inner wall layer of a spore), or a combination or plurality thereof.

Examples of the external surface of subjects, organisms, organs or body cavities include capillaries (e.g. capillaries in the heart tissue), mucous membranes that are continuous with skin (e.g. such as at the nostrils, the lips, the ears, the genital area, and the anus), outer surface of an organ (e.g. liver, lung, stomach, brain, kidney, heart, ear, eye, nose, mouth, tongue, colon, pancreas, gallbladder, duodenum, rectum stomach, colonrectum, intestine, vein, respiratory system, vascular, the anorectum and pruritus ani), skin, cuticle (e.g., dead layers of epidermal cells or keratinocytes or superficial layer of overlapping cells covering the hair shaft of an animal, a multi-layered structure outside the epidermis of many invertebrates, plant cuticles or polymers cutin and/or cutan), external layer of the wall of a pollen grain or the external wall layer of a spore), and a combination or plurality thereof.

In addition, a biological barrier further includes a sugar layer, a protein layer or any other biological layer, or a combination or plurality thereof. For example, skin is a biological barrier that has a plurality of biological layers. A skin comprises an epidermis layer (outer surface), a demis layer and a subcutaneous layer. The epidermis layer contains several layers including a basal cell layer, a spinous cell layer, a granular cell layer, and a stratum corneum. The cells in the epidermis are called keratinocytes. The stratum corneum (“horny layer”) is the outmost layer of the epidermis, wherein cells here are flat and scale-like (“squamous”) in shape. These cells contain a lot of keratin and are arranged in overlapping layers that impart a tough and oilproof and waterproof character to the skin's surface.

In certain embodiments, since the HPD of the present disclosure has enhanced ability of crossing one or more biological barriers, it can be administered locally (e.g., topically or transdermally) to reach a location where a condition occurs without the necessity of a systematic administration (e.g., oral or parenteral administration).

A local administration and penetration of the HPD allows it to reach the same level of local concentration of an agent or drug with a much smaller amount or dosage in comparison to a systematic administration of a parent drug; alternatively, a higher level of local concentration which may not be afforded in the systematic administration, or if possible, requires significantly higher dosage of an agent in the systematic administration.

The local administration of the HPD may allow a biological subject to reduce potential sufferings from a systemic administration, e.g., adverse reactions associated with the systematic exposure to the agent, gastrointestinal/renal effects. Additionally, the local administration may allow the HPD to cross a plurality of biological barriers and reach systematically through, for example, general circulation and thus avoid the needs for systematic administration (e.g., injection) and obviate the pain associated with the parenteral injection.

The HPD of this disclosure exhibited high penetration rate through a biological barrier (e.g., about >10 times, about >50 times, about >100 times, about >200 times, about >300 times, about >500 times, about >1,000 times, about >10,000 times or higher than the penetration rate of prostaglandins or prostaglandin analogs if administered alone). No side effect was observed from the subjects to which were administered a HPD, while side effects were observed from the subjects to which the parent drug or analog thereof was administered at the similar dosage.

It will be understood by those of skill in the art that numerous and various modifications can be made to the compounds, compositions, and/or methods of the present invention without departing from the spirit of the invention. Therefore, the various embodiments of the present invention described herein are illustrative only, and are not intended to limit the scope of the invention in any way. All patent or non-patent references cited herein are incorporated by reference in their entirety.

1. A method for improving the stability of a pharmaceutical composition which comprises a high penetration drug substance and a pharmaceutically acceptable carrier, the method comprising:
packaging the high penetration drug substance and the pharmaceutically acceptable carrier in separate containers; and
reconstituting a solution of the pharmaceutical composition by mixing the high penetration drug substance with the pharmaceutically acceptable carrier prior to administration to a patient in need thereof;
wherein the pH of the reconstitution solution of the pharmaceutical composition is kept within the range of 2 to 6; and wherein the high penetration drug substance comprises one or two protonated amine groups in its molecule when being administered to the patient.

2. (canceled)

3. The method according to claim 1, wherein the pharmaceutically acceptable carrier is an aqueous carrier.

4. The method according to claim 1, wherein the pharmaceutically acceptable carrier is water, alcohol, acetone, dimethyl sulfoxide (DMSO), or a mixture thereof.

5. The method according to claim 1, wherein the pharmaceutically acceptable carrier is an aqueous solution containing 0-70% ethanol by volume.

6. (canceled)

7. (canceled)

8. The method according to claim 1, further comprising storing the reconstitution solution in a refrigerator at a temperature of 2-8° C.

9. The method according to claim 1, wherein the pharmaceutical composition further comprises a pH adjusting and buffering agent in the pharmaceutically acceptable carrier.

10. The method according to claim 9, wherein the high penetration drug is high penetration peptide; and the pH adjusting and buffering agent is a sodium, potassium, calcium, lithium, or magnesium salt of an organic acid.

11. The method according to claim 9, wherein the pH adjusting and buffering agent is sodium, potassium, or lithium salt of an organic acid selected from the group consisting of acetic acid, propionic acid, butyric acid, valeric acid, benzoic acid, lactic acid, salicylic acid, citric acid, ascorbic acid, succinic acid, and maleic acid.

12. The method according to claim 1, wherein the pH of the reconstitution solution of the pharmaceutical composition is in the range of 3 to 6.

13. (canceled)

14. (canceled)

15. The method according to claim 1, wherein the concentration of the high penetration drug in the reconstitution solution is in the range of 1%-30% by weight.

16. (canceled)

17. (canceled)

18. The method according to claim 1, wherein the high penetration drug substance is selected from the group consisting of 2-(diethylamino)ethyl 2-(6-methoxy-2-naphthyl) propionate.HCl, 2-(diethylamino)ethyl (R,S)-2-(2-fluoro-4-biphenyl)propionate.HCl, 2-(diethylamino)ethyl 2-(p-isobutylphenyl)propionate.HCl, 2-(diethylamino)ethyl 1-(4-chlorobenzoyl)-5-methoxy-2-methyl-1H-indole-3-acetate.HCl, 2-(diethylamino)ethyl 5-fluoro-2-methyl-1-[[4-(methylsulfinyl)phenyl]methylene]-1H-indene-3-acetate.HCl, 2-(diethylamino)ethyl 1-methyl-5-(4-methylbenzoyl)-1H-pyrrole-2-acetate.HCl, 2-(diethylamino)ethyl 5-(4-chlorobenzoyl)-1,4-dimethyl-1H-pyrrole-2-acetate.HCl, 2-(diethylamino)ethyl 3-(6-methoxy-2-naphthyl)propionate.HCl, 2-(diethylamino)ethyl 4-(4-chlorophenyl)-2-phenyl-5-thiazoleacetate.HCl, 2-(diethylamino)ethyl 1-(4-chlorobenzoyl-5-methoxy-2-methyl-1H-indole-3-acetoxyacetate.HCl, 2-(diethylamino)ethyl [(1-benzyl-1H-indazol-3-yl)oxy]acetate.HCl, 2-(diethylamino)ethyl 2-[(4-chlorophenyl)-5-benzoxazole]propionate.HCl, 2-(diethylamino)ethyl 4,5-diphenyl-2-oxazolepropionate.HCl, 2-(diethylamino)ethyl 4-[bis(2-chloroethyl)amino]benzenebutyrate.HCl, 2-(diethylamino)ethyl 4-[bis(2-methylsulfonylethyl)amino]benzenebutyrate.HCl, 2-(diethylamino)ethyl acetylsalicylate.HCl, and 2-(diethylamino)ethyl 5-(2,4-difluorophenyl)-2-acetoxybenzoate.HCl.

19. The method according to claim 1, wherein the concentration of the high penetration drug in the reconstitution solution is 3-8% by weight, the pH of reconstitution solution is 3-5, and the pharmaceutically acceptable carrier is 15-35% ethanol in water by volume.

20. The method according to claim 1, wherein the high penetration drug is selected from the group consisting of H-Val-Pro-Gly-Pro-Arg(NO₂)—OCH(CH₃)₂.HCl, H-Ala-Pro-Gly-Pro-Arg(NO₂)—OCH₂CH₃.HCl, H-Val-Pro-Asp[OCH(CH₃)₂]-Pro-Arg(NO₂)—OCH(CH₃)₂.HCl, H-Tyr-Gly-Gly-Phe-Leu-OCH(CH₃)₂.HCl, and H-Tyr-Gly-Gly-Phe-Met-OCH(CH₃)₂.HCl.

21. The method according to claim 20, wherein the concentration of the high penetration drug in the reconstitution solution is 3-8%, the pH of reconstitution solution is 3-5, the pH adjusting and buffering agent is sodium acetate, and the pharmaceutically acceptable carrier is 15-35% ethanol in water by volume.

22. A pharmaceutical composition comprising the high penetration drug substance and pharmaceutically acceptable carrier obtained from the method of claim 1.

23. A method of treating a disease or disorder in a subject in need thereof, comprising administering to the subject a therapeutically effective amount of a pharmaceutical composition of claim 22, wherein the pharmaceutical composition is a freshly prepared reconstitution solution by mixing the high penetration drug substance with the pharmaceutically acceptable carrier from separate containers.

24. (canceled)

25. The method of claim 23, wherein the disease or disorder is selected from the group consisting of stroke, arthritis, depression, Alzheimer's disease, Parkinson's disease, migraine, sexual dysfunction, sepsis, drug-resistant bacterial infections, epilepsy, diabetes, psoriasis, lupus erythematosus, ulcerative enteritis, asthma, lower and upper respiratory tract infections, allergic rhinitis, allergic conjunctivitis, itchiness, and runny nose.

26. A treatment kit comprising: a high penetration drug substance in a first container, a pharmaceutically acceptable carrier in a second container, and a pH adjusting and buffering agent in the first container, the second container, or a separate third container, wherein the high penetration drug substance comprises one or two protonated amine groups, and wherein the high penetration drug substance, the pharmaceutically acceptable carrier, and the pH adjusting and buffering agent can be mixed together to form a reconstitution solution ready for administration to a subject in need thereof, wherein the reconstitution solution has a pH in the range of 2 to 6 and is stable for storage at a temperature in the range of 2-20° C. for a period of time prior to administration to the subject in need thereof.

27. The kit of claim 26, wherein the high penetration drug substance is selected from the group consisting of 2-(diethylamino)ethyl 2-(6-methoxy-2-naphthyl) propionate.HCl, 2-(diethylamino)ethyl (R,S)-2-(2-fluoro-4-biphenyl)propionate.HCl, 2-(diethylamino)ethyl 2-(p-isobutylphenyl)propionate.HCl, 2-(diethylamino)ethyl 1-(4-chlorobenzoyl)-5-methoxy-2-methyl-1H-indole-3-acetate.HCl, 2-(diethylamino)ethyl 5-fluoro-2-methyl-1-[[4-(methylsulfinyl)phenyl]methylene]-1H-indene-3-acetate.HCl, 2-(diethylamino)ethyl 1-methyl-5-(4-methylbenzoyl)-1H-pyrrole-2-acetate.HCl, 2-(diethylamino)ethyl 5-(4-chlorobenzoyl)-1,4-dimethyl-1H-pyrrole-2-acetate.HCl, 2-(diethylamino)ethyl 3-(6-methoxy-2-naphthyl)propionate.HCl, 2-(diethylamino)ethyl 4-(4-chlorophenyl)-2-phenyl-5-thiazoleacetate.HCl, 2-(diethylamino)ethyl 1-(4-chlorobenzoyl-5-methoxy-2-methyl-1H-indole-3-acetoxyacetate.HCl, 2-(diethylamino)ethyl [(1-benzyl-1H-indazol-3-yl)oxy]acetate.HCl, 2-(diethylamino)ethyl 2-[(4-chlorophenyl)-5-benzoxazole]propionate.HCl, 2-(diethylamino)ethyl 4,5-diphenyl-2-oxazolepropionate.HCl, 2-(diethylamino)ethyl 4-[bis(2-chloroethyl)amino]benzenebutyrate.HCl, 2-(diethylamino)ethyl 4-[bis(2-methylsulfonylethyl)amino]benzenebutyrate.HCl, 2-(diethylamino)ethyl acetylsalicylate.HCl, 2-(diethylamino)ethyl 5-(2,4-difluorophenyl)-2-acetoxybenzoate.HCl, H-Val-Pro-Gly-Pro-Arg(NO₂)—OCH(CH₃)₂.HCl, H-Ala-Pro-Gly-Pro-Arg(NO₂)—OCH₂CH₃.HCl, H-Val-Pro-Asp[OCH(CH₃)₂]-Pro-Arg(NO₂)—OCH(CH₃)₂.HCl, H-Tyr-Gly-Gly-Phe-Leu-OCH(CH₃)₂.HCl, and H-Tyr-Gly-Gly-Phe-Met-OCH(CH₃)₂.HCl; and the pharmaceutically acceptable carrier is a mixture of an aliphatic C₁-C₆ alcohol and water.

28. The kit of claim 26, wherein the concentration of the high penetration drug in the reconstitution solution is 3-8%, the pH of reconstitution solution is 3-5, the pH adjusting and buffering agent is sodium acetate, and the pharmaceutically acceptable carrier is 15-35% ethanol in water by volume.
